(19) United States
(12) Patent Application Publication
(10) Pub. No.: US 2026/0301904 A1
(43) Pub. Date: Oct. 1, 2026

(54) SYSTEMS AND METHODS FOR DETERMINING AND PERFORMING INTERVENTIONS USING AN ELECTROMECHANICAL MACHINE WHEREIN THE INTERVENTIONS ARE DETERMINED BY USING A LARGE LANGUAGE MODEL TO GENERATE AND RANK, BASED ON ONE OR MORE MEASURES OF EFFICACY, A LIST OF INTERVENTIONS

(71) Applicant: ROM Technologies, Inc., Brookfield, CT (US)

(72) Inventors: DANIEL CHARLES ALLOWAY, Bethel, CT (US); STEVEN MASON, Las Vegas, NV (US)

(73) Assignee: ROM Technologies, Inc., Brookfield, CT (US)

(21) Appl. No.: 19/702,726

(22) Filed: Jun. 9, 2026

Related U.S. Application Data
(63) Continuation-in-part of application No. 19/075,257, filed on Mar. 10, 2025.
Application No. 19/075,257 is a continuation of application No. 17/397,385, filed on Aug. 9, 2021, now Pat. No. 12,249,410.
Application No. 17/397,385 is a continuation of application No. 17/147,295, filed on Jan. 12, 2021, now Pat. No. 11,087,865.
Application No. 17/147,295 is a continuation-in-part of application No. 17/021,895, filed on Sep. 15, 2020, now Pat. No. 11,071,597.
(60) Provisional application No. 62/910,232, filed on Oct. 3, 2019.
(60) Provisional application No. 63/820,697, filed on Jun. 9, 2025.
(60) Provisional application No. 63/860,657, filed on Aug. 8, 2025.

(51) Int. Cl. G16H 20/10; G06F 3/048; G06N 3/08; G16H 20/30; G16H 40/67

(52) U.S. Cl. CPC G16H 20/10; G06F 3/048; G06N 3/08; G16H 20/30; G16H 40/67

(57) ABSTRACT

An electromechanical machine, such as a therapy apparatus enabled to improve one or more health conditions of a user, may be controlled based on specified instructions. Such instructions may be included in an action intervention that is generated in association with a treatment plan for a user. The action intervention may be generated using artificial intelligence (AI) techniques that leverage significant amounts of user data, including health-related data and outcome-related data. This data may include both objective and subjective data. An AI engine may generate an action intervention based on data from before, during, or after a therapy session. The action intervention may modify one or more operating parameters of the electromechanical machine, such as adjusting a level of resistance, increasing or decreasing a speed, adjusting an angle of a portion of the machine, adjusting a range of motion, etc. Action interventions generated by the AI engine may be transmitted to a variety of electronic devices.

### CROSS-REFERENCES TO RELATED APPLICATIONS

This application is a continuation-in-part and claims priority to and the benefit of U.S. patent application Ser. No. 19/075,257 (Atty. Docket No. 91346-5503), filed Mar. 10, 2025, titled “System and Method for Use of Treatment Device to Reduce Pain Medication Dependency,” which is a continuation of U.S. patent application Ser. No. 17/397,385 (Atty. Docket No. 91346-5502), filed Aug. 9, 2021, titled “System and Method for Use of Treatment Device to Reduce Pain Medication Dependency”, which is a continuation of and claims priority to and the benefit of U.S. patent application Ser. No. 17/147,295 (Atty. Docket No. 91346-5501), filed Jan. 12, 2021, titled “System and Method for Use of Treatment Device to Reduce Pain Medication Dependency”, which is a continuation-in-part of and claims priority to and the benefit of U.S. patent application Ser. No. 17/021,895 (Atty. Docket No. 91346-1410), filed Sep. 15, 2020, titled “Telemedicine for Orthopedic Treatment,” which claims priority to and the benefit of U.S. Provisional Patent Application Ser. No. 62/910,232 (Atty. Docket No. 1400), filed Oct. 3, 2019, titled “Telemedicine for Orthopedic Treatment,” the entire disclosures of which are hereby incorporated by reference for all purposes.

This application claims priority to and the benefit of U.S. Provisional Patent Application Ser. No. 63/820,697 (Atty. Docket No. 91346-80000) filed Jun. 9, 2025, titled “Systems and Methods for Use of Artificial Intelligence (AI) and Machine Learning to Perform AI-Driven Interventions at Scale” and U.S. Provisional Patent Application Ser. No. 63/860,657 (Att. Docket No. 91346-80001) filed Aug. 8, 2025, titled “Systems and Methods for Use of Artificial Intelligence (AI) and Machine Learning to Perform AI-Driven Interventions at Scale,” the entire disclosures of which are hereby incorporated by reference for all purposes.

### TECHNICAL FIELD

This disclosure relates generally to systems and methods for the use of artificial intelligence (AI) and machine learning to perform AI-driven interventions at scale.

### BACKGROUND

Conventional rehabilitation may consist of different providers (e.g., clinicians, therapists (including physical therapists, kineseologists, occupational therapists and the like), other medical professionals, etc.) and assistants treating patients over a certain number of sessions. The treatment plans generated may be highly varied and assigned based on each provider's independent judgment. The data that may be collected may include, without limitation, findings that the provider entered text in a non-standardized and/or unstructured data format that is non-queryable in a database which is not structured for research. Typically, providers findings (which are often written, transcribed, recorded electronically or the like) may be affected by their amount of engagement time with the patient, personal biases resulting from life experiences, educational background, direct interactions with the patient and personality compatibility or opposition, and/or approach to documentation or system for documentation. As a result, multitudes of varying subjective narrations may be generated that often contradict each other and/or erroneous. The result is that objective methods, suitable for comparison and suitable for measuring progress (or regression) are lacking and not practicable, leaving only unique-to-the-person, subjective assessments of a patient.

The opioid epidemic refers to the growing number of hospitalizations and deaths caused by people abusing opioids, including prescription drugs, illicit drugs, and analogous drugs. Annually in the United States, approximately 40,000 people die from an accidental overdose of opioids. Opioids, such as morphine, OxyContin, Vicodin, Percocet, codeine, fentanyl, and the like, are drugs that are often used to relieve severe pain (when properly prescribed by a medical professional), but they are also highly addictive drugs which may further cause biochemical changes in users' brains after continued use. Most people suffering from an opioid addiction initially began taking the drugs after they received, from a doctor, a prescription for pain medication (e.g., opioids) to alleviate pain resulting from an injury or a surgery. As patients engage in rehabilitation, their pain levels increase, which often leads to the patients taking more pain medication, even if this is contrary to the prescription. In addition, the increased pain levels may discourage the patients from diligently following their rehabilitation treatment plans. Such noncompliance may slow down the recovery progresses of the patients, leading to patients taking pain medication for even longer time periods. As the length of time (e.g., days, weeks, months, etc.) increases during which patients take their pain medication, the more likely the patients will become addicted to and/or dependent on opioids. For example, patients may become physically dependent on opioids and experience symptoms of tolerance (i.e., patients' bodies become desensitized to the drugs and patients need to take higher doses of the drug to relieve pain) and withdrawal (e.g., physical effects). Furthermore, patients may become mentally dependent on the opioids (i.e., the use of the opioids is a conditioned response to a feeling-a trigger- and the trigger sets off biochemical changes in the patients' brains that strongly potentiate addictive behavior).

Remote medical assistance, also referred to, inter alia, as remote medicine, telemedicine, telemed, telmed, tel-med, or telehealth, is an at least two-way communication between a healthcare professional or professionals, such as a physician and/or a physical therapist, and a patient using audio and/or audiovisual and/or other sensorial or perceptive (e.g., tactile, gustatory, haptic, pressure-sensing-based or electromagnetic (e.g., neurostimulative)) communications (e.g., via a computer, a smartphone, or a tablet). The patient may use a patient interface in communication with an assistant interface for receiving the remote medical assistance via audio, visual, audiovisual, or other communications described elsewhere herein. Any reference herein to any particular sensorial modality shall be understood to include and to disclose by implication a different one or more sensory modalities.

Doctors typically prescribe opioids to a patient after conducting a physical examination and/or communicating with the patient (e.g., to obtain the patient's rehabilitative progress and/or pain level). Telemedicine enables healthcare professionals to communicate with patients and provide patient care when the patients do not want to or cannot easily go to the healthcare professionals' offices. Telemedicine, however, has substantive limitations as the healthcare professionals cannot conduct physical examinations of the patients. Rather, the healthcare professionals must rely on verbal communication and/or limited remote observation of the patients. Therefore, improvements to telemedical protocols, the acquisition and delivery of patient-relevant information, and the ability to determine treatment plans or modifications to existing treatment plans all, without limitation, add greatly to the value and innovation of a telemedical approach, especially to the extent that they overcome the inherent limitations of telemedicine set forth above.

### SUMMARY

In general, the present disclosure provides systems and methods for the use of artificial intelligence (AI) and machine learning to perform AI-driven interventions at scale.

An aspect of the disclosed embodiments includes a computer-implemented system. The computer-implemented system includes, in one example, a treatment apparatus, a patient interface, and a computing device. The treatment apparatus is configured to be manipulated by a patient while the patient performs one or more treatment sessions. The patient interface includes an output device and an input device. The input device is configured to receive patient input correlating with at least one pain level of the patient during the one or more treatment sessions. The computing device is configured to receive a treatment plan for the patient. The treatment plan includes one or more exercise routines for the patient to complete on the treatment apparatus during the one or more treatment sessions. The computing device is further configured to receive treatment data pertaining to the patient. The computing device is also configured to receive patient input from the patient interface. The computing device is further configured to use the treatment plan, the treatment data, and the patient input to generate at least one threshold. Responsive to an occurrence of exceeding the at least one threshold, the computing device is configured to modify the treatment plan using an artificial intelligence engine.

Another aspect of the disclosed embodiments includes a method for modifying, by an artificial intelligence engine, a treatment plan for optimizing patient outcome and pain levels during one or more treatment sessions. The method includes receiving the treatment plan for a patient. The treatment plan includes one or more exercise routines for the patient to complete during the one or more treatment sessions. The method also includes receiving treatment data pertaining to the patient and receiving patient input correlating with at least one of the pain levels of the patient. The method further includes using the treatment plan, the treatment data, and the patient input to generate at least one threshold. Responsive to an occurrence of exceeding the at least one threshold, the method includes modifying the treatment plan.

Another aspect of the disclosed embodiments includes a tangible, non-transitory computer-readable medium storing instructions that, when executed, cause a processing device to receive a treatment plan for a patient. The treatment plan includes one or more exercise routines for the patient to complete during one or more treatment sessions. The instructions also cause the processing device to receive treatment data pertaining to the patient and receive patient input correlating with at least one pain level of the patient during the one or more treatment sessions. The instructions further cause the processing device to use the treatment plan, the treatment data, and the patient input to generate at least one threshold. Responsive to an occurrence of exceeding the at least one threshold, the instructions cause the processing device to modify the treatment plan.

Another aspect of the disclosed embodiments includes a system for modifying, by an artificial intelligence engine, a treatment plan for optimizing patient outcome and pain levels during one or more treatment sessions. The system includes, in one example, a memory device and a processing device. The memory device stores instructions. The processing device is communicatively coupled to the memory device. The processing device executes the instructions to receive a treatment plan for a patient. The treatment plan includes one or more exercise routines for the patient to complete during the one or more treatment sessions. The processing device also executes the instructions to receive treatment data pertaining to the patient and receive patient input correlating with at least one of the pain levels of the patient. The processing device further executes the instructions to use the treatment plan, the treatment data, and the patient input to generate at least one threshold. Responsive to an occurrence of exceeding the at least one threshold, the processing device executes the instructions to modify the treatment plan.

Another aspect of the disclosed embodiments includes a computer-implemented method that includes receiving, from one or more of an electromechanical machine, a sensor, and a computing device, data associated with a user that uses the electromechanical machine to perform a treatment plan. The method may include, based on the data, determining, using an artificial intelligence engine, a presence of at least one comorbidity associated with the user. The determining may include correlating at least two of a pain measurement, a pedal radius, a measurement of revolutions per minute, and an indicator of drug utilization, where each of the at least two correlations satisfies a threshold correlation level, and based on each of the at least two correlations satisfying the threshold correlation level, generating a unique data signature associated with the at least one comorbidity associated with the user. The method may include, based on the unique data signature associated with the user, generating, using the artificial intelligence engine, a modified treatment plan for the user; controlling, while the user uses the electromechanical machine; and using the modified treatment plan, the electromechanical machine.

Another aspect of the disclosed embodiments includes a computer-implemented method that may include receiving, from one or more of an electromechanical machine, a sensor, and a computing device, data associated with a user that uses the electromechanical machine to perform a treatment plan. The method may include, based on one or more correlations of one or more indicators included in the data, generating, using an artificial intelligence engine, a unique data signature associated with a regression of the user's condition. The unique data signature may be generated when the one or more indicators satisfy a respective threshold indicator level, and the one or more indicators may include a pain measurement, a measurement of revolutions per minute, and a session pedaling time. The method may include, based on the unique data signature associated with the regression of the user's condition, generating, using the artificial intelligence engine, a modified treatment plan that modifies a parameter or activity associated with at least one of the pain measurement, the measurement of revolutions per minute, and the session pedaling time, and controlling, while the user uses the electromechanical machine and using the modified treatment plan, the electromechanical machine.

Another aspect of the disclosed embodiments includes a computer-implemented method that may include receiving, from one or more of an electromechanical machine, a sensor, and a computing device, data associated with a user that uses the electromechanical machine to perform a treatment plan. The method may include, based on one or more correlations of one or more indicators included in the data, generating, using an artificial intelligence engine, a unique data signature associated with a life-threatening event of the user. The unique data signature is generated when the one or more indicators satisfy a respective urgent threshold level, and the one or more indicators comprise a pain measurement and a measurement of revolutions per minute. The method may include, based on the unique data signature associated with the one or more life-threatening events, performing one or more preventative actions.

Another aspect of the disclosed embodiments includes a computer-implemented method that may include receiving, from one or more of an electromechanical machine, a sensor, and a computing device, data associated with a user that uses the electromechanical machine to perform a treatment plan. The method may include correlating, using an artificial intelligence engine, at least two of a pain measurement, an indication of medication utilization, and a measurement of revolutions per minute to generate a unique data signature associated with a treatment gap associated with the treatment plan performed by the user, where the at least two of the pain measurement, the indication of medication utilization, and the measurement of revolutions per minute each satisfies a respective threshold level. The method may include, based on the unique data signature associated with the treatment gap associated with the treatment plan performed by the user, generating, using the artificial intelligence engine, one or more alternative treatment plans for the user. The method may include, based on a threshold compliance level associated with each of the one or more alternative treatment plans, selecting an alternative treatment plan from the one or more alternative treatment plans, and controlling, while the user uses the electromechanical machine and based on the alternative treatment plan, the electromechanical machine.

Another aspect of the disclosed embodiments includes a computer-implemented method that may include generating a first dataset by receiving user data related to one or more medical histories, diagnoses, measurements, real-time monitoring prompts, reports, outcomes, progressions, compliance-related information, documents, billing, or some combination thereof. The method may include generating a second dataset by importing a subset of data from the first dataset and, further, receiving data related to one or more risk adjusted outputs associated with quality improvement, user populations, experiment environments, experiment outcomes, libraries of prompts for large language models, or some combination thereof. The method may include, based on the first dataset and the second dataset, selecting, from a set of treatment plans and by using an artificial intelligence engine, a treatment plan for a user. The artificial intelligence engine may select the treatment plan by correlating one or more indicators of the first dataset and the second dataset and comparing the one or more correlated indicators to respective indicators obtained from one or more evidence-based selection protocols. The method may include controlling, using the treatment plan, operation of the electromechanical machine while the user uses the electromechanical machine.

Another aspect of the disclosed embodiments includes a computer-implemented method that may include receiving a treatment plan for a user. The treatment plan may include a schedule of treatments for infusion-based or electroconvulsive therapy (i.e., ECT) and one or more operating parameters of an electromechanical machine. The method may include receiving, from one or more of the electromechanical machine, sensor, and computing device, data associated with the user, and based on the data, generating, using an artificial intelligence engine, a modified treatment plan. The modified treatment plan may include a modified schedule of treatments for the therapy and one or more modified operating parameters of the electromechanical machine. The method may include, based on the modified treatment plan, causing operation of the electromechanical machine to be modified.

Another aspect of the disclosed embodiments may include a computer-implemented method that may include receiving data associated with a set of users having a similar medical condition. The data may be associated with one or more of a characteristic of the plurality of users and a performance metric of the plurality of users. The method may include, based on the data, generating, using an artificial intelligence engine and evidence-based research, at least two treatment plans for at least two of the set of users, where a first treatment plan of the at least two treatment plans includes instructions to inform a first user of an aspect of the first treatment plan and where, further, a second treatment plan of the at least two treatment plans excludes instructions to inform a second user of an aspect of the second treatment plan. The method may include, based on the at least two treatment plans, causing operation of at least two electromechanical machines associated respectively with the at least two treatment plans to be controlled.

Another aspect of the disclosed embodiments may include a method for modifying, by an artificial intelligence engine, a treatment plan for optimizing patient outcomes. An outcome may be partially based on one or more pain levels experienced or reported during one or more treatment sessions. The method may include receiving the treatment plan for a patient, where the treatment plan comprises one or more exercise routines for the patient to complete during the one or more treatment sessions. The method may include receiving treatment data pertaining to the patient, receiving a patient input including at least one of the pain levels of the patient, using the treatment plan, the treatment data, and the patient input to generate at least one threshold relating to pain level, and responsive to identifying that the at least one threshold has been exceeded, modifying the treatment plan to not exceed the at least one threshold. The method may include controlling, using the modified treatment plan, an electromechanical machine.

Another aspect of the disclosed embodiments includes a computer-implemented method that may include receiving, from one or more of an electromechanical machine, a sensor, and a computing device, data associated with a user who uses the electromechanical machine to perform a treatment plan. The method may include, based on the data, predicting, using an artificial intelligence engine, a risk probability that satisfies a risk probability threshold level associated with a regression or an undesired progression in rehabilitation. The method may include, based on the risk probability satisfying the risk probability threshold level, generating a modified treatment plan that includes information associated with modification of one or more operating parameters of the electromechanical machine, and based on the modified treatment plan, causing operation of the electromechanical machine to be controlled.

Another aspect of the disclosed embodiments includes a computer-implemented method that may include receiving, from one or more of an electromechanical machine, a sensor, and a computing device, data associated with a user who uses the electromechanical machine to perform a treatment plan. The data may include a number of exercise sessions, medication information associated with the user, pain level experienced or reported by the user, or some combination thereof. The method may include, based on the data, predicting, using an artificial intelligence engine, a state of the user's final progression, where the state describes or represents a result of the user's rehabilitation. The method may include, based on the user's final progression state of rehabilitation, generating, using the artificial intelligence engine, a modified treatment plan, wherein the modified treatment plan may include one or more different medical procedures, tests, rehabilitation exercises on the electromechanical machine, or some combination thereof. The method may include performing, using the artificial intelligence engine and the modified treatment plan, one or more intervention actions.

Another aspect of the disclosed embodiments includes a computer-implemented method that may include receiving, from one or more of an electromechanical machine, a sensor, and a computing device, data associated with a user who uses the electromechanical machine to perform a treatment plan. The method may include, based on the data, generating, using an artificial intelligence engine executing one or more large language models, one or more action interventions to perform. At least one of the one or more action interventions may include modifying one or more operating parameters of the electromechanical machine, and the one or more action interventions are generated based on one or more measures of efficacy. The method may include performing the one or more action interventions.

Another aspect of the disclosed embodiments includes a computer-implemented method that includes determining one or more individualized pain thresholds associated with one or more respective probabilities of a user progressing towards completing a treatment plan, automatically causing, using an artificial intelligence engine, one or more modifications to a component parameter of an electromechanical machine to reduce a user's pain within an individualized pain threshold level throughout a treatment session included in a treatment plan, receiving one or more pain measurement inputs, and determining, based on the one or more pain measurement inputs, whether the individualized pain threshold level is satisfied.

Another aspect of the disclosed embodiments includes a computer-implemented method that includes, during post-surgical rehabilitation, receiving, from one or more one or more life support devices, data pertaining to a user. The method includes, based on the at least the data, identifying one or more indicators and generating, using the one or more indicators and an artificial intelligence engine, a data signature associated with the user. The method also includes selecting, from a set of treatment plans and by using the artificial intelligence engine, a treatment plan for the user. The artificial intelligence engine may select the treatment plan by correlating the data signature with at least one of one or more data signatures obtained from one or more evidence-based selection protocols. The method may include performing, using the treatment plan and the artificial intelligence engine, one or more interventions.

Another aspect of the disclosed embodiments includes a computer-implemented method that includes receiving, from one or more of an electromechanical machine, a sensor, and a computing device, data associated with a user. The method includes, based on the at least the data, identifying one or more indicators and generating, using the one or more indicators and an artificial intelligence engine, a data signature associated with the user. The method also includes selecting, from a set of treatment plans and by using the artificial intelligence engine, a treatment plan for the user. The artificial intelligence engine may select the treatment plan by correlating the data signature with at least one of one or more data signatures obtained from one or more evidence-based selection protocols. The treatment plan may include treatment information pertaining to one or more internal or invasive life support devices associated with the user. The method may include performing, using the treatment plan and the artificial intelligence engine, one or more interventions including controlling at least one of the one or more internal or invasive life support devices associated with the user.

Another aspect of the disclosed embodiments includes a computer-implemented method that includes selecting, using an artificial intelligence engine, a set of treatment plans for a set of users. The artificial intelligence engine may select that set of treatment plans by correlating a set of characteristics of the set of users with one or more evidence-based studies. The method may include, based on the set of treatment plans, causing a set of electromechanical machine to be controlled while the set of users performs the set of treatment plans. The method may include receiving data, from a set of computing devices, a set of sensors, the set of electromechanical machines, or some combination thereof. The data may be associated with the set of users who use the set of electromechanical machines. The method may include determining, based on the data, a set of indications of effectiveness for the set of treatment plans.

Another aspect of the disclosed embodiments includes a computer-implemented method that includes initiating, using a first electromechanical machine, a first treatment session defined by a first treatment plan for a first user, and receiving, from at least one of a first sensor, a first computing device, and the first electromechanical machine, first data pertaining to the first user. While the first user performs the first treatment session, the first data is received during a first interaction with the first user. The first interaction is facilitated by a natural language processing agent executed by an artificial intelligence engine. The method includes, based on the first data, generating, using one or more large language models executed by the artificial intelligence engine, one or more first modifications to one or more first operating parameters of the first treatment session, wherein the one or more first modifications increase or decrease the one or more first operating parameters at a first certain minimum rate to enable a first gradually monotonically increasing progression associated with the user, and causing, using the one or more first modifications to the one or more first operating parameters of the first treatment session, the first electromechanical machine to be controlled.

Another aspect of the disclosed embodiments includes a computer-implemented method that includes storing, in a data storage device, a first data set including a set of data types. The method includes training, using the first data set, an artificial intelligence engine to correlate one or more subsets of data in the first data set with one or more qualitative or quantitative indications of probabilities of one or more patients experiencing medical events. The method includes receiving stability data of a patient performing the treatment plan. The stability data includes a gait of the patient, a balance of the patient, or both. The method includes correlating, using the artificial intelligence engine, the stability data with at least one qualitative or quantitative indication of a probability of the patient experiencing a medical event. Responsive to determining that the at least one qualitative or quantitative indication of the probability satisfies a first threshold, the method includes modifying the treatment plan such that the at least one qualitative or quantitative indication of the probability no longer satisfies that particular indication of the probability's respective threshold.

Another aspect of the disclosed embodiments includes a computer-implemented method including receiving, from one or more of at least one medical device, at least one sensor, and at least one computing device, objective information associated with a user that uses the at least one medical device during execution of a treatment plan. The objective information includes at least data comprising one or more objective measurements associated with the user. Based on the objective information, the method includes generating, using one or more terms defined in at least one medical terminology database, at least one input prompt. The method includes providing, to an at least artificial intelligence engine configured to provide one or more predictions indicating one or more treatment interventions, the at least one input prompt. The method includes receiving, from the at least artificial intelligence engine, at least one prediction indicating one or more treatment interventions, and controlling, while the user uses the at least one medical device, and based on at least one aspect of the one or more treatment interventions indicated by the at least one prediction, the at least one medical device.

Another aspect of the disclosed embodiments includes a computer-implemented method including, using one or more processing devices, to receive a plurality of sets of data corresponding to respective users of a plurality of users undergoing rehabilitation treatment using respective treatment devices. Each of the sets of data includes sensor data obtained while the respective users perform respective treatment plans and the plurality of sets of data is received in real-time or near real-time as the respective users perform the respective treatment plans. The method includes storing, as objective information, the plurality of sets of data in a queryable data format; receiving first sensor data associated with the performance, by a first user using a first treatment device, of a first treatment plan. The first treatment plan corresponds to a standardized treatment plan assigned to the user based on a classification of the user. The first sensor data correlate with the objective information stored in the queryable data format. The method includes providing the objective information and the first sensor data to a machine learning model trained to predict, based on the objective information and the first sensor data, a likelihood of the first user achieving a rehabilitation goal associated with the rehabilitation treatment. The method includes generating, based on the objective information, the first sensor data, and the likelihood of the first user achieving the rehabilitation goal, a second treatment plan; and controlling, based on the second treatment plan, the first treatment device.

Another aspect of the disclosed embodiments includes a computer-implemented method including receiving, from one or more of an electromechanical machine, a sensor, and a computing device, objective data associated with a user that uses the electromechanical machine to perform a treatment plan, receiving, from a user of the electromechanical machine, subjective outcome data, and based on the objective and subjective data, determining, using an artificial intelligence engine, a presence of at least one comorbidity associated with the user. The determining includes correlating at least one objective datum and at least one subjective datum, wherein each of the correlations satisfies a threshold correlation level. Based on each of the correlations satisfying the threshold correlation level, the method includes identifying at least one comorbidity associated with the user. Based on the at least one identified comorbidity associated with the user, the method includes generating, using the artificial intelligence engine, a modified treatment plan for the user, and controlling, while the user uses the electromechanical machine and using the modified treatment plan, the electromechanical machine.

Another aspect of the disclosed embodiments includes a computer-implemented method including storing, in a first database, first data associated with a first user, the first database having a first level of access control, receiving from the first database, the first data, and storing the first data in a second database having a second level of access control, storing, in the second database, objective data received from an electromechanical machine, selecting, using an artificial intelligence engine, a treatment protocol for the first user by analyzing at least the first data and the objective data, and generating real-time data related to the therapy session by monitoring a therapy session of the first user. The therapy session is performed according to the selected treatment protocol. The method includes correlating, using the artificial intelligence engine, the generated real-time data with data stored in the second database, and based on the correlation, determining whether intervention is needed.

Another aspect of the disclosed embodiments includes a computer-implemented method including storing, in a data storage device, a first data set comprising a plurality of data types, training, using the first data set, an artificial intelligence engine to correlate one or more subsets of data in the first data set with one or more qualitative or quantitative indications of probabilities of one or more patients experiencing medical events, and receiving stability data of a patient performing the treatment plan. The stability data comprises a gait of the patient, a balance of the patient, or both. The method includes correlating, using the artificial intelligence engine, the stability data with at least one qualitative or quantitative indication of a probability of the patient experiencing a medical event, and responsive to determining that the at least one qualitative or quantitative indication of the probability satisfies a first threshold, modifying the treatment plan such that the at least one qualitative or quantitative indication of the probability no longer satisfies that particular indication of the probability's respective threshold.

Another aspect of the disclosed embodiments includes a computer-implemented method including storing, in a data storage device, one or more data sets corresponding to a plurality of users. The one or more data sets comprise a plurality of data types. The method includes training, using the one or more data sets, an artificial intelligence engine to: (i) generate one or more data signatures for the plurality of users, wherein the one or more data signatures indicate one or more similarities in the data sets associated with each user, and (ii) correlate the one or more data signatures with one or more treatment outcomes for the plurality of users. The method includes receiving, from an electromechanical machine, a sensor, or a computing device, objective data associated with a user performing a treatment plan, generating, using the artificial intelligence engine and the objective data, a data signature of the user, and based on a correlation between the data signature of the user and the one or more data signatures of the plurality of users, determining, using the artificial intelligence engine, a qualitative or quantitative indication of a probability of a positive or improved treatment outcome. Responsive to the qualitative or quantitative indication of a probability of the positive or improved treatment outcome satisfying a threshold level, the method includes modifying the treatment plan to increase the probability of achieving the positive or improved treatment outcome.

Another aspect of the disclosed embodiments includes a computer-implemented method including receiving, from one or more of an electromechanical machine, a sensor, and a computing device, data associated with a user who uses the electromechanical machine to perform a treatment plan. Based on the data, the method includes determining, using an artificial intelligence engine, a presence of at least one kinesthetic impingement associated with the user. The determining includes correlating at least two of a pain measurement, a motion capture metric, a physiological marker, and a neural activity indicator, wherein each of the at least two correlations satisfies a threshold correlation level. Based on each of the at least two correlations satisfying the threshold correlation level, the method includes generating a unique data signature associated with the at least one kinesthetic impingement associated with the user. Based on the unique data signature associated with the user, the method includes generating, using the artificial intelligence engine, a modified treatment plan for the user, and controlling, while the user uses the electromechanical machine and using the modified treatment plan, the electromechanical machine.

Another aspect of the disclosed embodiments includes a computer-implemented method including receiving, from an electromechanical machine and a home automation device, data associated with a user who uses the electromechanical machine to perform a rehabilitation treatment plan. Based on one or more correlations of one or more indicators included in the data, the method includes generating a unique data signature associated with a rehabilitation optimization state for the user. Based on the unique data signature, the method includes generating, using the artificial intelligence engine, a modified treatment plan for the user. The modified treatment plan includes adjustments to at least one of an exercise parameter of the electromechanical machine or an environmental parameter controlled by the home automation device, and controlling, while the user performs the rehabilitation treatment plan and using the modified treatment plan, operation of at least one of the electromechanical machine and the home automation device.

Another aspect of the disclosed embodiments includes a computer-implemented method including receiving, from an electromechanical machine and a sensor, data associated with a user that uses the electromechanical machine to perform a treatment plan. The sensor comprises at least one of an audio sensor and a motion sensor. Based on one or more correlations of one or more indicators included in the data, the method includes generating, using an artificial intelligence engine, a unique data signature associated with a rehabilitation progression state or a rehabilitation difficulty state for the user. The one or more indicators comprise at least one of an audio event sensed by the audio sensor and a motion event sensed by the motion sensor. Based on the unique data signature, the method includes generating, using the artificial intelligence engine, a modified treatment plan for the user. The modified treatment plan includes adjustments to at least one of an exercise parameter of an electromechanical machine, and controlling, while the user uses the electromechanical machine and using the modified treatment plan, the electromechanical machine.

Another aspect of the disclosed embodiments includes a computer-implemented method includes receiving, from one or more internet-of-things (IOT) devices, data indicative of user interactions with the one or more IOT devices, wherein the user interactions with the one or more IoT devices are related to a prescribed rehabilitation treatment plan involving use of an electromechanical machine, and analyzing, using an artificial intelligence engine, the data to determine a compliance state of the user with the rehabilitation treatment plan. The compliance state is determined based correlations of one or more indicators included in the data. Based on the compliance state, the method includes generating, using the artificial intelligence engine, a unique data signature associated with the compliance state, and based on the unique data signature associated with the compliance state, performing one or more preventative actions.

Another aspect of the disclosed embodiments includes a computer-implemented method including receiving, at a control command center, data from a plurality of devices associated with a plurality of patients. Each device is configured to execute one or more interventions or protocols as part of a patient treatment plan. The method includes presenting, via a user interface of the control command center, a real-time display of patient status information and device status information for the plurality of patients and devices, analyzing, using an artificial intelligence engine, the data to identify, for each patient, a progression state, a compliance state, or a need for intervention based on one or more indicators included in the data, generating, using the artificial intelligence engine and based on the analysis, one or more recommended interventions or protocol adjustments for at least one of the patients, and enabling, via the user interface, a therapist or an artificial intelligence system to monitor the plurality of patients and devices, review the recommended interventions or protocol adjustments, and selectively initiate, approve, or modify interventions for the at least one of the patients.

Another aspect of the disclosed embodiments includes a computer-implemented method including receiving, for a user, data indicative of one or more identified conditions associated with the user, and selecting, using an artificial intelligence engine and based on the data, a plurality of rehabilitation protocols from a protocol library. Each rehabilitation protocol is targeted at improving or mitigating at least one of the identified conditions. The method includes assigning the user to at least one cohort based on shared characteristics with other users. The cohort assignment is used to inform protocol selection or adaptation. The method includes executing, for the user, the plurality of rehabilitation protocols in a sequence or combination determined by the artificial intelligence engine. The execution comprises controlling operation of at least one rehabilitation device in accordance with the selected protocols. The method includes monitoring, using one or more sensors and the artificial intelligence engine, user-performance data generated during execution of the rehabilitation protocols, and dynamically modifying, using the artificial intelligence engine and based on the user-performance data, at least one parameter of the rehabilitation protocols to optimize therapeutic outcomes for the user.

Another aspect of the disclosed embodiments includes a computer-implemented method including receiving, for a patient, data indicative of a plurality of comorbidities and associated physiological and performance information defining a comorbidity profile. The method includes analyzing, using an artificial intelligence engine, the data to objectively select, from a protocol library, a rehabilitation protocol optimized for the patient's comorbidity profile. The protocol selection is based on data associated with patients having similar comorbidity profiles. The method includes controlling, in accordance with the selected rehabilitation protocol, operation of at least one rehabilitation device during execution of the selected rehabilitation protocol, monitoring, using one or more sensors and the artificial intelligence engine, patient performance and physiological responses during execution of the selected rehabilitation protocol, and dynamically modifying, using the artificial intelligence engine and based on the monitored performance and physiological data, at least one parameter of the rehabilitation protocol in order to optimize therapeutic outcomes for the patient.

In some embodiments, a unique data signature may refer herein to a pointer (e.g., a hash value) pointing to a profile, database, data structure or structures, description of some set of attributes, qualities, properties, and/or measurements. A unique data signature may point to or be associated with a set of data (which may structured data or unstructured data), where the data includes a profile or other description of one or more conditions. In some embodiments, a unique data signature may refer to a uniquely encoded set of conditions, and the unique data signature may be dereferenced when generating one or more modified treatment plans. In some embodiments, a data signature may contain within itself encodings for any set or combination of conditions, such that the data signature includes data associated with a user or patient, and such data may be directly included in the data signature itself or it may be encoded in the data signature. All references to a data signature herein contemplate all such embodiments as well as other embodiments not expressly described but implementable. References herein to data or profiles associated with a data signature shall be interpreted to refer to such data and/or profiles when such data and profiles exist and are pointed to or at by the data signature, and shall be interpreted to refer to the data signature itself when such data and/or profiles are embedded or encoded within the data signature. Further, all references herein to rehabilitation herein may shall be deemed to refer, wherever applicable, also to prehabilitation.

Other technical features may be readily apparent to one skilled in the art from the following figures, descriptions, and claims.

### BRIEF DESCRIPTION OF THE DRAWINGS

For a more complete understanding of this disclosure and its advantages, reference is now made to the following description, taken in conjunction with the accompanying drawings. It is emphasized that, according to common practice, the various features of the drawings are not to-scale. On the contrary, the dimensions of the various features are arbitrarily expanded or reduced for clarity.

FIG. 1 generally illustrates a block diagram of an embodiment of a computer-implemented system for managing a treatment plan according to principles of the present disclosure.

FIG. 2 generally illustrates a perspective view of an embodiment of a treatment device according to principles of the present disclosure.

FIG. 3 generally illustrates a perspective view of an embodiment of pedal of the treatment device of FIG. 2 according to principles of the present disclosure.

FIG. 4 generally illustrates a perspective view of a person using the treatment device of FIG. 2 according to principles of the present disclosure.

FIG. 5 generally illustrates an example embodiment of an overview display of an assistant interface according to principles of the present disclosure.

FIG. 6 generally illustrates an example block diagram of training a machine learning model to output, based on data pertaining to the patient, a treatment plan for the patient according to principles of the present disclosure.

FIG. 7 generally illustrates an example overview display of the assistant interface presenting recommended treatment plans and excluded treatment plans in real-time during a telemedicine session according to principles of the present disclosure.

FIG. 8 generally illustrates an example overview display of the assistant interface presenting, in real-time during a telemedicine session, recommended treatment plans that have changed as a result of patient data changing according to principles of the present disclosure.

FIG. 9 is a flow diagram generally illustrating a method for modifying, by an artificial intelligence engine, a treatment plan for optimizing patient outcome and pain levels during one or more treatment sessions according to principles of the present disclosure.

FIG. 10 is a flow diagram generally illustrating a method for further modifying a treatment plan for optimizing patient outcome and pain levels, by an artificial intelligence engine, during one or more treatment sessions using updated pain levels according to principles of the present disclosure.

FIG. 11 generally illustrates a computer system according to principles of the present disclosure.

FIG. 12A is a flow diagram generally illustrating a method for optimizing treatment plans to support user progression during rehabilitation for the purpose of assisting in determining AI-driven interventions by using machine learning to generate at least one data signature associated with a comorbidity according to the principles of the present disclosure.

FIG. 12B is a flow diagram generally illustrating a method for optimizing treatment plans to support user progression during rehabilitation for the purpose of assisting in determining AI-driven interventions by using machine learning to generate at least one data signature associated with a regression of a user's medical condition according to the principles of the present disclosure.

FIG. 12C is a flow diagram generally illustrating a method for optimizing treatment plans to support user progression during rehabilitation for the purpose of assisting in determining AI-driven interventions by using machine learning to generate at least one data signature associated with a life-threatening event according to the principles of the present disclosure.

FIG. 12D is a flow diagram generally illustrating a method for optimizing treatment plans to support user progression during rehabilitation for the purpose of assisting in determining AI-driven interventions by using machine learning to generate at least one data signature associated with a treatment gap according to the principles of the present disclosure.

FIG. 12E illustrates an example overview display of the assistant interface presenting modified treatment plans based on comorbidities in real-time during a telemedicine session according to principles of the present disclosure.

FIG. 13A is a flow diagram generally illustrating a method for controlling, by an artificial intelligence engine, operation of an electromechanical machine while a user uses the machine according to principles of the present disclosure.

FIG. 13B is a flow diagram generally illustrating further optional aspects of the method described above in connection with FIG. 13A according to principles of the present disclosure.

FIG. 13C is a flow diagram generally illustrating a method for using artificial intelligence to control an electromechanical machine in order to adjust dosage amounts and frequencies for infusion-based or other medication-based therapies for the purpose of optimizing a treatment plan to shorten a user's recovery period according to principles of the present disclosure.

FIG. 13D is a flow diagram generally illustrating a method for incorporating into a treatment plan to optimize the user's recovery via the use of AI-driven interventions associated with reinforcement learning that rely on evidence-based experiments according to principles of the present disclosure.

FIG. 13E illustrates another exemplary embodiment, similar to the method illustrated in FIG. 13D according to principles of the present disclosure.

FIG. 13F illustrates example first and second datasets according to principles of the present disclosure.

FIG. 13G illustrates an example user interface illustrating tiles that are selectable by a user according to principles of the present disclosure.

FIG. 14A is a flow diagram generally illustrating a method for using artificial intelligence to modify, based on predictive analytics, a treatment plan for the purpose of optimizing patient outcomes and pain levels during treatment sessions according to principles of the present disclosure.

FIG. 14B is a flow diagram generally illustrating a method for using artificial intelligence and machine learning to enable, via AI-driven interventions, the early detection of patients deemed high risk due to a patient's slow progression in rehabilitation according to principles of the present disclosure.

FIG. 14C is a flow diagram generally illustrating a method for predicting a user's final progression state of rehabilitation based on number of sessions, medication intake, pain level, or some combination thereof and for using the predictions to perform AI-driven interventions according to principles of the present disclosure.

FIG. 14D is a flow diagram generally illustrating a method for determining and performing interventions using an electromechanical machine wherein the interventions are determined by using a large language model to generate and rank, based on one or more measures of efficacy, a list of interventions according to principles of the present disclosure.

FIG. 14E is a flow diagram generally illustrating a method for using artificial intelligence and machine learning to enhance patient treatment plan progression, reduce patient pain, and increase patient compliance by adjusting, based on one or more real-time inputs, a component parameter according to principles of the present disclosure.

FIG. 15A is a flow diagram generally illustrating a method of modifying a treatment plan based on analysis and correlation of both objective data and subjective data related to a user using an electromechanical machine according to principles of the present disclosure,

FIG. 15B is a flow diagram generally illustrating a method for, based on data lakes, using artificial intelligence and machine learning to reduce a probability for an event where a patient falls according to principles of the present disclosure.

FIG. 15C is a flow diagram generally illustrating a method for obtaining and using objective information pertaining to a performance of a patient performing a treatment plan on a rehabilitative device according to principles of the present disclosure.

FIG. 15D is a flow diagram generally illustrating a method of determining whether intervention is needed in connection with a treatment plan based on analysis and correlation of both first data associated with a user of an electromechanical machine and objective data received from the electromechanical machine according to principles of the present disclosure.

FIG. 15E illustrates additional steps which may be incorporated into the method described above in connection with FIG. 15D.

FIG. 16A shows an example embodiment of a method for analyzing objective information pertaining to the performance of a patient performing a treatment plan and for modifying the treatment plan using AI/ML.

FIG. 16B shows an example embodiment of another method for analyzing objective information pertaining to the performance of a patient performing a treatment plan and for modifying the treatment plan (e.g., using AI/ML).

FIG. 16C is a flow diagram generally illustrating a method for enabling evidence-based studies to determine the efficacy of different rehabilitation approaches according to principles of the present disclosure.

FIG. 17A is a flow diagram generally illustrating a method for objectively measuring phantom limb pain and associated body kinesthetic impingements to enable dynamic modification of a rehabilitation treatment plan according to principles of the present disclosure.

FIG. 17B is a flow diagram generally illustrating a method for integrating home automation devices with rehabilitation treatment plans to enhance user outcomes and experience according to principles of the present disclosure.

FIG. 17C is a flow diagram generally illustrating a method for using motion and audio sensing to objectively measure rehabilitation progress and identify factors that may hinder recovery according to principles of the present disclosure.

FIG. 17D is a flow diagram generally illustrating a method for leveraging IoT devices to monitor compliance with a rehabilitation treatment plan and to trigger preventative actions when non-compliance is detected according to principles of the present disclosure.

FIG. 17E is a flow diagram generally illustrating a method for providing an enhanced user interface as a control command center for monitoring and managing a plurality of patients and devices executing interventions and evidence-based protocols according to principles of the present disclosure.

FIG. 17F is a flow diagram generally illustrating a method for optimizing rehabilitation by dynamically selecting and executing a plurality of rehabilitation protocols, each targeted at improving or mitigating one or more identified conditions in a user according to principles of the present disclosure.

FIG. 17G is a flow diagram generally illustrating a method for optimizing rehabilitation by performing AI-driven interventions for patients with multiple comorbidities according to principles of the present disclosure.

FIG. 18A is a flow diagram generally illustrating a method for using a generative LLM with natural language processing (NLP)-assisted interactions to interact with a user during a treatment session in order to guide the user in the user's use of the electromechanical machine according to principles of the present disclosure.

FIG. 19 is a flow diagram generally illustrating a method for determining and performing interventions using objective-based measurements and specifying the interventions using a medical description language that is capable of service as input to a large language model, according to the principles of the present disclosure.

FIG. 20A is a flow diagram generally illustrating a method for using AI-driven devices where the rehabilitation process uses selective data signature-based data injection according to principles of the present disclosure.

FIG. 20B is a flow diagram generally illustrating a method for using AI-driven interventions via optimization of rehabilitation that uses selective data signature-based data injection for users with internal or invasive life support devices according to principles of the present disclosure.

FIG. 21A is a block diagram illustrating further aspects of a method flow related to determining and performing interventions using an electromechanical machine interventions according to principles of the present disclosure.

FIG. 21B is a block diagram that illustrates aspects of objective data and subjective data according to principles of the present disclosure.

FIG. 21C is a block of an example of prompts (e.g. questions) that may be asked to a user and that may relate to objective or subjective data according to principles of the present disclosure.

FIG. 21D is block diagram illustrating aspects of an artificial intelligence engine that may interact with a treatment apparatus according to principles of the present disclosure.

FIG. 21E is a block diagram depicting an example of a treatment plan as may be utilized in conjunction with artificial intelligence techniques to generate an action intervention in relation to a user who uses an electromechanical according to principles of the present disclosure.

FIG. 21F is a block diagram of a data flow relating to a method for determining and performing interventions using an electromechanical machine in which interventions are determined by using a large language model according to principles of the present disclosure.

### NOTATION AND NOMENCLATURE

Various terms are used to refer to particular system components. Different companies may refer to a component by different names—this document does not intend to distinguish between components that differ in name but not function. In the following discussion and in the claims, the terms “including” and “comprising” are used in an open-ended fashion, and thus should be interpreted to mean “including, but not limited to . . . ” Also, the term “couple” or “couples” is intended to mean either an indirect or direct connection. Thus, if a first device couples to a second device, that connection may be through a direct connection or through an indirect connection via other devices and connections.

The terminology used herein is for the purpose of describing particular example embodiments only and is not intended to be limiting. As used herein, the singular forms “a,” “an,” and “the” may be intended to include the plural forms as well, unless the context clearly indicates otherwise. The method steps, processes, and operations described herein are not to be construed as necessarily requiring their performance in the particular order discussed or illustrated, unless specifically identified as an order of performance. It is also to be understood that additional or alternative steps may be employed.

The terms first, second, third, etc. may be used herein to describe various elements, components, regions, layers and/or sections; however, these elements, components, regions, layers and/or sections should not be limited by these terms. These terms may be only used to distinguish one element, component, region, layer, or section from another region, layer, or section. Terms such as “first,” “second,” and other numerical terms, when used herein, do not imply a sequence or order unless clearly indicated by the context. Thus, a first element, component, region, layer, or section discussed below could be termed a second element, component, region, layer, or section without departing from the teachings of the example embodiments. The phrase “at least one of,” when used with a list of items, means that different combinations of one or more of the listed items may be used, and only one item in the list may be needed. For example, “at least one of: A, B, and C” includes any of the following combinations: A, B, C, A and B, A and C, B and C, and A and B and C. In another example, the phrase “one or more” when used with a list of items means there may be one item or any suitable number of items exceeding one.

Spatially relative terms, such as “inner,” “outer,” “beneath,” “below,” “lower,” “above,” “upper,” “top,” “bottom,” “inside,” “outside,” “contained within,” “superimposing upon,” and the like, may be used herein. These spatially relative terms can be used for ease of description to describe one element's or feature's relationship to another element(s) or feature(s) as illustrated in the figures. The spatially relative terms may also be intended to encompass different orientations of the device in use, or operation, in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptions used herein interpreted accordingly.

A “treatment plan” may include one or more treatment protocols, and each treatment protocol may include one or more treatment sessions. Each treatment session comprises several session periods, with each session period including a particular exercise for treating the body part of the patient. For example, a treatment plan for post-operative rehabilitation after a knee surgery may include an initial treatment protocol with twice daily stretching sessions for the first 3 days after surgery and a more intensive treatment protocol with active exercise sessions performed 4 times per day starting 4 days after surgery. A treatment plan may also include information pertaining to a medical procedure to perform on the patient, a treatment protocol for the patient using a treatment device, a diet regimen for the patient, a medication regimen for the patient, a sleep regimen for the patient, additional regimens, or some combination thereof.

The terms telemedicine, telehealth, telemed, teletherapeutic, telemedicine, etc. may be used interchangeably herein.

In some embodiments, a unique data signature may refer herein to a pointer (e.g., a hash value) pointing to a profile, database, data structure or structures, description of some set of attributes, qualities, properties, and/or measurements. A unique data signature may point to or be associated with a set of data (which may structured data or unstructured data), where the data includes a profile or other description of one or more conditions. In some embodiments, a unique data signature may refer to a uniquely encoded set of conditions, and the unique data signature may be dereferenced when generating one or more modified treatment plans. In some embodiments, a data signature may contain within itself encodings for any set or combination of conditions, such that the data signature includes data associated with a user or patient, and such data may be directly included in the data signature itself or it may be encoded in the data signature. All references to a data signature herein contemplate all such embodiments as well as other embodiments not expressly described but implementable. References herein to data or profiles associated with a data signature shall be interpreted to refer to such data and/or profiles when such data and profiles exist and are pointed to or at by the data signature, and shall be interpreted to refer to the data signature itself when such data and/or profiles are embedded or encoded within the data signature.

All references herein to rehabilitation herein may shall be deemed to refer, wherever applicable, also to prehabilitation.

### DETAILED DESCRIPTION

The following discussion is directed to various embodiments of the present disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Evidence-based rehabilitation experimentation, at scale, does not exist. “At scale” refers to, without limitation, a system or application that can handle a large volume of users, data, transactions, or operations efficiently, reliably, and cost-effectively-without a significant degradation in performance, availability, or maintainability (as used here, it means performing evidence-based rehabilitation experimentation consistent with these types of parameters). Further, without limitation, “at scale” may refer to: Software Systems able to serve millions of users or requests per second (e.g., Netflix® streaming to global users), where code must be modular, efficient, and horizontally scalable; Infrastructure systems deployed across distributed environments (multiple regions, data centers, or availability zones) to ensure reliability and redundancy; databases designed for sharding, replication, and load balancing to handle massive read/write operations or petabyte-scale data; cloud computing systems leveraging elastic resources (e.g., Amazon Web Services® Lambda, Kubernetes, auto-scaling groups) to dynamically handle workload spikes and minimize waste; and DevOps/continuous integration, delivery, and deployment to support hundreds or thousands of microservices and environments without manual bottlenecks.

Conventional infrastructure is designed to deliver rehabilitation treatment. Data in commercial outcome registries, such as the Focus on Therapeutic Outcomes patient database, and other registries, may be limited to a limited number (e.g., 12-30) of questions regarding patient reported outcome measures (PROM) and may not include treatment plan detail, objective data, including measures and metrics, patient reactions to treatment, or timing. PROM results are independent from the cause of the progression. The rehabilitation industry lacks the infrastructure needed to statistically correlate, using objective measures, patient progression with specific treatment plans and/or protocols. Standardized experimentation has not been implemented as an industry standard, protocol or de facto approach. As such, patient outcomes today have largely not advanced over those from 20-30 or even more years ago. Various technical problems have prevented rehabilitation from being optimized, such problems including, without limitation, a lack of data format standardization (i.e., canonical data representations), predictive analytics, generative narration, remote treatment, automated control of treatment apparatuses, and the like. This means that the rehabilitative environment in terms of measuring patient progress is the metaphorical equivalent of the Wild West. Whatever one sheriff (therapist, as defined supra) says goes for that person, but 500 different sheriffs (therapists) may all write, record and measure progress differently.

Accordingly, present embodiments provide one or more technical solutions to the technical problems associated with improving rehabilitation. For example, some embodiments may provide an improved rehabilitation infrastructure that enables performing predictive analytics on large data lakes of user information (e.g., treatment plans, treatment outcomes, treatment plan completion information, user pain levels, etc.) to use artificial intelligence and machine learning to perform interventions. The improved rehabilitation infrastructure may execute an artificial intelligence engine that leverages evidence-based treatment protocols to deliver real-time or near real-time changes to treatment plans. In some embodiments, an electromechanical machine being used by the user to perform a treatment plan may be controlled based on data collected from the electromechanical machine, one or more sensors, and/or one or more computing devices.

Treatment plans that may be selected and/or generated may be standardized and subsequently or ultimately assigned to patients based on patient classification. The data associated with the improved rehabilitation may be specific to diagnosis requirements, and data about the rehabilitation (e.g., treatment plan user progression, treatment outcomes, etc.) may be entered automatically in real-time or near real-time in a standardized format. A database may be used that is structured for real-time artificial intelligence and machine learning predictive analysis and/or control. In some embodiments, the improved rehabilitation infrastructure may enable outputting multitudes of double-blind experiment results per month.

In addition, the improved rehabilitation infrastructure may deliver, via the technical solutions described herein, industry-leading rehabilitation progression for a multitude of users in potentially less treatment sessions than associated with conventional convention rehabilitation. Further, the improved rehabilitation infrastructure may enable economic efficiencies by enhancing user progression to reducing the amount of money spent associated with user rehabilitation (e.g., reducing number of treatment sessions, reducing length of time users need to rent an electromechanical machine, reducing visits with a medical professional, etc.). These may result from broadly-focused (i.e., globally, nationwide, etc.), multi-disciplinary, evidence-based, double-blinded research that is dynamically performed in parallel with treatment, at scale. Further, beyond provider-based clinical improvement, the improved rehabilitation infrastructure may enable a full-scale rehabilitation delivery model designed to leverage continuous or continual monitoring of a treatment session for the purpose of modifying the treatment plan on a real-time basis. In addition, the research that is performed may enable identifying best practices using artificial intelligence and machine learning.

As patients engage in rehabilitation, their pain levels often increase. Patients may take more pain medication as their pain levels increase. In addition, the increased pain levels may discourage the patients from diligently following their rehabilitation treatment plans. Such noncompliance may slow down the recovery progresses of the patients, resulting in the patient taking pain medication for longer periods of time. As the quantity and the length of time (e.g., days, weeks, months, etc.) increases for which patients take their pain medication, the more likely the patients will become addicted to and/or dependent on their pain medication. For example, patients may become physically dependent on opioids and experience symptoms of tolerance (i.e., patients' bodies become desensitized to the drugs and patients need to take higher doses of the drug to relieve pain) and withdrawal (e.g., physical effects). Furthermore, patients may become mentally dependent on the opioids (i.e., the use of the opioids is a conditioned response to a feeling-a trigger- and the trigger sets off biochemical changes in the patients' brains that strongly influence addictive behavior). Prescribing an optimal type and quantity of pain medication for an optimal length of time can be challenging, especially when doctors are not provided with adequate patient input (e.g., a patient's pain level before, during, and after a rehabilitation session) as the patient rehabilitates. It may be desirable to modify a treatment plan for optimizing patient outcome and pain levels during one or more treatment sessions.

Determining optimal remote examination procedures to create a treatment plan for a patient having certain characteristics (e.g., vital-sign or other measurements; performance; demographic; psychographic; geographic; diagnostic; measurement- or test-based; medically historic; behavioral historic; cognitive; etiologic; cohort-associative; differentially diagnostic; surgical, physically therapeutic, pharmacologic and other treatment(s) recommended; etc.) may be a technically challenging problem. For example, a multitude of information may be considered when determining a treatment plan, which may result in inefficiencies and inaccuracies in the treatment plan selection process. In a rehabilitative setting, some of the multitude of information considered may include characteristics of the patient such as personal information, performance information, and measurement information. The personal information may include, e.g., demographic, psychographic or other information, such as an age, a weight, a gender, a height, a body mass index, a medical condition, a familial medication history, an injury, a medical procedure, a medication prescribed, or some combination thereof. The performance information may include, e.g., an elapsed time of using a treatment device, an amount of force exerted on a portion of the treatment device, a range of motion achieved on the treatment device, a movement speed of a portion of the treatment device, an indication of a plurality of pain levels using the treatment device, or some combination thereof. The measurement information may include, e.g., a vital sign, a respiration rate, a heartrate, a temperature, a blood pressure, or some combination thereof. It may be desirable to process the characteristics of a multitude of patients, the treatment plans performed for those patients, and the results of the treatment plans for those patients.

Doctors typically prescribe pain medication, such as opioids, to a patient after conducting a physical examination and/or communicating with the patient (e.g., to obtain the patient's rehabilitative progress and/or pain level). Another technical problem may involve distally treating, via a computing device during a telemedicine or telehealth session, a patient from a location different than a location at which the patient is located. An additional technical problem is controlling or enabling the control of, from the different location, a treatment device used by the patient at the location at which the patient is located. Oftentimes, when a patient undergoes rehabilitative surgery (e.g., knee surgery), a healthcare professional may prescribe a treatment device to the patient to use to perform a treatment protocol at their residence or any mobile location or temporary domicile. A healthcare professional may refer to a doctor, physician assistant, nurse, chiropractor, dentist, physical therapist, acupuncturet, physical trainer, coach, personal trainer, neurologist, cardiologist, or the like. A healthcare profession may refer to any person with a credential, license, degree, or the like in the field of medicine, physical therapy, rehabilitation, or the like.

When the healthcare professional is located in a location different from the patient and the treatment device, it may be technically challenging for the healthcare professional to monitor the patient's actual progress (as opposed to relying on the patient's word about the patient's progress) in using the treatment device, modify the treatment plan according to the patient's progress, adapt the treatment device to the personal characteristics of the patient as the patient performs the treatment plan, and the like.

Accordingly, systems and methods, such as those described herein, that use sensor data to modify a treatment plan and/or to adapt the treatment device while a patient performs the treatment plan using the treatment device, may be desirable.

In some embodiments, the systems and methods described herein may be configured to receive a treatment plan for a user, such as a patient. The treatment plan may correspond to a rehabilitation treatment plan, a prehabilitation treatment plan, an exercise treatment plan, or any other suitable treatment plan. The treatment plan may comprise one or more exercise routines for the patient to complete during one or more treatment sessions. The patient may include a person performing the one or more exercise routines. The person may perform the one or more exercise routines on a treatment device, such as a rehabilitation device. The system and methods may be configured to receive treatment data pertaining to the patient. The treatment data may include various characteristics of the patient, various measurement information pertaining to the patient while the patient uses the treatment device, various characteristics of the treatment device, the treatment plan, other suitable data, or a combination thereof. The system and methods may be configured to receive patient input correlating with at least one of the pain levels of the patient and use the treatment plan, the treatment data, and the patient input to generate at least one threshold. Responsive to an occurrence of exceeding the at least one threshold, the systems and methods can modify the treatment plan.

In some embodiments, while the patient uses the treatment device to perform the treatment plan, at least some of the treatment data may correspond to sensor data of a sensor configured to sense various characteristics of the treatment device and/or the measurement information of the patient. Additionally, or alternatively, while the patient uses the treatment device to perform the treatment plan, at least some of the treatment data may correspond to sensor data from a sensor associated with a wearable device configured to sense the measurement information of the patient.

The various characteristics of the treatment device may include one or more settings of the treatment device, a current revolutions per time period (e.g., such as one minute) of a rotating member (e.g., such as a wheel) of the treatment device, a resistance setting of the treatment device, other suitable characteristics of the treatment device, or a combination thereof. The measurement information may include one or more vital signs of the patient, a respiration rate of the patient, a heartrate of the patient, a temperature of the patient, a blood pressure of the patient, other suitable measurement information of the patient, or a combination thereof.

In some embodiments, the systems and methods described herein may be configured to generate treatment information using the treatment data. The treatment information may include a summary of the performance of the treatment plan by the patient while using the treatment device, such that the treatment data is presentable at a computing device of a healthcare professional responsible for the performance of the treatment plan by the patient. The healthcare professional may include a medical professional (e.g., such as a doctor, a nurse, a therapist, and the like), an exercise professional (e.g., such as a coach, a trainer, a nutritionist, and the like), or another professional sharing at least one of medical and exercise attributes (e.g., such as an exercise physiologist, a physical therapist, an occupational therapist, and the like). As used herein, and without limiting the foregoing, a “healthcare professional” may be a human being, a robot, a virtual assistant, a virtual assistant in a virtual and/or augmented reality, or an artificially intelligent entity, including a software program, integrated software and hardware, or hardware alone.

In some embodiments, the patient input may include a patient goal, a level of exhaustion, pain level, or any other suitable information or combination thereof. For example, the patient goal may include a target rehabilitation date, a maximum or a minimum length of time for one or more exercise sessions, a level of difficulty, or any other desired goal. The level of exhaustion may include the current level of exhaustion of the patient (e.g., based on a scale of pain level values from 1-10), the number of hours the patient slept during the previous night, or any other desired information. The pain level may include one or more levels of pain (e.g., based on a scale of pain level values from 1-10) the patient experiences before, during, and/or after the exercise sessions. The patient may input one or more pain level values correlating with one or more body parts. For example, a patient may be rehabilitating from a double knee surgery where each knee is recovering at different paces. The patient may be experiencing a pain level value of four on the right knee and a pain level value of seven on the left knee prior to an exercise session. The patient may experience an increase in pain levels during the exercise session (e.g., a pain level value of five on the right knee and a pain level value of nine on the left knee).

The threshold may include one or more threshold conditions. The one or more threshold conditions may be based on characteristics of the injury, the patient, the treatment plan, the recovery results, the examination results, the pain level, the level of exhaustion, the exercise session, any other suitable factors, or combination thereof. For example, a patient may be using a treatment device, such as an exercise bicycle, during a treatment session. The threshold may include a threshold condition that the patient cannot apply more than first and second amounts of measured force to right and left pedals, respectively. The treatment device may include one or more modes, such as an active-assisted mode, that can assist a user in cycling. The active-assisted mode may refer to a sensor of the treatment device receiving measurements of revolutions per minute of one or more radially-adjustable couplings, and causing the electric motor to drive the one or more radially-adjustable couplings rotationally coupled to the one or more pedals when the measured revolutions per minute satisfy a parameter (e.g., a threshold condition). The threshold condition may be configurable by the user and/or the physician, for example, as part of the treatment plan. The electric motor may be powered off while the user provides the driving force to the radially-adjustable couplings as long as the revolutions per minute are above a revolutions per minute threshold and the threshold condition is not satisfied. When the revolutions per minute are less than the revolutions per minute threshold then the threshold condition is satisfied and the electric motor may be controlled to drive the radially-adjustable couplings to maintain the revolutions per minute threshold.

Responsive to an occurrence of exceeding the at least one threshold, the artificial intelligence engine may be trained to modify the treatment plan. Modifying the treatment plan may comprise generating at least one updated exercise routine during one of the one or more treatment sessions. For example, if the patient's pain level exceeds a threshold during a treatment session, the artificial intelligence engine may generate an updated exercise routine. The updated exercise routine may include changes, such as changes to an amount of time of the treatment session, an amount of time between treatment sessions (e.g., for the patient to rest and for the patient's pain level to decrease), a type of exercise to be completed in the treatment session, a type of treatment device for the patient to perform on during the treatment session, any other desired modification, or combination thereof. The updated exercise routine may include changes to parameters of the treatment device, such as changes to a radius of one or more of the pedals, a level of assistance applied by the electric motor to assist the patient with cycling, an amount of resistance the electric motor applies to the one or more pedals, any other desired change to a parameter, or combination thereof.

In some embodiments, the systems and methods can control the treatment device while the patient uses the treatment device, including during a telemedicine session. The controlling can be based on parameters of the modified treatment plan. For example, the artificial intelligence engine may be configured to modify the treatment plan such that the treatment device changes a radius of rotation of one or more of the pedals, a level of assistance applied by the electric motor to assist the patient with cycling, an amount of resistance the electric motor applies to the one or more pedals, any other desired control, or combination thereof.

In some embodiments, the artificial intelligence engine can be configured to receive the modified patient input correlating with an updated pain level of the patient (e.g., the patient inputs a change in pain level during a treatment session) and use the modified treatment plan, the treatment data, and the modified patient input to generate at least one modified threshold. Responsive to an occurrence of exceeding the at least one modified threshold, the artificial intelligence engine can be configured to modify the modified treatment plan. The modified treatment plan can include one or more modifications that differ from the treatment plan. For example, the modified treatment plan may differ from the treatment plan by having one or more different exercise routines for the patient to perform on a treatment device, one or more different parameters for controlling the treatment device, one or more different thresholds, any other differences, or combinations thereof.

In some embodiments, at least one of the treatment data and the patient input can be received in real-time or near real-time and the treatment plan can be modified in real-time or near real-time. In some embodiments, at least one of the modified patient input and the modified patient input can be received in real-time or near real-time and the modified treatment plan can be modified in real-time or near real-time.

In some embodiments, the healthcare professional may review the treatment information and determine whether to modify the treatment plan and/or one or more characteristics of the treatment device. For example, the healthcare professional may review the treatment information and compare the treatment information to the treatment plan being performed by the patient.

Depending on what result is desired, the artificial intelligence engine may be trained to output several treatment plans. For example, one result may include recovering to a threshold level (e.g., 75% range of motion) in a fastest amount of time, while another result may include fully recovering (e.g., 100% range of motion) regardless of the amount of time. Another result may include recovering while not exceeding a threshold level for pain (e.g., at or below a specific pain level) between treatment sessions, while another result may include recovering while not exceeding a threshold level for pain during a treatment session.

The artificial intelligence engine may compare the following (i) expected information, which pertains to the patient while the patient uses the treatment device to perform the treatment plan to (ii) the measurement information (e.g., indicated by the treatment information), which pertains to the patient while the patient uses the treatment device to perform the treatment plan. The expected information may include one or more vital signs of the patient, a respiration rate of the patient, a heartrate of the patient, a temperature of the patient, a blood pressure of the patient, other suitable information of the patient, or a combination thereof. The artificial intelligence engine may determine that the treatment plan is optimal for the particular patient (i.e., the patient is having a desired rehabilitation result) if one or more parts or portions of the measurement information are within an acceptable range associated with one or more corresponding parts or portions of the expected information (e.g., within one or more thresholds). Conversely, the artificial intelligence engine may determine that the treatment plan is not optimal for the particular patient (i.e., the patient is not having a desired rehabilitation result) if one or more parts or portions of the measurement information are outside of the range associated with one or more corresponding parts or portions of the expected information (e.g., outside of the one or more thresholds).

For example, the artificial intelligence engine may determine whether a blood pressure value (e.g., systolic pressure, diastolic pressure, and/or pulse pressure) corresponding to the patient while the patient uses the treatment device (e.g., indicated by the measurement information) is within an acceptable range (e.g., plus or minus 1%, plus or minus 5%, or any suitable range) of an expected blood pressure value indicated by the expected information. The artificial intelligence engine may determine that the treatment plan is having the desired effect if the blood pressure value corresponding to the patient while the patient uses the treatment device is within the range of the expected blood pressure value. Conversely, the artificial intelligence engine may determine that the treatment plan is not having the desired effect if the blood pressure value corresponding to the patient while the patient uses the treatment device is outside of the range of the expected blood pressure value.

In another example, the artificial intelligence engine may determine whether a pain level corresponding to the patient while the patient uses the treatment device (e.g., indicated by the patient input) is within an acceptable range of an expected pain level value indicated by the expected information (e.g., a pain level value for a patient two days after surgery is expected to be higher than the pain level value of the patient two weeks after the surgery, a pain level value for a patient having right ankle surgery is expected to be higher during recovery than an uninjured left ankle). The artificial intelligence engine may determine that the treatment plan is having the desired effect if the pain level corresponding to the patient while the patient uses the treatment device is within the range of the expected pain level value (e.g., not exceeding the threshold). Conversely, the artificial intelligence engine may determine that the treatment plan is not having the desired effect if the pain level value corresponding to the patient while the patient uses the treatment device is outside of the range of the expected pain level value. If the artificial intelligence engine determines that an occurrence of exceeding the at least one threshold occurs, then the artificial intelligence engine may be configured to modify the treatment plan or any previously modified treatment plans.

In some embodiments, the artificial intelligence engine may compare the expected characteristics of the treatment device while the patient uses the treatment device to perform the treatment plan with characteristics of the treatment device indicated by the treatment information. For example, the artificial intelligence engine may compare an expected resistance setting of the treatment device with an actual resistance setting of the treatment device indicated by the treatment information. The artificial intelligence engine may determine that the user is performing the treatment plan properly if the actual characteristics of the treatment device indicated by the treatment information are within a range of corresponding ones of the expected characteristics of the treatment device. Conversely, the artificial intelligence engine may determine that the user is not performing the treatment plan properly if the actual characteristics of the treatment device indicated by the treatment information are outside the range of corresponding ones of the expected characteristics of the treatment device. If the artificial intelligence engine determines that an occurrence of exceeding the at least one threshold occurs, then the artificial intelligence engine may be configured to modify the treatment plan or any previously modified treatment plans.

If the artificial intelligence engine determines that the treatment information indicates that the user is performing the treatment plan properly and/or that the treatment plan is having the desired effect, the artificial intelligence engine may determine not to modify the treatment plan or the one or more characteristics of the treatment device. Conversely, while the patient uses the treatment device to perform the treatment plan, if the artificial intelligence engine determines that the treatment information indicates that the patient is not or has not been performing the treatment plan properly and/or that the treatment plan is not or has not been having the desired effect, the artificial intelligence engine may determine to modify the treatment plan and/or the one or more characteristics of the treatment device.

In some embodiments, the system may interact with a user interface to provide treatment plan input indicating one or more modifications to the treatment plan and/or to one or more characteristics of the treatment device if the artificial intelligence engine determines to modify the treatment plan and/or the one or more characteristics of the treatment device. For example, the interface may provide input indicating an increase or decrease in the resistance setting of the treatment device, an increase or decrease in an amount of time the user is required to use the treatment device according to the treatment plan, or other suitable modification to the one or more characteristics of the treatment device.

In some embodiments, the systems and methods described herein may be configured to modify the treatment plan based on one or more modifications indicated by the treatment plan input. Additionally, or alternatively, the systems and methods described herein may be configured to modify the one or more characteristics of the treatment device based on the modified treatment plan and/or the treatment plan input. For example, the treatment plan input may indicate modifying the one or more characteristics of the treatment device and/or the modified treatment plan may require or indicate adjustments to the treatment device in order for the user to achieve the desired results of the modified treatment plan.

In some embodiments, the systems and methods described herein may be configured to receive subsequent treatment data pertaining to the user while the user uses the treatment device to perform the modified treatment plan. For example, after the artificial intelligence engine modifies the treatment plan and/or controls the one or more characteristics of the treatment device, the user may continue use the treatment device to perform the modified treatment plan. The subsequent treatment data may correspond to treatment data generated while the user uses the treatment device to perform the modified treatment plan. In some embodiments, the subsequent treatment data may correspond to treatment data generated while the user continues to use the treatment device to perform the treatment plan, after the healthcare professional has received the treatment information and determined not to modify the treatment plan and/or control the one or more characteristics of the treatment device.

Based on subsequent (e.g., modified) treatment plan input generated by the artificial intelligence engine, the systems and methods described herein may be configured to further modify the treatment plan and/or control the one or more characteristics of the treatment device. The subsequent treatment plan input may correspond to input provided by the patient at the user interface, from treatment data corresponding to sensor data from a sensor of a wearable device worn by the patient during one of the one or more treatment sessions, from a sensor configured to detect treatment data pertaining to the patient, any other desired information, or combination thereof.

The healthcare professional may receive and/or review treatment information continuously or periodically while the user uses the treatment device to perform the treatment plan. Based on one or more trends indicated by the continuously and/or periodically received treatment information, the healthcare professional may determine whether to modify the treatment plan and/or control the one or more characteristics of the treatment device. For example, the one or more trends may indicate an increase in heart rate or other suitable trends indicating that the user is not performing the treatment plan properly and/or performance of the treatment plan by the person is not having the desired effect.

In some embodiments, the systems and methods described herein may be configured to use artificial intelligence and/or machine learning to assign patients to cohorts and to dynamically control a treatment device based on the assignment during an adaptive telemedicine session. In some embodiments, numerous treatment devices may be provided to patients. The treatment devices may be used by the patients to perform treatment plans in their residences, at a gym, at a rehabilitative center, at a hospital, or any suitable location, including permanent or temporary domiciles.

In some embodiments, the treatment devices may be communicatively coupled to a server. Characteristics of the patients, including the treatment data, may be collected before, during, and/or after the patients perform the treatment plans. For example, the personal information, the performance information, and the measurement information may be collected before, during, and/or after the patient performs the treatment plans. The results (e.g., improved performance or decreased performance) of performing each exercise may be collected from the treatment device throughout the treatment plan and after the treatment plan is performed. The parameters, settings, configurations, etc. (e.g., position of pedal, amount of resistance, etc.) of the treatment device may be collected before, during, and/or after the treatment plan is performed.

Each characteristic of the patient, each result, and each parameter, setting, configuration, etc. may be timestamped and may be correlated with a particular step in the treatment plan. Such a technique may enable determining which steps in the treatment plan will lead to desired results (e.g., improved muscle strength, range of motion, etc.) and which steps will lead to diminishing returns (e.g., continuing to exercise after 3 minutes actually delays or harms recovery).

Data may be collected from the treatment devices and/or any suitable computing device (e.g., computing devices where personal information is entered, such as the interface of the computing device described herein, a clinician interface, patient interface, and the like) over time as the patients use the treatment devices to perform the various treatment plans. The data that may be collected may include the characteristics of the patients, the treatment plans performed by the patients, the results of the treatment plans, any of the data described herein, any other suitable data, or a combination thereof.

In some embodiments, the data may be processed to group certain patients into cohorts. The patients may be grouped by patients having certain or selected similar characteristics, treatment plans, and results of performing the treatment plans. For example, athletic patients having no medical conditions who perform a treatment plan (e.g., use the treatment device for 30 minutes a day 5 times a week for 3 weeks) and who fully recover may be grouped into a first cohort. Older patients who are classified obese and who perform a treatment plan (e.g., use the treatment plan for 10 minutes a day 3 times a week for 4 weeks) and who improve their range of motion by 75 percent may be grouped into a second cohort.

In some embodiments, an artificial intelligence engine may include one or more machine learning models that are trained using the cohorts. For example, the one or more machine learning models may be trained to receive an input of characteristics of a new patient and to output a treatment plan for the patient that results in a desired result. The machine learning models may match a pattern between the characteristics of the new patient and at least one patient of the patients included in a particular cohort. When a pattern is matched, the machine learning models may assign the new patient to the particular cohort and select the treatment plan associated with the at least one patient. The artificial intelligence engine may be configured to control, distally and based on the treatment plan, the treatment device while the new patient uses the treatment device to perform the treatment plan.

As may be appreciated, the characteristics of the new patient (e.g., a new user) may change as the new patient uses the treatment device to perform the treatment plan. For example, the performance of the patient may improve quicker than expected for patients in the cohort to which the new patient is currently assigned. Accordingly, the machine learning models may be trained to dynamically reassign, based on the changed characteristics, the new patient to a different cohort that includes patients having characteristics similar to the now-changed characteristics as the new patient. For example, a clinically obese patient may lose weight and no longer meet the weight criterion for the initial cohort, result in the patient's being reassigned to a different cohort with a different weight criterion.

A different treatment plan may be selected for the new patient, and the treatment device may be controlled, distally (e.g., which may be referred to as remotely) and based on the different treatment plan, the treatment device while the new patient uses the treatment device to perform the treatment plan. Such techniques may provide the technical solution of distally controlling a treatment device.

Further, the systems and methods described herein may lead to faster recovery times and/or better results for the patients because the treatment plan that most accurately fits their characteristics is selected and implemented, in real-time, at any given moment. “Real-time” may also refer to near real-time, which may be less than 10 seconds or any reasonably proximate difference between two times. As described herein, the term “results” may refer to medical results or medical outcomes. Results and outcomes may refer to responses to medical actions. The term “medical action(s)” may refer to any suitable action(s) performed by the healthcare professional, and such action or actions may include diagnoses, prescriptions for treatment plans, prescriptions for treatment devices, and the making, composing and/or executing of appointments, telemedicine sessions, prescription of medicines, telephone calls, emails, text messages, and the like.

Further, the artificial intelligence engine may be trained to output treatment plans that are not optimal i.e., sub-optimal, nonstandard, or otherwise excluded (all referred to, without limitation, as “excluded treatment plans”) for the patient. For example, if a patient has high blood pressure, a particular exercise may not be approved or suitable for the patient as it may put the patient at unnecessary risk or even induce a hypertensive crisis and, accordingly, that exercise may be flagged in the excluded treatment plan for the patient. In some embodiments, the artificial intelligence engine may monitor the treatment data received while the patient (e.g., the user) with, for example, high blood pressure, uses the treatment device to perform an appropriate treatment plan and may modify the appropriate treatment plan to include features of an excluded treatment plan that may provide beneficial results for the patient if the treatment data indicates the patient is handling the appropriate treatment plan without aggravating, for example, the high blood pressure condition of the patient.

In some embodiments, the treatment plans and/or excluded treatment plans may be presented, during a telemedicine or telehealth session, to a healthcare professional. The healthcare professional may select a particular treatment plan for the patient to cause that treatment plan to be transmitted to the patient and/or to control, based on the treatment plan, the treatment device. In some embodiments, to facilitate telehealth or telemedicine applications, including remote diagnoses, determination of treatment plans and rehabilitative and/or pharmacologic prescriptions, the artificial intelligence engine may receive and/or operate distally from the patient and the treatment device.

In such cases, the recommended treatment plans and/or excluded treatment plans may be presented simultaneously with a video of the patient in real-time or near real-time during a telemedicine or telehealth session on a user interface of a computing device of a healthcare professional. The video may also be accompanied by audio, text and other multimedia information and/or sensorial or perceptive (e.g., tactile, gustatory, haptic, pressure-sensing-based or electromagnetic (e.g., neurostimulation)). Real-time may refer to less than or equal to 2 seconds. Near real-time may refer to any interaction of a sufficiently short time to enable two individuals to engage in a dialogue via such user interface, and will generally be less than 10 seconds (or any suitable proximate difference between two different times) but greater than 2 seconds.

Presenting the treatment plans generated by the artificial intelligence engine concurrently with a presentation of the patient video may provide an enhanced user interface because the healthcare professional may continue to visually and/or otherwise communicate with the patient while also reviewing the treatment plans on the same user interface. The enhanced user interface may improve the healthcare professional's experience using the computing device and may encourage the healthcare professional to reuse the user interface. Such a technique may also reduce computing resources (e.g., processing, memory, network) because the healthcare professional does not have to switch to another user interface screen to enter a query for a treatment plan to recommend based on the characteristics of the patient. The artificial intelligence engine may be configured to provide, dynamically on the fly, the treatment plans and excluded treatment plans.

In some embodiments, the treatment device may be adaptive and/or personalized because its properties, configurations, and positions may be adapted to the needs of a particular patient. For example, the pedals may be dynamically adjusted on the fly (e.g., via a telemedicine session or based on programmed configurations in response to certain measurements being detected) to increase or decrease a range of motion to comply with a treatment plan designed for the user. In some embodiments, a healthcare professional may adapt, remotely during a telemedicine session, the treatment device to the needs of the patient by causing a control instruction to be transmitted from a server to treatment device. Such adaptive nature may improve the results of recovery for a patient, furthering the goals of personalized medicine, and enabling personalization of the treatment plan on a per-individual basis.

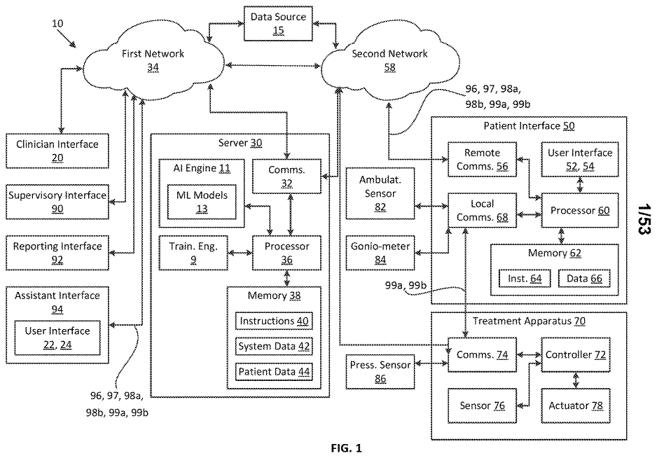

FIG. 1 shows a block diagram of a computer-implemented system 10, hereinafter called “the system” for managing a treatment plan. Managing the treatment plan may include using an artificial intelligence engine to recommend treatment plans and/or provide excluded treatment plans that should not be recommended to a patient.

The system 10 also includes a server 30 configured to store and to provide data related to managing the treatment plan. The server 30 may include one or more computers and may take the form of a distributed and/or virtualized computer or computers. The server 30 may also include a first communication interface 32 configured to communicate with the clinician interface 20 via a first network 34. In some embodiments, the first network 34 may include wired and/or wireless network connections such as Wi-Fi, Bluetooth, ZigBee, Near-Field Communications (NFC), cellular data networks, etc. The server 30 may include a first processor 36 and a first machine-readable storage memory 38 (the latter of which may be called a “memory” for short), holding first instructions 40 for performing the various actions of the server 30 for execution by the first processor 36. The server 30 may be configured to store data regarding the treatment plan. For example, the memory 38 includes a system data store 42 configured to hold system data, such as data pertaining to treatment plans for treating one or more patients. The server 30 may also be configured to store data regarding performance by a patient in following a treatment plan. For example, the memory 38 includes a patient data store 44 (e.g., database) configured to hold patient data, such as data pertaining to the one or more patients, including data representing each patient's performance within the treatment plan.

In addition, the characteristics (e.g., personal, performance, measurement, etc.) of the patients, the treatment plans followed by the patients, the level of compliance with the treatment plans, and the results of the treatment plans may use correlations and other statistical or probabilistic measures to enable the partitioning of or to partition the treatment plans into different patient cohort-equivalent databases in the patient data store 44. For example, the data for a first cohort of first patients having a first similar injury, a first similar medical condition, a first similar medical procedure performed, a first treatment plan followed by the first patient, and a first result of the treatment plan may be stored in a first patient database. The data for a second cohort of second patients having a second similar injury, a second similar medical condition, a second similar medical procedure performed, a second treatment plan followed by the second patient, and a second result of the treatment plan may be stored in a second patient database. Any single characteristic or any combination of characteristics may be used to separate the cohorts of patients. In some embodiments, the different cohorts of patients may be stored in different partitions or volumes of the same database. There is no specific limit to the number of different cohorts of patients allowed, other than as limited by mathematical combinatoric and/or partition theory.

This characteristic data, treatment plan data, and results data may be obtained from numerous treatment apparatuses and/or computing devices over time and stored in the patient data store 44. The characteristic data, treatment plan data, and results data may be correlated in the patient-cohort databases in the patient data store 44. The characteristics of the patients may include personal information, performance information, and/or measurement information.

In addition to the historical information about other patients stored in the patient cohort-equivalent databases, real-time or near-real-time information based on the current patient's characteristics about a current patient being treated may be stored in an appropriate patient cohort-equivalent database. The characteristics of the patient may be determined to match or be similar to the characteristics of another patient in a particular cohort (e.g., cohort A) and the patient may be assigned to that cohort.

In some embodiments, the server 30 may execute an artificial intelligence (AI) engine 11 that uses one or more machine learning models 13 to perform at least one of the embodiments disclosed herein. The server 30 may include a training engine 9 capable of generating the one or more machine learning models 13. The machine learning models 13 may be trained to assign patients to certain cohorts based on their characteristics, select treatment plans using real-time and historical data correlations involving patient cohort-equivalents, and control a treatment apparatus 70, among other things. The one or more machine learning models 13 may be generated by the training engine 9 and may be implemented in computer instructions executable by one or more processing devices of the training engine 9 and/or the servers 30. To generate the one or more machine learning models 13, the training engine 9 may train the one or more machine learning models 13. The one or more machine learning models 13 may be used by the artificial intelligence engine 11.

The training engine 9 may be a rackmount server, a router computer, a personal computer, a portable digital assistant, a smartphone, a laptop computer, a tablet computer, a netbook, a desktop computer, an Internet of Things (IOT) device, any other desired computing device, or any combination of the above. The training engine 9 may be cloud-based, a real-time software platform, or an embedded system (e.g., microcode-based and/or implemented) and it may include privacy software or protocols, and/or security software or protocols.

To train the one or more machine learning models 13, the training engine 9 may use a training data set of a corpus of the characteristics of the patients that used the treatment apparatus 70 to perform treatment plans, the details (e.g., treatment protocol including exercises, amount of time to perform the exercises, how often to perform the exercises, a schedule of exercises, parameters/configurations/settings of the treatment apparatus 70 throughout each step of the treatment plan, etc.) of the treatment plans performed by the patients using the treatment apparatus 70, and the results of the treatment plans performed by the patients. The one or more machine learning models 13 may be trained to match patterns of characteristics of a patient with characteristics of other patients assigned to a particular cohort. The term “match” may refer to an exact match, a correlative match, a substantial match, etc. The one or more machine learning models 13 may be trained to receive the characteristics of a patient as input, map the characteristics to characteristics of patients assigned to a cohort, and select a treatment plan from that cohort. The one or more machine learning models 13 may also be trained to control, based on the treatment plan, the treatment apparatus 70.

Different machine learning models 13 may be trained to recommend different treatment plans for different desired results. For example, one machine learning model may be trained to recommend treatment plans for most effective recovery, while another machine learning model may be trained to recommend treatment plans based on speed of recovery.

Using training data that includes training inputs and corresponding target outputs, the one or more machine learning models 13 may refer to model artifacts created by the training engine 9. The training engine 9 may find patterns in the training data wherein such patterns map the training input to the target output, and generate the machine learning models 13 that capture these patterns. In some embodiments, the artificial intelligence engine 11, a database, and/or the training engine 9 may reside on another component (e.g., assistant interface 94, clinician interface 20, etc.) depicted in FIG. 1.

The one or more machine learning models 13 may comprise, e.g., a single level of linear or non-linear operations (e.g., a support vector machine [SVM]) or the machine learning models 13 may be a deep network, i.e., a machine learning model comprising multiple levels of non-linear operations. Examples of deep networks are neural networks including generative adversarial networks, convolutional neural networks, recurrent neural networks with one or more hidden layers, and fully connected neural networks (e.g., each neuron may transmit its output signal to the input of the remaining neurons, as well as to itself). For example, the machine learning model may include numerous layers and/or hidden layers that perform calculations (e.g., dot products) using various neurons.

The system 10 also includes a patient interface 50 configured to communicate information to a patient and to receive feedback from the patient. Specifically, the patient interface includes an input device 52 and an output device 54, which may be collectively called a patient user interface 52, 54. The input device 52 may include one or more devices, such as a keyboard, a mouse, a touch screen input, a gesture sensor, and/or a microphone and processor configured for voice recognition. The output device 54 may take one or more different forms including, for example, a computer monitor or display screen on a tablet, smartphone, or a smart watch. The output device 54 may include other hardware and/or software components such as a projector, virtual reality capability, augmented reality capability, etc. The output device 54 may incorporate various and/or different visual, audio, or other presentation technologies. For example, the output device 54 may include a non-visual display, such as an audio signal, which may include spoken language and/or other sounds such as tones, chimes, and/or melodies, which may signal different conditions and/or directions. The output device 54 may comprise one or more different display screens presenting various data and/or interfaces or controls for use by the patient. The output device 54 may include graphics, which may be presented by a web-based interface and/or by a computer program or application (App.).

As shown in FIG. 1, the patient interface 50 includes a second communication interface 56 (one example of an “input device”, an “output device,” or both), which may also be called a remote communication interface configured to communicate with the server 30 and/or the clinician interface 20 via a second network 58. In some embodiments, the second network 58 may include a local area network (LAN), such as an Ethernet network. In some embodiments, the second network 58 may include the Internet, and communications between the patient interface 50 and the server 30 and/or the clinician interface 20 may be secured via encryption, such as, for example, by using a virtual private network (VPN). In some embodiments, the second network 58 may include wired and/or wireless network connections such as Wi-Fi, Bluetooth, ZigBee, Near-Field Communications (NFC), cellular data networks, etc. In some embodiments, the second network 58 may be the same as and/or operationally coupled to the first network 34.

The patient interface 50 may include a second processor 60 and a second machine-readable storage memory 62 holding second instructions 64 for execution by the second processor 60 for performing various actions of patient interface 50. The second machine-readable storage memory 62 also includes a local data store 66 configured to hold data, such as data pertaining to a treatment plan and/or patient data, such as data representing a patient's performance within a treatment plan. The patient interface 50 also includes a local communication interface 68 configured to communicate with various devices for use by the patient in the vicinity of the patient interface 50. The local communication interface 68 (one example of an “input device,” an “output device,” or both) may include wired and/or wireless communications. In some embodiments, the local communication interface 68 may include a local wireless network such as Wi-Fi, Bluetooth, ZigBee, Near-Field Communications (NFC), cellular data networks, etc.

The system 10 also includes a treatment apparatus 70 configured to be manipulated by the patient and/or to manipulate a body part of the patient for performing activities according to the treatment plan. In some embodiments, the treatment apparatus 70 may take the form of an exercise and rehabilitation apparatus configured to perform and/or to aid in the performance of a rehabilitation regimen, which may be an orthopedic rehabilitation regimen, and the treatment includes rehabilitation of a body part of the patient, such as a joint or a bone or a muscle group. The treatment apparatus 70 (one example of a “treatment device”) may be any suitable medical, rehabilitative, therapeutic, etc. apparatus configured to be controlled distally via another computing device to treat a patient and/or exercise the patient. The treatment apparatus 70 may be an electromechanical machine (e.g., including one or more pedals, motors, arms, levers, rotational axes, wheels, seats, benches, weights, etc.), an electromechanical bicycle, an electromechanical spin-wheel, a smart-mirror, a treadmill, or the like. The body part may include, for example, a spine, a hand, a foot, a knee, or a shoulder. The body part may include a part of a joint, a bone, or a muscle group, such as one or more vertebrae, a tendon, or a ligament. As shown in FIG. 1, the treatment apparatus 70 includes a controller 72, which may include one or more processors, computer memory, and/or other components. The treatment apparatus 70 may also include a fourth communication interface 74 configured to communicate with the patient interface 50 via the local communication interface 68. The treatment apparatus 70 may also include one or more internal sensors 76 and an actuator 78, such as a motor. The actuator 78 may be used, for example, for moving the patient's body part and/or for resisting forces by the patient.

The internal sensors 76 may measure one or more operating characteristics of the treatment apparatus 70 such as, for example, a force, a position, a speed, and/or a velocity. In some embodiments, the internal sensors 76 may include a position sensor configured to measure at least one of a linear motion or an angular motion of a body part of the patient. For example, an internal sensor 76 in the form of a position sensor may measure a distance that the patient is able to move a part of the treatment apparatus 70, where such distance may correspond to or translate into a range of motion that the patient's body part is able to achieve. In some embodiments, the internal sensors 76 may include a force sensor configured to measure a force applied by the patient. For example, an internal sensor 76 in the form of a force sensor may measure a force or weight the patient, using a particular body part, is able to apply to the treatment apparatus 70.

The system 10 shown in FIG. 1 also includes an ambulation sensor 82, which communicates with the server 30 via the local communication interface 68 of the patient interface 50. The ambulation sensor 82 may track and store a number of steps taken by the patient. In some embodiments, the ambulation sensor 82 may take the form of a wristband, wristwatch, or smart watch. In some embodiments, the ambulation sensor 82 may be integrated within a phone, such as a smartphone.

The system 10 shown in FIG. 1 also includes a goniometer 84, which communicates with the server 30 via the local communication interface 68 of the patient interface 50. The goniometer 84 measures an angle of the patient's body part. For example, the goniometer 84 may measure the angle of flex of a patient's knee or elbow or shoulder.

The system 10 shown in FIG. 1 also includes a pressure sensor 86, which communicates with the server 30 via the local communication interface 68 of the patient interface 50. The pressure sensor 86 measures an amount of pressure or weight applied by a body part of the patient. For example, pressure sensor 86 may measure an amount of force applied by a patient's foot when pedaling a stationary bike

The system 10 shown in FIG. 1 also includes a supervisory interface 90 which may be similar or identical to the clinician interface 20. In some embodiments, the supervisory interface 90 may have enhanced functionality beyond what is provided on the clinician interface 20. The supervisory interface 90 may be configured for use by a person having responsibility for the treatment plan, such as an orthopedic surgeon.

The system 10 shown in FIG. 1 also includes a reporting interface 92 which may be similar or identical to the clinician interface 20. In some embodiments, the reporting interface 92 may have less functionality than the clinician interface 20. For example, the reporting interface 92 may not have the ability to modify a treatment plan. Such a reporting interface 92 may be used, for example, by a biller to determine the use of the system 10 for billing purposes. In another example, the reporting interface 92 may not have the ability to display patient identifiable information, presenting only pseudonymized data and/or anonymized data for certain data fields concerning a data subject and/or for certain data fields concerning a quasi-identifier of the data subject. Such a reporting interface 92 may be used, for example, by a researcher to determine various effects of a treatment plan on different patients.

The system 10 includes an assistant interface 94 for an assistant, such as a doctor, a nurse, a physical therapist, a technician, or a healthcare professional, to remotely communicate with the patient interface 50 and/or the treatment apparatus 70. Such remote communications may enable the assistant to provide assistance or guidance to a patient using the system 10. More specifically, the assistant interface 94 is configured to communicate a telemedicine signal 96, 97, 98a, 98b, 99a, 99b with the patient interface 50 via a network connection such as, for example, via the first network 34 and/or the second network 58. The telemedicine signal 96, 97, 98a, 98b, 99a, 99b comprises one of an audio signal 96, an audiovisual signal 97, an interface control signal 98a for controlling a function of the patient interface 50, an interface monitor signal 98b for monitoring a status of the patient interface 50, an apparatus control signal 99a for changing an operating parameter of the treatment apparatus 70, and/or an apparatus monitor signal 99b for monitoring a status of the treatment apparatus 70. In some embodiments, each of the control signals 98a, 99a may be unidirectional, conveying commands from the assistant interface 94 to the patient interface 50. In some embodiments, in response to successfully receiving a control signal 98a, 99a and/or to communicate successful and/or unsuccessful implementation of the requested control action, an acknowledgement message may be sent from the patient interface 50 to the assistant interface 94. In some embodiments, each of the monitor signals 98b, 99b may be unidirectional, status-information commands from the patient interface 50 to the assistant interface 94. In some embodiments, an acknowledgement message may be sent from the assistant interface 94 to the patient interface 50 in response to successfully receiving one of the monitor signals 98b, 99b.

In some embodiments, the patient interface 50 may be configured as a pass-through for the apparatus control signals 99a and the apparatus monitor signals 99b between the treatment apparatus 70 and one or more other devices, such as the assistant interface 94 and/or the server 30. For example, the patient interface 50 may be configured to transmit an apparatus control signal 99a in response to an apparatus control signal 99a within the telemedicine signal 96, 97, 98a, 98b, 99a, 99b from the assistant interface 94.

In some embodiments, the assistant interface 94 may be presented on a shared physical device as the clinician interface 20. For example, the clinician interface 20 may include one or more screens that implement the assistant interface 94. Alternatively or additionally, the clinician interface 20 may include additional hardware components, such as a video camera, a speaker, and/or a microphone, to implement aspects of the assistant interface 94.

In some embodiments, one or more portions of the telemedicine signal 96, 97, 98a, 98b, 99a, 99b may be generated from a prerecorded source (e.g., an audio recording, a video recording, or an animation) for presentation by the output device 54 of the patient interface 50. For example, a tutorial video may be streamed from the server 30 and presented upon the patient interface 50. Content from the prerecorded source may be requested by the patient via the patient interface 50. Alternatively, via a control on the assistant interface 94, the assistant may cause content from the prerecorded source to be played on the patient interface 50.

The assistant interface 94 includes an assistant input device 22 and an assistant display 24, which may be collectively called an assistant user interface 22, 24. The assistant input device 22 may include one or more of a telephone, a keyboard, a mouse, a trackpad, or a touch screen, for example. Alternatively or additionally, the assistant input device 22 may include one or more microphones. In some embodiments, the one or more microphones may take the form of a telephone handset, headset, or wide-area microphone or microphones configured for the assistant to speak to a patient via the patient interface 50. In some embodiments, assistant input device 22 may be configured to provide voice-based functionalities, with hardware and/or software configured to interpret spoken instructions by the assistant by using the one or more microphones. The assistant input device 22 may include functionality provided by or similar to existing voice-based assistants such as Siri by Apple, Alexa by Amazon, Google Assistant, or Bixby by Samsung. The assistant input device 22 may include other hardware and/or software components. The assistant input device 22 may include one or more general purpose devices and/or special-purpose devices.

The assistant display 24 may take one or more different forms including, for example, a computer monitor or display screen on a tablet, a smartphone, or a smart watch. The assistant display 24 may include other hardware and/or software components such as projectors, virtual reality capabilities, or augmented reality capabilities, etc. The assistant display 24 may incorporate various different visual, audio, or other presentation technologies. For example, the assistant display 24 may include a non-visual display, such as an audio signal, which may include spoken language and/or other sounds such as tones, chimes, melodies, and/or compositions, which may signal different conditions and/or directions. The assistant display 24 may comprise one or more different display screens presenting various data and/or interfaces or controls for use by the assistant. The assistant display 24 may include graphics, which may be presented by a web-based interface and/or by a computer program or application (App.).

In some embodiments, the system 10 may provide computer translation of language from the assistant interface 94 to the patient interface 50 and/or vice-versa. The computer translation of language may include computer translation of spoken language and/or computer translation of text, wherein the text and/or spoken language may be any language, formal or informal, current or outdated, digital, quantum or analog, invented, human or animal (e.g., dolphin) or ancient, with respect to the foregoing, e.g., Old English, Zulu, French, Japanese, Klingon, Kobaïan, Attic Greek, Modern Greek, etc., and in any form, e.g., academic, dialectical, patois, informal, e.g., “electronic texting,” etc. Additionally or alternatively, the system 10 may provide voice recognition and/or spoken pronunciation of text. For example, the system 10 may convert spoken words to printed text and/or the system 10 may audibly speak language from printed text. The system 10 may be configured to recognize spoken words by any or all of the patient, the clinician, and/or the assistant. In some embodiments, the system 10 may be configured to recognize and react to spoken requests or commands by the patient. For example, the system 10 may automatically initiate a telemedicine session in response to a verbal command by the patient (which may be given in any one of several different languages).

In some embodiments, the server 30 may generate aspects of the assistant display 24 for presentation by the assistant interface 94. For example, the server 30 may include a web server configured to generate the display screens for presentation upon the assistant display 24. For example, the artificial intelligence engine 11 may generate recommended treatment plans and/or excluded treatment plans for patients and generate the display screens including those recommended treatment plans and/or external treatment plans for presentation on the assistant display 24 of the assistant interface 94. In some embodiments, the assistant display 24 may be configured to present a virtualized desktop hosted by the server 30. In some embodiments, the server 30 may be configured to communicate with the assistant interface 94 via the first network 34. In some embodiments, the first network 34 may include a local area network (LAN), such as an Ethernet network. In some embodiments, the first network 34 may include the Internet, and communications between the server 30 and the assistant interface 94 may be secured via privacy enhancing technologies, such as, for example, by using encryption over a virtual private network (VPN). Alternatively or additionally, the server 30 may be configured to communicate with the assistant interface 94 via one or more networks independent of the first network 34 and/or other communication means, such as a direct wired or wireless communication channel. In some embodiments, the patient interface 50 and the treatment apparatus 70 may each operate from a patient location geographically separate from a location of the assistant interface 94. For example, the patient interface 50 and the treatment apparatus 70 may be used as part of an in-home rehabilitation system, which may be aided remotely by using the assistant interface 94 at a centralized location, such as a clinic or a call center.

In some embodiments, the assistant interface 94 may be one of several different terminals (e.g., computing devices) that may be physically, virtually or electronically grouped together, for example, in one or more call centers or at one or more clinicians' offices. In some embodiments, a plurality of assistant interfaces 94 may be distributed geographically. In some embodiments, a person may work as an assistant remotely from any conventional office infrastructure, including a home office. Such remote work may be performed, for example, where the assistant interface 94 takes the form of a computer and/or telephone. This remote work functionality may allow for work-from-home arrangements that may include full-time, part-time and/or flexible work hours for an assistant.

In some embodiments, the one or more computer-implemented models 13 may comprise large language models. The large language model may involve deep learning in order to understand how characters, words, and sentences function together. Deep learning may involve probabilistic analyses of unstructured data, which eventually enables the deep learning model to recognize distinctions between pieces of content (e.g., words, sentences, paragraphs, documents, images, videos, audio, responses, questions, etc.).

Any of the computer-implemented models 13 described herein may reside one or more memory devices and/or be executed by one or more processing devices partially or wholly on one or more of any computing device (e.g., server 30, patient interface 50, treatment apparatus 70 (electromechanical machine), clinician interface 20, supervisory interface 90, reporting interface 92, assistant interface 94, ambulatory sensor 82, goniometer 84, pressure sensor 86, data source 15, etc.) described herein. For example, any artificial intelligent agent described below may be executed to perform any of the methods described herein on a local computing device (e.g., smartphone, tablet, etc.) and/or a remote computing device (e.g., a server), or a combination thereof.

Some embodiments may use generative artificial intelligence and agentic artificial intelligence. Generative artificial intelligence may focus on content generation from learned statistical patterns. Agentic artificial intelligence may focus on goal-oriented autonomous behavior using reasoning, planning, memory, and tool execution and/or action execution.

Generative artificial intelligence may execute one or more computer-implemented models 13 trained to determine a probability distribution over data. For example, language model may predict the next token:

P(xₜ|x_{<t})

The model may learn syntax, semantics, latent structure, correlations in massive datasets, etc. and then generate outputs such as text, images, code, audio, video, etc. Various generative artificial intelligence architectures may include transformer large language models, diffusion models, generative adversarial networks (GANs), etc. Generative artificial intelligence models may include a limited memory (e.g., context window, temporary session state).

Agentic artificial intelligence may add decision-making loops to generative artificial intelligence models. For example, in addition to generating content, agentic artificial intelligence computer-implemented models may interpret goals, plan actions, select tools, execute steps, evaluate outcomes, revise strategies, continue until objective completion, and the like. Agentic artificial intelligence pipelines may include a goal, planner, tool selection, execution environment, memory and state tracking, self-evaluation/reflection, and determination of a next action that forms a closed feedback loop. Agentic artificial intelligence models are stateful and they decompose tasks into subgoals and invoke external systems (e.g., application programming interfaces (APIs), databases, browsers, compilers, shell commands, robotics controllers, etc.). Agentic artificial intelligence maintains persistent memory (e.g., long-term memory, vector databases, task histories, environment states). Agentic artificial intelligence models may use reinforcement learning to determine whether goals were achieved via reward models, evaluators, critics, reinforcement loops, self-reflection mechanisms, and the like. Agentic artificial intelligence is more autonomous than generative artificial intelligence, as agentic artificial intelligence uses memory to monitor states of systems and plans actions that the agentic artificial intelligence it then executes; and, then, agentic artificial intelligence repeats this process iteratively.

From a functional representation viewpoint, generative AI approximates:

$$P ( Data )$$

or conditionally, the probability of output, given input:

$$P ( Output \vert Input )$$

Agentic AI instead resembles sequential decision systems:

$$π ( {a}_{t} \vert {s}_{t} )$$

where: sₜ=environment state, aₜ=action, and π=policy

Various real-world examples of using generative artificial intelligence models may include treatment plan generation, treatment apparatus 70 control, text generation, image synthesis, autocompletion features, code completion, and/or music generation using LLM chatbots and/or diffusion image models. Various real-world examples of using agentic artificial intelligence may include control agents for autonomous treatment apparatus 70, autonomous treatment plan generation agents, autonomous software engineering agents, artificial intelligence research assistants, robotic process automation with reasoning, and/or autonomous cybersecurity systems using software systems that browse the web and complete tasks, autonomous coding/debugging agents, and/or autonomous agents that manage cloud infrastructure.

In some embodiments, agentic artificial intelligence systems may be implemented using generative artificial intelligence models. For example, an agentic artificial intelligence architecture may include several orchestrated components and/or features such as one or more generative artificial intelligence models, planning components, memories, tools, and/or autonomous agents.

In addition, autonomous artificial intelligence systems may be implemented in some embodiments. Autonomous artificial intelligence may refer to artificial intelligence systems operating over a long period of time with minimal human supervision and may include (i) persistent memory, (ii) self-monitoring, (iii) long-term planning, (iv) adaptive learning from outcomes, (v) multi-agent collaboration, or (vi) continuous environment interaction. A distinction between autonomous artificial intelligence systems and non-autonomous artificial intelligence systems, is that autonomous artificial intelligence systems may create their own subtasks, evaluate their success, revise their strategies, and continue operating in a continuous manner.

In some embodiments, physical artificial intelligence (embodied artificial intelligence) systems may be implemented that combine artificial intelligence reasoning with robotics and real-world sensing (e.g., sensors). Instead of existing only in software, the artificial intelligence may interact physically through treatment apparatuses 70, cameras, light detection and ranging (LiDAR), tactile sensors, robotic actuators, autonomous vehicles, drones, manufacturing systems, etc. Examples that implement physical artificial intelligence may include warehouse robots, humanoid robots, autonomous surgical systems, self-driving vehicles, etc. Physical artificial intelligence may combine concepts from various technological fields including machine learning, computer vision, reinforcement learning, control systems, robotics, and/or sensor fusion. For example, some embodiments may be implemented via the treatment apparatus 70 and/or the assistant interface 94 that asks the user various questions to determine a level of pain the user is experiencing.

In some embodiments, multimodal general intelligence may be implemented using one or more generative artificial intelligence models that process text, image, audio, video, and/or code and unify these modalities into a single continuous reasoning architecture that understands spoken conversation, reads technical diagrams, analyzes live video, controls robotic systems, controls the treatment apparatus 70, and interacts in augmented reality environments.

In some embodiments, self-improving artificial intelligence may be implemented to evaluate its own outputs, generate synthetic training data, refine internal reasoning strategies, optimize models automatically, perform recursive improvements, optimize treatment plans, control treatment apparatuses 70, etc. These techniques may involve the use of meta-learning, reinforcement learning, neural architecture searching, automated machine learning, self-reflection, etc. The self-improving artificial intelligence models may evaluate their own performance and apply algorithmic modifications (e.g., modify weights, modify nodes, modify parameters, modify hidden layers, etc.) to increase future task effectiveness according to defined optimization objectives. In some embodiments, the self-improving artificial intelligence models may improve themselves by generating new computer instructions (e.g., code), testing code, benchmarking outputs, and/or selecting superior implementations via autonomous software engineering and/or evolutionary programming. Further, agentic systems may improve capabilities by adding application programming interfaces (APIs), using external memory, calling tools, building workflows, etc. In some embodiments, the self-improving artificial intelligence models may generate hypotheses, run simulations, evaluate outcomes, and refine their computer-implemented models automatically. The computer-implemented models may use objective functions (e.g., reward functions, loss functions, utility functions), evaluation systems to measure accuracy, efficiency, robustness, alignment, and generalization, and/or memory systems (e.g., persistent storage, retrieval mechanisms, historical performance tracking, etc.).

In some embodiments, scientific discovery-based artificial intelligence may be implemented that combines various techniques and/or models, such as symbolic reasoning, simulation, probabilistic inference, large language models, graph neural networks, physics-informed neural networks, etc.

In some embodiments, collective multi-agent artificial intelligence may be implemented with multiple specialized artificial intelligent agents cooperating together. A multi-agent artificial intelligence system may include planner agents, coding agents, verification agents, memory agents, simulation agents, negotiation agents, etc. These agents may coordinate similarly with distributed operating systems. Multi-agent artificial intelligence may enable parallel reasoning, specialization, fault tolerance, scalable task decomposition, etc.

In some embodiments, cognitive architecture artificial intelligence may be implemented to focus on modeling reasoning, memory, abstraction, attention, planning, learning, and causal understanding. Cognitive architectural artificial intelligence may generate world models, and/or use causal inference, counterfactuals, implement symbolic-neural hybrid systems, and/or implement dynamic memory systems.

In some embodiments, general artificial intelligence (AGI) may be implemented that is configured to transfer knowledge across domains, perform general reasoning, perform autonomous learning, perform flexible adaptations, and/or solve unfamiliar problems. The AGI system may not need to retrain for each new task domain. That is, the AGI system may be configured to perform tasks in new domains without retraining. The AGI system may be implemented using high-dimensional latent vector spaces via models that model language, visual information, physical environments, causal relationships, counterfactual reasoning, symbolic abstractions, temporal sequences, and/or goals and constraints. The computer-implemented models that are used in the AGI system may model and/or generate persistent world models, dynamic memory structures, cross-modal reasoning, etc. The computer-implemented models may maintain an internal predictive simulation of reality (e.g., world model) using techniques such as object permanence, causal inference, counterfactual reasoning, environmental state tracking, prediction under uncertainty via one or more probabilistic graphical models, recurrent memory architectures, latent state-space models, predictive processing systems, etc. The AGI system may perform multi-step planning using various techniques and/or models, such as task decomposition, subgoal generation, action sequencing, optimization, error correction, etc. via reinforcement learning models, Monte Carlo tree searching, hierarchical planning, model-predictive control, utility optimization, etc. The AGI system may include working memory (temporary context manipulation), episodic memory (event storage and retrieval), semantic memory (general knowledge storage), and/or procedural memory (learned behavioral patterns). In some embodiments, the AGI system may be trained once and then deployed statically; and it may also be configured for online learning, continuous adaptation, lifelong learning, etc.

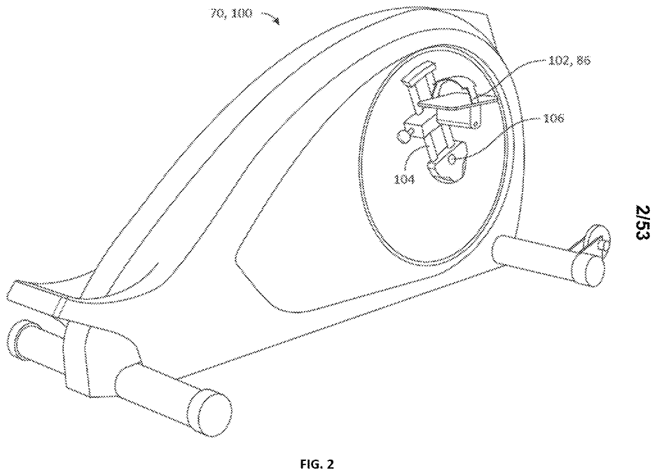

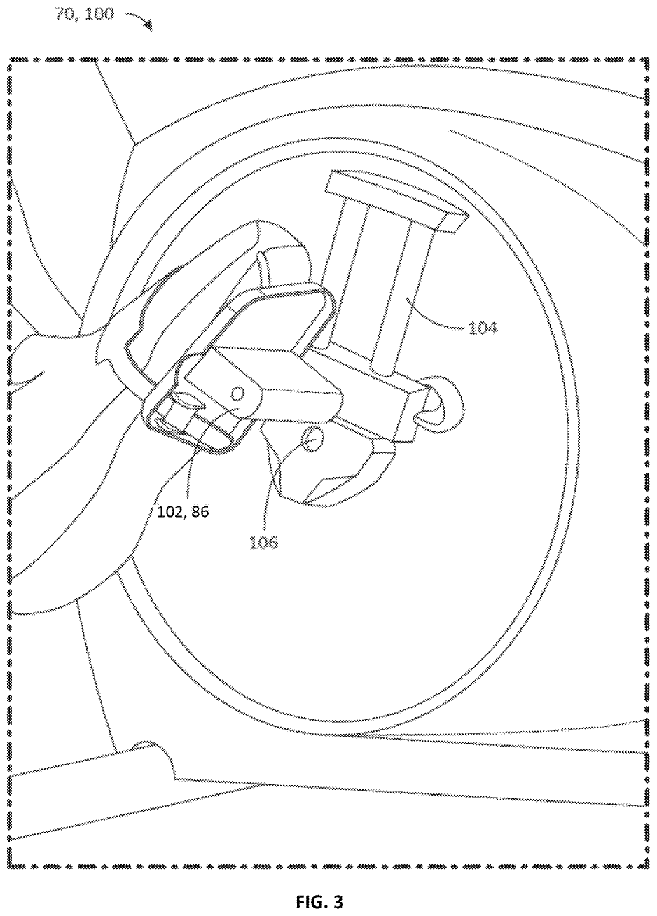

FIGS. 2-3 show an embodiment of a treatment apparatus 70. More specifically, FIG. 2 shows a treatment apparatus 70 in the form of a stationary cycling machine 100, which may be called a stationary bike, for short. The stationary cycling machine 100 includes a set of pedals 102 each attached to a pedal arm 104 for rotation about an axle 106. In some embodiments, and as shown in FIG. 2, the pedals 102 are movable on the pedal arms 104 in order to adjust a range of motion used by the patient in pedaling. For example, the pedals being located inwardly toward the axle 106 corresponds to a smaller range of motion than when the pedals are located outwardly away from the axle 106. A pressure sensor 86 is attached to or embedded within one of the pedals 102 for measuring an amount of force applied by the patient on the pedal 102. The pressure sensor 86 may communicate wirelessly to the treatment apparatus 70 and/or to the patient interface 50.

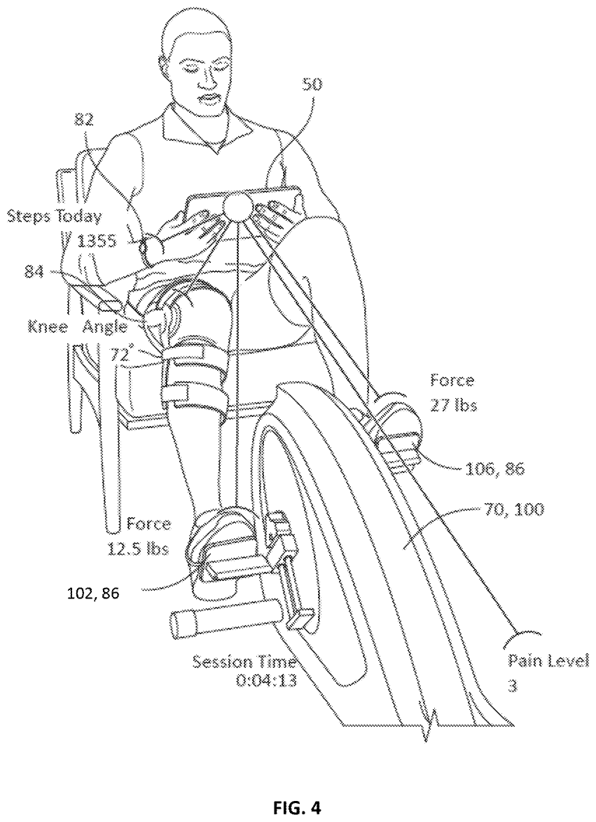

FIG. 4 shows a person (a patient) using the treatment apparatus of FIG. 2, and showing sensors and various data parameters connected to a patient interface 50. The example patient interface 50 may be a tablet computer or smartphone, or a phablet, such as an iPad, an iPhone, an Android device, a Surface tablet, or any other electronic device held manually by the patient. In some other embodiments, the patient interface 50 may be embedded within or attached to the treatment apparatus 70, obviating the need for the patient to hold the device manually, other than for the possible purpose of interacting with it. FIG. 4 shows the patient wearing the ambulation sensor 82 on his wrist, with a note showing “STEPS TODAY 1355”, indicating that the ambulation sensor 82 has recorded and transmitted that step count to the patient interface 50. FIG. 4 also shows the patient wearing the goniometer 84 on his right knee, with a note showing “KNEE ANGLE 72° “, indicating that the goniometer 84 is measuring and transmitting that knee angle to the patient interface 50. FIG. 4 also shows a right side of one of the pedals 102 with a pressure sensor 86 showing “FORCE 12.5 lbs.”, indicating that the right pedal pressure sensor 86 is measuring and transmitting that force measurement to the patient interface 50. FIG. 4 also shows a left side of one of the pedals 102 with a pressure sensor 86 showing “FORCE 27 lbs.”, indicating that the left pedal pressure sensor 86 is measuring and transmitting that force measurement to the patient interface 50. FIG. 4 also shows other patient data, such as an indicator of “SESSION TIME 0:04:13”, indicating that the patient has been using the treatment apparatus 70 for 4 minutes and 13 seconds. This session time may be determined by the patient interface 50 based on information received from the treatment apparatus 70. FIG. 4 also shows an indicator showing “PAIN LEVEL 3”. Such a pain level may be obtained from the patent in response to a solicitation or inquiry, such as a question, presented upon the patient interface 50.

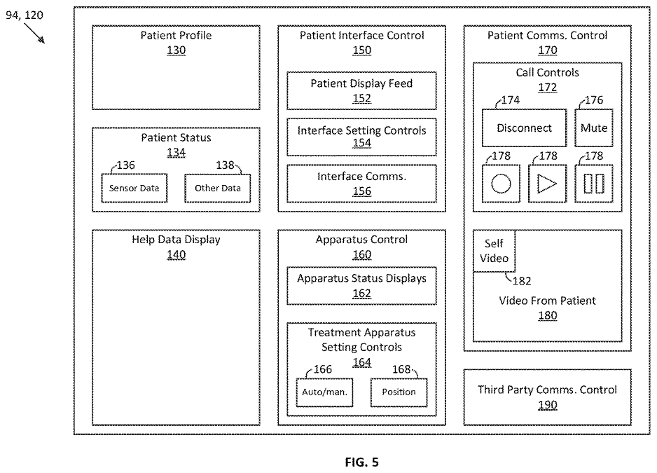

FIG. 5 is an example embodiment of an overview display 120 of the assistant interface 94. Specifically, the overview display 120 presents several different controls and interfaces for the assistant to remotely assist a patient with using the patient interface 50 and/or the treatment apparatus 70. This remote assistance functionality may comprise a type of functionality present in telemedicine systems.

Specifically, the overview display 120 includes a patient profile display 130 presenting biographical information regarding a patient using the treatment apparatus 70. The patient profile display 130 may take the form of a portion or region of the overview display 120, as shown in FIG. 5, although the patient profile display 130 may take other forms, such as a separate screen or a popup window. In some embodiments, the patient profile display 130 may include a limited subset of the patient's biographical information, health-related information, or both. More specifically, the data presented upon the patient profile display 130 may depend upon the assistant's need for that information. For example, a healthcare professional assisting the patient with a medical issue may be provided with medical history information regarding the patient, whereas a technician troubleshooting an issue with the treatment apparatus 70 may be provided with a much more limited set of information regarding the patient. The technician, for example, may be given only the patient's name. The patient profile display 130 may include pseudonymized data and/or anonymized data or use any privacy enhancing technology to prevent confidential patient data from being communicated in a way that could violate patient confidentiality requirements. Such privacy enhancing technologies may enable compliance with laws, regulations, or other rules of governance such as, but not limited to, the Health Insurance Portability and Accountability Act (HIPAA), or the General Data Protection Regulation (GDPR), wherein the patient may be deemed a “data subject.”

In some embodiments, the patient profile display 130 may present information regarding the treatment plan for the patient to follow in using the treatment apparatus 70. Such treatment plan information may be limited to an assistant who is a healthcare professional, such as a doctor or physical therapist. For example, a healthcare professional assisting the patient with an issue regarding the treatment regimen may be provided with treatment plan information, whereas a technician troubleshooting an issue with the treatment apparatus 70 may not be provided with any information regarding the patient's treatment plan.

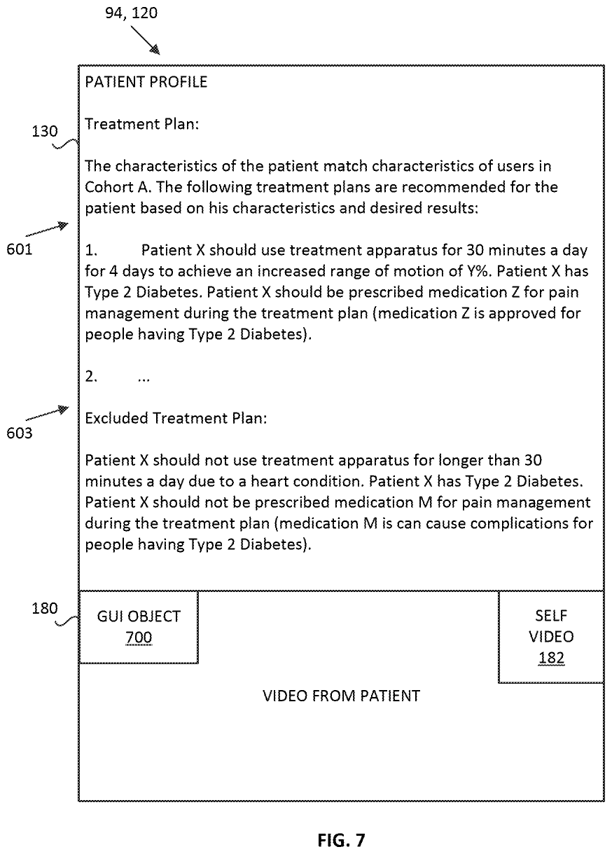

In some embodiments, one or more recommended treatment plans and/or excluded treatment plans may be presented in the patient profile display 130 to the assistant. The one or more recommended treatment plans and/or excluded treatment plans may be generated by the artificial intelligence engine 11 of the server 30 and received from the server 30 in real-time during, inter alia, a telemedicine session. An example of presenting the one or more recommended treatment plans and/or ruled-out treatment plans is described below with reference to FIG. 7.

The example overview display 120 shown in FIG. 5 also includes a patient status display 134 presenting status information regarding a patient using the treatment apparatus. The patient status display 134 may take the form of a portion or region of the overview display 120, as shown in FIG. 5, although the patient status display 134 may take other forms, such as a separate screen or a popup window. The patient status display 134 includes sensor data 136 from one or more of the external sensors 82, 84, 86, and/or from one or more internal sensors 76 of the treatment apparatus 70. In some embodiments, the patient status display 134 may present other data 138 regarding the patient, such as last reported pain level, or progress within a treatment plan.

User access controls may be used to limit access, including which data is available to be viewed and/or modified, on any or all of the user interfaces 20, 50, 90, 92, and 94 of the system 10. In some embodiments, user access controls may be employed to control which information is available to any given person, wherein the given person is using the system 10. For example, data presented on the assistant interface 94 may be controlled by user access controls, with permissions set depending on the assistant/user's need for and/or qualifications to view that information.

The example overview display 120 shown in FIG. 5 also includes a help data display 140 presenting information for the assistant to use in assisting the patient. The help data display 140 may take the form of a portion or region of the overview display 120, as shown in FIG. 5. The help data display 140 may take other forms, such as a separate screen or a popup window. The help data display 140 may include, for example, presenting answers to frequently asked questions regarding use of the patient interface 50 and/or the treatment apparatus 70. The help data display 140 may also include research data or best practices. In some embodiments, the help data display 140 may present scripts for answers or explanations in response to patient questions. In some embodiments, the help data display 140 may present flow charts or walk-throughs for the assistant to use in determining a root cause and/or solution to a patient's problem. In some embodiments, the assistant interface 94 may present two or more help data displays 140, which may be the same or different, for simultaneous presentation of help data for use by the assistant. For example, a first help data display may be used to present a troubleshooting flowchart to determine the source of a patient's problem, and a second help data display may present script information for the assistant to read to the patient, such information to preferably include directions for the patient to perform some action, which may help to narrow down or solve the problem. In some embodiments, based upon inputs to the troubleshooting flowchart in the first help data display, the second help data display may automatically populate with script information.

The example overview display 120 shown in FIG. 5 also includes a patient interface control 150 presenting information regarding the patient interface 50, and/or for modifying one or more settings of the patient interface 50. The patient interface control 150 may take the form of a portion or region of the overview display 120, as shown in FIG. 5. The patient interface control 150 may take other forms, such as a separate screen or a popup window. The patient interface control 150 may present information communicated to the assistant interface 94 via one or more of the interface monitor signals 98b. As shown in FIG. 5, the patient interface control 150 includes a display feed 152 of the display presented by the patient interface 50. In some embodiments, the display feed 152 may include a live copy of the display screen currently being presented to the patient by the patient interface 50. In other words, the display feed 152 may present an image of what is presented on a display screen of the patient interface 50. In some embodiments, the display feed 152 may include abbreviated information regarding the display screen currently being presented by the patient interface 50, such as a screen name or a screen number. The patient interface control 150 may include a patient interface setting control 154 for the assistant to adjust or to control one or more settings or aspects of the patient interface 50. In some embodiments, the patient interface setting control 154 may cause the assistant interface 94 to generate and/or to transmit an interface control signal 98 for controlling a function or a setting of the patient interface 50.

In some embodiments, the patient interface setting control 154 may include a collaborative browsing or co-browsing capability for the assistant to remotely view and/or control the patient interface 50. For example, the patient interface setting control 154 may enable the assistant to remotely enter text to one or more text entry fields on the patient interface 50 and/or to remotely control a cursor on the patient interface 50 using a mouse or touchscreen of the assistant interface 94.

In some embodiments, using the patient interface 50, the patient interface setting control 154 may allow the assistant to change a setting that cannot be changed by the patient. For example, the patient interface 50 may be precluded from enabling access to a language setting in order to prevent a patient from inadvertently switching, on the patient interface 50, the language used for the displays, whereas the patient interface setting control 154 may enable the assistant to change the language setting of the patient interface 50. In another example, the patient interface 50 may not be able to change a font size setting to a smaller size in order to prevent a patient from inadvertently switching the font size used for the displays on the patient interface 50 such that the display would become illegible or unintelligible to the patient, whereas the patient interface setting control 154 may provide for the assistant to change the font size setting of the patient interface 50.

The example overview display 120 shown in FIG. 5 also includes an interface communications display 156 showing the status of communications between the patient interface 50 and one or more other devices 70, 82, 84, such as the treatment apparatus 70, the ambulation sensor 82, and/or the goniometer 84. The interface communications display 156 may take the form of a portion or region of the overview display 120, as shown in FIG. 5. The interface communications display 156 may take other forms, such as a separate screen or a popup window. The interface communications display 156 may include controls for the assistant to remotely modify communications with one or more of the other devices 70, 82, 84. For example, the assistant may remotely command the patient interface 50 to reset communications with one of the other devices 70, 82, 84, or to establish communications with a new or replacement one of the other devices 70, 82, 84. This functionality may be used, for example, where the patient has a problem with one of the other devices 70, 82, 84, or where the patient receives a new or a replacement one of the other devices 70, 82, 84.

The example overview display 120 shown in FIG. 5 also includes an apparatus control 160 for the assistant to view and/or to control information regarding the treatment apparatus 70. The apparatus control 160 may take the form of a portion or region of the overview display 120, as shown in FIG. 5. The apparatus control 160 may take other forms, such as being enabled through or presented on a separate screen or a popup window. The apparatus control 160 may include an apparatus status display 162 with information regarding the current status of the apparatus. The apparatus status display 162 may present information communicated to the assistant interface 94 via one or more of the apparatus monitor signals 99b. The apparatus status display 162 may indicate whether the treatment apparatus 70 is currently communicating with the patient interface 50. The apparatus status display 162 may present other current and/or historical information regarding the status of the treatment apparatus 70.

The apparatus control 160 may include an apparatus setting control 164 for the assistant to adjust or control one or more aspects of the treatment apparatus 70. The apparatus setting control 164 may cause the assistant interface 94 to generate and/or to transmit an apparatus control signal 99 for changing an operating parameter of the treatment apparatus 70 (e.g., a pedal radius setting, a resistance setting, a target revolutions per minute (RPM), etc.). The apparatus setting control 164 may include a mode button 166 and a position control 168, which may be used in conjunction for the assistant to place an actuator 78 of the treatment apparatus 70 in a manual mode, after which a setting, such as a position or a speed of the actuator 78, can be changed using the position control 168. The mode button 166 may provide for a setting, such as a position, to be toggled between automatic and manual modes. In some embodiments, one or more settings may be adjustable at any time, but without a necessity of having an associated auto/manual mode. In some embodiments, the assistant may change an operating parameter of the treatment apparatus 70, such as a pedal radius setting, while the patient is actively using the treatment apparatus 70. Such “on the fly” adjustment may or may not be available to the patient using the patient interface 50. In some embodiments, the apparatus setting control 164 may allow the assistant to change a setting that cannot be changed by the patient using the patient interface 50. For example, the patient interface 50 may be precluded from changing a preconfigured setting, such as a height or a tilt setting of the treatment apparatus 70, whereas the apparatus setting control 164 may provide for the assistant to change the height or tilt setting of the treatment apparatus 70.

The example overview display 120 shown in FIG. 5 also includes a patient communications control 170 for controlling an audio or an audiovisual communications session with the patient interface 50. The communications session with the patient interface 50 may comprise a live feed from the assistant interface 94 for presentation on or by the output device of the patient interface 50. The live feed may take the form of an audio feed and/or a video feed. In some embodiments, the patient interface 50 may be configured to provide two-way audio or audiovisual communications with a person using the assistant interface 94. Specifically, the communications session with the patient interface 50 may include bidirectional (two-way) video or audiovisual feeds, with each of the patient interface 50 and the assistant interface 94 presenting video of the other one. In some embodiments, the patient interface 50 may present video from the assistant interface 94, while the assistant interface 94 presents only audio or the assistant interface 94 presents no live audio or visual signal from the patient interface 50. In some embodiments, the assistant interface 94 may present video from the patient interface 50, while the patient interface 50 presents only audio or the patient interface 50 presents no live audio or visual signal from the assistant interface 94.

In some embodiments, the audio or an audiovisual communications session with the patient interface 50 may take place, at least in part, while the patient is performing the rehabilitation regimen upon the body part. The patient communications control 170 may take the form of a portion or region of the overview display 120, as shown in FIG. 5. The patient communications control 170 may take other forms, such as being enabled by or on a separate screen or a popup window. The audio and/or audiovisual communications may be processed and/or directed by the assistant interface 94 and/or by another device or devices, such as a telephone system, or a videoconferencing system (e.g., Zoom, WebEx, etc.) used by the assistant while the assistant uses the assistant interface 94. Alternatively or additionally, the audio and/or audiovisual communications may include communications with a third party. For example, the system 10 may enable the assistant to initiate a 3-way conversation with the patient and a subject matter expert, such as a healthcare professional or a specialist, regarding use of a particular piece of hardware or software. The example patient communications control 170 shown in FIG. 5 includes call controls 172 for the assistant to use in managing various aspects of the audio or audiovisual communications with the patient. The call controls 172 include a disconnect button 174 for the assistant to end the audio or audiovisual communications session. The call controls 172 also include a mute button 176 to temporarily mute or attenuate an audio or audiovisual signal from the assistant interface 94. In some embodiments, the call controls 172 may include other features, such as a hold button (not shown). The call controls 172 may also include one or more record/playback controls 178, such as record, play, and pause buttons to control, with the patient interface 50, recording and/or playback of audio and/or video from the teleconference session. The call controls 172 may also include a video feed display 180 for presenting still and/or video images from the patient interface 50, and a self-video display 182 for showing the current image of the assistant using the assistant interface. The self-video display 182 may be presented as a picture-in-picture (PiP) format, such PiP format being within a section of the video feed display 180, as shown in FIG. 5. Alternatively or additionally, the self-video display 182 may be presented separately and/or independently from the video feed display 180.

The example overview display 120 shown in FIG. 5 also includes a third-party communications control 190 for use in conducting audio and/or audiovisual communications with a third party. The third-party communications control 190 may take the form of a portion or region of the overview display 120, as shown in FIG. 5. The third-party communications control 190 may take other forms, such as enabling or presenting on a display on a separate screen or a popup window. The third-party communications control 190 may include one or more controls, such as a contact list and/or buttons or controls to contact a third party regarding use of a particular piece of hardware or software, e.g., a subject matter expert, such as a healthcare professional or a specialist. The third-party communications control 190 may include a conference-calling capability for the third party to simultaneously communicate with both the assistant via the assistant interface 94, and the patient via the patient interface 50. For example, the system 10 may provide for the assistant to initiate a 3-way conversation with the patient and the third party.

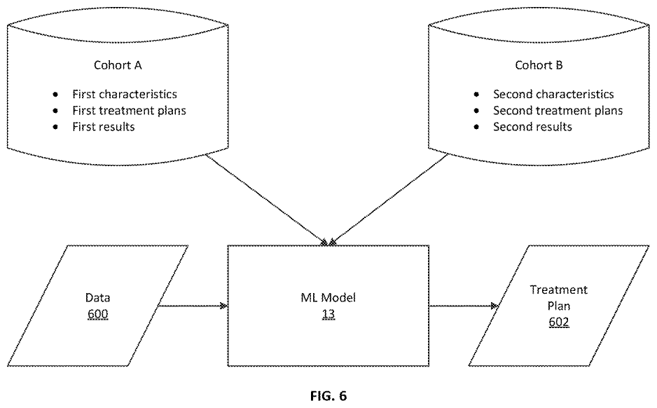

FIG. 6 shows an example block diagram of training a machine learning model 13 to output, based on data 600 pertaining to the patient, a treatment plan 602 for the patient according to the present disclosure. Data pertaining to other patients may be received by the server 30. The other patients may have used various treatment apparatuses to perform treatment plans. The data may include characteristics of the other patients, the details of the treatment plans performed by the other patients, and/or the results of performing the treatment plans (e.g., a percentage of recovery of a portion of the patients' bodies, an amount of recovery of a portion of the patients' bodies, an amount of increase or decrease in muscle strength of a portion of patients' bodies, an amount of increase or decrease in range of motion of a portion of patients' bodies, etc.).

As depicted in FIG. 6, the data has been assigned to different cohorts. Cohort A includes data for patients having similar first characteristics, first treatment plans, and first results. Cohort B includes data for patients having similar second characteristics, second treatment plans, and second results. For example, cohort A may include first characteristics of patients in their twenties without any medical conditions, and wherein such patients underwent surgery for a broken limb; their treatment plans may include a certain treatment protocol (e.g., use the treatment apparatus 70 for 30 minutes 5 times a week for 3 weeks, wherein values for the properties, configurations, and/or settings of the treatment apparatus 70 are set to X (where X is a numerical value) for the first two weeks and to Y (where Y is a numerical value) for the last week).

As further depicted in FIG. 6, Cohort A and cohort B may be included in a training dataset used to train the machine learning model 13. The machine learning model 13 may be trained to match a pattern between one or more characteristics for each cohort and output the treatment plan that provides the result, i.e., the best match. Accordingly, when the data 600 for a new patient is input into the trained machine learning model 13, the trained machine learning model 13 may match the one or more characteristics included in the data 600 with one or more characteristics in either cohort A or cohort B and output the appropriate treatment plan 602. In some embodiments, the machine learning model 13 may be trained to output one or more excluded treatment plans that should not be performed by the new patient.

FIG. 7 shows an embodiment of an overview display 120 of the assistant interface 94 presenting in real-time during a telemedicine session recommended treatment plans and excluded treatment plans according to the present disclosure. As depicted in FIG. 7, the overview display 120 just includes sections for the patient profile display 130 and the video feed display 180, including the self-video display 182. Any suitable configuration of controls and interfaces of the overview display 120 described with reference to FIG. 5 may be presented in addition to or instead of the patient profile display 130, the video feed display 180, and the self-video display 182.

As further depicted in FIG. 7, the assistant (e.g., healthcare professional) using the assistant interface 94 (e.g., computing device) during the telemedicine session may be presented in the self-video display 182 in a portion of the overview display 120 (e.g., user interface presented on a assistant display 24) that also presents a video from the patient in the video feed display 180. Further, the video feed display 180 may also include a graphical user interface (GUI) object 700 (e.g., a button) that enables the healthcare professional to share on the patient interface 50, in real-time or near real-time during the telemedicine session, the recommended treatment plans and/or the excluded treatment plans with the patient. The healthcare professional may select the GUI object 700 to share the recommended treatment plans and/or the excluded treatment plans. As depicted, another portion of the overview display 120 includes the patient profile display 130.

The patient profile display 130 illustrated in FIG. 7 presents two example recommended treatment plans 601 and one example excluded treatment plan 603. As described herein, the treatment plans may be recommended in view of characteristics of the patient being treated. To generate the recommended treatment plans 601 the patient should follow to achieve a desired result, a pattern between the characteristics of the patient being treated and a cohort of other patients who have used the treatment apparatus 70 to perform a treatment plan may be matched by one or more machine learning models 13 of the artificial intelligence engine 11. Each of the recommended treatment plans may be generated based on different desired results, i.e., different desired outcomes or best matches.

For example, as depicted in FIG. 7, the patient profile display 130 presents “The characteristics of the patient match characteristics of patients in Cohort A. The following treatment plans are recommended for the patient based on his characteristics and desired results.” Then, the patient profile display 130 presents recommended treatment plans from cohort A, and each treatment plan provides different results.

As depicted in FIG. 7, treatment plan “1” indicates “Patient X should use treatment apparatus for 30 minutes a day for 4 days to achieve an increased range of motion of Y %; Patient X has Type 2 Diabetes; and Patient X should be prescribed medication Z for pain management during the treatment plan (medication Z is approved for patients having Type 2 Diabetes).” Accordingly, the treatment plan generated achieves increasing the range of motion of Y %. As may be appreciated, the treatment plan also includes a recommended medication (e.g., medication Z) to prescribe to the patient to manage pain in view of a known medical disease (e.g., Type 2 Diabetes) of the patient. That is, the recommended patient medication not only does not conflict with the medical condition of the patient but thereby improves the probability of a superior patient outcome. This specific example and all such examples elsewhere herein are not intended to limit in any way the generated treatment plan from recommending multiple medications, or from handling the acknowledgement, view, diagnosis and/or treatment of comorbid conditions or diseases.

As illustrated in FIG. 7, recommended treatment plan “2” may specify, based on a different desired result of the treatment plan, a different treatment plan including a different treatment protocol for a treatment apparatus, a different medication regimen, etc.

As depicted in FIG. 7, the patient profile display 130 may also present the excluded treatment plans 603. These types of treatment plans are shown to the assistant using the assistant interface 94 to alert the assistant not to recommend certain portions of a treatment plan to the patient. For example, the excluded treatment plan 603 could specify the following: “Patient X should not use treatment apparatus for longer than 30 minutes a day due to a heart condition; Patient X has Type 2 Diabetes; and Patient X should not be prescribed medication M for pain management during the treatment plan (in this scenario, medication M can cause complications for patients having Type 2 Diabetes). Specifically, the excluded treatment plan 603 points out a limitation of a treatment protocol where, due to a heart condition, Patient X should not exercise for more than 30 minutes a day. The ruled-out treatment plan also points out that Patient X should not be prescribed medication M because it conflicts with the medical condition Type 2 Diabetes.

As further depicted in FIG. 7, the assistant may select the treatment plan for the patient on the overview display 120. For example, the assistant may use an input peripheral (e.g., mouse, touchscreen, microphone, keyboard, etc.) to select from the treatment plans 601 for the patient. In some embodiments, during the telemedicine session, the assistant may discuss the pros and cons of the recommended treatment plans 601 with the patient.

In any event, the assistant may select, as depicted in FIG. 7, the treatment plan for the patient to follow to achieve the desired result. The selected treatment plan may be transmitted to the patient interface 50 for presentation. The patient may view the selected treatment plan on the patient interface 50. In some embodiments, the assistant and the patient may discuss during the telemedicine session the details (e.g., treatment protocol using treatment apparatus 70, diet regimen, medication regimen, etc.) in real-time or in near real-time. In some embodiments, the server 30 may control, based on the selected treatment plan and during the telemedicine session, the treatment apparatus 70 as the user uses the treatment apparatus 70.

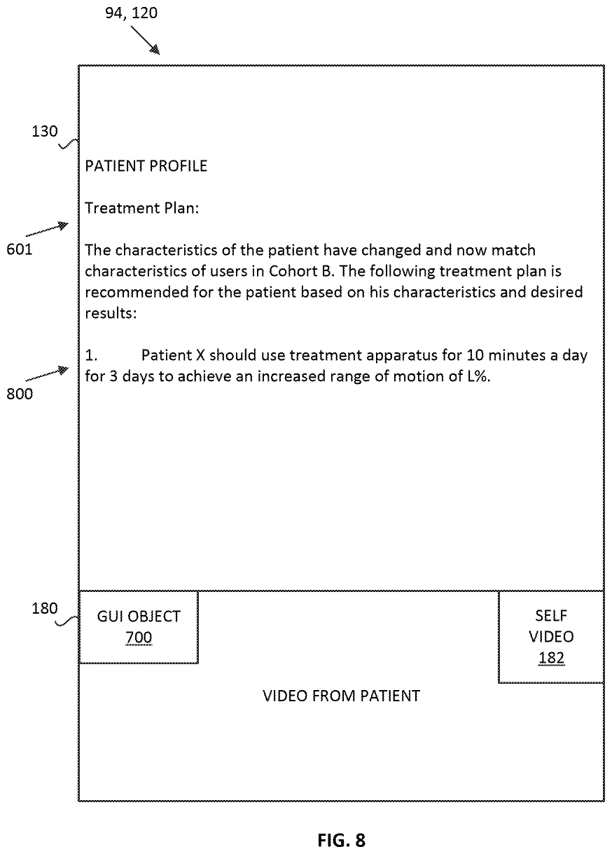

FIG. 8 shows an embodiment of the overview display 120 of the assistant interface 94 presenting, in real-time during a telemedicine session, recommended treatment plans that have changed due to patient data changing according to the present disclosure. As may be appreciated, the treatment apparatus 70 and/or any computing device (e.g., patient interface 50) may transmit data while the patient uses the treatment apparatus 70 to perform a treatment plan. The data may include updated characteristics of the patient. For example, the updated characteristics may include new performance information and/or measurement information related to the patient, the apparatus, the environment, etc. The performance information may include a speed of a portion of the treatment apparatus 70, a range of motion achieved by the patient, a force exerted on a portion of the treatment apparatus 70, a heartrate of the patient, a blood pressure of the patient, a respiratory rate of the patient, and so forth.

In one embodiment, the data received at the server 30 may be input into the trained machine learning model 13, which may determine that the characteristics indicate the patient is on track to achieve one or more goals associated with or part of the current treatment plan. Determining the patient is on track for the current treatment plan may cause the trained machine learning model 13 to adjust a parameter of the treatment apparatus 70. The adjustment may be based on a next step of the treatment plan to further improve the performance of the patient during that next step so as to more quickly achieve the one or more goals associated with or part of the current treatment plan or to surpass said one or more goals based on the adjustment.

In one embodiment, the data received at the server 30 may be input into the trained machine learning model 13, which may determine that the characteristics indicate the patient is not on track (e.g., behind schedule, not able to maintain a speed, not able to achieve a certain range of motion, is in too much pain, etc.) for the current treatment plan or is ahead of schedule (e.g., exceeding a certain speed, exercising longer than specified with no pain, exerting more than a specified force, etc.) for the current treatment plan. The trained machine learning model 13 may determine, due to the patient's not being on track or being ahead of schedule, that the characteristics of the patient no longer match the characteristics of the patients in the cohort to which the patient is assigned. Accordingly, the trained machine learning model 13 may reassign the patient to another cohort that includes as qualifying characteristics the patient's then-current characteristics. As such, the trained machine learning model 13 may select a new treatment plan from the new cohort and control, based on the new treatment plan, the treatment apparatus 70. In some embodiments, the trained machine learning model 13 may directly control the treatment apparatus 70 based on the new treatment plan. In other embodiments, the trained machine learning model 13 may control the treatment apparatus 70 based on the new treatment plan by updating one or more programs being executed on the treatment apparatus 70 itself.

In some embodiments, prior to controlling the treatment apparatus 70, the server 30 may provide the new treatment plan 800 to the assistant interface 94 for presentation in the patient profile display 130. As depicted in FIG. 8, the patient profile display 130 indicates “The characteristics of the patient have changed and now match characteristics of patients in Cohort B. The following treatment plan is recommended for the patient based on his characteristics and desired results.” Then, the patient profile display 130 presents the new treatment plan 800 (“Patient X should use treatment apparatus for 10 minutes a day for 3 days to achieve an increased range of motion of L %”). The assistant (healthcare professional) may select the new treatment plan 800, and the server 30 may receive the selection. The server 30 may control the treatment apparatus 70 based on the new treatment plan 800. In some embodiments, the new treatment plan 800 may be transmitted to the patient interface 50 such that the patient may view the details of the new treatment plan 800.

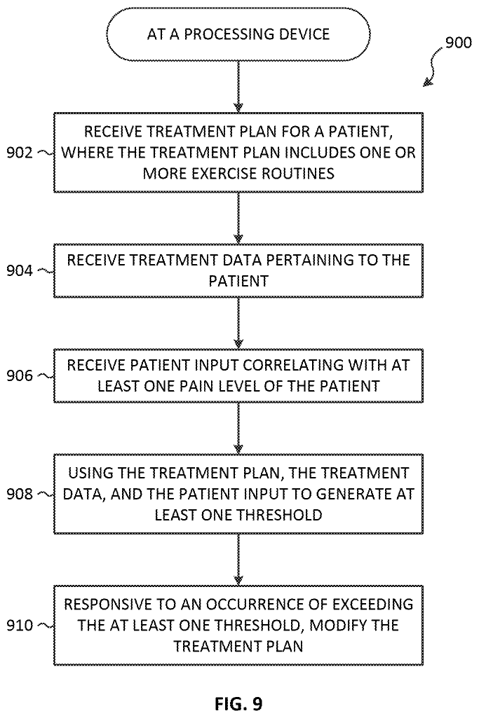

FIG. 9 shows an example embodiment of a method 900 for modifying, by an artificial intelligence engine, a treatment plan for optimizing patient outcome and pain levels during one or more treatment sessions according to the present disclosure. The method 900 is performed by processing logic that may include hardware (circuitry, dedicated logic, etc.), software (such as is run on a general-purpose computer system or a dedicated machine), or a combination of both. The method 900 and/or each of its individual functions, routines, subroutines, methods (as the term is used in object-oriented programming), or operations may be performed by one or more processors of a computing device (e.g., any component of FIG. 1, such as server 30 executing the artificial intelligence engine 11). In certain implementations, the method 900 may be performed by a single processing thread. Alternatively, the method 900 may be performed by two or more processing threads, each thread implementing one or more individual functions, routines, subroutines, or operations of the methods.

For simplicity of explanation, the method 900 is depicted in FIG. 9 and described as a series of operations. However, operations in accordance with this disclosure can occur in various orders and/or concurrently, and/or with other operations not presented and described herein. For example, the operations depicted in the method 900 in FIG. 9 may occur in combination with any other operation of any other method disclosed herein. Furthermore, not all illustrated operations may be required to implement the method 900 in accordance with the disclosed subject matter. In addition, those skilled in the art will understand and appreciate that the method 900 could alternatively be represented as a series of interrelated states via a state diagram or event diagram.

At block 902, the processing device may receive a treatment plan for a patient. The treatment plan may include one or more exercise routines. In some embodiments, the treatment plane may include one or more exercise routines for the patient to perform on a treatment device or treatment apparatus (e.g. on treatment apparatus 70).

At block 904, the processing device may receive treatment data pertaining to the patient. Treatment data may include, for example, characteristics of the patient, measurement information pertaining to the patient while the patient uses the treatment device, characteristics of the treatment device, the treatment plan, or a combination thereof. Characteristics of the patient may include, for example, age, health history, fitness level, etc. The measurement information may include, for example, one or more vital signs of the user, a respiration rate of the user, a heartrate of the user, a temperature of the user, a blood pressure of the user, other suitable measurement information of the user, or a combination thereof. Various characteristics of the treatment device may include, for example, one or more settings of the treatment device, a current revolutions per minute of a rotating member (e.g., such as a wheel) of the treatment device, a resistance setting of the treatment device, an angular or rotational velocity of the treatment device or components thereof, other suitable characteristics of the treatment device, or a combination thereof.

At block 906, the processing device may receive patient input correlating with at least one pain level of the patient. Patient input may also include, for example, a patient goal, an exhaustion level, etc. The patient may have different pain levels for different parts of the patient's body (e.g., a double knee replacement wherein one knee is recovering faster than the other).

At block 908, the processing device may use the treatment plan, the treatment data, and the patient input to generate at least one threshold. For example, the processing device may generate one or more thresholds described previously herein.

At block 910, responsive to an occurrence (e.g., detecting an occurrence) of exceeding the at least one threshold, the treatment plan may be modified. For example, in some embodiments, at least one updated exercise routine during one of the one or more treatment sessions is generated. Further, the treatment plan may be modified according to one or more of the examples described previously herein.

In some embodiments, the processing device may control, based on the modified treatment plan, the treatment device while the patient uses the treatment device (e.g., during a telemedicine session). For example, the processing device may cause the treatment device to modify at least one of a volume, a pressure, a resistance, an angle, an angular or rotational velocity, a speed, and a time period. A patient's use of a treatment device may include uses in a clinician's office, in a physical therapy center, in a gym, in a home office, at home, in an exercise or other workout studio, or any of the foregoing when directed by a clinician or person distal to the location of the treatment device, such as when the direction is for the purposes of telemedicine.

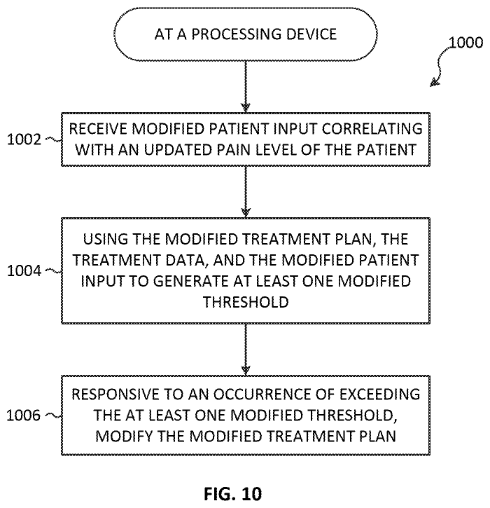

FIG. 10 shows an example embodiment of a method 1000 for further modifying the treatment plan, by an artificial intelligence engine, using one or more updated pain levels of the patient according to the present disclosure. The method 1000 includes operations performed by processors of a computing device (e.g., any component of FIG. 1, such as server 30 executing the artificial intelligence engine 11). In some embodiments, one or more operations of the method 1000 are implemented in computer instructions stored on a memory device and executed by a processing device. The method 1000 may be performed in the same or a similar manner as described above in regard to method 900. The operations of the method 1000 may be performed in some combination with any of the operations of any of the methods described herein. In some embodiments, the method 1000 may occur after block 910 in the method 900 depicted in FIG. 9. That is, the method 1000 may occur after the treatment plan is modified responsive to an occurrence of exceeding the determined threshold.

Regarding the method 1000, at block 1002, the processing device may receive modified patient input correlating with an updated pain level of the patient. At block 1004, the processing device may use the modified treatment plan, the treatment data, and the modified patient input to generate at least one modified threshold. At block 1006, responsive to an occurrence of exceeding the at least one modified threshold, the modified treatment plan may be modified (e.g., further modified). In some embodiments, the difficulty of the treatment plan may be changed based on the patient's pain level. For example, if the pain level is low or nonexistent, then the difficulty of the workout may be increased for the patient to recover faster. However, if there is too much pain, then the difficulty of the workout may be decreased to decrease the patient's pain level during the exercise session.

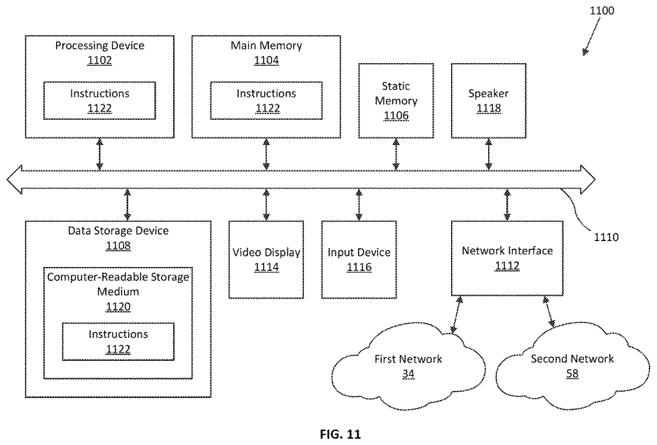

FIG. 11 shows an example computer system 1100 which can perform any one or more of the methods described herein, in accordance with one or more aspects of the present disclosure. In one example, computer system 1100 may include a computing device and correspond to one or more of the assistance interface 94, reporting interface 92, supervisory interface 90, clinician interface 20, server 30 (including the AI engine 11), patient interface 50, ambulation sensor 82, goniometer 84, treatment apparatus 70, pressure sensor 86, or any suitable component of FIG. 1. The computer system 1100 may be capable of executing instructions implementing the one or more machine learning models 13 of the artificial intelligence engine 11 of FIG. 1. The computer system may be connected (e.g., networked) to other computer systems in a LAN, an intranet, an extranet, or the Internet, including via the cloud or a peer-to-peer network. The computer system may operate in the capacity of a server in a client-server network environment. The computer system may be a personal computer (PC), a tablet computer, a wearable (e.g., wristband), a set-top box (STB), a personal Digital Assistant (PDA), a mobile phone, a smartphone, a camera, a video camera, an Internet of Things (IOT) device, or any device capable of executing a set of instructions (sequential or otherwise) that specify actions to be taken by that device. Further, while only a single computer system is illustrated, the term “computer” shall also be taken to include any collection of computers that individually or jointly execute a set (or multiple sets) of instructions to perform any one or more of the methods discussed herein.

The computer system 1100 (one example of a “computing device”) includes a processing device 1102, a main memory 1104 (e.g., read-only memory (ROM), flash memory, solid state drives (SSDs), dynamic random access memory (DRAM) such as synchronous DRAM (SDRAM)), a static memory 1106 (e.g., flash memory, solid state drives (SSDs), static random access memory (SRAM)), and a data storage device 1108, which communicate with each other via a bus 1110.

Processing device 1102 represents one or more general-purpose processing devices such as a microprocessor, central processing unit, or the like. More particularly, the processing device 1102 may be a complex instruction set computing (CISC) microprocessor, reduced instruction set computing (RISC) microprocessor, very long instruction word (VLIW) microprocessor, or a processor implementing other instruction sets or processors implementing a combination of instruction sets. The processing device 1102 may also be one or more special-purpose processing devices such as an application specific integrated circuit (ASIC), a system on a chip, a field programmable gate array (FPGA), a digital signal processor (DSP), network processor, or the like. The processing device 1102 may be configured to execute instructions for performing any of the operations and steps discussed herein.

The computer system 1100 may further include a network interface device 1112. The computer system 1100 also may include a video display 1114 (e.g., a liquid crystal display (LCD), a light-emitting diode (LED), an organic light-emitting diode (OLED), a quantum LED, a cathode ray tube (CRT), a shadow mask CRT, an aperture grille CRT, a monochrome CRT), one or more input devices 1116 (e.g., a keyboard and/or a mouse or a gaming-like control), and one or more speakers 1118 (e.g., a speaker). In one illustrative example, the video display 1114 and the input device(s) 1116 may be combined into a single component or device (e.g., an LCD touch screen).

The data storage device 1108 may include a computer-readable storage medium 1120 on which the instructions 1122 embodying any one or more of the methods, operations, or functions described herein is stored. The instructions 1122 may also reside, completely or at least partially, within the main memory 1104 and/or within the processing device 1102 during execution thereof by the computer system 1100. As such, the main memory 1104 and the processing device 1102 also constitute computer-readable media. The instructions 1122 may further be transmitted or received over a network via the network interface device 1112.

While the computer-readable storage medium 1120 is shown in the illustrative examples to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any medium capable of storing, encoding or carrying out a set of instructions for execution by the machine and that cause the machine to perform any one or more of the methodologies of the present disclosure. The term “computer-readable storage medium” shall accordingly be taken to include, but not be limited to, solid-state memories, optical media, and magnetic media.

Systems and Methods for Optimizing Treatment Plans to Support User Progression During Rehabilitation for the Purpose of Assisting in Determining AI-Driven Interventions by Using Machine Learning to Generate at Least One Data Signature Associated with a Comorbidity

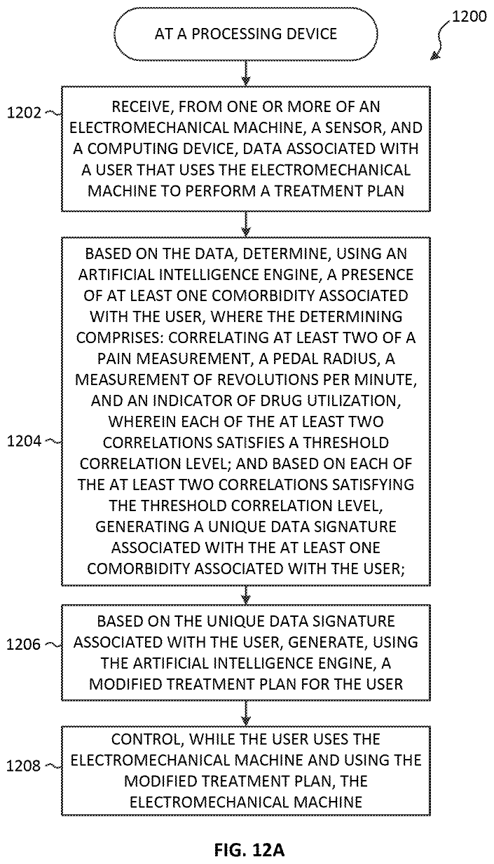

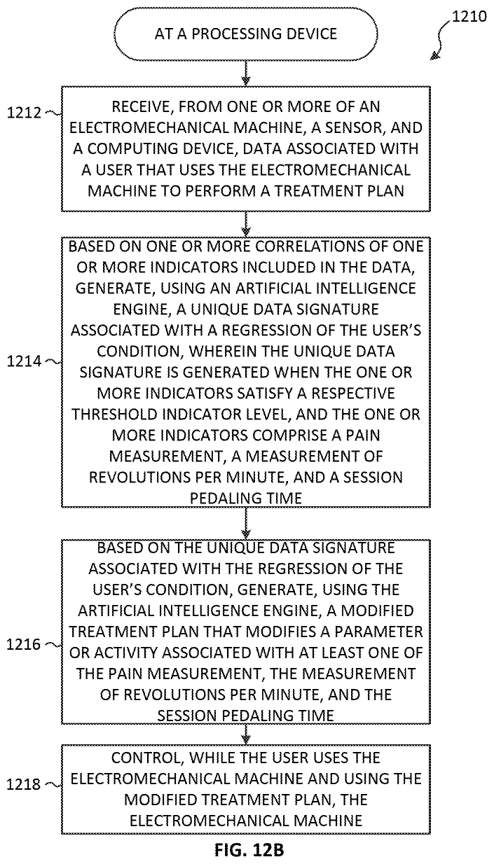

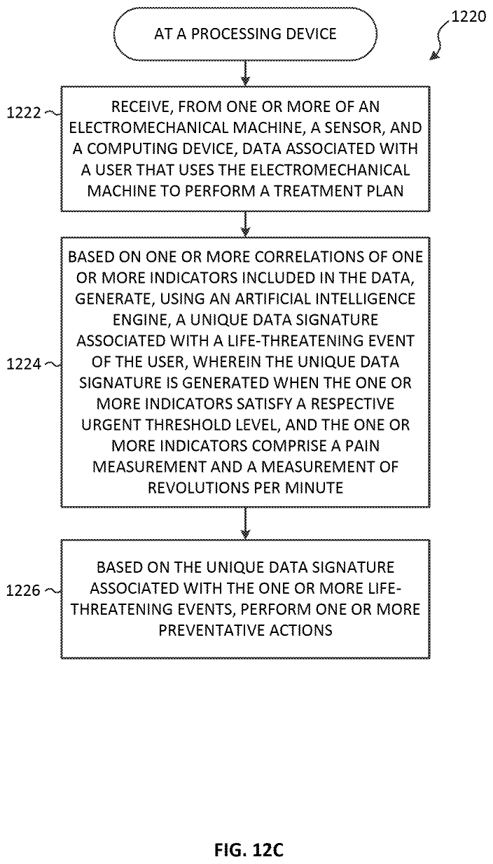

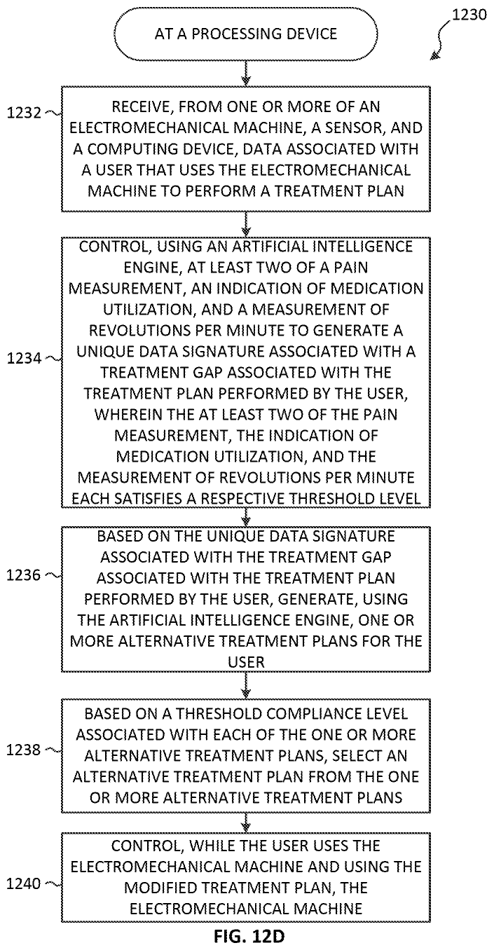

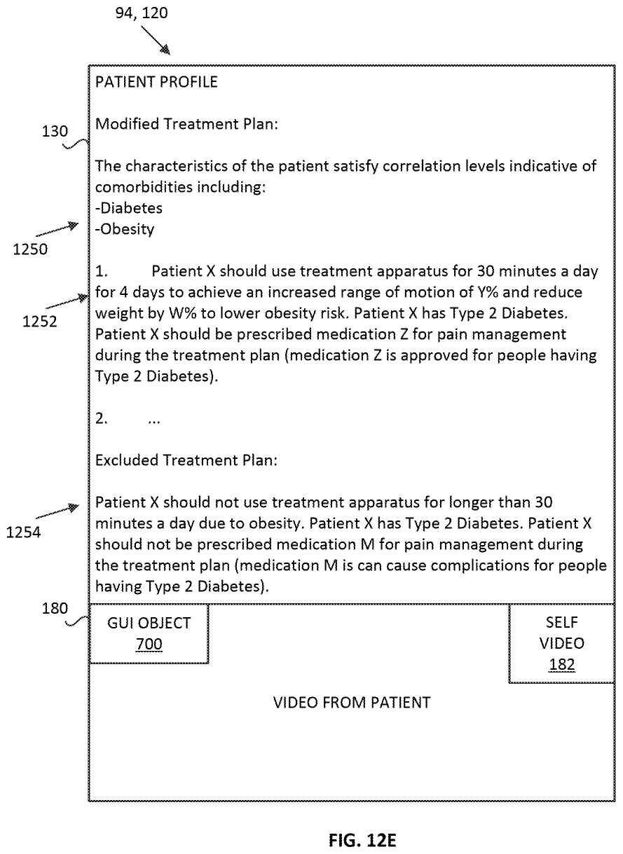

FIG. 12A shows an example embodiment of a method 1200 for optimizing treatment plans to support user progression during rehabilitation for the purpose of assisting in determining artificial intelligence driver interventions by using machine learning to generate at least one data signature associated with a comorbidity. The method 1200 is performed by processing logic that may include hardware (circuitry, dedicated logic, etc.), software (such as is run on a general-purpose computer system or a dedicated machine), or a combination of both. The method 1200 and/or each of its individual functions, routines, subroutines, methods (as the term is used in object-oriented programming), or operations may be performed by one or more processing devices of a computing device (e.g., any component of FIG. 1, such as assistant interface 94, patient interface 50, and/or server 30 executing the artificial intelligence engine 11, training engine 9, machine learning models 13, etc.) implementing the method 1200. The method 1200 may be implemented as computer instructions stored on a memory device and able to be executed by the one or more processing devices. In certain implementations, the method 1200 may be performed by a single processing thread. Alternatively, the method 1200 may be performed by two or more processing threads, each thread implementing one or more individual functions, routines, subroutines, or operations of the methods. In some embodiments, one or more accelerators may be used to increase the performance of a processing device by offloading various functions, routines, subroutines, or operations from the processing device.

For simplicity of explanation, the method 1200 is depicted in FIG. 12A and described as a series of operations. However, operations in accordance with this disclosure can occur in various orders and/or concurrently, and/or with other operations not presented and described herein. For example, the operations depicted in the method 1200 in FIG. 12A may occur in combination with any other operation of any other method disclosed herein. Furthermore, not all illustrated operations may be required to implement the method 1200 in accordance with the disclosed subject matter. In addition, those skilled in the art will understand and appreciate that the method 1200 could alternatively be represented as a series of interrelated states via a state diagram or event diagram.

In some embodiments, one or more computer-implemented models (e.g., machine learning models, neural networks, expert systems, etc.) may be generated and trained by the artificial intelligence engine 11 and/or the training engine 9 to perform one or more of the operations of the methods described herein. For example, to perform the one or more operations, the processing device may execute the one or more computer-implemented models. In some embodiments, the one or more computer-implemented models may be iteratively retrained to select different features capable of enabling optimization of output. The features that may be modified may include a number of nodes included in each layer of the models, an objective function executed at each node, a number of layers, various weights associated with outputs of each node, and the like. The objective function (e.g., a Mean Squared Error (MSE)) may be a function that maps model parameters and data to a numerical value representing the model's performance. In some embodiments, the objective function may be used during training machine learning executing optimization algorithms to adjust model parameters iteratively until they achieve a minimum or maximum value, indicating the best possible model performance. The term “objective function,” “loss function,” and “cost function” may be used interchangeably herein.

According to the method illustrated in FIG. 12A, at step 1202, the processing device receives, from one or more of an electromechanical machine, a sensor, and a computing device, data associated with a user that uses the electromechanical machine to perform a treatment plan. In some embodiments, the data may include performance data such as objective measurements (e.g., pedal radius, pedal force, heart rate, perspiration rate, blood oxygen level, activity duration, range of motion, speed, revolutions per minute, electromechanical machine component temperature, eye movement, gait data, etc.) as well as qualitative data (e.g., user-reported pain levels, fatigue, mood, etc.). The sensor may include an ambulation sensor, pressure sensor, temperature sensor, accelerometer, strain gauge, microphone, haptic sensor, etc.

At step 1204, the processing device, based on the data, determines, using an artificial intelligence engine, a presence of at least one comorbidity associated with the user. In some embodiments, determining a presence of the at least one comorbidity associated with the user may include the processing device correlating at least two of a pain measurement, a pedal radius, a measurement of revolutions per minute, and an indicator of drug utilization (e.g., where each of the at least two correlations satisfies a threshold correlation level). In some embodiments, the artificial intelligence engine may execute one or more computer-implemented models (e.g., machine learning models 13) implemented in computer instructions stored on one or more memory devices and executed by one or more processing devices. The one or more computer-implemented models may be trained using training data. In some embodiments, the computer-implemented models may be trained to predict the at least one comorbidity of the user. In some embodiments, the training data may include training inputs (e.g., pain measurements, pedal radii, measurements of revolutions per minute, indicators of drug utilization, etc.) associated with other users and/or electromechanical machines and training outputs (e.g., probabilities or likelihoods of certain comorbidity or comorbidities) associated with other users.

In some embodiments, the computer-implemented models may process input data and perform calculations based on learned or trained parameters. The calculations may generate scores and/or confidence values for each possible outcome, category, class, result, etc. The computer-implemented models may be trained to apply one or more mathematical functions (e.g., sigmoid, softmax, calibration method, etc.) to the scores or confidence values to convert and/or transform them into one or more values (e.g., between 0 and 1) that represent the probability of a specific prediction (e.g., having a certain comorbidity).

In some embodiments, for example, the computer-implemented models may perform logistic regression using a sigmoid function to map a linear combination of input features (e.g., pain measurements, pedal radii, measurements of revolutions per minute, indicators of drug utilization, etc.) to a value between 0 and 1, representing the probability of belonging to a specific class (e.g., comorbidity). In some embodiments, the computer-implemented models may include naïve Bayes probabilistic models trained to determine probabilities of a class (e.g., comorbidities) given observed features. In some embodiments, the computer-implemented models may be trained to perform generative artificial intelligence techniques, such as executing a generative adversarial network (GAN) and/or a variational autoencoder. A GAN may execute two neural networks. The two neural networks, a generator and a discriminator, may be trained simultaneously. The discriminator may receive an input and then output a scalar indicating whether a comorbidity is an actual and/or viable comorbidity. In some embodiments, the discriminator may resemble an energy function that outputs a low value (e.g., close to 0) when input is a valid comorbidity and a positive value when the input is not a valid comorbidity (e.g., if it is not based on certain symptoms or characteristics associated with a user).

There are two functions that may be used, the generator function (G(V)), and the discriminator function (D(Y)). The generator function may be denoted as G (V), where V is generally a vector randomly sampled in a standard distribution (e.g., Gaussian). The vector may be any suitable dimension and may be referred to as an embedding herein. The role of the generator is to produce comorbidities to train the discriminator function (D(Y)) to output the values indicating the comorbidity is valid (e.g., a low value). The discriminator function may be denoted as D (Y) where Y generally represents the true or target label that the model is trying to predict. The discriminator's goal is to correctly classify an input as real or fake and the Y variable provides the ground truth for this classification task.

During training, the discriminator is presented with a valid comorbidity and adjusts its parameters (e.g., weights and biases) to output a value indicative of the validity of the comorbidities that associated with real symptoms and/or characteristics of a real user. Next, the discriminator may receive a different comorbidity (e.g., modified using counterfactuals) generated by the generator and adjust its parameters to output a value indicative of whether the different comorbidity is associated with the real symptoms and/or characteristics of the real user.

The discriminator may use a gradient of an objective function to increase the value of the output. The discriminator may be trained as an unsupervised “density estimator,” i.e., a contrast function produces a low value for desired data and higher output for undesired data. The generator may receive the gradient of the discriminator with respect to each comorbidity it produces for users' symptoms and/or characteristics. The generator may use the gradient to train itself to produce comorbidities that the discriminator determines symptoms and/or characteristics associated with a user.

Recurrent neural networks include the functionality, in the context of a hidden layer, to process information sequences and store information about previous computations. As such, recurrent neural networks may have or exhibit a “memory.” Recurrent neural networks may include connections between nodes that form a directed graph along a temporal sequence. Keeping and analyzing information about previous states enables recurrent neural networks to process sequences of inputs to recognize patterns (e.g., such as user symptoms and/or user characteristics and correlations with certain comorbidities). Recurrent neural networks may be similar to Markov chains. For example, Markov chains may refer to stochastic models describing sequences of possible events in which the probability of any given event depends only on the state information contained in the previous event. Thus, Markov chains also use an internal memory to store at least the state of the previous event. These models may be useful in determining causal inference, such as whether an event at a current node changes as a result of the state of a previous node changing.

Based on each of the at least two correlations satisfying the threshold correlation level, the processing device may generate a unique data signature associated with the at least one comorbidity associated with the user. In some embodiments, the threshold correlation level may be a configured percentage of correlation level (e.g., 40 percent, 50 percent, 60 percent, 70 percent, 80 percent, etc.). For example, if at least two of a pain measurement, a pedal radius, a measurement of revolutions per minute, and an indicator of drug utilization satisfy the threshold correlation level of having at least 50 percent correlation level, then the processing device may generate a unique data signature for a certain comorbidity (e.g., high blood pressure, diabetes, obesity, chronic lung disease, kidney disease, heart disease, arthritis, cancer, mental health condition, etc.) or secondary condition/complication. The computer-implemented models may generate the unique data signature that represents one or more of the certain comorbidities based on which factors and/or how many factors satisfy the threshold correlation level.

At step 1206, the processing device, based on the unique data signature associated with the user, generates, using the artificial intelligence engine, a modified treatment plan for the user. In some embodiments, the modified treatment plan may include one or more modified exercises that specifies one or more operating parameters for various physical components of the electromechanical machine. For example, the operating parameters may include a position of a pedal to enable a certain range of motion. The operating parameter may cause one or more actuators, motors, pulleys, etc. of the electromechanical machine to operate to move the pedal to the position. The operating parameters may include a force and/or resistance provided by the pedal, such that an actuator of the electromechanical machine actuates to provide the amount of force and/or resistance by the pedal. The operating parameter may include a speed and/or revolutions per minute of a wheel and a motor of the electromechanical machine may be controlled to provide that speed and/or revolutions per minute. A goal of the modified treatment plan may be to tailored to optimize treatment for the comorbidity associated with the unique data signature associated with the user.

At step 1208, the processing device controls, while the user uses the electromechanical machine and using the modified treatment plan, the electromechanical machine. In some embodiments, controlling the electromechanical machine may include generating one or more control instructions that include the one or more operating parameters described herein. The processing device may transmit the one or more control instructions to a control system of the electromechanical machine and the control system may execute (e.g., using one or more processing devices) the control instructions to operate one or more physical components (e.g., actuator, pedal, motor, computing device, etc.).

In some embodiments, the method 1200 may generate the modified treatment plan by modifying a parameter or activity associated with the at least two of the pain measurement, the pedal radius, the measurement of revolutions per minute, and the indicator of drug utilization. In some embodiments, the computing device transmits, using an input peripheral associated with the computing device, the pain measurement input by the user. In some embodiments, the artificial intelligence engine uses one or more trained computer-implemented models to generate the unique data signature associated with the at least one comorbidity associated with the user. In some embodiments, the pain measurement comprises a pain level at onset of a treatment session, an average beginning pain for one or more sessions of the treatment plan, a pain level after a final session, an average pain after the one or more sessions, or some combination thereof.

In some embodiments, the pedal radius comprises a pedal radius of a final session of the treatment plan, an average pedal radius of one or more sessions of the treatment plan, or both. In some embodiments, the measurement of revolutions per minute comprises an average revolutions per minute associated with one or more sessions of the treatment plan. In some embodiments, the indicator of drug utilization comprises a medication utilization after a final session of the treatment plan, an average medication utilization for one or more sessions of the treatment plan, or both. In some embodiments, the at least one comorbidity comprises a secondary condition comprising hypertension, sepsis, a mental disorder, a mood disorder, diabetes, lung disease, obesity, heart disease, liver disease, kidney disease, vascular disease, arthritis, sleep apnea, osteoarthritis, anemia, stroke, asthma, any other physiological or anatomical disease, or some combination thereof. In some embodiments, the at least one comorbidity comprises a symptom comprising involvement or disorder of any physiological system in the user, any anatomical part of the user, any particular organ of the user, or some combination thereof. In some embodiments, the symptom is associated with the gastrointestinal system or the organ and wherein such symptoms comprise nausea, vomiting, diarrhea or any combination thereof. In some embodiments, the at least two correlations comprise one or more indicators of drug utilization.

Symptoms means as used herein: conditions manifested by any combination of the following signs or symptoms: constitutional findings (fatigue, malaise, fever, chills, night sweats, unexplained weight change); cutaneous or appendage alterations (rash, pruritus, hives, jaundice, pallor, cyanosis, petechiae, ulcers, alopecia, brittle nails, abnormal sweating or photosensitivity); head-and-neck or head-eyes-ears-nose-throat (HEENT) complaints (headache, visual disturbance, tinnitus, vertigo, nasal congestion, sore throat, dysphagia, cervical lymphadenopathy); respiratory manifestations (dry or productive cough, dyspnoea, wheeze, pleuritic chest pain); cardiovascular indicators (chest pain, palpitations, syncope, peripheral oedema, claudication); gastrointestinal and hepatobiliary symptoms (nausea, vomiting, abdominal pain, diarrhoea, constipation, melena, jaundice, dark urine); genitourinary findings (dysuria, hematuria, flank pain, menstrual irregularity, erectile dysfunction); endocrino-metabolic disturbances (polyuria, polydipsia, heat or cold intolerance, unexplained hypoglycaemia or hyperglycemia); neurological or psychiatric features (paresthesias, seizures, cognitive decline, insomnia, anxiety, depression, psychosis); musculoskeletal complaints (myalgia, arthralgia, morning stiffness, bone pain); haematologic-lymphatic signs (easy bruising, prolonged bleeding, lymphadenopathy, recurrent infections); immunologic or allergic reactions (anaphylaxis, angio-oedema, urticaria); and peripheral vascular or rheumatologic phenomena (Raynaud's, livedo reticularis, digital ulcers). By encompassing these diverse administration routes and symptomatologies, the present disclosure affords flexible dosing strategies and broad therapeutic applicability across a spectrum of infectious, inflammatory, metabolic, autoimmune, vascular, neurologic and oncologic disorders.

In some embodiments, a telemedicine session may be initiated between a computing device associated with the user and/or the electromechanical machine and a computing device associated with a medical professional. The telemedicine session may present information on a user interface of the computing device associated with the medical professional. For example, the user interface may present the comorbidity information 1250 (e.g., Diabetes, Obesity) determined using the artificial intelligence engine and the correlations identified in the data associated with the user, as shown in FIG. 12E.

As depicted, FIG. 12E shows an embodiment of an overview display 120 of the assistant interface 94 presenting in real-time during a telemedicine session a modified treatment plan and excluded treatment plans according to the present disclosure. As depicted in FIG. 12E, the overview display 120 just includes sections for the patient profile display 130 and the video feed display 180, including the self-video display 182. Any suitable configuration of controls and interfaces of the overview display 120 described with reference to FIG. 5 may be presented in addition to or instead of the patient profile display 130, the video feed display 180, and the self-video display 182.

As further depicted in FIG. 12E, the assistant (e.g., healthcare professional) using the assistant interface 94 (e.g., computing device) during the telemedicine session may be presented in the self-video display 182 in a portion of the overview display 120 (e.g., user interface presented on the assistant display 24) that also presents a video from the patient in the video feed display 180. Further, the video feed display 180 may also include a graphical user interface (GUI) object 700 (e.g., a button) that enables the healthcare professional to share on the patient interface 50, in real-time or near real-time during the telemedicine session, the recommended treatment plans and/or the excluded treatment plans with the patient. The healthcare professional may select the GUI object 700 to share the modified treatment plans and/or the excluded treatment plans. As depicted, another portion of the overview display 120 includes the patient profile display 130.

The patient profile display 130 illustrated in FIG. 12E presents two example recommended treatment plans 1252 and one example excluded treatment plan 1254. As described herein, the modified treatment plan may be generated based on the unique data signature associated with the user having the identified comorbidities. Each of the recommended treatment plans may be generated based on different desired results, i.e., different desired outcomes or best matches.

For example, as depicted in FIG. 12E, the patient profile display 130 presents “The characteristics of the patient satisfy correlation levels indicative of comorbidities-Diabetes,—Obesity.” Then, the patient profile display 130 presents modified treatment plans, and each treatment plan provides different results.

As depicted in FIG. 12E, treatment plan “1” indicates “Patient X should use treatment apparatus for 30 minutes a day for 4 days to achieve an increased range of motion of Y % and reduce weight by W % to lower obesity risk; Patient X has Type 2 Diabetes; and Patient X should be prescribed medication Z for pain management during the treatment plan (medication Z is approved for patients having Type 2 Diabetes).” Accordingly, the modified treatment plan generated achieves increasing the range of motion of Y %. As may be appreciated, the treatment plan also includes a recommended medication (e.g., medication Z) to prescribe to the patient to manage pain in view of a determined comorbidity (e.g., Type 2 Diabetes) of the patient. That is, the recommended patient medication not only does not conflict with the medical condition of the patient but thereby improves the probability of a superior patient outcome. This specific example and all such examples elsewhere herein are not intended to limit in any way the generated treatment plan from recommending multiple medications, or from handling the acknowledgement, view, diagnosis and/or treatment of comorbid conditions or diseases.

As illustrated in FIG. 12E, recommended treatment plan “2” may specify, based on a different desired result of the treatment plan, a different treatment plan including a different treatment protocol for a treatment apparatus, a different medication regimen, etc.

As depicted in FIG. 12E, the patient profile display 130 may also present the excluded treatment plans 1254. These types of treatment plans are shown to the assistant using the assistant interface 94 to alert the assistant not to recommend certain portions of a treatment plan to the patient. For example, the excluded treatment plan could specify the following: “Patient X should not use treatment apparatus for longer than 30 minutes a day due to obesity; Patient X has Type 2 Diabetes; and Patient X should not be prescribed medication M for pain management during the treatment plan (in this scenario, medication M can cause complications for patients having Type 2 Diabetes). Specifically, the excluded treatment plan points out a limitation of a treatment protocol where, due to a heart condition, Patient X should not exercise for more than 30 minutes a day. The ruled-out treatment plan also points out that Patient X should not be prescribed medication M because it conflicts with the medical condition Type 2 Diabetes.

As further depicted in FIG. 12E, the assistant may select the modified treatment plan for the patient on the overview display 120. For example, the assistant may use an input peripheral (e.g., mouse, touchscreen, microphone, keyboard, etc.) to select from the treatment plans 1252 for the patient. In some embodiments, during the telemedicine session, the assistant may discuss the pros and cons of the modified treatment plans 1252 with the patient.

In any event, the assistant may select, as depicted in FIG. 12E, the treatment plan for the patient to follow to achieve the desired result. The selected treatment plan may be transmitted to the patient interface 50 for presentation. The patient may view the selected treatment plan on the patient interface 50. In some embodiments, the assistant and the patient may discuss during the telemedicine session the details (e.g., treatment protocol using treatment apparatus 70, diet regimen, medication regimen, etc.) in real-time or in near real-time. In some embodiments, the server 30 may control, based on the selected treatment plan and during the telemedicine session, the treatment apparatus 70 as the user uses the treatment apparatus 70.

In some embodiments, the artificial intelligence engine may select the modified treatment plan automatically and begin controlling the treatment apparatus 70. The server 30 may cause the modified treatment plan to be presented or announced to the user via the patient interface 50 such that the user is made aware of the new operating parameters and details of the modified treatment plan. Further, the patient interface 50 may explain the reasons for the modified treatment plan being implemented, such as the user being diagnosed with one or more comorbidities.

As depicted in FIG. 12F, correlation data is depicted for a multitude (e.g., 40, 50, 60, 70, 80, 90, 100 users) that use the electromechanical machine to perform a treatment plan. As depicted, the correlation data is represented in a graph 1260 with “#Days”, “#Session”, “Onset Pain”, “Pain before final session”, “Average beginning pain, all sessions”, “Average beginning pain, all sessions”, “Med. Utilization”, “Pedal Radius”, “RPM”, “Average pedaling time, all sessions”, and “Pain Meas.” identifiers along the X and Y axes. The graph depicts that the cross-sections where a correlation is between 30%-39% is shaded a first darkness, correlations between 40%-69% is shaded a second darker darkness, and correlations greater than 70% are shaded a third darkest darkness in the graph. The comorbidities may be present when at least two correlations are identified between at least two of pain measurements, pedal radii, revolutions per minute measurements, and/or drug utilization (e.g., Med Utilization).

Consistent with the above disclosure, the examples of systems and methods enumerated in the following clauses are specifically contemplated and are intended as a non-limiting set of examples.
Clause 1.1 A computer-implemented method comprising: receiving, from one or more of an electromechanical machine, a sensor, and a computing device, data associated with a user that uses the electromechanical machine to perform a treatment plan; based on the data, determining, using an artificial intelligence engine, a presence of at least one comorbidity associated with the user, wherein the determining comprises: correlating at least two of a pain measurement, a pedal radius, a measurement of revolutions per minute, and an indicator of drug utilization, wherein each of the at least two correlations satisfies a threshold correlation level; based on each of the at least two correlations satisfying the threshold correlation level, generating a unique data signature associated with the at least one comorbidity associated with the user; based on the unique data signature associated with the user, generating, using the artificial intelligence engine, a modified treatment plan for the user; and controlling, while the user uses the electromechanical machine and using the modified treatment plan, the electromechanical machine.
Clause 2.1 The computer-implemented method of clause 1.1, further comprising generating the modified treatment plan by modifying a parameter or activity associated with the at least two of the pain measurement, the pedal radius, the measurement of revolutions per minute, and the indicator of drug utilization.
Clause 3.1 The computer-implemented method of clause 1.1, wherein the computing device transmits, using an input peripheral associated with the computing device, the pain measurement input by the user.
Clause 4.1 The computer-implemented method of clause 1.1, wherein the artificial intelligence engine uses one or more trained computer-implemented models to generate the unique data signature associated with the at least one comorbidity associated with the user.
Clause 5.1 The computer-implemented method of clause 1.1, wherein the pain measurement comprises a pain level at onset of a treatment session, an average beginning pain for one or more sessions of the treatment plan, a pain level after a final session, an average pain after the one or more sessions, or some combination thereof.
Clause 6.1 The computer-implemented method of clause 1.1, wherein the pedal radius comprises a pedal radius of a final session of the treatment plan, an average pedal radius of one or more sessions of the treatment plan, or both.
Clause 7.1 The computer-implemented method of clause 1.1, wherein the measurement of revolutions per minute comprises an average revolutions per minute associated with one or more sessions of the treatment plan.
Clause 8.1 The computer-implemented method of clause 1.1, wherein the indicator of drug utilization comprises a medication utilization after a final session of the treatment plan, an average medication utilization for one or more sessions of the treatment plan, or both.
Clause 9.1 The computer-implemented method of clause 1.1, wherein the at least one comorbidity comprises a secondary condition comprising hypertension, sepsis, a mental disorder, a mood disorder, diabetes, lung disease, obesity, heart disease, liver disease, kidney disease, vascular disease, arthritis, sleep apnea, osteoarthritis, anemia, stroke, asthma, any other physiological or anatomical disease, or some combination thereof.
Clause 10.1 The computer-implemented method of clause 1.1, wherein the at least one comorbidity comprises a symptom comprising involvement or disorder of any physiological system in the user, any anatomical part of the user, any particular organ of the user, or some combination thereof.
Clause 11.1 The computer-implemented method of clause 1.10, wherein the symptom is associated with the gastrointestinal system or the order and wherein such symptoms comprise nausea, vomiting, diarrhea or any combination thereof.
Clause 12.1 The computer-implemented method of clause 1.1, wherein the at least two correlations comprise one or more indicators of drug utilization.
Clause 13.1 A tangible, non-transitory computer-readable medium storing computer instructions that, when executed, cause one or more processing devices to:
   receive, from one or more of an electromechanical machine, a sensor, and a computing device, data associated with a user that uses the electromechanical machine to perform a treatment plan;
   based on the data, determine, using an artificial intelligence engine, a presence of at least one comorbidity associated with the user, wherein the determining comprises:
correlating at least two of a pain measurement, a pedal radius, a measurement of revolutions per minute, and an indicator of drug utilization, wherein each of the at least two correlations satisfies a threshold correlation level;
based on each of the at least two correlations satisfying the threshold correlation level, generating a unique data signature associated with the at least one comorbidity associated with the user;
   based on the unique data signature associated with the user, generate, using the artificial intelligence engine, a modified treatment plan for the user; and
   control, while the user uses the electromechanical machine and using the modified treatment plan, the electromechanical machine.
Clause 14.1 The computer-readable medium of clause 13.1, wherein the one or more processing devices generate the modified treatment plan by modifying a parameter or activity associated with the at least two of the pain measurement, the pedal radius, the measurement of revolutions per minute, and the indicator of drug utilization.
Clause 15.1 The computer-readable medium of clause 13.1, wherein the computing device transmits, using an input peripheral associated with the computing device, the pain measurement input by the user.
Clause 16.1 The computer-readable medium of clause 13.1, wherein the artificial intelligence engine uses one or more trained computer-implemented models to generate the unique data signature associated with the at least one comorbidity associated with the user.
Clause 17.1 The computer-readable medium of clause 13.1, wherein the pain measurement comprises a pain level at onset of a treatment session, an average beginning pain for one or more sessions of the treatment plan, a pain level after a final session, an average pain after the one or more sessions, or some combination thereof.
Clause 18.1 The computer-readable medium of clause 13.1, wherein the pedal radius comprises a pedal radius of a final session of the treatment plan, an average pedal radius of one or more sessions of the treatment plan, or both.
Clause 19.1 The computer-readable medium of clause 13.1, wherein the measurement of revolutions per minute comprises an average revolutions per minute associated with one or more sessions of the treatment plan.
Clause 20.1 A system comprising:
   one or more memory devices storing instructions; and
   one or more processing devices communicatively coupled to the one or more memory devices, wherein the one or more processing devices execute the instructions to:
receive, from one or more of an electromechanical machine, a sensor, and a computing device, data associated with a user that uses the electromechanical machine to perform a treatment plan;
   based on the data, determine, using an artificial intelligence engine, a presence of at least one comorbidity associated with the user, wherein the determining comprises:
correlating at least two of a pain measurement, a pedal radius, a measurement of revolutions per minute, and an indicator of drug utilization, wherein each of the at least two correlations satisfies a threshold correlation level;
   based on each of the at least two correlations satisfying the threshold correlation level, generating a unique data signature associated with the at least one comorbidity associated with the user;
   based on the unique data signature associated with the user, generate, using the artificial intelligence engine, a modified treatment plan for the user; and
   control, while the user uses the electromechanical machine and using the modified treatment plan, the electromechanical machine.
      Systems and Methods for Optimizing Treatment Plans to Support User Progression During Rehabilitation for the Purpose of Assisting in Determining AI-Driven Interventions by Using Machine Learning to Generate at Least One Data Signature Associated with a Regression of a User's Medical Condition

FIG. 12B shows an example embodiment of a method 1210 for optimizing treatment plans to support user progression during rehabilitation for the purpose of assisting in determining artificial intelligence driven interventions by using machine learning to generate at least one data signature associated with a regression of a user's medical condition. The method 1210 is performed by processing logic that may include hardware (circuitry, dedicated logic, etc.), software (such as is run on a general-purpose computer system or a dedicated machine), or a combination of both. The method 1210 and/or each of its individual functions, routines, subroutines, methods (as the term is used in object-oriented programming), or operations may be performed by one or more processing devices of a computing device (e.g., any component of FIG. 1, such as assistant interface 94, patient interface 50, and/or server 30 executing the artificial intelligence engine 11, training engine 9, machine learning models 13, etc.) implementing the method 1210. The method 1210 may be implemented as computer instructions stored on a memory device and able to be executed by the one or more processing devices. In certain implementations, the method 1210 may be performed by a single processing thread. Alternatively, the method 1210 may be performed by two or more processing threads, each thread implementing one or more individual functions, routines, subroutines, or operations of the methods. In some embodiments, one or more accelerators may be used to increase the performance of a processing device by offloading various functions, routines, subroutines, or operations from the processing device.

For simplicity of explanation, the method 1210 is depicted in FIG. 12B and described as a series of operations. However, operations in accordance with this disclosure can occur in various orders and/or concurrently, and/or with other operations not presented and described herein. For example, the operations depicted in the method 1210 in FIG. 12B may occur in combination with any other operation of any other method disclosed herein. Furthermore, not all illustrated operations may be required to implement the method 1210 in accordance with the disclosed subject matter. In addition, those skilled in the art will understand and appreciate that the method 1210 could alternatively be represented as a series of interrelated states via a state diagram or event diagram.

In some embodiments, one or more computer-implemented models (e.g., machine learning models, neural networks, expert systems, etc.) may be generated and trained by the artificial intelligence engine 11 and/or the training engine 9 to perform one or more of the operations of the methods described herein. For example, to perform the one or more operations, the processing device may execute the one or more computer-implemented models. In some embodiments, the one or more computer-implemented models may be iteratively retrained to select different features capable of enabling optimization of output. The features that may be modified may include a number of nodes included in each layer of the models, an objective function executed at each node, a number of layers, various weights associated with outputs of each node, and the like.

According to the method illustrated in FIG. 12B, at step 1212, the processing device receives, from one or more of an electromechanical machine, a sensor, and a computing device, data associated with a user that uses the electromechanical machine to perform a treatment plan.

At step 1214, the processing device, based on one or more correlations of one or more indicators included in the data, generates, using an artificial intelligence engine, a unique data signature associated with a regression of the user's condition. The unique data signature may be generated when the one or more indicators satisfy a respective threshold indicator level. The one or more indicators may include a pain measurement, a measurement of revolutions per minute, and a session pedaling time.

At step 1216, the processing device, based on the unique data signature associated with the regression of the user's condition, generates, using the artificial intelligence engine, a modified treatment plan that modifies a parameter or activity associated with at least one of the pain measurement, the measurement of revolutions per minute, and the session pedaling timer.

At step 1218, the processing device controls, while the user uses the electromechanical machine and using the modified treatment plan, the electromechanical machine.

In some embodiments, the method 1210, based on the unique data signature associated with the regression of the user's condition, generates the modified treatment plan may include an action of readmitting the user to a healthcare facility. In some embodiments, the computing device transmits, using an input peripheral of the computing device, the pain measurement that has been input by the user. In some embodiments, the artificial intelligence engine uses one or more trained computer-implemented models to generate the unique data signature associated with the regression of the user's condition. In some embodiments, the regression results in a hospital readmission. In some embodiments, the pain measurement comprises pain after a final session of the treatment plan, average pain after all of one or more sessions of the treatment plan, or some combination thereof. In some embodiments, the session pedaling time is determined based on an average of one or more sessions of the treatment plan. In some embodiments, the measurement of revolutions per minute comprises an average revolutions per minute associated with one or more sessions of the treatment plan. In some embodiments, the method 1210, based on the unique data signature associated with the regression of the user's condition, initiates a telehealth session between the computing device and a second computing device associated with a second user.

Consistent with the above disclosure, the examples of systems and methods enumerated in the following clauses are specifically contemplated and are intended as a non-limiting set of examples.
Clause 1.2 A computer-implemented method comprising: receiving, from one or more of an electromechanical machine, a sensor, and a computing device, data associated with a user that uses the electromechanical machine to perform a treatment plan; based on one or more correlations of one or more indicators included in the data, generating, using an artificial intelligence engine, a unique data signature associated with a regression of the user's condition, wherein the unique data signature is generated when the one or more indicators satisfy a respective threshold indicator level, and the one or more indicators comprise a pain measurement, a measurement of revolutions per minute, and a session pedaling time; based on the unique data signature associated with the regression of the user's condition, generating, using the artificial intelligence engine, a modified treatment plan that modifies a parameter or activity associated with at least one of the pain measurement, the measurement of revolutions per minute, and the session pedaling time; and controlling, while the user uses the electromechanical machine and using the modified treatment plan, the electromechanical machine.
Clause 2.2 The computer-implemented method of clause 1.2, wherein, based on the unique data signature associated with the regression of the user's condition, generating the modified treatment plan comprises an action of readmitting the user to a healthcare facility.
Clause 3.2 The computer-implemented method of clause 1.2, wherein the computing device transmits, using an input peripheral of the computing device, the pain measurement that has been input by the user.
Clause 4.2 The computer-implemented method of clause 1.2, wherein the artificial intelligence engine uses one or more trained computer-implemented models to generate the unique data signature associated with the regression of the user's condition.
Clause 5.2 The computer-implemented method of clause 1.2, wherein the regression results in a hospital readmission.
Clause 6.2 The computer-implemented method of clause 1.2, wherein the pain measurement comprises pain after a final session of the treatment plan, average pain after all of one or more sessions of the treatment plan, or some combination thereof.
Clause 7.2 The computer-implemented method of clause 1.2, wherein the session pedaling time is determined based on an average of one or more sessions of the treatment plan.
Clause 8.2 The computer-implemented method of clause 1.2, wherein the measurement of revolutions per minute comprises an average revolutions per minute associated with one or more sessions of the treatment plan.
Clause 9.2 The computer-implemented method of clause 1.2, further comprising, based on the unique data signature associated with the regression of the user's condition, initiating a telehealth session between the computing device and a second computing device associated with a second user.
Clause 10.2 A tangible, non-transitory computer-readable medium storing instructions that, when executed, cause one or more processing devices to:
   receive, from one or more of an electromechanical machine, a sensor, and a computing device, data associated with a user that uses the electromechanical machine to perform a treatment plan;
   based on one or more correlations of one or more indicators included in the data, generate, using an artificial intelligence engine, a unique data signature associated with a regression of the user's condition, wherein the unique data signature is generated when the one or more indicators satisfy a respective threshold indicator level, and the one or more indicators comprise a pain measurement, a measurement of revolutions per minute, and a session pedaling time;
   based on the unique data signature associated with the regression of the user's condition, generate, using the artificial intelligence engine, a modified treatment plan that modifies a parameter or activity associated with at least one of the pain measurement, the measurement of revolutions per minute, and the session pedaling time; and
   control, while the user uses the electromechanical machine and using the modified treatment plan, the electromechanical machine.
Clause 11.2 The computer-readable medium of clause 10.2, wherein, based on the unique data signature associated with the regression of the user's condition, generating the modified treatment plan comprises an action of readmitting the user to a healthcare facility.
Clause 12.2 The computer-readable medium of clause 10.2, wherein the computing device transmits, using an input peripheral of the computing device, the pain measurement that has been input by the user.
Clause 13.2 The computer-readable medium of clause 10.2, wherein the artificial intelligence engine uses one or more trained computer-implemented models to generate the unique data signature associated with the regression of the user's condition.
Clause 14.2 The computer-readable medium of clause 10.2, wherein the regression results in a hospital readmission.
Clause 15.2 The computer-readable medium of clause 10.2, wherein the pain measurement comprises pain after a final session of the treatment plan, average pain after all of one or more sessions of the treatment plan, or some combination thereof.
Clause 16.2 The computer-readable medium of clause 10.2, wherein the session pedaling time is determined based on an average of one or more sessions of the treatment plan.
Clause 17.2 The computer-readable medium of clause 10.2, wherein the measurement of revolutions per minute comprises an average revolutions per minute associated with one or more sessions of the treatment plan.
Clause 18.2 The computer-readable medium of clause 10.2, further comprising, based on the unique data signature associated with the regression of the user's condition, initiating a telehealth session between the computing device and a second computing device associated with a second use
Clause 19.2 A system comprising:
   one or more memory devices storing instructions; and
   one or more processing devices communicatively coupled to the one or more memory devices, wherein the one or more processing devices execute the instructions to:
   receive, from one or more of an electromechanical machine, a sensor, and a computing device, data associated with a user that uses the electromechanical machine to perform a treatment plan;
   based on one or more correlations of one or more indicators included in the data, generate, using an artificial intelligence engine, a unique data signature associated with a regression of the user's condition, wherein the unique data signature is generated when the one or more indicators satisfy a respective threshold indicator level, and the one or more indicators comprise a pain measurement, a measurement of revolutions per minute, and a session pedaling time;
   based on the unique data signature associated with the regression of the user's condition, generate, using the artificial intelligence engine, a modified treatment plan that modifies a parameter or activity associated with at least one of the pain measurement, the measurement of revolutions per minute, and the session pedaling time; and
   control, while the user uses the electromechanical machine and using the modified treatment plan, the electromechanical machine.
Clause 20.2 The system of clause 10.2, wherein, based on the unique data signature associated with the regression of the user's condition, generating the modified treatment plan comprises an action of readmitting the user to a healthcare facility.
   Systems and Methods for Optimizing Treatment Plans to Support User Progression During Rehabilitation for the Purpose of Assisting in Determining AI-Driven Interventions by Using Machine Learning to Generate at Least One Data Signature Associated with a Life-Threatening Event

FIG. 12C shows an example embodiment of a method 1220 for optimizing treatment plans to support user progression during rehabilitation for the purpose of assisting in determining artificial intelligence driver interventions by using machine learning to generate at least one data signature associated with a life-threatening event. The method 1220 is performed by processing logic that may include hardware (circuitry, dedicated logic, etc.), software (such as is run on a general-purpose computer system or a dedicated machine), or a combination of both. The method 1220 and/or each of its individual functions, routines, subroutines, methods (as the term is used in object-oriented programming), or operations may be performed by one or more processing devices of a computing device (e.g., any component of FIG. 1, such as assistant interface 94, patient interface 50, and/or server 30 executing the artificial intelligence engine 11, training engine 9, machine learning models 13, etc.) implementing the method 1220. The method 1220 may be implemented as computer instructions stored on a memory device and able to be executed by the one or more processing devices. In certain implementations, the method 1220 may be performed by a single processing thread. Alternatively, the method 1220 may be performed by two or more processing threads, each thread implementing one or more individual functions, routines, subroutines, or operations of the methods. In some embodiments, one or more accelerators may be used to increase the performance of a processing device by offloading various functions, routines, subroutines, or operations from the processing device.

For simplicity of explanation, the method 1220 is depicted in FIG. 12C and described as a series of operations. However, operations in accordance with this disclosure can occur in various orders and/or concurrently, and/or with other operations not presented and described herein. For example, the operations depicted in the method 1220 in FIG. 12C may occur in combination with any other operation of any other method disclosed herein. Furthermore, not all illustrated operations may be required to implement the method 1220 in accordance with the disclosed subject matter. In addition, those skilled in the art will understand and appreciate that the method 1220 could alternatively be represented as a series of interrelated states via a state diagram or event diagram.

In some embodiments, one or more computer-implemented models (e.g., machine learning models, neural networks, expert systems, etc.) may be generated and trained by the artificial intelligence engine 11 and/or the training engine 9 to perform one or more of the operations of the methods described herein. For example, to perform the one or more operations, the processing device may execute the one or more computer-implemented models. In some embodiments, the one or more computer-implemented models may be iteratively retrained to select different features capable of enabling optimization of output. The features that may be modified may include a number of nodes included in each layer of the models, an objective function executed at each node, a number of layers, various weights associated with outputs of each node, and the like.

According to the method illustrated in FIG. 12C, at step 1222, the processing device the processing device receives, from one or more of an electromechanical machine, a sensor, and a computing device, data associated with a user that uses the electromechanical machine to perform a treatment plan.

At step 1224, the processing device, based on one or more correlations of one or more indicators included in the data, generates, using an artificial intelligence engine, a unique data signature associated with a life-threatening event of the user. The unique data signature may be generated when the one or more indicators satisfy a respective urgent threshold level. The one or more indicators may include a pain measurement and a measurement of revolutions per minute.

At step 1226, the processing device, based on the unique data signature associated with the one or more life-threatening events, perform one or more preventative actions.

In some embodiments, the preventative actions are urgent. In some embodiments, the one or more preventative actions may include transmitting one or more messages to or through one or more emergency service systems, controlling operation of the electromechanical machine, transmitting one or more messages to the computing device, initiating a telehealth session between the computing device and a second computing device, or some combination thereof. In some embodiments, the one or more preventative actions may include generating, using the artificial intelligence engine, a modified treatment plan that modifies a parameter or activity associated with at least one of the pain measurement and the measurement of revolutions per minute, and controlling, while the user uses the electromechanical machine and using the modified treatment plan, the electromechanical machine. In some embodiments, the one or more life-threatening events may include, without limitation, deep-vein thrombosis, pulmonary embolism, myocardial infarction (heart attack), cardiac arrest, stroke (ischemic or hemorrhagic), subarachnoid hemorrhage, intracranial hemorrhage, cerebral edema, status epilepticus, severe traumatic brain injury, spinal-cord injury, any heart arrhythmia including atrial fibrillation, supraventricular tachycardia, ventricular tachycardia, ventricular fibrillation, tachycardia, bradycardia, torsades de pointes, congestive heart failure, cardiomyopathy, myocarditis, pericarditis, pericardial tamponade, heart-valve disease with acute decompensation, congenital heart disease, advanced arteriosclerosis or atherosclerosis with critical stenosis, hypertensive crisis, aortic dissection or rupture, tension pneumothorax, massive hemothorax, acute respiratory distress syndrome, status asthmaticus, acute exacerbation of chronic obstructive pulmonary disease, respiratory failure of any etiology, sepsis, septic shock, toxic shock syndrome, anaphylaxis or anaphylactoid reaction, severe hemorrhage (gastro-intestinal, obstetric, traumatic or postsurgical), upper or lower gastrointestinal bleed, perforated viscus, acute bowel obstruction, mesenteric ischemia, acute pancreatitis with systemic complications, fulminant hepatic failure, acute kidney injury requiring emergent dialysis, renal colic from obstructing kidney stones with septic obstruction, diabetic ketoacidosis, hyperosmolar hyperglycemic state, adrenal crisis, thyroid storm, malignant hyperthermia, heat stroke, severe hypothermia, severe electrolyte imbalance (hyper-/hypokalemia, hyper-/hyponatremia, hyper-/hypocalcemia, or severe acidosis/alkalosis), rhabdomyolysis, severe burns, high-voltage electrical injury, drowning, near-drowning, smoke or carbon-monoxide inhalation, acute poisoning or drug overdose (including opioid toxicity), serotonin syndrome, neuroleptic malignant syndrome, hyperventilation with impending syncope, panic attack with syncope risk, obstetric emergencies (eclampsia, placental abruption, amniotic-fluid embolism, postpartum hemorrhage), pulmonary hypertension crisis, sickle-cell crisis with acute chest syndrome, severe immunologic cytokine-release syndrome, or any combination thereof. In some embodiments, the computing device transmits, using an input peripheral of the computing device, the pain measurement that has been input by the user. In some embodiments, the artificial intelligence engine uses one or more trained computer-implemented models to generate the unique data signature associated with the one or more life-threatening events. In some embodiments, the pain measurement comprises pain after a final session of the treatment plan, average pain after all of one or more sessions of the treatment plan, or some combination thereof. In some embodiments, the measurement of revolutions per minute comprises an average revolutions per minute of one or more sessions of the treatment plan.

In some embodiments, the method 1220 may convert a format of the data to a standardized or canonical format and generate training data from the converted data. The training data may be used to train one or more computer-implemented models executed by the artificial intelligence engine.

In some embodiments, the method 1220 may, based on the unique data signature associated with the life-threatening event of the user, initiate a telehealth session between the computing device and a second computing device.

Consistent with the above disclosure, the examples of systems and methods enumerated in the following clauses are specifically contemplated and are intended as a non-limiting set of examples.
Clause 1.3 A computer-implemented method comprising: receiving, from one or more of an electromechanical machine, a sensor, and a computing device, data associated with a user that uses the electromechanical machine to perform a treatment plan; based on one or more correlations of one or more indicators included in the data, generating, using an artificial intelligence engine, a unique data signature associated with a life-threatening event of the user, wherein the unique data signature is generated when the one or more indicators satisfy a respective urgent threshold level, and the one or more indicators comprise a pain measurement and a measurement of revolutions per minute; and based on the unique data signature associated with the one or more life-threatening events, performing one or more preventative actions.
Clause 2.3. The computer-implemented method of clause 1.3, wherein the preventative actions are urgent.
Clause 3.3 The computer-implemented method of clause 1.3, wherein the one or more preventative actions comprise: transmitting one or more messages to or through one or more emergency service systems, controlling operation of the electromechanical machine, transmitting one or more messages to the computing device, initiating a telehealth session between the computing device and a second computing device, or some combination thereof.
Clause 4.3 The computer-implemented method of clause 1.3, wherein the one or more preventative actions comprise: generating, using the artificial intelligence engine, a modified treatment plan that modifies a parameter or activity associated with at least one of the pain measurement and the measurement of revolutions per minute, and controlling, while the user uses the electromechanical machine and using the modified treatment plan, the electromechanical machine.
Clause 5.3 The computer-implemented method of clause 1.3, wherein the one or more life-threatening events comprise deep vein thrombosis, heart attack, a heart arrhythmia of the user, an atrial fibrillation of the user, tachycardia, bradycardia, supraventricular tachycardia, congestive heart failure, heart valve disease, arteriosclerosis, atherosclerosis, pericardial disease, pericarditis, myocardial disease, myocarditis, cardiomyopathy, congenital heart disease, stroke, cardiac arrest, hyperventilation, panic attack, kidney stones, or some combination thereof.
Clause 6.3 The computer-implemented method of clause 1.3, wherein the computing device transmits, using an input peripheral of the computing device, the pain measurement that has been input by the user.
Clause 7.3 The computer-implemented method of clause 1.3, wherein the artificial intelligence engine uses one or more trained computer-implemented models to generate the unique data signature associated with the one or more life-threatening events.
Clause 8.3 The computer-implemented method of clause 1.3, wherein the pain measurement comprises pain after a final session of the treatment plan, average pain after all of one or more sessions of the treatment plan, or some combination thereof.
Clause 9.3 The computer-implemented method of clause 1.3, further comprising converting a format of the data to a standardized or canonical format and generating training data from the converted data, wherein the training data is used to train one or more computer-implemented models executed by the artificial intelligence engine.
Clause 10.3 The computer-implemented method of clause 1.3, wherein the measurement of revolutions per minute comprises an average revolutions per minute of one or more sessions of the treatment plan.
Clause 11.3 The computer-implemented method of clause 1.3, further comprising, based on the unique data signature associated with the life-threatening event of the user, initiating a telehealth session between the computing device and a second computing device.
Clause 12.3 A tangible, non-transitory computer-readable medium storing instructions that, when executed, cause one or more processing devices to:
receive, from one or more of an electromechanical machine, a sensor, and a computing device, data associated with a user that uses the electromechanical machine to perform a treatment plan;
based on one or more correlations of one or more indicators included in the data, generate, using an artificial intelligence engine, a unique data signature associated with a life-threatening event of the user, wherein the unique data signature is generated when the one or more indicators satisfy a respective urgent threshold level, and the one or more indicators comprise a pain measurement and a measurement of revolutions per minute; and
based on the unique data signature associated with the one or more life-threatening events, perform one or more preventative actions.
Clause 13.3 The computer-readable medium of clause 12.3, wherein the preventative actions are urgent.
Clause 14.3 The computer-readable medium of clause 12.3, wherein the one or more preventative actions comprise:
   transmitting one or more messages to or through one or more emergency service systems,
   controlling operation of the electromechanical machine,
   transmitting one or more messages to the computing device,
   initiating a telehealth session between the computing device and a second computing device, or
   some combination thereof.
Clause 15.3 The computer-readable medium of clause 12.3, wherein the one or more preventative actions comprise:
   generating, using the artificial intelligence engine, a modified treatment plan that modifies a parameter or activity associated with at least one of the pain measurement and the measurement of revolutions per minute, and
   controlling, while the user uses the electromechanical machine and using the modified treatment plan, the electromechanical machine.
Clause 16.3 The computer-readable medium of clause 12.3, wherein the one or more life-threatening events comprise deep vein thrombosis, heart attack, a heart arrhythmia of the user, an atrial fibrillation of the user, tachycardia, bradycardia, supraventricular tachycardia, congestive heart failure, heart valve disease, arteriosclerosis, atherosclerosis, pericardial disease, pericarditis, myocardial disease, myocarditis, cardiomyopathy, congenital heart disease, stroke, cardiac arrest, hyperventilation, panic attack, kidney stones, or some combination thereof.
Clause 17.3 The computer-readable medium of clause 12.3, wherein the computing device transmits, using an input peripheral of the computing device, the pain measurement that has been input by the user.
Clause 18.3 The computer-readable medium of clause 12.3, wherein the artificial intelligence engine uses one or more trained computer-implemented models to generate the unique data signature associated with the one or more life-threatening events.
Clause 19.3 The computer-readable medium of clause 12.3, wherein the pain measurement comprises pain after a final session of the treatment plan, average pain after all of one or more sessions of the treatment plan, or some combination thereof.
Clause 20.3 A system comprising:
   one or more memory devices storing instructions; and
   one or more processing devices communicatively coupled to the one or more memory devices, wherein the one or more processing devices execute the instructions to:
receive, from one or more of an electromechanical machine, a sensor, and a computing device, data associated with a user that uses the electromechanical machine to perform a treatment plan;
   based on one or more correlations of one or more indicators included in the data, generate, using an artificial intelligence engine, a unique data signature associated with a life-threatening event of the user, wherein the unique data signature is generated when the one or more indicators satisfy a respective urgent threshold level, and the one or more indicators comprise a pain measurement and a measurement of revolutions per minute; and
   based on the unique data signature associated with the one or more life-threatening events, perform one or more preventative actions.
      Systems and Methods for Optimizing Treatment Plans to Support User Progression During Rehabilitation for the Purpose of Assisting in Determining AI-Driven Interventions by Using Machine Learning to Generate at Least One Data Signature Associated with a Treatment Gap

FIG. 12D shows an example embodiment of a method 1230 for optimizing treatment plans to support user progression during rehabilitation for the purpose of assisting in determining AI-driven interventions by using machine learning to generate at least one data signature associated with a treatment gap. The method 1230 is performed by processing logic that may include hardware (circuitry, dedicated logic, etc.), software (such as is run on a general-purpose computer system or a dedicated machine), or a combination of both. The method 1230 and/or each of its individual functions, routines, subroutines, methods (as the term is used in object-oriented programming), or operations may be performed by one or more processing devices of a computing device (e.g., any component of FIG. 1, such as assistant interface 94, patient interface 50, and/or server 30 executing the artificial intelligence engine 11, training engine 9, machine learning models 13, etc.) implementing the method 1230. The method 1230 may be implemented as computer instructions stored on a memory device and able to be executed by the one or more processing devices. In certain implementations, the method 1230 may be performed by a single processing thread. Alternatively, the method 1230 may be performed by two or more processing threads, each thread implementing one or more individual functions, routines, subroutines, or operations of the methods. In some embodiments, one or more accelerators may be used to increase the performance of a processing device by offloading various functions, routines, subroutines, or operations from the processing device.

For simplicity of explanation, the method 1230 is depicted in FIG. 12D and described as a series of operations. However, operations in accordance with this disclosure can occur in various orders and/or concurrently, and/or with other operations not presented and described herein. For example, the operations depicted in the method 1230 in FIG. 12D may occur in combination with any other operation of any other method disclosed herein. Furthermore, not all illustrated operations may be required to implement the method 1230 in accordance with the disclosed subject matter. In addition, those skilled in the art will understand and appreciate that the method 1230 could alternatively be represented as a series of interrelated states via a state diagram or event diagram.

In some embodiments, one or more computer-implemented models (e.g., machine learning models, neural networks, expert systems, etc.) may be generated and trained by the artificial intelligence engine 11 and/or the training engine 9 to perform one or more of the operations of the methods described herein. For example, to perform the one or more operations, the processing device may execute the one or more computer-implemented models. In some embodiments, the one or more computer-implemented models may be iteratively retrained to select different features capable of enabling optimization of output. The features that may be modified may include a number of nodes included in each layer of the models, an objective function executed at each node, a number of layers, various weights associated with outputs of each node, and the like.

According to the method illustrated in FIG. 12D, at step 1232, the processing device receives, from one or more of an electromechanical machine, a sensor, and a computing device, data associated with a user that uses the electromechanical machine to perform a treatment plan.

At step 1234, the processing device correlates, using an artificial intelligence engine, at least two of a pain measurement, an indication of medication utilization, and a measurement of revolutions per minute to generate a unique data signature associated with a treatment gap associated with the treatment plan performed by the user. The at least two of the pain measurement, the indication of medication utilization, and the measurement of revolutions per minute may each satisfy a respective threshold level.

At step 1236, the processing device, based on the unique data signature associated with the treatment gap associated with the treatment plan performed by the user, generates, using the artificial intelligence engine, one or more alternative treatment plans for the user.

At step 1238, the processing device, based on a threshold compliance level associated with each of the one or more alternative treatment plans, selects an alternative treatment plan from the one or more alternative treatment plans.

At step 1240, the processing device controls, while the user uses the electromechanical machine and using the modified treatment plan, the electromechanical machine.

In some embodiments, the artificial intelligence engine selects the alternative treatment plan based on one or more available resources, psychographics of the user, demographics of the user, geographic data associated with the user, or some combination thereof. In some embodiments, the treatment gap represents a difference between what is prescribed in the treatment plan and a characteristic of the user, wherein the characteristic comprises performance, physical condition, medical conditions or both. In some embodiments, the computing device transmits, using an input peripheral of the computing device, the pain measurement that is input by the user. In some embodiments, the artificial intelligence engine uses one or more trained computer-implemented models to generate the unique data signature associated with the treatment gap. In some embodiments, the pain measurement comprises pain after a final session of the treatment plan, average pain after all of one or more sessions of the treatment plan, or some combination thereof. In some embodiments, the measurement of revolutions per minute comprises an average revolutions per minute of one or more sessions of the treatment plan.

In some embodiments, the method 1230 may convert a format of the data to a standardized or canonical format and generate training data from the converted data, wherein the training data is used to train one or more computer-implemented models executed by the artificial intelligence engine.

In some embodiments, the method 1230 may, based on the unique data signature associated with the treatment gap, initiate a telehealth session between the computing device and a second computing device associated with a second user.

Consistent with the above disclosure, the examples of systems and methods enumerated in the following clauses are specifically contemplated and are intended as a non-limiting set of examples.
Clause 1.4 A computer-implemented method comprising: receiving, from one or more of an electromechanical machine, a sensor, and a computing device, data associated with a user that uses the electromechanical machine to perform a treatment plan; correlating, using an artificial intelligence engine, at least two of a pain measurement, an indication of medication utilization, and a measurement of revolutions per minute to generate a unique data signature associated with a treatment gap associated with the treatment plan performed by the user, wherein the at least two of the pain measurement, the indication of medication utilization, and the measurement of revolutions per minute each satisfies a respective threshold level; based on the unique data signature associated with the treatment gap associated with the treatment plan performed by the user, generating, using the artificial intelligence engine, one or more alternative treatment plans for the user; based on a threshold compliance level associated with each of the one or more alternative treatment plans, selecting an alternative treatment plan from the one or more alternative treatment plans; and controlling, while the user uses the electromechanical machine and based on the alternative treatment plan, the electromechanical machine.
Clause 2.4 The computer-implemented method of clause 1.4, wherein the artificial intelligence engine selects the alternative treatment plan based on one or more available resources, psychographics of the user, demographics of the user, geographic data associated with the user, or some combination thereof.
Clause 3.4 The computer-implemented method of clause 1.4, wherein the treatment gap represents a difference between what is prescribed in the treatment plan and a characteristic of the user, wherein the characteristic comprises performance, physical condition, medical condition or both.
Clause 4.4 The computer-implemented method of clause 1.4, wherein the computing device transmits, using an input peripheral of the computing device, the pain measurement that is input by the user.
Clause 5.4 The computer-implemented method of clause 1.4, wherein the artificial intelligence engine uses one or more trained computer-implemented models to generate the unique data signature associated with the treatment gap.
Clause 6.4 The computer-implemented method of clause 1.4, wherein the pain measurement comprises pain after a final session of the treatment plan, average pain after all of one or more sessions of the treatment plan, or some combination thereof.
Clause 7.4 The computer-implemented method of clause 1.4, further comprising converting a format of the data to a standardized or canonical format and generating training data from the converted data, wherein the training data is used to train one or more computer-implemented models executed by the artificial intelligence engine.
Clause 8.4 The computer-implemented method of clause 1.4, wherein the measurement of revolutions per minute comprises an average revolutions per minute of one or more sessions of the treatment plan.
Clause 9.4 The computer-implemented method of clause 1.4, further comprising, based on the unique data signature associated with the treatment gap, initiating a telehealth session between the computing device and a second computing device associated with a second user.
Clause 10.4 A tangible, non-transitory computer-readable medium storing instructions that, when executed, cause one or more processing devices to:
   receive, from one or more of an electromechanical machine, a sensor, and a computing device, data associated with a user that uses the electromechanical machine to perform a treatment plan;
   correlate, using an artificial intelligence engine, at least two of a pain measurement, an indication of medication utilization, and a measurement of revolutions per minute to generate a unique data signature associated with a treatment gap associated with the treatment plan performed by the user, wherein the at least two of the pain measurement, the indication of medication utilization, and the measurement of revolutions per minute each satisfies a respective threshold level;
   based on the unique data signature associated with the treatment gap associated with the treatment plan performed by the user, generate, using the artificial intelligence engine, one or more alternative treatment plans for the user;
   based on a threshold compliance level associated with each of the one or more alternative treatment plans, select an alternative treatment plan from the one or more alternative treatment plans; and
   control, while the user uses the electromechanical machine and based on the alternative treatment plan, the electromechanical machine.
Clause 11.4 The computer-readable medium of clause 10.4, wherein the artificial intelligence engine selects the alternative treatment plan based on one or more available resources, psychographics of the user, demographics of the user, geographic data associated with the user, or some combination thereof.
Clause 12.4 The computer-readable medium of clause 10.4, wherein the treatment gap represents a difference between what is prescribed in the treatment plan and a characteristic of the user, wherein the characteristic comprises performance, physical condition, medical condition or both.
Clause 13.4 The computer-readable medium of clause 10.4, wherein the computing device transmits, using an input peripheral of the computing device, the pain measurement that is input by the user.
Clause 14.4 The computer-readable medium of clause 10.4, wherein the artificial intelligence engine uses one or more trained computer-implemented models to generate the unique data signature associated with the treatment gap.
Clause 15.4 The computer-readable medium of clause 10.4, wherein the pain measurement comprises pain after a final session of the treatment plan, average pain after all of one or more sessions of the treatment plan, or some combination thereof.
Clause 16.4 The computer-readable medium of clause 10.4, further comprising converting a format of the data to a standardized or canonical format and generating training data from the converted data, wherein the training data is used to train one or more computer-implemented models executed by the artificial intelligence engine.
Clause 17.4 The computer-readable medium of clause 10.4, wherein the measurement of revolutions per minute comprises an average revolutions per minute of one or more sessions of the treatment plan.
Clause 18.4 The computer-readable medium of clause 10.4, wherein, based on the unique data signature associated with the treatment gap, the one or more processing devices initiates a telehealth session between the computing device and a second computing device associated with a second user.
Clause 19.4 A system comprising:
   one or more memory devices storing instructions; and
   one or more processing devices communicatively coupled to the one or more memory devices, wherein the one or more processing devices executes the instructions to:
receive, from one or more of an electromechanical machine, a sensor, and a computing device, data associated with a user that uses the electromechanical machine to perform a treatment plan;
   correlate, using an artificial intelligence engine, at least two of a pain measurement, an indication of medication utilization, and a measurement of revolutions per minute to generate a unique data signature associated with a treatment gap associated with the treatment plan performed by the user, wherein the at least two of the pain measurement, the indication of medication utilization, and the measurement of revolutions per minute each satisfies a respective threshold level;
   based on the unique data signature associated with the treatment gap associated with the treatment plan performed by the user, generate, using the artificial intelligence engine, one or more alternative treatment plans for the user;
   based on a threshold compliance level associated with each of the one or more alternative treatment plans, select an alternative treatment plan from the one or more alternative treatment plans; and
   control, while the user uses the electromechanical machine and based on the alternative treatment plan, the electromechanical machine.
Clause 20.4 The system of clause 19.4, wherein the artificial intelligence engine selects the alternative treatment plan based on one or more available resources, psychographics of the user, demographics of the user, geographic data associated with the user, or some combination thereof.

### Systems and Methods for Performing AI-Driven Interventions Via Optimization of Treatment Plans to Support User Progression During Rehabilitation by Performing, Based On Data Signatures Related to Medically-Associated Events, Selective Data Injection

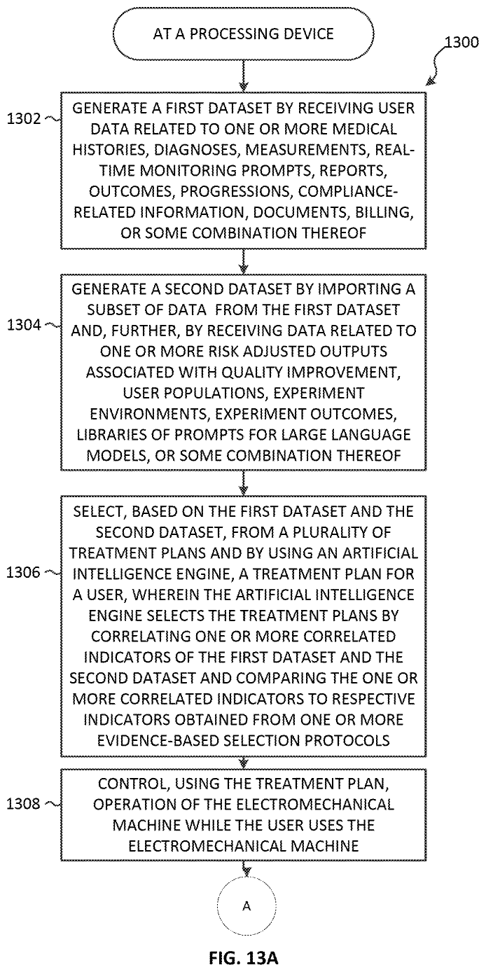

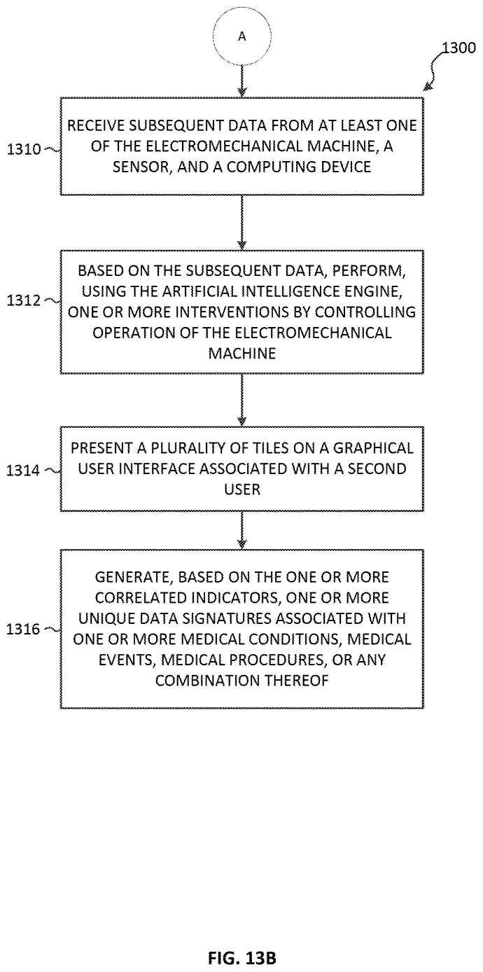

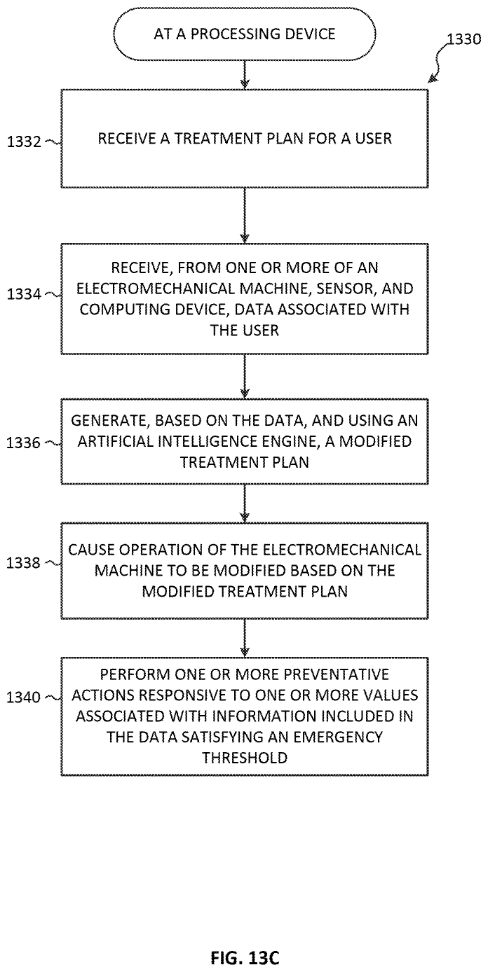

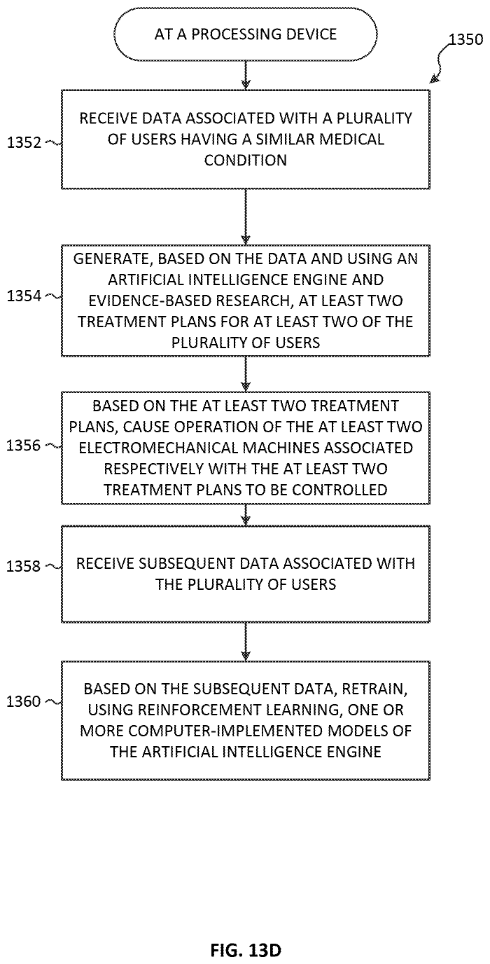

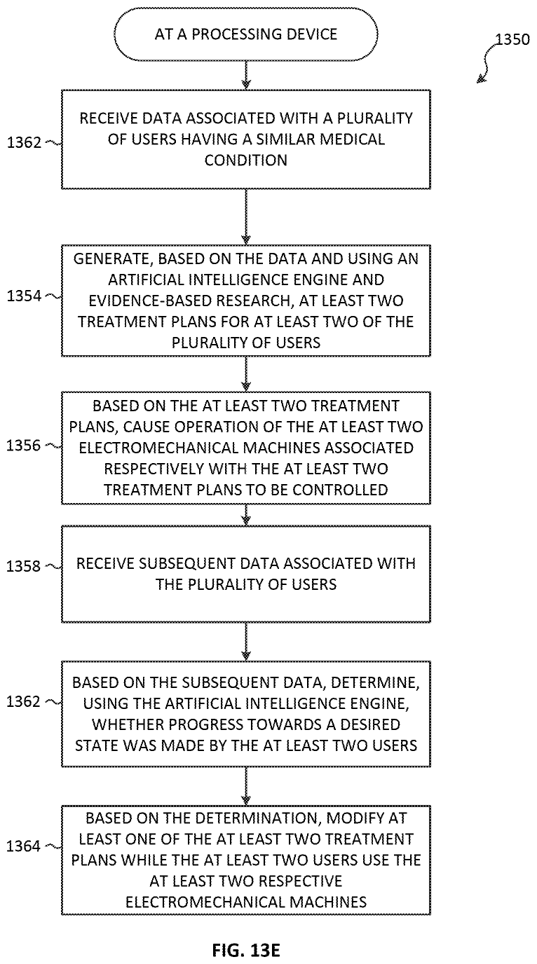

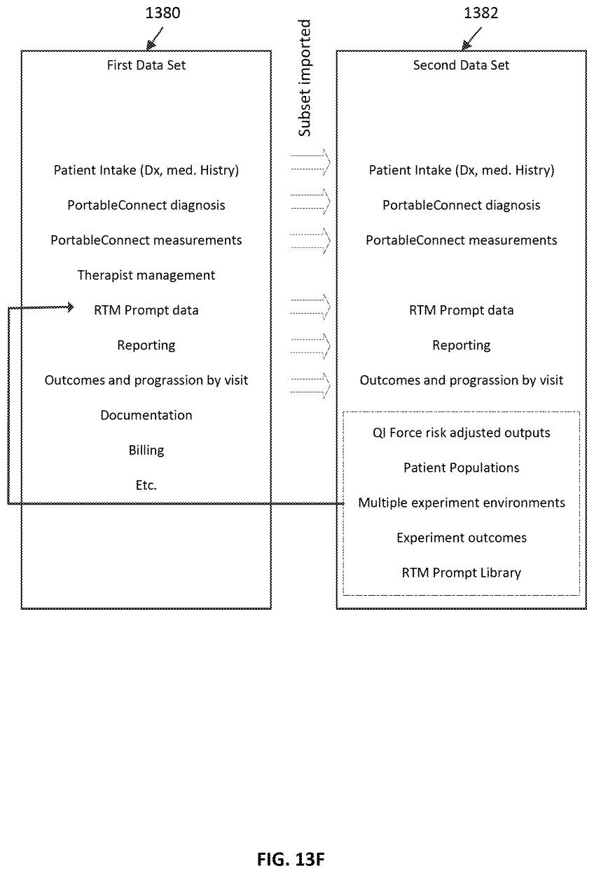

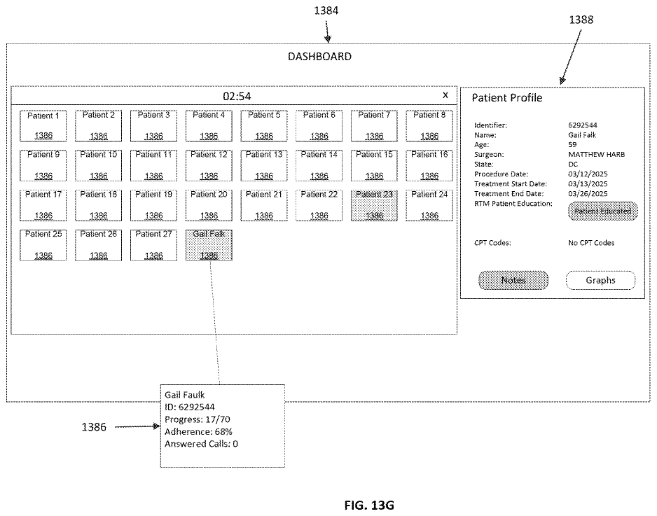

FIG. 13A shows an example embodiment of a method 1300 for controlling, by an artificial intelligence engine, operation of an electromechanical machine while a user uses the machine. The method 1300 is performed by processing logic that may include hardware (circuitry, dedicated logic, etc.), software (such as is run on a general-purpose computer system or a dedicated machine), or a combination of both. The method 1300 and/or each of its individual functions, routines, subroutines, methods (as the term is understood in the field of object-oriented programming) or operations may be performed by one or more processing devices of a computing device (e.g., any component of FIG. 1, such as assistant interface 94, patient interface 50, and/or server 30 executing the artificial intelligence engine 11, training engine 9, machine learning models 13, etc.) implementing the method 1300. The method 1300 may be implemented as computer instructions stored on a memory device and able to be executed by the one or more processing devices. In certain implementations, the method 1300 may be performed by a single processing thread. Alternatively, the method 1300 may be performed by two or more processing threads, each thread implementing one or more individual functions, routines, subroutines, or operations of the methods. In some embodiments, one or more accelerators may be used to increase the performance of a processing device by offloading various functions, routines, subroutines, methods or operations from the processing device.

For simplicity of explanation, the method 1300 is depicted in FIG. 13A and described as a series of operations. However, operations in accordance with this disclosure can occur in various orders and/or concurrently, and/or with other operations not presented and described herein. For example, the operations depicted in the method 1300 in FIG. 13A may occur in combination with any other operation of any other method disclosed herein. Furthermore, not all illustrated operations may be required to implement the method 1300 in accordance with the disclosed subject matter. In addition, those skilled in the art will understand and appreciate that the method 1300 could alternatively be represented as a series of interrelated states via a state diagram or event diagram.

In some embodiments, one or more computer-implemented models (e.g., machine learning models, neural networks, expert systems, etc.) may be generated and trained by the artificial intelligence engine 11 and/or the training engine 9 to perform one or more of the operations of the methods described herein. For example, to perform the one or more operations, the processing device may execute the one or more computer-implemented models. In some embodiments, the one or more computer-implemented models may be iteratively retrained to select different features capable of enabling optimization of output. The features that may be modified may include a number of nodes included in each layer of the models, an objective function executed at each node, a number of layers, various weights associated with outputs of each node, and the like.

At block 1302 the processing device receives user data related to one or more medical histories, diagnoses, measurements, real-time monitoring prompts, reports, outcomes, progressions, compliance-related information documents, billing, or any combination thereof. The processing device generates a first data set based on the received user data.

At block 1304, the processing device generates a second dataset by importing a subset of data from the first dataset and, by receiving additional data related to one or more risk adjusted outputs associated with quality improvement, user populations, experiment environments, experiment outcomes, libraries of prompts for large language models, or any combination thereof. FIG. 13F illustrates exemplary first and second datasets. As shown in the example, the first dataset 1380 comprises patient intake information such as medical histories, diagnoses, measurements, therapist management data, real-time monitoring prompts, reports, outcomes, progressions, compliance-related information documents, documentation, billing, and so on. The first dataset may be a corporate access database, for example, and be utilized in a conventional manner, although the embodiments are not so limited. A subset of the first dataset 1380 is imported into the second dataset 1382. Once imported, the data may be processed or altered. The second dataset 1382 is preferably not accessed directly by the entity storing and accessing the first dataset, although the embodiments are not necessarily so limited. The second dataset is utilized by an artificial intelligence engine which may use machine learning to improve rehabilitation outcomes. Specifically, the data relating to quality improvements, user populations, experiment environments, experiment outcomes, and/or libraries of prompts for large language models, are utilized by the artificial intelligence engine and fed back as updated real-time monitoring prompts into the first dataset 1380.

At block 1306, the processing device selects, based on the first dataset and the second dataset, from a plurality of treatment plans and by using an artificial intelligence engine, as described above, a treatment plan for a user. The artificial intelligence engine may select the treatment plan by correlating one or more indicators of the first dataset and the second dataset and comparing the one or more correlated indicators to respective indicators obtained from one or more evidence-based selection protocols.

At block 1308, using the selected treatment plan, the processing device controls operation of the electromechanical machine while the user uses the machine.

FIG. 13B describes further optional aspects of the method described above in connection with FIG. 13A. At block 1310, the processing device receives subsequent data from at least one of the electromechanical machine, a sensor, and a computing device. At step 1312, the processing device performs, based on the subsequent data and using the artificial intelligence engine, one or more interventions by controlling the operation of the electromechanical machine. As an example of an intervention, the processing device may execute a virtual agent on a computing device that, while the user uses the electromechanical machine, uses natural language processing or generative artificial intelligence to communicate with the user. As another example of an intervention, the processing device may modify an operating parameter of the electromechanical machine to accelerate the user's rehabilitation progression. As yet another example of an intervention, the processing device may perform a blind experiment by controlling operating parameters of at least the electromechanical machine and a second electromechanical machine to determine which modifications to which operating parameters result in increasing rehabilitation progression, decreasing rehabilitation progression, or maintaining rehabilitation progression.

It will be appreciated that the treatment plan discussed herein may include modifying one or more operating parameters of an electromechanical machine associated with one or more exercises for the user to perform using the electromechanical machine.

At block 1314, a plurality of tiles is presented on a graphical user interface of a computing device associated with a second user. At least one of the plurality of tiles preferably comprises a multimedia feed associated with the user's performance of the treatment plan using the electromechanical machine. FIG. 13G illustrates an example user interface 1384, the user interface 1384 illustrating a plurality of tiles 1386 that are selectable by the second user. Information associated with the selected patient tile is highlighted in window 1388. The patient information highlighter in window 1388, and/or each of the patient tiles 1386 preferably comprise a multimedia feed associated with the user's performance of the treatment plan using the electromechanical machine. It should be appreciated that although the tiles 1386 are illustrated in FIG. 13G with only a patient name, in practice each of the tiles 1386 preferably includes additional information such as patient name, ID, progress, a characterization (qualitative and/or quantitative, narrative or statistical), of adherence to the treatment plan, and answered calls, as illustrated in the enlarged version of the tile for patient “Gail Falk”.

Further, the second user may use an input peripheral (e.g., mouse, keyboard, microphone, touchscreen, etc.) to select one or more of the tiles to initiate a telehealth session or telemedicine session with one of the users via real-time or near real-time multimedia (e.g., video and audio) conferencing. In some embodiments, the telehealth session or telemedicine session may be initiated and conducted while the user is performing a treatment plan using the electromechanical machine.

At block 1316, the processing device generates, based on the one or more correlated indicators, one or more unique data signatures associated with one or more medical conditions, medical events, medical procedures, or any combination thereof.

Consistent with the above disclosure, the examples of systems and methods enumerated in the following clauses are specifically contemplated and are intended as a non-limiting set of examples.
Clause 1.5 A computer-implemented method comprising:
   generating a first dataset by receiving user data related to one or more medical histories, diagnoses, measurements, real-time monitoring prompts, reports, outcomes, progressions, compliance-related information, documents, billing, or some combination thereof;
   generating a second dataset by importing a subset of data from the first dataset and, further, by receiving data related to one or more risk adjusted outputs associated with quality improvement, user populations, experiment environments, experiment outcomes, libraries of prompts for large language models, or some combination thereof.
   based on the first dataset and the second dataset, selecting, from a plurality of treatment plans and by using an artificial intelligence engine, a treatment plan for a user, wherein the artificial intelligence engine selects the treatment plan by correlating one or more indicators of the first dataset and the second dataset and comparing the one or more correlated indicators to respective indicators obtained from one or more evidence-based selection protocols; and
   controlling, using the treatment plan, operation of the electromechanical machine while the user uses the electromechanical machine.
Clause 2.5 The computer-implemented method of clause 1.5, further comprising:
   receiving subsequent data from at least one of the electromechanical machine, a sensor, and a computing device; and
   based on the subsequent data, performing, using the artificial intelligence engine, one or more interventions by controlling the operation of the electromechanical machine.
Clause 3.5 The computer-implemented method of clause 2.5, wherein the one or more interventions comprise executing a virtual agent on a computing device that, while the user uses the electromechanical machine, uses natural language processing or generative artificial intelligence to communicate with the user.
Clause 4.5 The computer-implemented method of clause 2.5, wherein the one or more interventions comprise modifying an operating parameter of the electromechanical machine to accelerate the user's rehabilitation progression.
Clause 5.5 The computer-implemented method of clause 2.5, wherein the one or more interventions comprise performing a blind experiment by controlling operating parameters of at least the electromechanical machine and a second electromechanical machine to determine which operating parameters result in increasing rehabilitation progression, decreasing rehabilitation progression, or maintaining rehabilitation progression.
Clause 6.5 The computer-implemented method of clause 1.5, wherein the treatment plan comprises modifying one or more operating parameters of an electromechanical machine associated with one or more exercises for the user to perform using the electromechanical machine.
Clause 7.5 The computer-implemented method of clause 1.5, wherein a plurality of tiles is presented on a graphical user interface of a computing device associated with a second user, wherein at least one of the plurality of tiles comprises a multimedia feed associated with the user's performance of the treatment plan using the electromechanical machine.
Clause 8.5 The computer-implemented method of clause 1.5, further comprising generating, based on the one or more correlated indicators, one or more unique data signatures associated with one or more medical conditions, medical events, medical procedures, or some combination thereof.
Clause 9.5 A tangible, non-transitory computer-readable medium storing instructions, when executed, cause one or more processing devices to:
   generate a first dataset by receiving user data related to one or more medical histories, diagnoses, measurements, real-time monitoring prompts, reports, outcomes, progressions, compliance-related information, documents, billing, or some combination thereof;
   generate a second dataset by importing a subset of data from the first dataset and, further, by receiving data related to one or more risk adjusted outputs associated with quality improvement, user populations, experiment environments, experiment outcomes, libraries of prompts for large language models, or some combination thereof.
   based on the first dataset and the second dataset, select, from a plurality of treatment plans and by using an artificial intelligence engine, a treatment plan for a user, wherein the artificial intelligence engine selects the treatment plan by correlating one or more indicators of the first dataset and the second dataset and comparing the one or more correlated indicators to respective indicators obtained from one or more evidence-based selection protocols; and
   control, using the treatment plan, operation of the electromechanical machine while the user uses the electromechanical machine.
Clause 10.5 The computer-readable medium of clause 9.5, wherein the one or more processing devices:
   receive subsequent data from at least one of the electromechanical machine, a sensor, and a computing device; and
   based on the subsequent data, perform, using the artificial intelligence engine, one or more interventions by controlling the operation of the electromechanical machine.
Clause 11.5 The computer-readable medium of clause 10.5, wherein the one or more interventions comprise executing a virtual agent on a computing device that, while the user uses the electromechanical machine, uses natural language processing or generative artificial intelligence to communicate with the user.
Clause 12.5 The computer-readable medium of clause 10.5, wherein the one or more interventions comprise modifying an operating parameter of the electromechanical machine to accelerate the user's rehabilitation progression.
Clause 13.5 The computer-readable medium of clause 10.5, wherein the one or more interventions comprise performing a blind experiment by controlling operating parameters of at least the electromechanical machine and a second electromechanical machine to determine which operating parameters result in increasing rehabilitation progression, decreasing rehabilitation progression, or maintaining rehabilitation progression.
Clause 14.5 The computer-readable medium of clause 9.5, wherein the treatment plan comprises modifying one or more operating parameters of an electromechanical machine associated with one or more exercises for the user to perform using the electromechanical machine.
Clause 15.5 The computer-implemented method of clause 9.5, wherein a plurality of tiles is presented on a graphical user interface of a computing device associated with a second user, wherein at least one of the plurality of tiles comprises a multimedia feed associated with the user's performance of the treatment plan using the electromechanical machine.
Clause 16.5 The computer-implemented method of clause 9.5, wherein the one or more processing devices generate, based on the one or more correlated indicators, one or more unique data signatures associated with one or more medical conditions, medical events, medical procedures, or some combination thereof.
Clause 17.5 A system comprising:
   one or more memory devices storing instructions; and
   one or more processing devices communicatively coupled to the one or more memory devices, wherein the one or more processing devices executes the instructions to:
generate a first dataset by receiving user data related to one or more medical histories, diagnoses, measurements, real-time monitoring prompts, reports, outcomes, progressions, compliance-related information, documents, billing, or some combination thereof,
   generate a second dataset by importing a subset of data from the first dataset and, further, by receiving data related to one or more risk adjusted outputs associated with quality improvement, user populations, experiment environments, experiment outcomes, libraries of prompts for large language models, or some combination thereof.
   based on the first dataset and the second dataset, select, from a plurality of treatment plans and by using an artificial intelligence engine, a treatment plan for a user, wherein the artificial intelligence engine selects the treatment plan by correlating one or more indicators of the first dataset and the second dataset and comparing the one or more correlated indicators to respective indicators obtained from one or more evidence-based selection protocols; and
   control, using the treatment plan, operation of the electromechanical machine while the user uses the electromechanical machine.
Clause 18.5 The system of clause 17.5, wherein the one or more processing devices:
   receive subsequent data from at least one of the electromechanical machine, a sensor, and a computing device; and
   based on the subsequent data, perform, using the artificial intelligence engine, one or more interventions by controlling the operation of the electromechanical machine.
Clause 19.5 The system of clause 18.5, wherein the one or more interventions comprise executing a virtual agent on a computing device that, while the user uses the electromechanical machine, uses natural language processing or generative artificial intelligence to communicate with the user.
Clause 20.5 The system of clause 18.5, wherein the one or more interventions comprise modifying an operating parameter of the electromechanical machine to accelerate the user's rehabilitation progression.
   Systems and Methods for Using Artificial Intelligence to Control an Electromechanical Machine in Order to Adjust Dosage Amounts and Frequencies for Infusion-Based or Other Medication-Based Therapies for the Purpose of Optimizing a Treatment Plan to Shorten The User's Recovery Period

FIG. 13C shows an example embodiment of a method 1330 for using artificial intelligence to control an electromechanical machine in order to adjust dosage amounts and frequencies for infusion-based or other medication-based therapies for the purpose of optimizing a treatment plan to shorten a user's recovery period. The method 1330 is performed by processing logic that may include hardware (circuitry, dedicated logic, etc.), software (such as is run on a general-purpose computer system or a dedicated machine), or a combination of both. The method 1330 and/or each of its individual functions, routines, subroutines, methods (as the term is used in object-oriented programming), or operations may be performed by one or more processing devices of a computing device (e.g., any component of FIG. 1, such as the assistant interface 94, patient interface 50, and/or server 30 executing the artificial intelligence engine 11, training engine 9, machine learning models 13, etc.) implementing the method 1330. The method 1330 may be implemented as computer instructions stored on a memory device and able to be executed by the one or more processing devices. In certain implementations, the method 1330 may be performed by a single processing thread. Alternatively, the method 1330 may be performed by two or more processing threads, each thread implementing one or more individual functions, routines, subroutines, methods or operations of the methods. In some embodiments, one or more accelerators may be used to increase the performance of a processing device by offloading various functions, routines, subroutines, methods or operations from the processing device.

For simplicity of explanation, the method 1330 is depicted in FIG. 13C and described as a series of operations. However, operations in accordance with this disclosure can occur in various orders and/or concurrently, and/or with other operations not presented and described herein. For example, the operations depicted in the method 1330 in FIG. 13C may occur in combination with any other operation of any other method disclosed herein. Furthermore, not all illustrated operations may be required to implement the method 1330 in accordance with the disclosed subject matter. In addition, those skilled in the art will understand and appreciate that the method 1330 could alternatively be represented as a series of interrelated states via a state diagram or event diagram.

In some embodiments, one or more computer-implemented models (e.g., machine learning models, neural networks, expert systems, etc.) may be generated and trained by the artificial intelligence engine 11 and/or the training engine 9 to perform one or more of the operations of the methods described herein. For example, to perform the one or more operations, the processing device may execute the one or more computer-implemented models. In some embodiments, the one or more computer-implemented models may be iteratively retrained to select different features capable of enabling optimization of output. The features that may be modified may include a number of nodes included in each layer of the models, an objective function executed at each node, a number of layers, various weights associated with outputs of each node, and the like.

According to the method illustrated in FIG. 13C, at step 1332, the processing device receives a treatment plan for a user. The treatment plan preferably comprises a schedule of treatments for infusion-based or electroconvulsive therapy (i.e., ECT) and one or more operating parameters of an electromechanical machine.

At step 1334, the processing device receives, from one or more of the electromechanical machine, a sensor, and a computing device, data associated with the user. Then at step 1336, the processing device generates, based on the data, and using an artificial intelligence engine, a modified treatment plan. The modified treatment plan preferably comprises a modified scheduled of treatments for the therapy and one or more modified operating parameters of the electromechanical machine. At step 1338, based on the modified treatment plan, the processing device causes operation of the electromechanical machine to be modified.

The schedule of treatments preferably comprises one or more of a treatment modality, a treatment frequency, a treatment timing, medications or procedures associated with the treatment, dosages, time lengths or other parameters associated with the medications or procedures, or any combination thereof. The modified schedule of treatments preferably comprises information pertaining to a measure of a substance to inject, inhale, ingest, apply, absorb, swallow, or parenterally infuse into the user, timing of the treatments, and control instructions for one or more pumps associated with the substance.

A therapeutic agent (e.g., substance) may be administered parenterally. Parenterally administration may refer to any route that bypasses the alimentary canal, including intradermal, sub-cutaneous, intramuscular, intravenous, intra-arterial, intrathecal or other direct injection/infusion techniques. Also, a therapeutic agent may administered by any clinically accepted non-parenteral route, such as: (i) enteral delivery, encompassing oral ingestion (i.e., PO), sublingual or buccal absorption across the oral mucosa, rectal administration of suppositories or enemata, and administration through a nasogastric or percutaneous endoscopic gastrostomy tube; (ii) mucosal application, for example intranasal sprays or drops, ophthalmic instillations, otic preparations, intravaginal rings, creams or pessaries, and intra-urethral pellets; (iii) cutaneous routes, including topical dermatological formulations that act locally on the skin and true transdermal systems (e.g., adhesive patches) designed for systemic absorption; and (iv) implantable or depot systems, such as sub-dermal contraceptive rods, intra-uterine drug-releasing devices, intravitreal ocular implants, or programmable pumps configured for sustained intrathecal, epidural, intraperitoneal or subcutaneous release.

At step 1340, the processing device performs one or more preventative actions response to one or more values associated with information included in the data satisfying an emergency threshold. The one or more preventative actions preferably comprise causing operation of the electromechanical machine to stop.

When the schedule of treatments includes infusion-based therapy, the therapy preferably includes using one or more pumps, electrical devices, computing devices, or any combination thereof. Electroconvulsive-based therapy preferably comprises using one or more electrical devices, magnetic or electromagnetic devices, sensor-based devices, biometric measuring devices, computing devices, or any combination thereof.

In some embodiments, the processing device transmits, using an input peripheral, a pain measurement that has been input by the user. In some embodiments, the pain measurement may be determined based on what the user is experiencing, in addition to or instead of, what the user reported. The pain measurement may be determined based on video of the user performing a treatment plan using the electromechanical machine. For example, facial recognition techniques may be used to determine when the person's face is grimacing and the artificial intelligence engine may use the computer-implemented models to determine a pain level of the user. Further, computer vision may be utilized to analyze video or image data of the user performing a treatment plan and to determine whether the user is in pain. Additionally, based on the pain determination, the artificial intelligence engine may determine a pain level of the user.

The modified treatment plan according to the method shown in FIG. 13C preferably comprises one or more operating parameters of the electromechanical machine.

Consistent with the above disclosure, the examples of systems and methods enumerated in the following clauses are specifically contemplated and are intended as a non-limiting set of examples.
Clause 1.6 A computer-implemented method comprising:
   receiving a treatment plan for a user, wherein the treatment plan comprises a schedule of treatments for infusion-based or electroconvulsive therapy (i.e., ECT) and one or more operating parameters of an electromechanical machine;
   receiving, from one or more of the electromechanical machine, sensor, and computing device, data associated with the user;
   based on the data, generating, using an artificial intelligence engine, a modified treatment plan, wherein the modified treatment plan comprises a modified schedule of treatments for the therapy and one or more modified operating parameters of the electromechanical machine; and
   based on the modified treatment plan, causing operation of the electromechanical machine to be modified.
Clause 2.6 The computer-implemented method of clause 1.6, wherein the schedule of treatments comprises one or more of a treatment modality, a treatment frequency, a treatment timing, medications or procedures associated with the treatment, dosages, time lengths or other parameters associated with the medications or procedures, or any combination thereof.
Clause 3.6 The computer-implemented method of clause 1.6, wherein the modified schedule of treatments comprises information pertaining to a measure of a substance to inject, inhale, ingest, apply, absorb, swallow, enteral deliver, mucosal apply, cutaneous route, or parenterally infuse into the user, timing of the treatments, and control instructions for one or more pumps associated with the substance.
Clause 4.6 The computer-implemented method of clause 1.6, further comprising, responsive to one or more values associated with information included in the data satisfying an emergency threshold, performing one or more preventative actions.
Clause 5.6 The computer-implemented method of clause 4.6, wherein the one or more preventative actions comprise causing operation of the electromechanical machine to stop.
Clause 6.6 The computer-implemented method of clause 1.6, wherein the infusion-based therapy comprises using one or more pumps, electrical devices, computing devices, or some combination thereof.
Clause 7.6 The computer-implemented method of clause 1.6, wherein the electroconvulsive-based therapy comprises using one or more electrical devices, biometric measuring devices, computing devices, or some combination thereof.
Clause 8.6 The computer-implemented method of clause 1.6, wherein the computing device transmits, using an input peripheral of the computing device, a pain measurement that has been input by the user.
Clause 9.6 The computer-implemented method of clause 1.6, wherein the modified treatment plan comprises one or more operating parameters of the electromechanical machine.
Clause 10.6 A tangible, non-transitory computer-readable medium storing instructions that, when executed, cause one or more processing devices to:
   receive a treatment plan for a user, wherein the treatment plan comprises a schedule of treatments for infusion-based or electroconvulsive therapy (i.e., ECT) and one or more operating parameters of an electromechanical machine;
   receive, from one or more of the electromechanical machine, sensor, and computing device, data associated with the user;
   based on the data, generate, using an artificial intelligence engine, a modified treatment plan, wherein the modified treatment plan comprises a modified schedule of treatments for the therapy and one or more modified operating parameters of the electromechanical machine; and
   based on the modified treatment plan, cause operation of the electromechanical machine to be modified.
Clause 11.6 The computer-readable medium of clause 10.6, wherein the schedule of treatments comprises one or more of a treatment modality, a treatment frequency, a treatment timing, medications or procedures associated with the treatment, dosages, time lengths or other parameters associated with the medications or procedures, or any combination thereof.
Clause 12.6 The computer-readable medium of clause 10.6, wherein the modified schedule of treatments comprises information pertaining to a measure of a substance to inject, inhale, ingest, apply, absorb, swallow, or parenterally infuse into the user, timing of the treatments, and control instructions for one or more pumps associated with the substance.
Clause 13.6 The computer-readable medium of clause 10.6, wherein, responsive to one or more values associated with information included in the data satisfying an emergency threshold, the one or more processing devices perform one or more preventative actions.
Clause 14.6 The computer-readable medium of clause 13.6, wherein the one or more preventative actions comprise causing operation of the electromechanical machine to stop.
Clause 15.6 The computer-readable medium of clause 10.6, wherein the electroconvulsive-based therapy comprises using one or more electrical devices, biometric measuring devices, computing devices, or some combination thereof.
Clause 16.6 The computer-readable medium of clause 10.6, wherein the computing device transmits, using an input peripheral of the computing device, a pain measurement that has been input by the user.
Clause 17.6 The computer-readable medium of clause 10.6, wherein the modified treatment plan comprises one or more operating parameters of the electromechanical machine.
Clause 18.6 A system comprising:
   one or more memory devices storing instructions; and
   one or more processing devices communicatively coupled to the one or more memory devices, wherein the one or more processing devices execute the instructions to:
receive a treatment plan for a user, wherein the treatment plan comprises a schedule of treatments for infusion-based or electroconvulsive therapy (i.e., ECT) and one or more operating parameters of an electromechanical machine;
   receive, from one or more of the electromechanical machine, sensor, and computing device, data associated with the user;
   based on the data, generate, using an artificial intelligence engine, a modified treatment plan, wherein the modified treatment plan comprises a modified schedule of treatments for the therapy and one or more modified operating parameters of the electromechanical machine; and
   based on the modified treatment plan, cause operation of the electromechanical machine to be modified.
Clause 19.6 The system of clause 18.6, wherein the schedule of treatments comprises one or more of a treatment modality, a treatment frequency, a treatment timing, medications or procedures associated with the treatment, dosages, time lengths or other parameters associated with the medications or procedures, or any combination thereof.
Clause 20.6 The system of clause 18.6, wherein the modified schedule of treatments comprises information pertaining to a measure of a substance to inject, inhale, ingest, apply, absorb, swallow, or parenterally infuse into the user, timing of the treatments, and control instructions for one or more pumps associated with the substance.
   Systems and Methods for Incorporating into a Treatment Plan to Optimize the User's Recovery Via the Use of AI-Driven Interventions Associated with Reinforcement Learning that Rely on Evidence-Based Experiments

FIG. 13D shows an example embodiment of a method 1350 for using artificial intelligence to control an electromechanical machine in order to adjust dosage amounts and frequencies for infusion-based or other medication-based therapies for the purpose of optimizing a treatment plan to shorten a user's recovery period. The method 1350 is performed by processing logic that may include hardware (circuitry, dedicated logic, etc.), software (such as is run on a general-purpose computer system or a dedicated machine), or a combination of both. The method 1350 and/or each of its individual functions, routines, subroutines, methods (as the term is used in object-oriented programming), or operations may be performed by one or more processing devices of a computing device (e.g., any component of FIG. 1, such as assistant interface 94, patient interface 50, and/or server 30 executing the artificial intelligence engine 11, training engine 9, machine learning models 13, etc.) implementing the method 1350. The method 1350 may be implemented as computer instructions stored on a memory device and able to be executed by the one or more processing devices. In certain implementations, the method 1350 may be performed by a single processing thread. Alternatively, the method 1350 may be performed by two or more processing threads, each thread implementing one or more individual functions, routines, subroutines, methods or operations of the methods. In some embodiments, one or more accelerators may be used to increase the performance of a processing device by offloading various functions, routines, subroutines, methods or operations from the processing device.

For simplicity of explanation, the method 1350 is depicted in FIG. 13D and described as a series of operations. However, operations in accordance with this disclosure can occur in various orders and/or concurrently, and/or with other operations not presented and described herein. For example, the operations depicted in the method 1350 in FIG. 13D may occur in combination with any other operation of any other method disclosed herein. Furthermore, not all illustrated operations may be required to implement the method 1350 in accordance with the disclosed subject matter. In addition, those skilled in the art will understand and appreciate that the method 1350 could alternatively be represented as a series of interrelated states via a state diagram or event diagram.

In some embodiments, one or more computer-implemented models (e.g., machine learning models, neural networks, expert systems, etc.) may be generated and trained by the artificial intelligence engine 11 and/or the training engine 9 to perform one or more of the operations of the methods described herein. For example, to perform the one or more operations, the processing device may execute the one or more computer-implemented models. In some embodiments, the one or more computer-implemented models may be iteratively retrained to select different features capable of enabling optimization of output. The features that may be modified may include a number of nodes included in each layer of the models, an objective function executed at each node, a number of layers, various weights associated with outputs of each node, and the like.

According to the exemplary method shown in FIG. 13D, at step 1352, the processing device receives data associated with a plurality of users having a similar medical condition. The data is preferably associated with one or more of a characteristic of the plurality of users and a performance metric of the plurality of users.

At step 1354, the processing device generates, based on the data and using an artificial intelligence engine and evidence-based research, at least two treatment plans for at least two of the plurality of users. A first treatment plan of the at least two treatment plans preferably comprises instructions to inform a first user of an aspect of the first treatment plan. A second treatment plan of the at least two treatment plans preferably excludes instructions to inform a second user of an aspect of the second treatment plan.

At step 1356, based on the at least two treatment plans, the processing device causes operation of at least two electromechanical machines associated respectively with the at least two treatment plans to be controlled.

The method of FIG. 13D further optionally includes receiving subsequent data associated with the plurality of users at step 1358, and at step 1360, based on the subsequent data, retraining, using reinforcement learning, one or more computer implemented models of the artificial intelligence engine.

Responsive to receiving subsequent data at step 1358, if the subsequent data satisfies an emergency threshold, the processing device may perform one or more preventative actions. The one or more preventative actions preferably comprise causing operation of at least one of the at least two electromechanical machines to stop.

According to the method shown in FIG. 13D, the artificial intelligence engine preferably comprises one or more trained computer-implemented models.

According to the method of FIG. 13D, the first treatment plan and the second treatment plan are executed while the at least two of the plurality of users are using the at least two respective electromechanical machines.

FIG. 13E illustrates another exemplary embodiment, similar to the method illustrated in FIG. 13D, but with step 1360 replaced with steps 1362 and 1364. In the method according to FIG. 13E, at step 1362, the processing device determines, based on the subsequent data and using the artificial intelligence engine, whether progress towards a desired state was made by the at least two users. At step 1364, based on the determination, the processing device modifies at least one of the at least two treatment plans while the at least two users use the at least two respective electromechanical machines.

Consistent with the above disclosure, the examples of systems and methods enumerated in the following clauses are specifically contemplated and are intended as a non-limiting set of examples.
Clause 1.7 A computer-implemented method comprising:
   receiving data associated with a plurality of users having a similar medical condition, wherein the data is associated with one or more of a characteristic of the plurality of users and a performance metric of the plurality of users;
   based on the data, generating, using an artificial intelligence engine and evidence-based research, at least two treatment plans for at least two of the plurality of users, wherein a first treatment plan of the at least two treatment plans comprises instructions to inform a first user of an aspect of the first treatment plan and wherein, further, a second treatment plan of the at least two treatment plans excludes instructions to inform a second user of an aspect of the second treatment plan; and
   based on the at least two treatment plans, causing operation of at least two electromechanical machines associated respectively with the at least two treatment plans to be controlled.
Clause 2.7 The computer-implemented method of clause 1.7, further comprising:
   receiving subsequent data associated with the plurality of users; and
   based on the subsequent data, retraining, using reinforcement learning, one or more computer-implemented models of the artificial intelligence engine.
Clause 3.7 The computer-implemented method of clause 1.7, wherein, responsive to receiving subsequent data that satisfies an emergency threshold level, performing one or more preventative actions.
Clause 4.7 The computer-implemented method of clause 3.7, wherein the one or more preventative actions comprise causing operation of at least one of the at least two electromechanical machines to stop.
Clause 5.7 The computer-implemented method of clause 1.7, wherein the artificial intelligence engine comprises one or more trained computer-implemented models.
Clause 6.7 The computer-implemented method of clause 1.7, wherein the first treatment plan and the second treatment plan are executed while the at least two of the plurality of users are using the at least two respective electromechanical machines.
Clause 7.7 The computer-implemented method of clause 1.7, further comprising:
   receiving subsequent data associated with the plurality of users;
based on the subsequent data, determining, using the artificial intelligence engine, whether progress towards a desired state was made by the at least two users of the plurality of users;
based on the determination, modifying at least one of the at least two treatment plans while the at least two users use the at least two respective electromechanical machines.
Clause 8.7 A tangible, non-transitory computer-readable medium storing instructions that, when executed, cause one or more processing devices to:
   receive data associated with a plurality of users having a similar medical condition, wherein the data is associated with one or more of a characteristic of the plurality of users and a performance metric of the plurality of users;
   based on the data, generate, using an artificial intelligence engine and evidence-based research, at least two treatment plans for at least two of the plurality of users, wherein a first treatment plan of the at least two treatment plans comprises instructions to inform a first user of an aspect of the first treatment plan and wherein, further, a second treatment plan of the at least two treatment plans excludes instructions to inform a second user of an aspect of the second treatment plan; and
   based on the at least two treatment plans, cause operation of at least two electromechanical machines associated respectively with the at least two treatment plans to be controlled.
Clause 9.7 The computer-readable medium of clause 8.7, wherein the one or more processing devices:
   receive subsequent data associated with the plurality of users; and
   based on the subsequent data, retrain, using reinforcement learning, one or more computer-implemented models of the artificial intelligence engine.
Clause 10.7 The computer-readable medium of clause 8.7, wherein, responsive to receiving subsequent data that satisfies an emergency threshold level, performing one or more preventative actions.
Clause 11.7 The computer-readable medium of clause 10.7, wherein the one or more preventative actions comprise causing operation of at least one of the at least two electromechanical machines to stop.
Clause 12.7 The computer-readable medium of clause 8.7, wherein the artificial intelligence engine comprises one or more trained computer-implemented models.
Clause 13.7 The computer-readable medium of clause 8.7, wherein the first treatment plan and the second treatment plan are executed while the at least two of the plurality of users are using the at least two respective electromechanical machines.
Clause 14.7 The computer-implemented method of clause 8.7, wherein the one or more processing devices:
   receive subsequent data associated with the plurality of users;
based on the subsequent data, determine, using the artificial intelligence engine, whether progress towards a desired state was made by the at least two users of the plurality of users;
based on the determination, modifying at least one of the at least two treatment plans while the at least two users use the at least two respective electromechanical machines.
Clause 15.7 A system comprising:
   one or more memory devices storing instructions; and
   one or more processing devices communicatively coupled to the one or more memory devices, wherein the one or more processing devices execute the instructions to:
   receive data associated with a plurality of users having a similar medical condition, wherein the data is associated with one or more of a characteristic of the plurality of users and a performance metric of the plurality of users;
   based on the data, generate, using an artificial intelligence engine and evidence-based research, at least two treatment plans for at least two of the plurality of users, wherein a first treatment plan of the at least two treatment plans comprises instructions to inform a first user of an aspect of the first treatment plan and wherein, further, a second treatment plan of the at least two treatment plans excludes instructions to inform a second user of an aspect of the second treatment plan; and
   based on the at least two treatment plans, cause operation of at least two electromechanical machines associated respectively with the at least two treatment plans to be controlled.
Clause 16.7 The system of clause 15.7, wherein the one or more processing devices:
   receive subsequent data associated with the plurality of users; and
   based on the subsequent data, retrain, using reinforcement learning, one or more computer-implemented models of the artificial intelligence engine.
Clause 17.7 The system of clause 15.7, wherein, responsive to receiving subsequent data that satisfies an emergency threshold level, performing one or more preventative actions.
Clause 18.7 The system of clause 17.7, wherein the one or more preventative actions comprise causing operation of at least one of the at least two electromechanical machines to stop.
Clause 19.7 The system of clause 15.7, wherein the artificial intelligence engine comprises one or more trained computer-implemented models.
Clause 20.7 The system of clause 15.7, wherein the first treatment plan and the second treatment plan are executed while the at least two of the plurality of users are using the at least two respective electromechanical machines.

Any of the systems and methods described in this disclosure may be used in connection with rehabilitation. Unless expressly stated otherwise, is to be understood that rehabilitation includes prehabilitation (also referred to as “pre-habilitation” or “prehab”). Prehabilitation may be used as a preventative procedure or as a pre-surgical or pre-treatment procedure. Prehabilitation may include any action performed by or on a patient (or directed to be performed by or on a patient, including, without limitation, remotely or distally through telemedicine) to, without limitation, prevent or reduce a likelihood of injury (e.g., prior to the occurrence of the injury); improve recovery time subsequent to surgery; improve strength subsequent to surgery; or any of the foregoing with respect to any non-surgical clinical treatment plan to be undertaken for the purpose of ameliorating or mitigating injury, dysfunction, or other negative consequence of surgical or non-surgical treatment on any external or internal part of a patient's body. For example, a mastectomy may require prehabilitation to strengthen muscles or muscle groups affected directly or indirectly by the mastectomy. As a further non-limiting example, the removal of an intestinal tumor, the repair of a hernia, open-heart surgery or other procedures performed on internal organs or structures, whether to repair those organs or structures, to excise them or parts of them, to treat them, etc., can require cutting through and harming numerous muscles and muscle groups in or about, without limitation, the abdomen, the ribs and/or the thoracic cavity. Prehabilitation can improve a patient's speed of recovery, measure of quality of life, level of pain, etc. in all the foregoing procedures. In one embodiment of prehabilitation, a pre-surgical procedure or a pre-non-surgical-treatment may include one or more sets of exercises for a patient to perform prior to such procedure or treatment. The patient may prepare an area of his or her body for the surgical procedure by performing the one or more sets of exercises, thereby strengthening muscle groups, improving existing and/or establishing new muscle memory, enhancing mobility, improving blood flow, and/or the like.

In some embodiments, the systems and methods described herein may use artificial intelligence and/or machine learning to generate a prehabilitation treatment plan for a user. Additionally, or alternatively, the systems and methods described herein may use artificial intelligence and/or machine learning to recommend an optimal exercise machine configuration for a user. For example, a data model may be trained on historical data such that the data model may be provided with input data relating to the user and may generate output data indicative of a recommended exercise machine configuration for a specific user. Additionally, or alternatively, the systems and methods described herein may use machine learning and/or artificial intelligence to generate other types of recommendations relating to prehabilitation, such as recommended reading material to educate the patient, a recommended medical professional specialist to contact, and/or the like.

### System and Method for Using Artificial Intelligence to Modify, Based on Predictive Analytics, a Treatment Plan for the Purpose of Optimizing Patient Outcomes and Pain Levels During Treatment Sessions

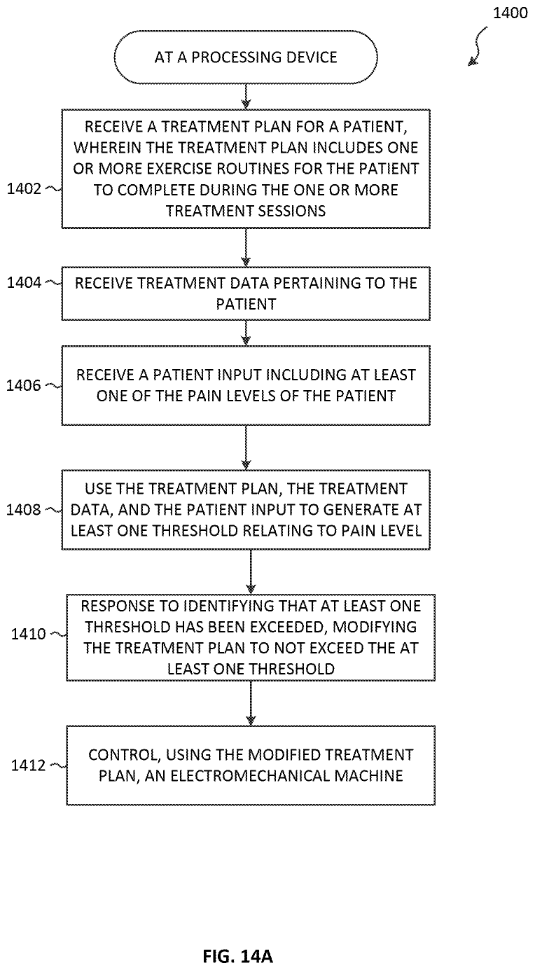

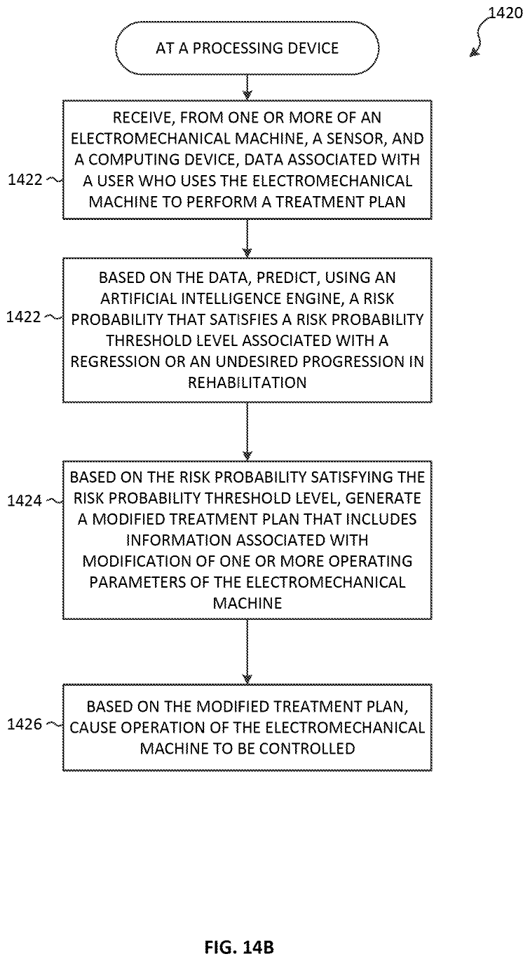

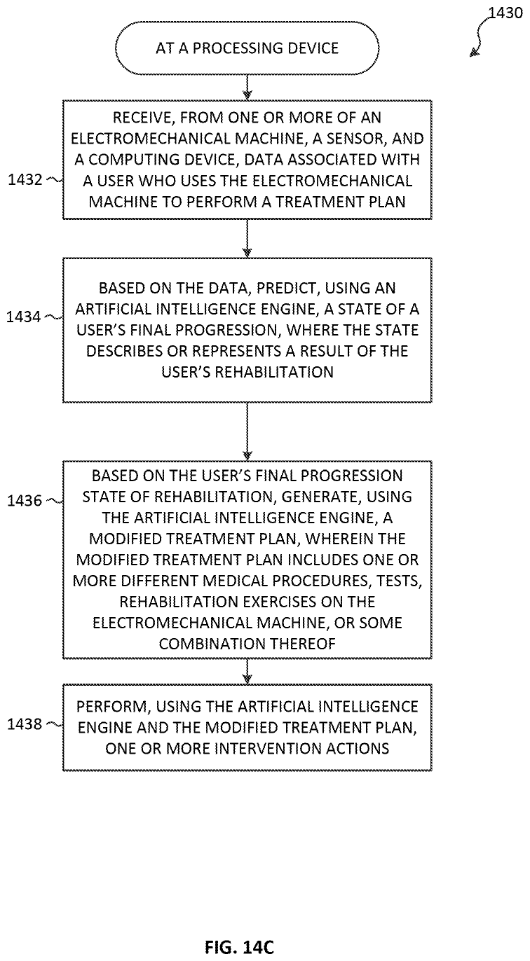

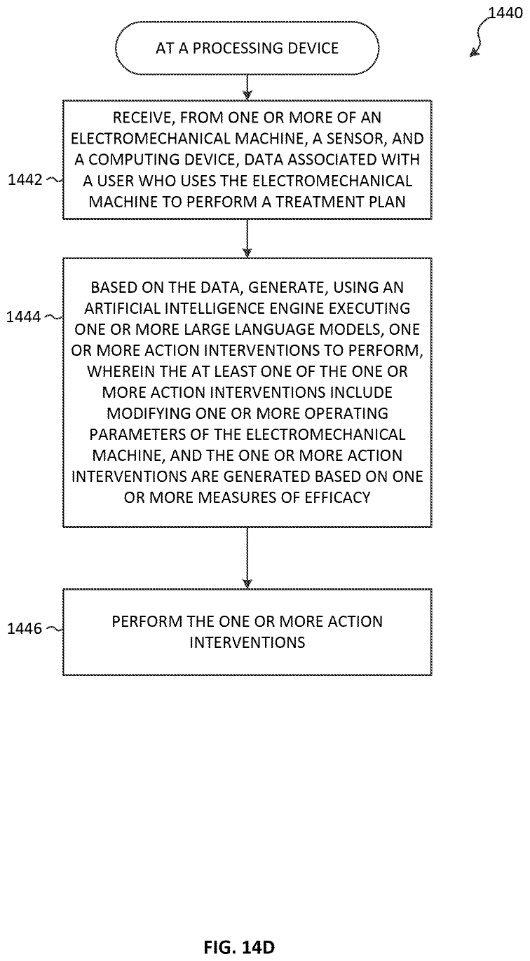

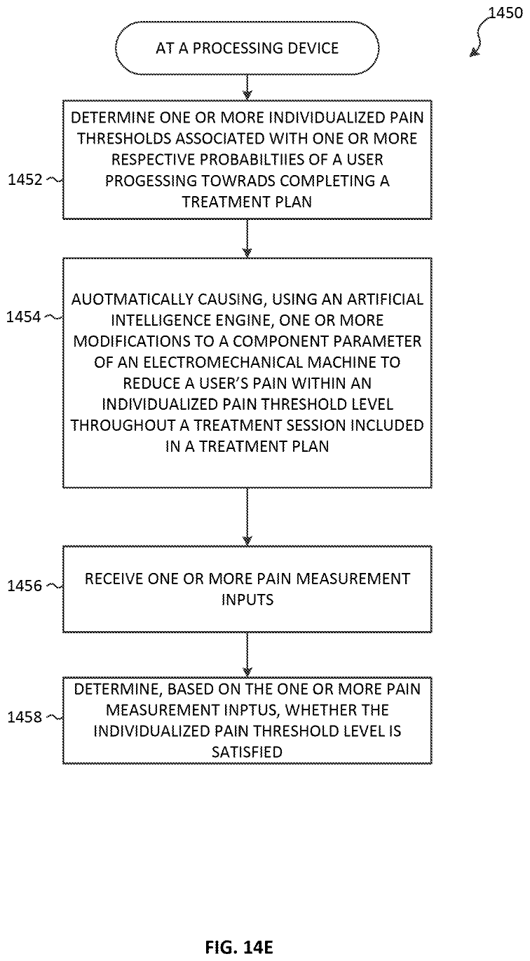

FIG. 14A shows an example embodiment of a method 1400 for using artificial intelligence to modify, based on predictive analytics, a treatment plan for the purpose of optimizing patient outcomes and pain levels during treatment sessions. The method 1400 is performed by processing logic that may include hardware (circuitry, dedicated logic, etc.), software (such as is run on a general-purpose computer system or a dedicated machine), or a combination of both. The method 1400 and/or each of its individual functions, routines, subroutines, methods (as the term is used in object-oriented programming), or operations may be performed by one or more processing devices of a computing device (e.g., any component of FIG. 1, such as the assistant interface 94, patient interface 50, and/or server 30 executing the artificial intelligence engine 11, training engine 9, machine learning models 13, etc.) implementing the method 400. The method 1400 may be implemented as computer instructions stored on a memory device and able to be executed by the one or more processing devices. In certain implementations, the method 1400 may be performed by a single processing thread. Alternatively, the method 1400 may be performed by two or more processing threads, each thread implementing one or more individual functions, routines, subroutines, methods or operations of the methods. In some embodiments, one or more accelerators may be used to increase the performance of a processing device by offloading various functions, routines, subroutines, methods or operations from the processing device.

For simplicity of explanation, the method 1400 is depicted in FIG. 14A and described as a series of operations. However, operations in accordance with this disclosure can occur in various orders and/or concurrently, and/or with other operations not presented and described herein. For example, the operations depicted in the method 1400 in FIG. 14A may occur in combination with any other operation of any other method disclosed herein. Furthermore, not all illustrated operations may be required to implement the method 1400 in accordance with the disclosed subject matter. In addition, those skilled in the art will understand and appreciate that the method 1400 could alternatively be represented as a series of interrelated states via a state diagram or event diagram.

In some embodiments, one or more computer-implemented models (e.g., machine learning models, neural networks, expert systems, etc.) may be generated and trained by the artificial intelligence engine 11 and/or the training engine 9 to perform one or more of the operations of the methods described herein. For example, to perform the one or more operations, the processing device may execute the one or more computer-implemented models. In some embodiments, the one or more computer-implemented models may be iteratively retrained to select different features capable of enabling optimization of output. The features that may be modified may include a number of nodes included in each layer of the models, an objective function executed at each node, a number of layers, various weights associated with outputs of each node, and the like.

The method 1400 may include one or more steps for modifying, by the artificial intelligence engine 11, a treatment plan for optimizing patient or user outcomes. The outcome may be partially based on one or more pain levels experienced or reported during one or more treatment sessions of the treatment plan. In some embodiments, the treatment plan may include one or more exercise routines for the patient or user to perform on the electromechanical machine.

At step 1402, the processing device may receive a treatment plan for a patient or user. The treatment plan may include one or more exercise routines for the patient or user to complete during the one or more treatment sessions.

At step 1404, the processing device may receive treatment data pertaining to the patient or user. In some embodiments, the treatment data may include at least one of characteristics of the patient or user, measurement information pertaining to the patient or user while the patient or user uses the electromechanical machine, and characteristics of the electromechanical machine. In some embodiments, at least some of the treatment data may correspond to sensor data from a sensor of a wearable device (e.g., goniometer, watch, ring, necklace, belt, strap, bracelet, sock, etc.) worn by the patient during one of the one or more treatment sessions.

At step 1406, the processing device may receive a patient input including at least one of the pain levels of the patient. For example, a user may use a computing device presenting a user interface to input a selected pain level that the user is currently experiencing, and the computing device may transmit the selected pain level to the processing device.

At step 1408, the processing device may use the treatment plan, the treatment data, and the patient input to generate at least one threshold relating to pain level. In some embodiments, the at least one threshold relating to pain may be tailored to the patient or user in view of cohort data related to multiple other patients and users executing similar treatment plans, having similar treatment data, and entering similar patient inputs. In some embodiments, one or more computer-implemented models (e.g., machine learning models 13) may be trained to generate the at least one threshold level. The computer-implemented models may be trained with training data including inputs (e.g., treatment plans, treatment data, patient inputs, or some combination thereof) mapped to outputs (e.g., pain level threshold).

At step 1410, responsive to identifying that the at least one threshold has been exceeded, the processing device may modify the treatment plan to not exceed the at least one threshold. In some embodiments, one or more computer-implemented models (e.g., machine learning models 13) may be trained to modify the treatment plan to not exceed the at least one threshold. The computer-implemented models may be trained with training data including inputs (e.g., pain level thresholds) mapped to outputs (e.g., treatment plans, treatment data, patient inputs, or some combination thereof). In some embodiments, the one or more computer-implemented models may modify the treatment plan by generating, during one of the one or more treatment sessions, at least one updated exercise routine. In some embodiments, at least one of the treatment data and the patient input may be received in real-time (e.g., less than 2 seconds) or near real-time (e.g., at 2 seconds or 10 seconds exactly and also between 2 and 10 seconds), and the treatment plan may be correspondingly modified in real-time or near real-time.

At step 1412, the processing device may control, using the modified treatment plan, an electromechanical machine. For example, the processing device may control, while the patient or user uses the electromechanical machine band based on the modified treatment plan, the electromechanical machine.

In some embodiments, a telehealth or telemedicine session may be initiated between a computing device associated with the user and/or the electromechanical machine and with a computing device associated with a medical professional, therapist, coach, trainer, etc. The processing device may control, while the patient uses the electromechanical machine during a telehealth or telemedicine session and based on the modified treatment plan, the electromechanical machine.

In some embodiments, the processing device may receive modified patient input correlating with an updated pain level of the patient or user. The processing device may use the modified treatment plan, the treatment data, and the modified patient input to generate at least one modified threshold. In some embodiments, responsive to a subsequent occurrence of exceeding the at least one modified threshold, the processing device may further modify the modified treatment plan.

Consistent with the above disclosure, the examples of systems and methods enumerated in the following clauses are specifically contemplated and are intended as a non-limiting set of examples.
Clause 1.25 A method for modifying, by an artificial intelligence engine, a treatment plan for optimizing patient outcomes, wherein an outcome is partially based on one or more pain levels experienced or reported during one or more treatment sessions, the method comprising:
   receiving the treatment plan for a patient, wherein the treatment plan comprises one or more exercise routines for the patient to complete during the one or more treatment sessions;
   receiving treatment data pertaining to the patient;
   receiving a patient input comprising at least one of the pain levels of the patient;
   using the treatment plan, the treatment data, and the patient input to generate at least one threshold relating to pain level;
   responsive to identifying that the at least one threshold has been exceeded, modifying the treatment plan to not exceed the at least one threshold; and
   controlling, using the modified treatment plan, an electromechanical machine.
Clause 2.25 The method of clause 1.25, wherein the treatment plan comprises the one or more exercise routines for the patient to perform on the electromechanical machine.
Clause 3.25 The method of clause 2.25, wherein the treatment data comprises at least one of characteristics of the patient, measurement information pertaining to the patient while the patient uses the electromechanical machine, and characteristics of the electromechanical machine.
Clause 4.25 The method of clause 2.25, further comprising controlling, while the patient uses the electromechanical machine and based on the modified treatment plan, the electromechanical machine.
Clause 5.25 The method of clause 2.25, further comprising controlling, while the patient uses the electromechanical machine during a telemedicine session and based on the modified treatment plan, the electromechanical machine.
Clause 6.25 The method of clause 1.25, further comprising:
   receiving modified patient input correlating with an updated pain level of the patient;
   using the modified treatment plan, the treatment data, and the modified patient input to generate at least one modified threshold; and
   responsive to a subsequent occurrence of exceeding the at least one modified threshold, further modifying the modified treatment plan.
Clause 7.25 The method of clause 1.25, wherein at least some of the treatment data corresponds to sensor data from a sensor of a wearable device worn by the patient during one of the one or more treatment sessions.
Clause 8.25 The method of clause 1.25, wherein modifying the treatment plan comprises generating, during one of the one or more treatment sessions, at least one updated exercise routine.
Clause 9.25 The method of clause 1.25, wherein at least one of the treatment data and the patient input is received in real-time or near real-time, and wherein the treatment plan is correspondingly modified in real-time or near real-time.
Clause 10.25 A tangible, non-transitory computer-readable medium storing instructions that, when executed, cause one or more processing devices to:
   receive the treatment plan for a patient, wherein the treatment plan comprises one or more exercise routines for the patient to complete during the one or more treatment sessions;
   receive treatment data pertaining to the patient;
   receive a patient input comprising at least one of the pain levels of the patient;
   use the treatment plan, the treatment data, and the patient input to generate at least one threshold relating to pain level;
   responsive to identifying that the at least one threshold has been exceeded, modify the treatment plan to not exceed the at least one threshold; and
   control, using the modified treatment plan, an electromechanical machine.
Clause 11.25 The computer-readable medium of clause 10.25, wherein the treatment plan comprises the one or more exercise routines for the patient to perform on the electromechanical machine.
Clause 12.25 The computer-readable medium of clause 11.25, wherein the treatment data comprises at least one of characteristics of the patient, measurement information pertaining to the patient while the patient uses the electromechanical machine, and characteristics of the electromechanical machine.
Clause 13.25 The computer-readable medium of clause 11.25, further comprising controlling, while the patient uses the electromechanical machine and based on the modified treatment plan, the electromechanical machine.
Clause 14.25 The computer-readable medium of clause 11.25, further comprising controlling, while the patient uses the electromechanical machine during a telemedicine session and based on the modified treatment plan, the electromechanical machine.
Clause 15.25 The computer-readable medium of clause 10.25, wherein the one or more processing devices:
   receive modified patient input correlating with an updated pain level of the patient;
   use the modified treatment plan, the treatment data, and the modified patient input to generate at least one modified threshold; and
   responsive to a subsequent occurrence of exceeding the at least one modified threshold, further modify the modified treatment plan.
Clause 16.25 The computer-readable medium of clause 10.25, wherein at least some of the treatment data corresponds to sensor data from a sensor of a wearable device worn by the patient during one of the one or more treatment sessions.
Clause 17.25 The computer-readable medium of clause 10.25, wherein modifying the treatment plan comprises generating, during one of the one or more treatment sessions, at least one updated exercise routine.
Clause 18.25 The computer-readable medium of clause 10.25, wherein at least one of the treatment data and the patient input is received in real-time or near real-time, and wherein the treatment plan is correspondingly modified in real-time or near real-time.
Clause 19.25 A system comprising:
   one or more memory devices storing instructions; and
   one or more processing devices communicatively coupled to the one or more memory devices, wherein the one or more processing devices execute the instructions to:
receive the treatment plan for a patient, wherein the treatment plan comprises one or more exercise routines for the patient to complete during the one or more treatment sessions;
receive treatment data pertaining to the patient;
receive a patient input comprising at least one of the pain levels of the patient;
use the treatment plan, the treatment data, and the patient input to generate at least one threshold relating to pain level;
responsive to identifying that the at least one threshold has been exceeded, modify the treatment plan to not exceed the at least one threshold; and
control, using the modified treatment plan, an electromechanical machine.
Clause 20.25 The system of clause 19.25, wherein the treatment plan comprises the one or more exercise routines for the patient to perform on the electromechanical machine.

### Systems and Methods for Using AI/ML to Enable, Via AI-Driven Interventions, the Early Detection of Patients Deemed High Risk Due to Slow Progression in Rehabilitation

FIG. 14B shows an example embodiment of a method 1420 for using artificial intelligence (AI) and machine learning to enable, via AI-driven interventions, the early detection of patients deemed high risk due to slow progression in rehabilitation. The method 1420 is performed by processing logic that may include hardware (circuitry, dedicated logic, etc.), software (such as is run on a general-purpose computer system or a dedicated machine), or a combination of both. The method 1300 and/or each of its individual functions, routines, subroutines, methods (as the term is used in object-oriented programming), or operations may be performed by one or more processing devices of a computing device (e.g., any component of FIG. 1, such as assistant interface 94, patient interface 50, and/or server 30 executing the artificial intelligence engine 11, training engine 9, machine learning models 13, etc.) implementing the method 1420. The method 1420 may be implemented as computer instructions stored on a memory device and able to be executed by the one or more processing devices. In certain implementations, the method 1420 may be performed by a single processing thread. Alternatively, the method 1420 may be performed by two or more processing threads, each thread implementing one or more individual functions, routines, subroutines, methods or operations of the methods. In some embodiments, one or more accelerators may be used to increase the performance of a processing device by offloading various functions, routines, subroutines, methods or operations from the processing device.

For simplicity of explanation, the method 1420 is depicted in FIG. 14B and described as a series of operations. However, operations in accordance with this disclosure can occur in various orders and/or concurrently, and/or with other operations not presented and described herein. For example, the operations depicted in the method 1420 in FIG. 14B may occur in combination with any other operation of any other method disclosed herein. Furthermore, not all illustrated operations may be required to implement the method 1420 in accordance with the disclosed subject matter. In addition, those skilled in the art will understand and appreciate that the method 1420 could alternatively be represented as a series of interrelated states via a state diagram or event diagram.

In some embodiments, one or more computer-implemented models (e.g., machine learning models, neural networks, expert systems, etc.) may be generated and trained by the artificial intelligence engine 11 and/or the training engine 9 to perform one or more of the operations of the methods described herein. For example, to perform the one or more operations, the processing device may execute the one or more computer-implemented models. In some embodiments, the one or more computer-implemented models may be iteratively retrained to select different features capable of enabling optimization of output. The features that may be modified may include a number of nodes included in each layer of the models, an objective function executed at each node, a number of layers, various weights associated with outputs of each node, and the like.

At step 1422, the processing device may receive, from one or more of an electromechanical machine, a sensor, and a computing device (e.g., a computing device associated with a user, a medical professional, a clinician, a therapist, a trainer, a coach, etc.), data associated with a user who uses the electromechanical machine to perform a treatment plan. In some embodiments, the electromechanical machine may include at least one pedal.

At step 1424, based on the data, the processing device may predict, using the artificial intelligence engine 11, a risk probability that satisfies a risk probability threshold level associated with a regression or an undesired progression in rehabilitation. In some embodiments, the risk probability threshold level may be configured by a physician, a therapist, a clinician, another medical professional, or a third-party individual.

At step 1426, based on the risk probability satisfying the risk probability threshold level, the processing device may generate a modified treatment plan that includes information associated with modification of one or more operating parameters of the electromechanical machine. In some embodiments, the artificial intelligence engine 11 may generate the modified treatment plan. In some embodiments, one or more computer-implemented models (e.g., machine learning models 13) may be executed or used by the artificial intelligence engine 11 to generate the modified treatment plan.

At step 1428, based on the modified treatment plan, the processing device may cause operation of the electromechanical machine to be controlled. In some embodiments, based on the modified treatment plan, the artificial intelligence engine 11 may execute or use one or more computer-implemented models (e.g., machine learning models 13) to generate one or more control instructions that are transmitted to the electromechanical machine to cause operation of the electromechanical machine to be controlled. For example, the control instructions may specify certain operating parameters (e.g., revolutions per minute, amount of resistance, speed, etc.) of certain exercises included in the modified treatment plan, and the electromechanical machine may execute the control instructions to set the certain operating parameters of the certain exercises.

In some embodiments, the processing device may receive, from one or more of the electromechanical machine, the sensor, and the computing device, subsequent data associated with the user who uses the electromechanical machine to perform the modified treatment plan. Based on the subsequent data, the processing device may predict, using the artificial intelligence engine 11, a subsequent risk probability that does not satisfy the risk probability threshold level associated with the regression or the undesired progression in rehabilitation. In some embodiments, based on the modified treatment plan, the processing device may cause operation of the electromechanical machine to be maintained by using the one or more modified operating parameters.

Consistent with the above disclosure, the examples of systems and methods enumerated in the following clauses are specifically contemplated and are intended as a non-limiting set of examples.
Clause 1.9 A computer-implemented method comprising:
   receiving, from one or more of an electromechanical machine, a sensor, and a computing device, data associated with a user who uses the electromechanical machine to perform a treatment plan;
   based on the data, predicting, using an artificial intelligence engine, a risk probability that satisfies a risk probability threshold level associated with a regression or an undesired progression in rehabilitation;
   based on the risk probability satisfying the risk probability threshold level, generating a modified treatment plan that comprises information associated with modification of one or more operatinameters of the electromechanical machine; and
   based on the modified treatment plan, causing operation of the electromechanical machine to be controlled.
Clause 2.9 The computer-implemented method of clause 1.9, wherein the modified treatment plan is generated using the artificial intelligence engine.
Clause 3.9 The computer-implemented method of clause 1.9, further comprising:
   receiving, from one or more of the electromechanical machine, the sensor, and the computing device, subsequent data associated with the user who uses the electromechanical machine to perform the modified treatment plan;
   based on the subsequent data, predicting, using the artificial intelligence engine, a subsequent risk probability that does not satisfy the risk probability threshold level associated with the regression or the undesired progression in rehabilitation; and
   based on the modified treatment plan, causing operation of the electromechanical machine to be maintained using the one or more modified operating parameters.
Clause 4.9 The computer-implemented method of clause 1.9, wherein the artificial intelligence engine comprises one or more trained computer-implemented models wherein the computer-implemented models comprise at least one machine learning model.
Clause 5.9 The computer-implemented method of clause 1.9, further comprising by using the artificial intelligence engine, the operation of the electromechanical machine to be controlled.
Clause 6.9 The computer-implemented method of clause 1.9, wherein the risk probability threshold level is configured by a physician, a therapist, a clinician, another medical professional, or a third-party individual.
Clause 7.9 The computer-implemented method of clause 1.9, wherein the electromechanical machine comprises at least one pedal.
Clause 8.9 A tangible, non-transitory computer-readable medium storing instructions that, when executed, cause one or more processing devices to:
   receive, from one or more of an electromechanical machine, a sensor, and a computing device, data associated with a user who uses the electromechanical machine to perform a treatment plan;
   based on the data, predicting, use an artificial intelligence engine, a risk probability that satisfies a risk probability threshold level associated with a regression or an undesired progression in rehabilitation;
   based on the risk probability satisfying the risk probability threshold level, generate a modified treatment plan that comprises information associated with modification of one or more operating parameters of the electromechanical machine; and
   based on the modified treatment plan, cause operation of the electromechanical machine to be controlled.
Clause 9.9 The computer-readable medium of clause 8.9, wherein the modified treatment plan is generated using the artificial intelligence engine.
Clause 10.9 The computer-readable medium of clause 8.9, wherein the one or more processing devices executes the instructions to:
   receive, from one or more of the electromechanical machine, the sensor, and the computing device, subsequent data associated with the user who uses the electromechanical machine to perform the modified treatment plan;
   based on the subsequent data, predict, using the artificial intelligence engine, a subsequent risk probability that does not satisfy the risk probability threshold level associated with the regression or the undesired progression in rehabilitation; and
   based on the modified treatment plan, cause operation of the electromechanical machine to be maintained using the one or more modified operating parameters.
Clause 11.9 The computer-readable medium of clause 8.9, wherein the artificial intelligence engine comprises one or more trained computer-implemented models wherein the computer-implemented models comprise at least one machine learning model.
Clause 12.9 The computer-readable medium of clause 8.9, wherein by using the artificial intelligence engine, the operation of the electromechanical machine to be controlled by the one or more processing devices.
Clause 13.9 The computer-readable medium of clause 8.9, wherein the risk probability threshold level is configured by a physician, a therapist, a clinician, another medical professional, or a third-party individual.
Clause 14.9 The computer-readable medium of clause 8.9, wherein the electromechanical machine comprises at least one pedal.
Clause 15.9 A system comprising:
   one or more memory devices storing instructions; and
   one or more processing devices communicatively coupled to the one or more memory devices, wherein the one or more processing devices execute the instructions to:
receive, from one or more of an electromechanical machine, a sensor, and a computing device, data associated with a user who uses the electromechanical machine to perform a treatment plan;
based on the data, predicting, use an artificial intelligence engine, a risk probability that satisfies a risk probability threshold level associated with a regression or an undesired progression in rehabilitation;
based on the risk probability satisfying the risk probability threshold level, generate a modified treatment plan that comprises information associated with modification of one or more operating parameters of the electromechanical machine; and
based on the modified treatment plan, cause operation of the electromechanical machine to be controlled.
Clause 16.9 The system of clause 15.9, wherein the modified treatment plan is generated using the artificial intelligence engine.
Clause 17.9 The system of clause 15.9, wherein the one or more processing devices executes the instructions to:
   receive, from one or more of the electromechanical machine, the sensor, and the computing device, subsequent data associated with the user who uses the electromechanical machine to perform the modified treatment plan;
   based on the subsequent data, predict, using the artificial intelligence engine, a subsequent risk probability that does not satisfy the risk probability threshold level associated with the regression or the undesired progression in rehabilitation; and
   based on the modified treatment plan, cause operation of the electromechanical machine to be maintained using the one or more modified operating parameters.
Clause 18.9 The system of clause 15.9, wherein the artificial intelligence engine comprises one or more trained computer-implemented models wherein the computer-implemented models comprise at least one machine learning model.
Clause 19.9 The system of clause 15.9, wherein by using the artificial intelligence engine, the operation of the electromechanical machine to be controlled by the one or more processing devices.
Clause 20.9 The system of clause 15.9, wherein the risk probability threshold level is configured by a physician, a therapist, a clinician, another medical professional, or a third-party individual.

### Systems and Methods for Predicting a User's Final Progression State of Rehabilitation Based on Number of Sessions, Medication Intake, Pain Level, or Some Combination Thereof And for Using the Predictions to Perform AI-Driven Interventions

FIG. 14C shows an example embodiment of a method 1430 predicting a user's final progression state of rehabilitation based on number of sessions, medication intake, pain level, or some combination thereof and for using the predictions to perform AI-driven interventions. The method 1430 is performed by processing logic that may include hardware (circuitry, dedicated logic, etc.), software (such as is run on a general-purpose computer system or a dedicated machine), or a combination of both. The method 1430 and/or each of its individual functions, routines, subroutines, methods (as the term is used in object-oriented programming), or operations may be performed by one or more processing devices of a computing device (e.g., any component of FIG. 1, such as assistant interface 94, patient interface 50, and/or server 30 executing the artificial intelligence engine 11, training engine 9, machine learning models 13, etc.) implementing the method 1430. The method 1430 may be implemented as computer instructions stored on a memory device and able to be executed by the one or more processing devices. In certain implementations, the method 1430 may be performed by a single processing thread. Alternatively, the method 1430 may be performed by two or more processing threads, each thread implementing one or more individual functions, routines, subroutines, methods or operations of the methods. In some embodiments, one or more accelerators may be used to increase the performance of a processing device by offloading various functions, routines, subroutines, methods or operations from the processing device.

For simplicity of explanation, the method 1430 is depicted in FIG. 14C and described as a series of operations. However, operations in accordance with this disclosure can occur in various orders and/or concurrently, and/or with other operations not presented and described herein. For example, the operations depicted in the method 1430 in FIG. 14C may occur in combination with any other operation of any other method disclosed herein. Furthermore, not all illustrated operations may be required to implement the method 1430 in accordance with the disclosed subject matter. In addition, those skilled in the art will understand and appreciate that the method 1430 could alternatively be represented as a series of interrelated states via a state diagram or event diagram.

In some embodiments, one or more computer-implemented models (e.g., machine learning models, neural networks, expert systems, etc.) may be generated and trained by the artificial intelligence engine 11 and/or the training engine 9 to perform one or more of the operations of the methods described herein. For example, to perform the one or more operations, the processing device may execute the one or more computer-implemented models. In some embodiments, the one or more computer-implemented models may be iteratively retrained to select different features capable of enabling optimization of output. The features that may be modified may include a number of nodes included in each layer of the models, an objective function executed at each node, a number of layers, various weights associated with outputs of each node, and the like.

At step 1432, the processing device may receive, from one or more of an electromechanical machine, a sensor, and a computing device, data associated with a user who uses the electromechanical machine to perform a treatment plan. The data may include a number of exercise sessions, medication information associated with the user, pain level experienced or reported by the user, or some combination thereof. In some embodiments, the electromechanical machine may include at least one pedal. In some embodiments, the processing device may receive the treatment plan from a computing device associated with a medical professional. In some embodiments, the processing device may receive the treatment plan and, using the treatment plan and the artificial intelligence engine 11, cause the electromechanical machine to be controlled.

At step 1434, based on the data, the processing device may predict, using an artificial intelligence engine 11, a state of the user's final progression, where the state may describe or represent a result of the user's rehabilitation. In some embodiments, the artificial intelligence may execute or use one or more computer-implemented models (e.g., machine learning models 13). The computer-implemented models may be trained with training data including inputs (e.g., rehabilitation progression information associated with the user, characteristics of the user, measurements associated with user performance, medical information associated with the user, etc.) mapped to outputs (e.g., a state of the user's final progression).

In some embodiments, the processing device may transmit a message to the computing device associated with the user, a therapist, a clinician, another medical professional, a third-party individual, or some combination thereof. The message may include the state of the user's final progression as the result of the user's rehabilitation and the state may be presented via a user interface or emitted via an audio device (e.g., speaker).

At step 1436, based on the user's final progression state of rehabilitation, the processing device may generate, using the artificial intelligence engine 11, a modified treatment plan. The modified treatment plan may include one or more different medical procedures, tests, rehabilitation exercises on the electromechanical machine, or some combination thereof. In some embodiments, the artificial intelligence may execute or use one or more computer-implemented models (e.g., machine learning models 13, etc.). The computer-implemented models may be trained with training data including inputs (e.g., a state of the user's final progression, etc.) mapped to outputs (e.g., a modified treatment plan).

At step 1438, the processing device may perform, using the artificial intelligence engine 11 and the modified treatment plan, one or more intervention actions. In some embodiments, the one or more intervention actions may include causing operation of the electromechanical machine to be controlled. In some embodiments, the one or more intervention actions may include modifying one or more operating parameters of the electromechanical machine, transmitting a notification to one or more emergency services, transmitting a notification to the computing device, transmitting a notification to a medical professional, transmitting a notification to at least one designated emergency contact of the user, or some combination thereof.

Consistent with the above disclosure, the examples of systems and methods enumerated in the following clauses are specifically contemplated and are intended as a non-limiting set of examples.
Clause 1.10 A computer-implemented method, comprising:
   receiving, from one or more of an electromechanical machine, a sensor, and a computing device, data associated with a user who uses the electromechanical machine to perform a treatment plan, wherein the data comprises a number of exercise sessions, medication information associated with the user, pain level experienced or reported by the user, or some combination thereof;
   based on the data, predicting, using an artificial intelligence engine, a state of the user's final progression, where the state describes or represents a result of the user's rehabilitation;
   based on the user's final progression state of rehabilitation, generating, using the artificial intelligence engine, a modified treatment plan, wherein the modified treatment plan comprises one or more different medical procedures, tests, rehabilitation exercises on the electromechanical machine, or some combination thereof; and
   performing, using the artificial intelligence engine and the modified treatment plan, one or more intervention actions.
Clause 2.10 The computer-implemented method of clause 1.10, wherein the one or more intervention actions comprise causing operation of the electromechanical machine to be controlled.
Clause 3.10 The computer-implemented method of clause 1.10, wherein the one or more intervention actions comprise modifying one or more operating parameters of the electromechanical machine, transmitting a notification to one or more emergency services, transmitting a notification to the computing device, transmitting a notification to a medical professional, transmitting a notification to at least one designated emergency contact of the user, or some combination thereof.
Clause 4.10 The computer-implemented method of clause 1.10, wherein the electromechanical machine comprises at least one pedal.
Clause 5.10 The computer-implemented method of clause 1.10, wherein the artificial intelligence engine comprises at least one trained computer-implemented model comprising at least one machine learning model.
Clause 6.10 The computer-implemented method of clause 1.10, further comprising receiving the treatment plan and, using the treatment plan and the artificial intelligence engine, causing the electromechanical machine to be controlled.
Clause 7.10 The computer-implemented method of clause 1.10, further comprising transmitting a message to the computing device, wherein the message comprises the state of the user's final progression as the result of the user's rehabilitation and the state is presented via a user interface or emitted via an audio device.
Clause 8.10 A tangible, non-transitory computer-readable medium storing instructions that, when executed, cause one or more processing devices to:
   receive, from one or more of an electromechanical machine, a sensor, and a computing device, data associated with a user who uses the electromechanical machine to perform a treatment plan, wherein the data comprises a number of exercise sessions, medication information associated with the user, pain level experienced or reported by the user, or some combination thereof;
   based on the data, predict, using an artificial intelligence engine, a state of the user's final progression, where the state describes or represents a result of the user's rehabilitation;
   based on the user's final progression state of rehabilitation, generate, using the artificial intelligence engine, a modified treatment plan, wherein the modified treatment plan comprises one or more different medical procedures, tests, rehabilitation exercises on the electromechanical machine, or some combination thereof; and
   perform, using the artificial intelligence engine and the modified treatment plan, one or more intervention actions.
Clause 9.10 The computer-readable medium of clause 8.10, wherein the one or more intervention actions comprise causing operation of the electromechanical machine to be controlled.
Clause 10.10 The computer-readable medium of clause 8.10, wherein the one or more intervention actions comprise modifying one or more operating parameters of the electromechanical machine, transmitting a notification to one or more emergency services, transmitting a notification to the computing device, transmitting a notification to a medical professional, transmitting a notification to at least one designated emergency contact of the user, or some combination thereof.
Clause 11.10 The computer-readable medium of clause 8.10, wherein the electromechanical machine comprises at least one pedal.
Clause 12.10 The computer-readable medium of clause 8.10, wherein the artificial intelligence engine comprises at least one trained computer-implemented model comprising at least one machine learning model.
Clause 13.10 The computer-readable medium of clause 8.10, further comprising receiving the treatment plan and, using the treatment plan and the artificial intelligence engine, causing the electromechanical machine to be controlled.
Clause 14.10 The computer-readable medium of clause 8.10, further comprising transmitting a message to the computing device, wherein the message comprises the state of the user's final progression as the result of the user's rehabilitation and the state is presented via a user interface or emitted via an audio device.
Clause 15.10 A system comprising
   one or more memory devices storing instructions; and
   one or more processing devices communicatively coupled to the one or more memory devices, wherein the one or more processing devices executes the instructions to:
receive, from one or more of an electromechanical machine, a sensor, and a computing device, data associated with a user who uses the electromechanical machine to perform a treatment plan, wherein the data comprises a number of exercise sessions, medication information associated with the user, pain level experienced or reported by the user, or some combination thereof;
based on the data, predict, using an artificial intelligence engine, a state of the user's final progression, where the state describes or represents a result of the user's rehabilitation;
based on the user's final progression state of rehabilitation, generate, using the artificial intelligence engine, a modified treatment plan, wherein the modified treatment plan comprises one or more different medical procedures, tests, rehabilitation exercises on the electromechanical machine, or some combination thereof; and
perform, using the artificial intelligence engine and the modified treatment plan, one or more intervention actions.
Clause 16.10 The system of clause 15.10, wherein the one or more intervention actions comprise causing operation of the electromechanical machine to be controlled.
Clause 17.10 The system of clause 15.10, wherein the one or more intervention actions comprise modifying one or more operating parameters of the electromechanical machine, transmitting a notification to one or more emergency services, transmitting a notification to the computing device, transmitting a notification to a medical professional, transmitting a notification to at least one designated emergency contact of the user, or some combination thereof.
Clause 18.10 The system of Clause 15.10, wherein the electromechanical machine comprises at least one pedal.
Clause 19.10 The system of clause 15.10, wherein the artificial intelligence engine comprises at least one trained computer-implemented model comprising at least one machine learning model.
Clause 20.10 The system of clause 15.10, further comprising receiving the treatment plan and, using the treatment plan and the artificial intelligence engine, causing the electromechanical machine to be controlled.
   Systems and Methods for Determining and Performing Interventions Using an Electromechanical Machine Wherein the Interventions are Determined by Using a Large Language Model to Generate and Rank, Based on One or More Measures of Efficacy, a List Of Interventions

FIG. 14D shows an example embodiment of a method 1440 for determining and performing interventions using an electromechanical machine where the interventions may be determined by using a large language model to generate and rank, based on one or more measures of efficacy, a list of interventions. The method 1440 is performed by processing logic that may include hardware (circuitry, dedicated logic, etc.), software (such as is run on a general-purpose computer system or a dedicated machine), or a combination of both. The method 1440 and/or each of its individual functions, routines, subroutines, methods (as the term is used in object-oriented programming), or operations may be performed by one or more processing devices of a computing device (e.g., any component of FIG. 1, such as the assistant interface 94, patient interface 50, and/or server 30 executing the artificial intelligence engine 11, training engine 9, machine learning models 13, etc.) implementing the method 1440. The method 1440 may be implemented as computer instructions stored on a memory device and able to be executed by the one or more processing devices. In certain implementations, the method 1440 may be performed by a single processing thread. Alternatively, the method 1440 may be performed by two or more processing threads, each thread implementing one or more individual functions, routines, subroutines, methods or operations of the methods. In some embodiments, one or more accelerators may be used to increase the performance of a processing device by offloading various functions, routines, subroutines, methods or operations from the processing device.

For simplicity of explanation, the method 1440 is depicted in FIG. 14D and described as a series of operations. However, operations in accordance with this disclosure can occur in various orders and/or concurrently, and/or with other operations not presented and described herein. For example, the operations depicted in the method 1440 in FIG. 14D may occur in combination with any other operation of any other method disclosed herein. Furthermore, not all illustrated operations may be required to implement the method 1440 in accordance with the disclosed subject matter. In addition, those skilled in the art will understand and appreciate that the method 1440 could alternatively be represented as a series of interrelated states via a state diagram or event diagram.

In some embodiments, one or more computer-implemented models (e.g., machine learning models, neural networks, expert systems, etc.) may be generated and trained by the artificial intelligence engine 11 and/or the training engine 9 to perform one or more of the operations of the methods described herein. For example, to perform the one or more operations, the processing device may execute the one or more computer-implemented models. In some embodiments, the one or more computer-implemented models may be iteratively retrained to select different features capable of enabling optimization of output. The features that may be modified may include a number of nodes included in each layer of the models, an objective function executed at each node, a number of layers, various weights associated with outputs of each node, and the like.

At step 1442, the processing device may receive, from one or more of an electromechanical machine, a sensor, and a computing device, data associated with a user who uses the electromechanical machine to perform a treatment plan. In some embodiments, the data may be occluded, transformed, or otherwise de-identified due to the use of privacy enhancing techniques. Also, in some embodiments, the data may be processed to remove any personally identifiable information.

At step 1444, based on the data, the processing device may generate, using an artificial intelligence engine executing one or more large language models, one or more action interventions to perform. Large language models (i.e., LLMs) are a type of deep learning model that uses large amounts of training data to analyze and understand natural language. LLMs are used as the computational engine for services like ChatGPT, perplexity.ai and grok, among other things (e.g., other generative pre-trained transformers (GPTs)). The LLMs may be trained with action interventions, queries, statuses, recommendations, alerts, messages, insights, actions, etc.

In some embodiments, action interventions may refer to operations implemented in computer instructions, and the operations may include modifying operating parameters of an electromechanical machine, generating messages using the LLM, controlling applications, transmitting messages, and the like.

The artificial intelligence engine 11 may execute or use one or more computer-implemented models (e.g., machine learning models 13). The one or more computer-implemented models may be trained using training data including inputs (e.g., characteristics of the user, measurements of user performance, medical history of the user, etc.) mapped to outputs (e.g., action interventions).

In some embodiments, the one or more action interventions may be generated based on one or more measures of efficacy. In some embodiments, the artificial intelligence engine 11 may rank the one or more action interventions.

In some embodiments, the artificial intelligence engine 11 may use one or more secondary conditions identified and explained by the one or more LLMs. In some embodiments, the one or more LLMs may generate a description that the user has one or more secondary conditions that are inhibiting the progress of their rehabilitation and may provide one or more recommendations, diagnoses, suggestions, or the like to treat or care for the one or more secondary condition. In some embodiments, the one or more action interventions may include transmitting a message to the computing device associated with the user, a therapist, a clinician, another medical professional, a third-party individual, emergency service personnel, an emergency contact, or the like. Transmitting the message may cause the message to be displayed via a user interface of the computing device, audibly emitted via a speaker of the computing device, and/or the like.

At step 1446, the processing device may perform one or more action interventions. In some embodiments, the one or more action interventions may include causing operation of the electromechanical machine to be controlled. For example, in some embodiments, one or more control instructions may be transmitted by the processing device to the electromechanical machine (treatment apparatus 70) and/or patient interface 50 via the first network 34 and/or second network 58. The control instructions may be executed by the controller 72 to change one or more operating parameters of the electromechanical machine. In some embodiments, the operating parameters may be modified while the user or patient is using the electromechanical machine and/or during a telehealth session or telemedicine session between the user or patient and a clinician or another medical professional.

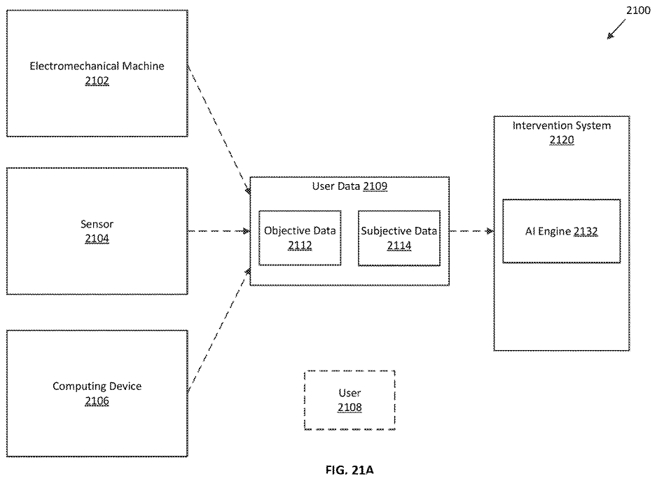

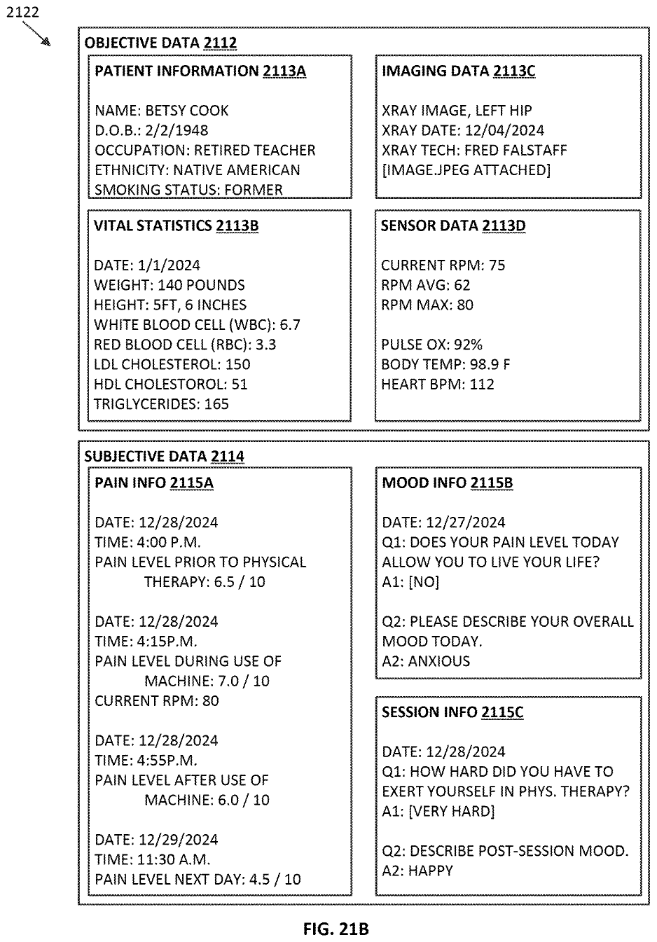

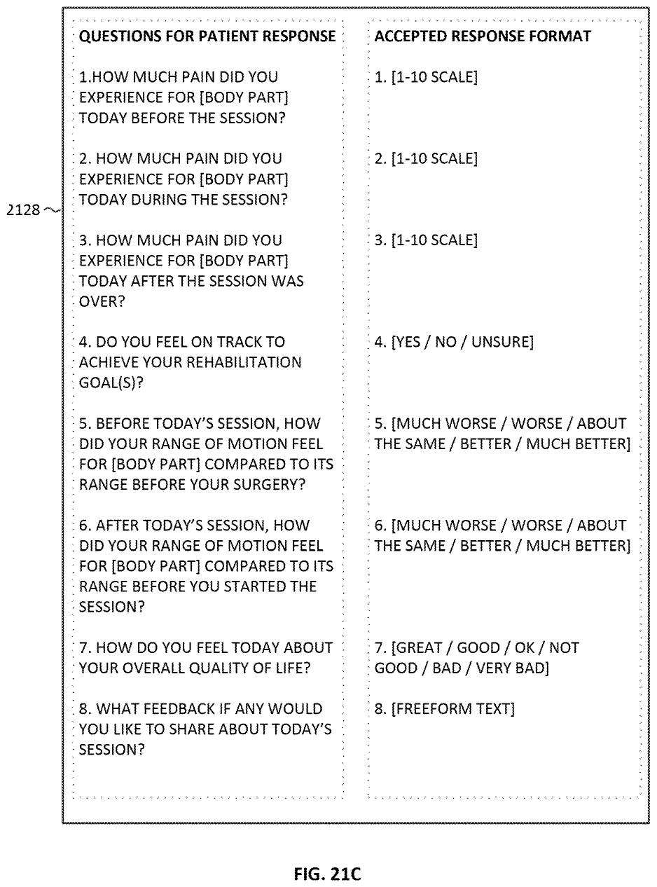

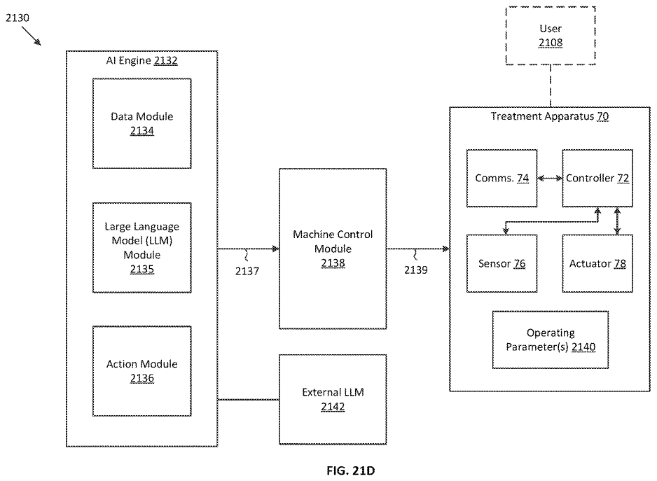

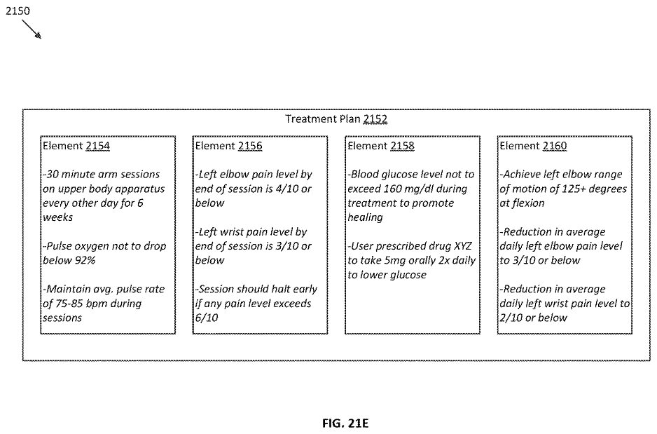

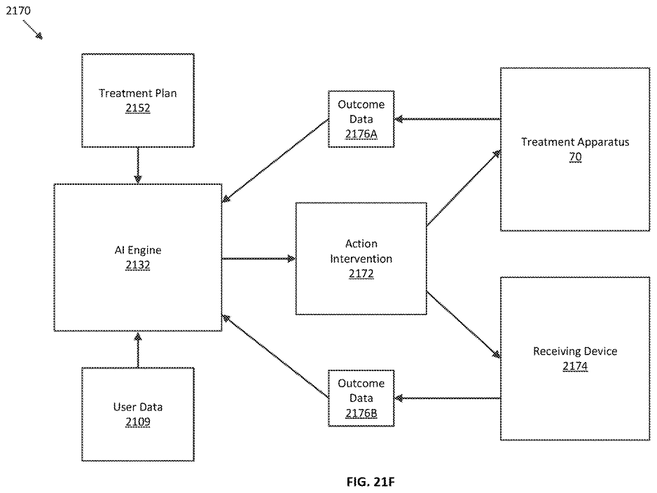

Turning ahead to FIG. 21A, a block diagram 2100 is shown further illustrating certain aspects of method 1440 (corresponding to FIG. 14D), according to various embodiments.

In this figure, user data 2109 may be received by intervention system 2120 as may occur in accordance with step 1442 of method 1440 according to some embodiments. User data 2109 may be associated with user 2108 and include objective data 2112 and/or subjective data 2114 in the embodiment shown, but may also include other types of data. Examples of objective data 2112 and subjective data 2114 are shown in FIG. 21B, as discussed further below. User data 2109 may be obtained and/or sent to intervention system 2120 by one or more of electromechanical machine 2102, sensor 2104, and/or computing device 2106 according to various embodiments.

Intervention system 2120 is a computing device that has one or more non-transitory computer-readable media capable of storing program instructions and one or more processors capable of executing program instructions to cause various operations to be performed, according to some embodiments. Intervention system 2120 may perform all or a portion of one or more steps of method 1440 in various embodiments, though in other embodiments, all or a portion of one or more steps of method 1440 may be performed by a different computing system.

Intervention system 2120 may correspond to one or more embodiments of server 30, and may include any or all features, structures, capabilities, functionality, configurations, etc., of server 30 as described elsewhere herein. In other embodiments, intervention system 2120 may correspond to a computing device other than server 30, however. Such a computing device may be integrated with, attached to, and/or configured to communicate with treatment apparatus 70, stationary cycling machine 100 and/or another treatment apparatus having one or more electromechanical components. Intervention system 2120 may thus be a standalone system or may be part of another device, according to various embodiments. Intervention system 2120 may also include multiple computing devices in some instances (as may any computer system or computing device described herein).

Electromechanical machine 2102 corresponds to treatment apparatus 70 in some embodiments, and corresponds to stationary cycling machine 100 in some other embodiments, but is not limited as such and may include, without limitation, rowing machines, treadmills, and stair-climbers. Electromechanical machine 2102 may variously include a combination of any or all features, structures, capabilities, functionality, configurations, etc., as described with respect to treatment apparatus 70 and/or stationary cycling machine 100. Electromechanical machine 2102 thus includes one or more components that may be adjusted or modified with respect to a therapy session of a user, according to some embodiments.

Computing device 2106 corresponds to a device for patient interface 50 in at least one embodiment, for treatment apparatus 70 in at least another embodiment, and for server 30 in at least one further embodiment. Computing device 2106 may variously include a combination of any or all features, structures, capabilities, configurations, etc., described relative to patient interface 50, treatment apparatus 70, and/or server 30, according to some embodiments.

Sensor 2104 corresponds to internal sensor 76 in at least one embodiment, ambulation sensor 82 in at least another embodiment, and pressure sensor 86 in at least one further embodiment. Sensor 2104 may variously include a combination of any or all features, structures, capabilities, functionality, configurations, etc., of sensors 76, 82, and/or 86. In some embodiments, sensor 2104 may be a temperature sensor, a perspiration sensor, a current sensor, a voltage sensor, a proximity sensor, a chemical sensor, a gas sensor, a light sensor, a humidity sensor, and the like. Sensor 2104 may also combine multiple functionalities in some embodiments—e.g., a single sensor may be configured to sense both temperature and perspiration, as one example. Sensor 2104 may comprise a device that is wearable by user 2108 according to various embodiments. Sensor 2104 may also comprise a camera device configured to visually capture one or more portions of a user (e.g. a position or a movement of all or a portion of the user's body during a therapy session).

Sensor 2104 is configured to perform various operations related to artificial intelligence (AI) and machine learning (ML) according to some embodiments. These operations include gathering data that may be utilized by an AI engine, such as AI engine 2132 (discussed further below in relation to FIG. 21D). Sensor operations performed in connection with AI engine functionality including gathering and/or transmitting data as may relate to determining and/or performing an action intervention that includes modifying one or more operating parameters of an electromechanical machine (also discussed elsewhere herein in greater detail). Data collected via sensor 2104 may also be used for purposes of data training operations, including building or updating a trained model that may be used by AI engine 2132 in association with determining and/or performing an action intervention. AI engine 2132 may include a large learning model (LLM) in some embodiments. (Note: for the avoidance of doubt, “AI” as used herein refers to Artificial Intelligence and “AI” is not intended as an abbreviation for “action intervention.”)

In some embodiments, electromechanical machine 2102 or computing device 2106 is operatively connected to or includes sensor 2104. Thus, while user data 2109 may be sent directly to intervention system 2120 by sensor 2104, user data 2109 may also be transmitted to intervention system 2120 by electromechanical machine 2102 or computing device 2106 in some embodiments. User data that is present at machine 2102 or at computing device 2106 may have originated elsewhere, such as a sensor other than sensor 2104 or an external database, according to some examples. In some instances, user data 2109 may thus be transmitted to intervention system 2120 indirectly by electromechanical machine 2102 or computing device 2106, either of which may have acquired the user data from another source in various ways, including via a sensor.

Objective data 2112 includes, in one embodiment, all or a portion of information contained in patient data store 44 as described elsewhere herein. Objective data 2112 may also include other data associated with a user in various embodiments, including but not limited to heart rate, respiratory rate, blood pressure, blood oxygen level, other blood data (e.g. red blood cell count, white blood cell count, electrolyte levels, cholesterol levels, protein levels, liver function tests, kidney function tests, A1c levels, glucose levels, level of one or more specific chemicals and/or pharmaceutical drugs in blood, etc.), biopsy, histology or other tissue-based findings and results pertaining to any anatomical or physiological system, organ, or other part of a human body, physiologic measurements (e.g. height, weight, body fat percentage, lung capacity (e.g., pulmonary function tests or PFTs), range(s) of motion including multiple categorizations and/or specified degrees of motion for one or more orientations of body parts such as neck, shoulders, arms, wrists, hands, back, waist, hips, legs, knees, ankles, feet), objective or subjective pain levels (e.g. on a scale of 1-10), or medication data such as one or more specific prescription or non-prescription medications the user is currently taking or has previously taken, dosage levels, date and/or time of prior doses, frequency of dose(s), or routes of administration (e.g. oral sublingual, subcutaneous, parenteral, other intravenous means, mucosal, suppository). Medication data may also include similar data for vitamins and/or other nutraceutical or similar supplements.

Objective data 2112 may include instant data (e.g. data collected in some last number of recently occurring seconds or other period of time measurement (e.g., last 2 seconds, last 5 seconds, last 20 seconds, last minute, etc.) Such instant data may be related to the user, such as a heart rate of the user while using a treatment apparatus. Objective data 2112 may also include historical data related to the user (e.g. previously collected data about the user's heart rate, as an example) that is associated with one or more particular periods of time in the past, including, without limitation, a past period of time measured in years, months, days, hours, minutes, seconds, milliseconds, microseconds, etc. Objective data 2112 may include raw data readings (e.g. from a sensor) and may also include processed data (e.g. data that has been produced as a product of one or more computer-assisted calculations). Accordingly as just one illustrative example, objective data 2112 might include an instant heart rate of a user of treatment apparatus 70 at the time the user is engaging in a current treatment sessions on treatment apparatus 70 (e.g. operating the pedals), one or more average heart rates corresponding to one or more previous treatment sessions of the user, high and low (e.g., maximum and minimum) heart rates for the current treatment session or for some portion thereof (e.g., a maximum pulse rate during a 2-minute interval of high intensity interval training), and one or more high and low heart rates for one or more previous treatment sessions of the user. More generally, averages, high or low data points, or other statistical or computational calculations for various of the examples given above may be included in objective data 2112. A broad and substantial range of different data (and different data types) may thus be included in objective data 2112.

In some instances, objective data 2112 may include data such as described above for one or more additional users other than user 2108 (subject to user consent and/or any applicable health or data privacy laws or regulations). Thus, in one embodiment, anonymized and/or de-identified cohort data could be included in objective data 2112, e.g., if the user is a 34-year-old female of Asian descent, then objective data 2112 could include an average blood pressure value for females of Asian descent between the ages of 30 and 35 (as just one example). Objective data 2112 may include, in some embodiments, purely objective information or purely objective information mixed with subjective or qualitative information as described elsewhere herein, e.g., in paragraphs 0102-0106.

In some embodiments, objective data 2112 may be in an unstructured data format, such as medical professional handwritten notes entered using a tablet and input peripheral, scanner, or the like. One or more processors may perform transformations or conversions on the unstructured data to output the objective data 2112 in a structured and/or normalized data format that is used to train and/or be processed by one or more computer-implemented models of the artificial intelligence engine.

Subjective data 2114 may also be received by intervention system 2120. Generally, subjective data 2114 may include or relate to any of the data described herein with regard to objective data 2112, and any or all aspects described herein with respect to objective data 2112 are also applicable to subjective data 2114 according to various embodiments. Subjective data 2114 may include, in various embodiments, subjective or qualitative information as described elsewhere herein (e.g., in paragraphs 1118-1119).

Subjective data 2114 may originate via user 2108 in some embodiments. Subjective data 2114 may thus be received and/or generated via electromechanical machine 2102 in some instances, or via sensor 2104 or computing device 2106 in other instances. Electromechanical machine 2102, sensor 2104, or computing device 2106 may thus have an input mechanism that allows a user to provide such subjective data, according to various embodiments. Such an input mechanism may be mechanical and/or electrical in nature, and may comprise a microphone, touchscreen, camera, keyboard, mouse, dial, one or more depressable buttons, etc. As one example, sensor 2104 might include a touchscreen that has a display asking a user to rate his or her pain level on a scale of 1-10 at one or more particular times during a session of the user with treatment apparatus 70.

Subjective data 2114 therefore includes information about a user's perceived level of pain, their mood, their opinion relative to an aspect of their health or treatment of their health, or other subjective data in various embodiments. Subjective data may include qualitative or quantitative data. One detailed example of subjective data 2114 is described below relative to FIG. 21B.

Turning to FIG. 21B, a block diagram 2122 is shown that illustrates further aspects of objective data 2112 and subjective data 2114, according to various embodiments. It should be understood that the type of data and data values present in objective data 2112 and subjective data 2114 may vary widely, and this figure merely presents examples of such data for purposes of illustration.

As shown in FIG. 21B, objective data 2112 includes patient information 2113A, vital statistics 2113B, imaging data 2113C, and sensor data 2113D. This information may be stored temporarily or permanently at electromechanical machine 2102, sensor 2104, computing device 2106, and/or comorbidity analysis system 2120, according to various embodiments. In one embodiment, objective data 2112 may be received via a wearable device worn by the user, which may be a smart watch or a measurement instrument attached to the user such as an EKG pad for measuring heart rate, a body temperature sensor, or a perspiration sensor. Objective data received from a wearable device may include data relating to a specific body part of a user, such as a knee angle measurement corresponding to a knee angle of the user. The objective data received from a wearable device may include a step count (e.g. a number of steps or step-equivalents taken during an treatment session with treatment apparatus 70) or may include a heart rate.

Patient information 2113A may include a variety of information relating to an individual's identity, background, health, or history. The present example shows a patient's name, date of birth (D.O.B.), as well as the patient's occupation and ethnicity information. The present example also shows health history information indicating the patient was a former smoker of tobacco. This portion of data in objective data 2112, as well as any or all portions of objective data 2112 or subjective data 2114, may be relevant to determining generating and/or ranking one or more action interventions as may be performed by AI Engine 2132 (as further described below).

As shown, vital statistics 2113B may include information relating to a user's general health at one or more specified dates or times. In this example, height and weight information is shown, as well as count information for white blood cells and red blood cells. Additional information is also shown regarding cholesterol and triglycerides. Vital statistics may include information about a present date (e.g. a date on which a user is participating in a physical therapy session) or any past date. A past or present date may be associated with one more particular activities of the user, including without limitation a hospital visit, laboratory visit, doctor visit, physical therapy session, etc.

Imaging data 2113C includes data and/or metadata regarding one or more images in the example shown. This may include an image description (e.g. X-ray, left hip), a time or date of the image, and the identities, employee numbers or other identifying information of one or more persons associated with the image (e.g. a technician who took an X-ray image, a doctor who ordered the image, etc.). Imaging data 2113C may include a digital image file comprising the actual image itself in a particular digital format (e.g. a JPEG file, a GIF file, etc.) This data may include multiple images taken on one or more particular dates or during a same session or appointment, such as a same body part captured at different angles. Different types of images are also contemplated in various embodiments. Thus, there may be imaging data for X-rays, magnetic resonance imaging (MRI), computed tomography (CT) scans, etc. Imaging data 2113C and objective data 2112 more generally may include any kind of media, and is not limited to two-dimensional graphical images, however. Thus, videos or other animations may be included in the objective data and may correspond to a particular digital format (such as an MPEG standard). Three-dimensional visualizations or projections may also be included, such as a three-dimensional reconstruction of a patient's knee joint including tendon and ligament connections, for example.

Sensor data 2113D may include data relating to one or more current or past therapeutic sessions. This may include information regarding parameters of a therapy apparatus and/or a user's interaction with the apparatus. As shown, sensor data 2113D includes a current rotations per minute (RPM) of a portion of a therapy apparatus (e.g. a variable resistance bicycle pedal component), and an average and maximum RPM for a current session. Related historical data may also be present in sensor data 2113D. The sensor data shown also includes a pulse oximeter-measured blood oxygen level (SpO2), body temperature, and heartrate as measured in beats per minute (BPM).

In the example of FIG. 21B, subjective data 2114 includes pain information (info) 2115A, mood information (info) 2115B, and session information (info) 2115C. Note that according to various embodiments, any or all portions of subjective data 2114 may include any or all characteristics described with respect to any or all portions of objective data 2112. Thus, any statement made herein with regard to objective data 2112 also applies to subjective data 2114 and vice versa, according to one or more embodiments.

As shown, pain information 2115A includes a variety of data relating to pain levels of a user. Such data may be collected relative to a current or past therapeutic session. Thus, the pain information shown in this figure includes a pain level prior to beginning a session, a pain level at a time during the session, and a pain level shortly after the end of the session. In the example shown, pain information 2115A further includes a pain level measurement taken on the next day after a session. As noted above, a variety of formats may be used for pain information or other subjective data 2114. As shown, pain levels are measured on a zero to ten (0-10) scale, but other formats are possible, such as a particular defined qualitative and/or enumerated set such as [“none”, “low”, “moderate”, “high”, “severe,” or “debilitating”].

In the example shown, mood information 2115B includes data relating to one or more moods of a user (e.g. information relating to one or more emotions the user is experiencing and/or has experienced). As shown, mood information 2115B includes user responses to one or more particular prompts or questions, such as whether a pain level of a user allows them to live a normal life, or what an overall mood of the user is. The responses to such prompts may be represented in freeform text, or may be one of a plurality of predetermined responses (e.g. [yes/no], or [happy/sad/anxious/upset/depressed]). As with other user data, many different formats are possible. An example of such prompts is described further below relative to FIG. 21C.

Session information 2115C includes subjective data related to one or more therapeutic sessions in various embodiments. This data may include information related to a current or recent session, as well as to one or more past sessions. As shown, session information 2115C includes answers to two prompts, one regarding how hard a user had to exert themselves in a therapeutic session conducted that day, as well as a post-session mood. Note that while mood information is included in mood information 2115B, mood information may also be included in session information 2115C. More generally, subjective data 2114 may be organized according to a variety of different data organization schemes, and thus it is not required that one or more particular data items or types of data be stored relative to a particular category. Rather, any subjective data may be generally stored relative to any of one or more categories of information, according to various embodiments.

Subjective data 2114 may correspond to one or more specific operations and/or movements of all or a portion of treatment apparatus 70. Further, each such specific operation and/or movement may correspond to one or more operating parameters of treatment apparatus 70 (or another electromechanical machine). Adjusting such operating parameters may change an operation or movement of treatment apparatus 70, and may occur when performing an action intervention as may be generated by AI engine 2132. Subjective data 2114 may thus be recorded in conjunction with one or more particular operating parameters of treatment apparatus 70 (e.g., user reported a pain level of 4/10 when a resistance level for a portion of treatment apparatus 70 was set to 5.60 at a speed of 55 RPM, but reported a pain level of 6/10 when the resistance level for that portion of treatment apparatus 70 was set to 7.50 at a speed of 60 RPM). Pain information 2115A shows one such example where an RPM of 80 is recorded in association with a user's pain level during the course of a session.

Turning to FIG. 21C, a diagram is shown of an example of prompts (e.g. questions) 2128 that may be provided to a user. User responses to prompts 2128 may be recorded and/or used as subjective data 2114 or as objective data 2112 in various embodiments. Such prompts may be presented to a user via one or more interfaces as may be present on treatment apparatus 70 or a user computing device, such as a user computing device is computing device 2106 in some embodiments. In some instances, sensor 2104 may also include a user interface configured to display prompts and collect user responses to such prompts. Note that as used herein the term “display” does not indicate a visual only display unless specifically stated, but may include other presentations of media, such as an audio presentation.

User responses to prompts 2128 may be recorded at multiple different times and/or dates (e.g. before a session with treatment apparatus 70, after a session with treatment apparatus 70, or during a session with treatment apparatus 70). A user may be asked a question designed to produce subjective outcome data multiple times in a day in some instances. For example, a user might be asked “How much pain are you in right now?” every five minutes during a session, during other particular portions of or fixed or variable intervals during a session, or at specified times of day for one or more days of the week.

In various embodiments, prompts 2128 may have one or more specified formats for the responses of a user. Such formats may include a 1-10 scale, selecting from a set of one or more defined values, or a freeform response (which may be in a natural language such as English), according to some embodiments. Prompts 2128 may be provided to a user via intervention system 2120 or another computing device, in some embodiments, via a display screen (which may include a touchscreen) that shows the user the question as well as allowable response choices. User responses to prompts 2128 may also be collected via intervention system 2120 or another computing device, e.g., when a user selects a response choice on a touchscreen. Other input mechanisms for collection of user responses may be used according to various embodiments. In one embodiment, user responses may be recorded via a microphone corresponding to intervention system 2120 or another computing device. A microphone input mechanism may accommodate freeform responses, but may also be used when a specific response format is requested (e.g. a listing of pain on a 1-10 scale). The user may thus also be prompted to provide a response via audio, such as a speaker device corresponding to computing device 2106. A resulting audio recording of the user's speech may be stored as part of subjective data 2114, and may be converted into text by a speech recognition algorithm and/or generative artificial intelligence.

Turning to FIG. 21D a block diagram 2130 is shown that illustrates further aspects of method 1440, according to various embodiments.

In this diagram, AI Engine 2132 comprises an artificial intelligence engine, and in some embodiments, corresponds to AI Engine 11. AI Engine 2132 may include any or all features, structures, capabilities, functionality, configurations, etc., as described with respect to AI Engine 11.

In some embodiments, AI Engine 2132 comprises one or more computing devices that each may include one or more processors and one or more non-transitory storage media. AI Engine 2132 may thus be a computer system configured to execute instructions that cause the computer system to perform certain operations (e.g. computations related to artificial intelligence computing tasks such as training a model, updating a model, or generating an action intervention). In one embodiment, AI Engine 2132 thus corresponds to server 30 and may include any or all features, structures, capabilities, configurations, etc., described with respect to server 30. In another embodiment, AI Engine 2132 comprises a processor and one or more non-transitory storage media included within intervention system 2120.

In some embodiments, AI Engine 2132 may refer to one or more computer programs executable on a computer system. AI Engine 2132 thus may include one or more executable computer instructions, which may be stored on one or more computer-readable media, that when executed, cause a computer system to perform one or more particular operations. Thus, in one embodiment, AI Engine 2132 comprises one or more computer instructions stored within intervention system 2120. AI Engine 2132 may also comprise data other than executable computer instructions in various embodiments (e.g. configuration data, log data, or other data).

As shown, AI Engine 2132 includes a data module 2134, a large language model (LLM) module 2135, and an action module 2136. Machine control module 2138 is not included within AI Engine 2132 in the example shown, but machine control module 2138 may be present on a same computer system as AI Engine 2132 or on a different computer system, according to various embodiments.

Data module 2134, LLM module 2135, action module 2136, and machine control module 2138 each comprise executable computer instructions in various embodiments, and these instructions as well as any associated data used for computing operations may be stored on one or more non-transitory computer-readable media. These executable instructions and data may be partially or wholly integrated within AI Engine 2132 in some instances.

Thus, modules 2134, 2135, and 2136 are included within AI Engine 2132 in the embodiment shown, but in other embodiments, one or more of these modules may be wholly or partially separate from AI Engine 2132. In such embodiments, executable functionality, data, and/or features of module 2134, 2135 and/or 2136 may be accessed by AI Engine 2132 via local access and/or networked access, and may be accessed via an application programming interface (API) in some instances.

Similar remarks also apply to machine control module 2138 in some embodiments; that is, module 2138 may be entirely separate from AI engine 2132, may be entirely integrated with AI engine 2132, or may be partially integrated with AI engine 2132 and also partially integrated into one or more other computer systems and/or computer-readable media. Machine control module 2138 may thus be integrated with or otherwise in local connection with treatment apparatus 70 in some embodiments. Further, data module 2134, LLM module 2135, action module 2136, and/or machine control module 2138 may be partially or wholly implemented in hardware in some instances. Thus, as non-limiting examples, machine control module 2138 may be a custom ASIC chip located within or otherwise in local connection to treatment apparatus 70, or machine control module 2138 may be a custom ASIC chip located within server 30.

AI Engine 2132 may send information as one or more communications 2137 to machine control module 2138 in an embodiment. Communication 2137 may include control instructions based on one or more action interventions that include modifications of one or more operating parameters 2140 of an electromechanical machine, such as treatment apparatus 70, in the example shown. Information in communication 2137 may thus be usable by machine control module 2138 to control an electromechanical machine, for example, by modifying one or more operating parameters of treatment apparatus 70. Machine control module 2138 may likewise send one or more communications 2139 to treatment apparatus 70 according to some embodiments. Communication 2139 may be the same or substantially the same as communication 2137. In some embodiments, however, machine control module 2138 may generate communication 2139 based on specific parameters and on control and/or data interfaces that are applicable to treatment apparatus 70 or another device comprising an electromechanical machine. Communications 2137 and 2139 may be transmitted locally (e.g. via a data bus or cable) or via one or more networks in various embodiments. Communications 2137 and 2139 may thus be sent via one or more APIs specific to module 2138 and/or treatment apparatus 70, or they may otherwise be in a digital format that is customized to particular hardware of treatment apparatus 70, according to some embodiments.

Data module 2134, in various instances, includes executable logic to perform functions related to receiving, processing, transforming, and transmitting data. This may include receiving user data 2109 from electromechanical machine 2102, sensor 2104, or computing device 2106 in some embodiments. Data module 2134 also includes executable logic to interact with (e.g. transmit data to and receive data from) other modules such as LLM module 2135, action module 2136, and machine control module 2138, in various embodiments.

LLM module 2135 includes executable logic to generate and/or rank one or more action interventions via one or more LLMs in the example shown. LLM module 2135 may contain an instance of an LLM, but may also communicate with one or more other LLMs not contained within LLM module 2135. One such LLM is external LLM 2142, which may be hosted by a computer system different from a computer system on which AI engine 2132 is present. In some embodiments, external LLM 2142 may be hosted in a cloud computing environment and may be accessed by LLM module 2135 via a network. In such instances, LLM module 2135 may transmit data and/or instructions (which may be natural language instructions) to external LLM 2142, and in turn may receive output from external LLM 2142. LLM output may include natural language text or other data (including but not limited to binary data, data formatted according to one or more particular predefined data formats, multimedia data, image or video data, audio data, etc.)

Action module 2136 is configured in some embodiments to perform one or more action interventions. This may include interacting with treatment apparatus 70 (e.g. via machine control module 2138) to cause modification of one or more operating parameters 2140 of the apparatus. In some instances, action module 2136 may thus initiate one or more communications 2137.

Turning to FIG. 21E, a block diagram 2150 is shown of an example of a treatment plan 2152 as may be utilized in conjunction with artificial intelligence techniques to generate an action intervention in relation to a user who uses an electromechanical machine (such as treatment apparatus 70) to perform all or a portion of the treatment plan.

In this example, treatment plan 2152 corresponds to a user 2108 who is rehabilitating a left arm after injuring it in an accident. The user in this example also has two pre-existing secondary conditions, i.e., comorbidities, in the form of chronic obstructive pulmonary disease (COPD) and diabetes. Treatment plan 2152 may have multiple elements, each of which may also include sub-elements. In the example shown, treatment plan 2152 comprises elements 2154, 2156, 2158, and 2160.

Treatment plan 2152 may be executed in association with treatment apparatus 70 or another electromechanical machine in order to achieve one or more treatment goals. Treatment plan 2152 may be created by any combination of a physician, a nurse, a physical therapist, a user, another medical professional, or another qualified person, according to various embodiments. Treatment plan 2152 may be stored as structured and/or unstructured electronic data on one or more computer readable media according to various embodiments. In some instances, elements and/or sub-elements of treatment plan 2152 may be structured in a format that is usable in conjunction with artificial intelligence and machine learning techniques. Thus, one or more elements of treatment plan 2152 may be vectorized and converted to one or more specified vector formats in order to allow for AI and ML processing of data within the treatment plan. One example of a vector format includes a one-dimensional array of data (e.g., an array of byte data having a size of 1×4096), as arranging data in such a vector format may allow for more efficient processing by AI engine 2132.

As shown, element 2154 of the treatment plan includes sub-elements relating to therapy session parameters. In this example, because the user has COPD, a specification is made for the user's blood oxygen level not to drop below 88% during treatment. Note that while pulse oximeter-measured blood oxygen level (SpO2) might normally be higher for most users (e.g. 95-100% at rest, dropping to 90-95% during physical exertion), this particular user may have a lower baseline due to the particular user's COPD. Element 2154 includes another specification for the use of an upper body apparatus, which might include an arm cycle or another component designed to allow rehabilitation of an arm or portions of an arm. Element 2152 further includes a specification regarding average heart rate during a session. Pulse oximeter-measured blood oxygen level (SpO2) and heart rate may be monitored by one or more sensors 2104.

As shown, element 2156 of treatment plan 2152 includes specifications relating to pain level for the user. These specifications include pain level relative to one or more specific body parts (left elbow and left wrist), with a goal of pain being below a certain threshold at the end of each therapy session. Element 2156 also includes a directive to halt a therapy session if pain exceeds a threshold level during the session.

As shown, element 2158 includes specifications relating to a condition of the user (in this case, diabetes). Thus, element 2158 indicates that, in order to promote healing, the user's blood glucose level should not exceed a threshold level, and that the user should take a particular drug at a specified frequency or interval and at a particular dose or doses to lower the user's glucose level.

As shown, element 2160 includes specifications relating to overall goals of treatment. In this instance, one goal is to achieve a particular range of motion of a body part of the user (left elbow). Element 2160 also includes goals regarding pain level for the user with respect to one or more body parts (left elbow, left wrist).

Many possible treatment plans may be formulated for different users who may require treatment for a number of different reasons, and the treatment plan shown in FIG. 21E is merely intended to illustrate one example. Treatment plans may include different elements and sub-elements in various embodiments, and may include different data and/or different types of data than those shown in this example. Treatment plans generally may have elements and/or sub-elements that are related to any data discussed herein, including but not limited to user data 2109, objective data 2112, and subjective data 2114. Treatment plans may thus be organized and/or stored according to any number of various data schemas according to some embodiments.

Turning to FIG. 21F, a block diagram 2170 is shown of a data flow relating, in some embodiments, to a method for determining and performing action interventions using an electromechanical machine in which interventions are determined by using a large language model to generate and/or rank, based on one or more measures of efficacy, a list of interventions. In one embodiment, FIG. 21F relates to method 1440 as discussed herein (relative to FIG. 14D, for example).

In the example of FIG. 21F, AI engine 2132 may receive data inputs that include treatment plan 2152 and user data 2109. Based on such input, AI engine 2132 may generate one or more action interventions 2172 and may also rank those action interventions. In some instances, action intervention 2172 may then be performed, thus causing an effect on treatment apparatus 70. Action intervention 2172 may thus include a control action that affects operation of treatment apparatus 70. Action intervention 2172 may also cause an effect on receiving device 2174, such as causing a message to be displayed to a person. Note that in general, any or all portions of method 1440 may be performed by AI engine 2132 and/or by one or more other computing devices and/or computer programs executing on a computing device.

Referring to method 1440, in some embodiments, step 1442 may thus include AI engine 2132 receiving user data 2109 and/or treatment plan 2152. In association with performing certain computing operations (further described below), AI engine 2132 may receive or access other data in addition to (or instead of) all or a portion of user data 2109 and/or treatment plan 2152. As one example, AI engine 2132 may be configured to access one or more networked other data sources. Such data sources may include websites on the internet, a database corresponding to a physician's office, hospital, or laboratory, or information stored on a distributed ledger, e.g., a blockchain. Such data sources may include data generated by or obtained via electromechanical machine 2102, sensor 2104, or computing device 2016.

Data from such other sources may be used in combination with user data 2109 and/or treatment plan 2152 to perform all or a portion of any functionality described herein with respect to action interventions, or with respect to any AI and ML techniques generally. As such, any references herein to user data 2109 and/or treatment plan 2152 should be understood to also include data from one or more such other data sources according to various embodiments. Fetching or accessing data from other sources may be performed by data module 2134 (see FIG. 21D) in some instances.

Step 1444 of method 1440, in some embodiments, includes AI engine 2132 performing one or more computing operations on user data 2109 and/or treatment plan 2152 to generate one or more action interventions 2172. Such an action intervention comprises data and/or instructions configured to cause a real-world effect on a treatment apparatus and/or computing device, according to various embodiments. Such a real-world effect may directly relate to one or more of a user (e.g. user 2108), a current, past, or future therapy session for the user, one or more elements or sub-elements of a treatment plan for the user, or an operating parameter of apparatus 70, though the real-world effect is not limited to these examples.

An action intervention 2172 that is generated by AI engine 2132 may relate to an actual or potential emergency situation. In one example, AI engine 2132 may determine that a user is experiencing conditions during a therapy session that place them at risk of a negative health event or is experiencing conditions that indicate that a negative health event has occurred or currently is occurring). Referring back to the example of FIG. 21E, if a pulse oximeter-measured blood oxygen level (SpO2) of the user during the therapy session has dropped to 90%, this may indicate that the user is (or is about to be) in active respiratory failure, a condition that represents a medical emergency. One or more action interventions 2172 generated by AI engine 2132 in this case may be sent to one or more devices, as described further below.

Upon receipt of action intervention 2172 at treatment apparatus 70, the treatment apparatus may perform one or more actions as specified by data and/or instructions in the action intervention. Based on action intervention 2172, treatment apparatus 70 may halt an ability of the user to physically exert himself with respect to one or more components of the apparatus. For example, the action intervention may cause the treatment apparatus to electromechanically lock a rotating cycling mechanism that is designed to interact with a user's arms, legs, or other body part. Based on action intervention 2172, apparatus 70 might also display an audible or visual alert to the user and/or to another person nearby. Such an alert might include speech or visual text that states “WARNING: Stop and seek medical attention immediately! Oxygen level is dangerously low.” An audible alert may also include other sounds besides speech—for example, a loud siren or buzzing noise. A visual alert likewise may include visual displays other than text, such as a red blinking light situated on or near a portion of apparatus 70 such that others in the vicinity might easily see that an emergency is occurring.

Continuing this example, an action intervention 2172 may also be sent to receiving device 2174. Receiving device may be a rackmount server, a router computer, a personal computer, a portable digital assistant, a smartphone or other phone device, an analog phone, a laptop computer, a tablet computer, a netbook, a desktop computer, an Internet of Things (IOT) device, any other computing device, or any combination of the above.

Receiving device 2174 may thus be a phone device or computer system associated with an emergency services provider. Upon receipt of action intervention 2172, receiving device 2174 may display a message alerting a dispatcher or other personnel to a medical emergency. Receiving device 2174 may be a phone device or computing device in an ambulance, fire truck, or other emergency response vehicle. Upon receipt of the action intervention, the receiving device may thus alert a driver of the emergency response vehicle. Action intervention 2172 may include physical location information regarding user 2108, and receiving device 2174 may display such information to facilitate a rapid response. Thus, receiving device 2174 could cause an audio and/or visual message to appear within a crewed ambulance, with such a message stating “Emergency: Respiratory failure in progress at 123 Main St. Blood oxygen level 85%. Respond immediately. Subject is Caucasian male, age 60. Subject is allergic to sulfa drugs.”

Various information about user 2108 may thus be included within action intervention 2172 according to some embodiments. The information may relate to a description or other visual characteristics of the user (e.g. as indicated in the emergency response example above), but may also include medical information relating to the user. Such medical information can be useful in a variety of situations, including both emergency and non-emergency situations such as an informational message to a physician. More broadly, information in action intervention 2172 may include any information that is available to AI engine 2132 in various embodiments, including but not limited to any or all information in treatment plan 2152, user data 2109, or information generated by or obtained via treatment apparatus 70.

A different example of action intervention 2172 in a non-emergency situation relates to tracking progress of a user throughout a treatment plan. Action intervention 2172 might be generated after each therapy session is concluded and contain data related to both the user and treatment apparatus 70. In this example, action intervention 2172 might include data related to a treatment plan for the user, such as pain level or range of motion of a body part. Action intervention 2172 may also include historical progress information—for example, showing range of motion of a body part over time (e.g. left elbow flexion of 75 degrees in week 1 of treatment, 80 degrees in week 2 of treatment, 90 degrees in week 3 of treatment, etc.).

Another example of action intervention 2172 relates to operation of treatment apparatus 70 (or another device comprising an electromechanical machine). While treatment apparatus 70 may halt operation of one or more components in an emergency situation as described above, its operation may also be changed in other ways as may correspond to various non-emergency situations, for example.

Consider a user that has a treatment plan in which average heart rate during the course of a therapy session is supposed to be in the range of 75-85 beats per minute (BPM). Based on session monitoring information (e.g. received from treatment apparatus 70), AI engine 2132 may determine that a current session for a user is likely to fall outside of the parameters of the treatment plan. For example, at the 10-minute mark of a 40-minute session, a user's time-averaged heart rate is determined by AI engine 2132 to be 60 BPM. This observational data falls significantly short of the range of 75-85 BPM indicated by the treatment plan, and AI engine 2132 may determine that this range is likely to be missed (e.g., by a 90% confidence threshold or some other threshold) unless parameters for the current session are changed.

AI engine 2132 may then generate and send an action intervention to treatment apparatus 70 with instructions that will cause the apparatus to alter one or more operating parameters in such a manner that a level of physical exertion for the user is likely to increase. For example, a variable resistance level of an apparatus component, such as an arm or leg cycle mechanism, might be increased (e.g. from 6.3 to 7.9). Another effect might be to adjust a speed of a walking treadmill, a rower, or a stair-climbing apparatus from 1.8 miles per hour up to 2.2 miles per hour, or to change an angle of an apparatus component such that the user is likely to be forced to increase exertion.

At a later time in the session (e.g. the 20-minute mark of a 40-minute session), AI engine 2132 could then re-analyze new data received during the session to determine if the action intervention appears to have been effective. If the action intervention did not appear effective (e.g., the user's average heart rate has increased to only 67 BPM, indicating the minimum target of 75 BPM will still not be met), AI engine 2132 could generate and transmit a second action intervention to treatment apparatus 70. Such an iterative approach may be employed any number of times and at any desired frequency (e.g., every 30 seconds, every 5 minutes, or some other time period) to ensure a higher probability that a user will meet a goal of their treatment plan and that the user is likelier to have an overall faster and more effective treatment process leading, among other things, to an improved quality of life and a fuller recovery.

Note that generally, AI engine 2132 may record and store prediction data, outcome data, and/or action intervention data in order to analyze the efficacy of action interventions and/or improve one or more models associated with generating and/or ranking action interventions. Operations described above may also be based on specifics of a treatment plan; for example, AI engine 2132 may perform data analysis that indicates a treatment plan already specifies parameters that will greatly increase the exertion of the user, and thus determine that generating an action intervention is unnecessary. Action interventions may therefore be generated in view of an already existing treatment plan and/or parameters associated with one or more past, current, or future therapy sessions.

Continuing the example above, an active intervention 2172 may also be generated during a session and sent to a destination device associated with a rehabilitation specialist (or other person) that is assisting or supervising a user. Thus, at the 10-minute mark of a 40-minute session, a mobile phone device of the rehabilitation specialist might receive a text (SMS) message or voice call with a message stating “Patient Sally Smith looks like she is falling short of her target goal of 75-85 pulse BPM. Encourage her to moderately increase her level of effort and ask if there is anything you can do to help her work harder.” A message to a rehabilitation specialist (or other person associated with a therapy session) may vary widely in different scenarios. Thus, another such message could state “Patient Sally Smith appears to be holding her right elbow in an incorrect posture while doing her weight exercise. Please review with her and correct as appropriate.” Such a message might be sent based on camera imaging data obtained from a camera attached to or associated with treatment apparatus 70, for example.

In some instances, a predicted success probability may be utilized by AI Engine 2132 in order to generate and/or rank one or more action interventions. Such a success probability may involve using a model trained with machine learning and/or AI techniques based on one or more suitable data sets. Before explaining an example of such training, a success probability is defined herein as a numerical value falling within a specified range, such as 0 to 1.00, according to various embodiments. In various embodiments, a success probability threshold level should likewise be understood to mean a numerical value also falling within a specified range, such as 0 to 1.00.

AI Engine 2132 may include machine learning model 13 in various embodiments, and be trained accordingly as described elsewhere herein. As just one example of training in accordance with predicting success of an action intervention, consider a user currently participating in a therapy session involving their left arm. In this example, AI Engine 2132 may have a machine learning model that was trained with data corresponding to a quantity of other users who have also participated previously in therapy sessions in which data was collected related to their left arm or another body part. Such training data may include any or all of user data 2109. Accordingly, AI Engine 2132 has access, in various embodiments, to a prediction model that is based on hundreds, thousands, millions, billions, or even larger numbers of therapy session data sets relevant to a particular prediction of success probability for an action intervention. Note that training of a model usable by AI Engine 2132 may include segregation of data according to various techniques, including segregating training data by different periods of time. As one example, the machine learning model may be trained on session-related data corresponding to minutes 0-5 of a treatment plan (a first period of time), and further trained on treatment-related data corresponding to minutes 5-10, minutes 10-15, etc. of a therapy session (second and third periods of time, etc.). Accuracy of overall predictions may be improved by such segregation of training data—for example, an action intervention generated based on a first period of time may have different success rates than an action intervention generated based on a different period of time. Many different segmentations of training data are possible (e.g. differing in lengths of time, differing in segment size, segmenting some training data in a different manner than other training data, segmenting on one or more criteria other than time, etc.)

AI Engine 2132 can simulate, based on its trained model, a variety of possible outcomes for an action intervention. When generating one or more action interventions, AI engine 2132 may generate multiple candidate action interventions, and continue processing until one or more candidate action interventions are predicted to achieve at least a threshold probability of success (e.g. 80%, 90%, or some other threshold.

Note that while FIG. 21F depicts a single action intervention being sent to treatment apparatus 70 and receiving device 2174 for ease of explanation, multiple scenarios exist with regard to generation and/or transmission of action interventions by AI engine 2132 to one or more destinations. In some instances, a single action intervention may be generated and sent to a single destination (e.g. treatment apparatus 70). In other instances, however, a single action intervention may be generated and transmitted to multiple destinations (e.g. both treatment apparatus 70 and receiving device 2174). In yet other instances, multiple action interventions may be generated, and one or more of these action interventions may be transmitted to one or more destinations. Thus, a first action intervention could be transmitted to treatment apparatus 70 while second and third action interventions could be transmitted to receiving device 2174; two action interventions could be transmitted to apparatus 70 but not to any other destination; or three action interventions could be transmitted to three different destinations (e.g. treatment apparatus 70, a phone and/or other computing device of a doctor, and a phone and/or other computing device of an emergency services provider, such as the 911 system). Various combinations, extensions, and permutations of such transmissions are thus possible and contemplated.

Action intervention 2172 may be generated according to one or more specific parameters of a destination device. An action intervention destined for treatment apparatus 70, for example, may include data and/or instructions that are in one or more formats compatible with processing by treatment apparatus 70. An action intervention destined for receiving device 2174 may include data and/or instructions that are in one or more different formats compatible with that device. In some instances, an action intervention generated by AI engine 2132 may include multiple portions of data and/or instructions, and one or more of these portions may be compatible with a particular destination device while one or more other portions are not compatible with that particular destination device. Thus, a single action intervention might be sent to both treatment apparatus 70 and receiving device 2174. Treatment apparatus 70 might process some but not all portions of the action intervention (thereby causing real-world effects), while receiving device 2174 might process other, different portions of the action intervention. More generally, any or all portions of an action intervention may be compatible with and processable by multiple different destination devices, while one or more portions of an action intervention may be incompatible with one or more of multiple different destination device (and in such instances, those incompatible portions may be ignored or otherwise discarded by the destination device).

Generation of action intervention 2172 by AI engine 2132 may be performed based on one or more measures of efficacy in some embodiments. Efficacy of an action intervention may be determined in multiple ways, but one such technique includes AI learning based on observational data from one or more previously conducted action interventions. A predicted probability of success for an action intervention (as described above) is one such measure of efficacy, according to some embodiments.

Consider the above example of a user who appears likely to miss a target goal for a session involving a specific desired range of average pulse BPM. Dozens, hundreds, thousands, millions, billions or some other number of action interventions may have been previously generated by AI engine 2132 (or another system) in which a user also appeared likely to miss a target goal. Thus, based on observational data related to measured results corresponding to the previous outcomes of previous action interventions, a current action 2172 intervention can be generated based on optimal and/or best-available parameters.

In one example, with respect to the relevant data (e.g. historical session data), a first group of 30% of users may have received a textual alert message asking them to work harder, a second group of 30% of users may have had their treatment apparatus adjusted automatically (e.g. without the user taking direct action themselves), while a third group of 40% of users may have received both a textual alert message and also had their treatment apparatus adjusted automatically. Continuing the example, session data for these user groups may indicate that for the first group, 64% of users achieved their target goal, 69% of users in the second group achieved their target goal, and 82% of users in the third group achieved their target goal. AI engine 2132 can then readily conclude that the third approach is most effective, and generate an action intervention based on that approach.

User demographics, health information, and other information may be used in determining efficacy of action interventions as well. In the example above, it may be the case that while the third approach showed best overall results, analysis of historical data indicates that for males between the ages of 18-29, the first approach (textual alert message only, without automatic adjustment of the treatment apparatus) outperforms the other two approaches (e.g., 85% vs. 76% vs 74%). In this instance, AI engine 2132 might generate a different action intervention based on user data 2109, thereby indicating this is the best approach for the particular user. Many such different analyses may be performed using the considerable amount of data available to AI engine 2132 in order to generate highly effective or optimal action interventions according to characteristics of a specific user, apparatus, and/or treatment plan. Self-learning may also be performed with regard to measuring effectiveness of an action intervention. AI engine 2132 may thus receive and analyze outcome data 2176A and/or 2176B, after causing a first action intervention to be implemented, to determine an effectiveness of that particular action intervention. Such outcome data may be generated and transmitted by treatment apparatus 70 and/or receiving device 2174 in various embodiments.

Step 1446 of method 1440 includes performing one or more action interventions 2172 according to some embodiments. Performing an action intervention may include transmitting an action intervention to a destination device that is configured to take an action responsive to information contained in the action intervention. Thus, performing an action intervention (e.g. by AI engine 2132 or a system associated therewith) may include transmitting information that causes another system to perform a real-world action, such as modifying an operation of an electromechanical device or displaying a specified message. Performing an action intervention may also include causing AI engine 2132 to take one or more particular actions by itself, where such actions are ones other than transmission of information (e.g. recording or computationally processing data associated with the action intervention, such as outcome data).

In some embodiments, method 1440 may include ranking of one or more action interventions 2172 that are generated by AI engine 2132. Ranking may be performed in order to determine suitability and/or importance of the one or more action interventions, in various embodiments. In an example where a user may be experiencing a medical emergency, a first action intervention may include contacting emergency services (e.g., the 911 system in the United States), while a second action intervention may include contacting a doctor of the user. In this instance, the first action intervention (911 contact) may be ranked higher than the intervention of contacting the doctor. Accordingly, in this example, the 911 action intervention may be executed (e.g. transmitted to another computer system) before transmitting the action intervention to the doctor. Generally, the order of execution of action interventions may thus be based on a ranking of the interventions. In some cases, however, a plurality of action interventions may be executed simultaneously (or substantially simultaneously) in parallel.

Method 1440 may also include the use of one or more secondary conditions, i.e., comorbidities, by AI engine 2132. Presence of one or more secondary conditions for a user may affect how AI engine 2132 generates and/or ranks action interventions, in various embodiments. If a user has asthma, for example, AI engine 2132 may generate an action intervention that may involve increasing a resistance setting on treatment apparatus 70 more slowly than it would otherwise, in order to avoid putting a strain on the pulmonary system of the user. In such a case, AI engine 2132 might also send an action intervention to the user with a message stating “Warning: difficulty will be increasing. Use your prescription inhaler if needed for asthma treatment.” A secondary condition may be a comorbidity such as hypertension, sepsis, a mental disorder, a mood disorder, diabetes, lung disease, obesity, heart disease, liver disease, kidney disease, vascular disease, arthritis, sleep apnea, osteoarthritis, anemia, stroke, asthma, any other physiological or anatomical disease, or some combination thereof. A secondary condition may also comprise a symptom indicating involvement or disorder of any physiological system in a user, any anatomical part of the user, any particular organ of the user, or some combination thereof.

Method 1440 may also include the use of user data 2109 as described above. More specifically, user data 2109 may be occluded, transformed, or otherwise de-identified using privacy enhancing techniques. User data 2109 may be de-identified prior to its receipt at AI engine 2132 in some instances. In other instances, AI engine 2132 may de-identify user data 2109 or any part thereof after receiving the user data. In various embodiments, all or a portion of data processed, stored, and/or transmitted by AI engine 2132 is de-identified. Thus, various data handled by AI engine 2132 may be processed to remove certain or all of any personally identifiable information for one or more users. Removal of personally identifiable information may include deleting, transforming, obfuscating, and/or editing information such as a name, address, demographic data, etc. De-identification of data may more generally involve deleting, transforming, obfuscating, and/or editing any information included in user data 2109, according to various embodiments.

Consistent with the above disclosure, the examples of systems and methods enumerated in the following clauses are specifically contemplated and are intended as a non-limiting set of examples.
Clause 1.22 A computer-implemented method comprising:
   receiving, from one or more of an electromechanical machine, a sensor, and a computing device, data associated with a user who uses the electromechanical machine to perform a treatment plan;
   based on the data, generating, using an artificial intelligence engine executing one or more large language models, one or more action interventions to perform, wherein at least one of the one or more action interventions comprise modifying one or more operating parameters of the electromechanical machine, and the one or more action interventions are generated based on one or more measures of efficacy;
      performing the one or more action interventions;
Clause 2.22 The computer-implemented method of clause 1.22, further comprising ranking, by the artificial intelligence engine, the one or more action interventions.
Clause 3.22 The computer-implemented method of clause 2.22, wherein the artificial intelligence engine uses one or more secondary conditions identified and explained by the large language model.
Clause 4.22 The computer-implemented method of clause 3.22, wherein the data is occluded, transformed, or otherwise de-identified due to the use of privacy enhancing techniques.
Clause 5.22 The computer-implemented method of clause 1.22, wherein the artificial intelligence engine executes at least one or more computer-implemented models comprising a machine learning model.
Clause 6.22 The computer-implemented method of clause 1.22, wherein the data is processed to remove any personally identifiable information.
Clause 7.22 The computer-implemented method of clause 1.22, wherein the one or more action interventions comprises causing operation of the electromechanical machine to be controlled.
Clause 8.22 A tangible, non-transitory computer-readable medium storing instructions that, when executed, cause one or more processing devices to:
   receive, from one or more of an electromechanical machine, a sensor, and a computing device, data associated with a user who uses the electromechanical machine to perform a treatment plan;
   based on the data, generate, using an artificial intelligence engine executing one or more large language models, one or more action interventions to perform, wherein at least one of the one or more action interventions comprise modifying one or more operating parameters of the electromechanical machine, and the one or more action interventions are generated based on one or more measures of efficacy; and perform the one or more action interventions.
Clause 9.22 The computer-readable medium of clause 8.22, wherein the one or more processing devices ranks, by the artificial intelligence engine, the one or more action interventions.
Clause 10.22 The computer-readable medium of clause 9.22, wherein the artificial intelligence engine uses one or more secondary conditions identified and explained by the large language model.
Clause 11.22 The computer-readable medium of clause 10.22, wherein the data is occluded, transformed, or otherwise de-identified due to the use of privacy enhancing techniques.
Clause 12.22 The computer-readable medium of clause 8.22, wherein the artificial intelligence engine executes at least one or more computer-implemented models comprising a machine learning model.
Clause 13.22 The computer-readable medium of clause 8.22, wherein the data is processed to remove any personally identifiable information.
Clause 14.22 The computer-readable medium of clause 8.22, wherein the one or more action interventions comprises causing operation of the electromechanical machine to be controlled.
Clause 15.22 A system comprising:
   one or more memory devices storing instructions; and
   one or more processing devices communicatively coupled to the one or more memory devices, wherein the one or more processing devices executes the instructions to:
receive, from one or more of an electromechanical machine, a sensor, and a computing device, data associated with a user who uses the electromechanical machine to perform a treatment plan;
based on the data, generate, using an artificial intelligence engine executing one or more large language models, one or more action interventions to perform, wherein at least one of the one or more action interventions comprise modifying one or more operating parameters of the electromechanical machine, and the one or more action interventions are generated based on one or more measures of efficacy; and perform the one or more action interventions.
Clause 16.22 The system of clause 15.22, wherein the one or more processing devices ranks, by the artificial intelligence engine, the one or more action interventions.
Clause 17.22 The system of clause 16.22, wherein the artificial intelligence engine uses one or more secondary conditions identified and explained by the large language model.
Clause 18.22 The system of clause 17.22, wherein the data is occluded, transformed, or otherwise de-identified due to the use of privacy enhancing techniques.
Clause 19.22 The system of clause 15.22, wherein the artificial intelligence engine executes at least one or more computer-implemented models comprising a machine learning model.
Clause 20.22 The system of clause 15.22, wherein the data is processed to remove any personally identifiable information.

### Method and System for Using AI/ML to Enhance Patient Treatment Plan Progression, Reduce Patient Pain, and Increase Patient Compliance by Adjusting, Based on One or More Real-Time Inputs, a Component Parameter

FIG. 14E shows an example embodiment of a method 1450 for determining and performing interventions using an electromechanical machine where the interventions may be determined by using a LLM to generate and rank, based on one or more measures of efficacy, a list of interventions. The method 1450 is performed by processing logic that may include hardware (circuitry, dedicated logic, etc.), software (such as is run on a general-purpose computer system or a dedicated machine), or a combination of both. The method 1450 and/or each of its individual functions, routines, subroutines, methods (as the term is used in object-oriented programming), or operations may be performed by one or more processing devices of a computing device (e.g., any component of FIG. 1, such as the assistant interface 94, patient interface 50, and/or server 30 executing the artificial intelligence engine 11, training engine 9, machine learning models 13, etc.) implementing the method 1450. The method 1450 may be implemented as computer instructions stored on a memory device and able to be executed by the one or more processing devices. In certain implementations, the method 1450 may be performed by a single processing thread. Alternatively, the method 1450 may be performed by two or more processing threads, each thread implementing one or more individual functions, routines, subroutines, methods or operations of the methods. In some embodiments, one or more accelerators may be used to increase the performance of a processing device by offloading various functions, routines, subroutines, methods or operations from the processing device.

For simplicity of explanation, the method 1450 is depicted in FIG. 14E and described as a series of operations. However, operations in accordance with this disclosure can occur in various orders and/or concurrently, and/or with other operations not presented and described herein. For example, the operations depicted in the method 1450 in FIG. 14E may occur in combination with any other operation of any other method disclosed herein. Furthermore, not all illustrated operations may be required to implement the method 1450 in accordance with the disclosed subject matter. In addition, those skilled in the art will understand and appreciate that the method 1450 could alternatively be represented as a series of interrelated states via a state diagram or event diagram.

In some embodiments, one or more computer-implemented models (e.g., machine learning models, neural networks, expert systems, etc.) may be generated and trained by the artificial intelligence engine 11 and/or the training engine 9 to perform one or more of the operations of the methods described herein. For example, to perform the one or more operations, the processing device may execute the one or more computer-implemented models. In some embodiments, the one or more computer-implemented models may be iteratively retrained to select different features capable of enabling optimization of output. The features that may be modified may include a number of nodes included in each layer of the models, an objective function executed at each node, a number of layers, various weights associated with outputs of each node, and the like.

At step 1452, the processing device may determine one or more individualized pain thresholds associated with one or more respective probabilities of a user progressing towards completing a treatment plan. In some embodiments, an artificial intelligence engine may determine the one or more individualized pain thresholds associated with one or more respective probabilities of a user progressing towards completing a treatment plan. In some embodiments, the artificial intelligence engine may execute or use one or more computer-implemented models (e.g., machine learning models 13) to determine the one or more individualized pain thresholds based on inputs (e.g., data related to one or more treatment plans performed by users having similar characteristics and performance measurements as the user) mapped to output (e.g., probabilities of users that progress towards completing a treatment plan, towards a desired rehabilitation goal, towards a desired rehabilitation level, etc.). In some embodiments, a therapist, clinician, and/or another medical professional may determine the one or more individualized pain thresholds.

At step 1454, the processing device may automatically cause, using the artificial intelligence engine 11, one or more modifications to an operating parameter or a component parameter of an electromechanical machine to reduce a user's pain within an individualized pain threshold level throughout a treatment session included in a treatment plan. In some embodiments, the artificial intelligence engine 11 may execute or use one or more computer-implemented models (e.g., machine learning models 13) that are trained using data related to one or more probabilities of the user progressing towards completing the treatment plan and/or achieving a rehabilitation goal or level.

At step 1456, the processing device may receive one or more pain measurement inputs. In some embodiments, the user may input the one or more pain measurement inputs into a computing device by using an input peripheral (e.g., microphone, keyboard, mouse, touchscreen, etc.). For example, the user may use a user interface presented via the computing device to select one or more pain measurement inputs for one or more physical and/or mental portions of the user's body and/or mind. In some embodiments, one or more sensors and/or cameras may use measurements related to the user's performance and/or video of the user performing a treatment plan to determine the one or more pain measurement inputs. The one or more pain measurement inputs may be determined using the artificial intelligence engine 11 executing or using one or more trained computer-implemented models (e.g., machine learning models 13).

At step 1458, the processing device may determine, based on the one or more pain measurement inputs, whether the individualized pain threshold level is satisfied. In some embodiments, based on the individualized pain threshold being satisfied, the processing device may modify a range of motion of at least one pedal of the electromechanical machine by 1 to 5 millimeters, 0.1 to 0.5 centimeters, 0.1 to 0.5 inches, 0.5 to 1 inch, or any other suitable range. In some embodiments, based on the individualized pain threshold being satisfied, the processing device may gradually (e.g., by .1 inch per second or per several seconds) modify one or more operating parameters (e.g., range of motion of pedals, resistance provided by the electric motor controlling the pedals, speed of the pedals, etc.) of the electromechanical machine.

In some embodiments, responsive to determining that the individualized pain threshold is not satisfied, the processing device may continue to use similar operating parameters or component parameters of the electromechanical machine while the user performs the treatment plan. To ensure progression towards completing the treatment plan and/or reaching a rehabilitation goal or level, the processing device may continuously monitor the pain measurement inputs to modify and/or maintain, using the artificial intelligence engine 11, the one or more operating parameters or component parameters of the electromechanical machine.

Consistent with the above disclosure, the examples of systems and methods enumerated in the following clauses are specifically contemplated and are intended as a non-limiting set of examples.
Clause 1.26 A computer-implemented method, comprising:
   determining one or more individualized pain thresholds associated with one or more respective probabilities of a user progressing towards completing a treatment plan;
   automatically causing, using an artificial intelligence engine, one or more modifications to a component parameter of an electromechanical machine to reduce a user's pain within an individualized pain threshold level throughout a treatment session included in a treatment plan; receiving one or more pain measurement inputs; and
   determining, based on the one or more pain measurement inputs, whether the individualized pain threshold level is satisfied.
Clause 2.26 The computer-implemented method of clause 1.26, wherein the user inputs, into a computing device, the one or more pain measurements inputs.
Clause 3.26 The computer-implemented method of clause 1.26, further comprising, based on the individualized pain threshold being satisfied, modifying a range of motion of at least one pedal by 0.1 inch to 0.5 inches.
Clause 4.26 The computer-implemented method of clause 1.26, further comprising, based on the individualized pain threshold being satisfied, gradually modifying one or more operating parameters of the electromechanical machine.
Clause 5.26 The computer-implemented method of clause 1.26, wherein the artificial intelligence engine comprises one or more computer-implemented models that are trained using data related to the one or more probabilities of the user progressing towards completing the treatment plan.
Clause 6.26 The computer-implemented method of clause 1.26, wherein the electromechanical machine includes at least one pedal.
Clause 7.26 The computer-implemented method of clause 1.26, further comprising, responsive to determining that the individualized pain threshold is not satisfied, continuing to use similar operating parameters of the electromechanical machine.
Clause 8.26 A tangible, non-transitory computer-readable medium storing instructions that, when executed, cause one or more processing devices to:
   determine one or more individualized pain thresholds associated with one or more respective probabilities of a user progressing towards completing a treatment plan;
   automatically cause, using an artificial intelligence engine, one or more modifications to a component parameter of an electromechanical machine to reduce a user's pain within an individualized pain threshold level throughout a treatment session included in a treatment plan; receive one or more pain measurement inputs; and
   determine, based on the one or more pain measurement inputs, whether the individualized pain threshold level is satisfied.
Clause 9.26 The computer-readable medium of clause 8.26, wherein the user inputs, into a computing device, the one or more pain measurements inputs.
Clause 10.26 The computer-readable medium of clause 8.26, wherein, based on the individualized pain threshold being satisfied, the one or more processing devices modifies a range of motion of at least one pedal by 0.1 inch to 0.5 inches.
Clause 11.26 The computer-readable medium of clause 8.26, wherein, based on the individualized pain threshold being satisfied, the one or more processing devices gradually modifies one or more operating parameters of the electromechanical machine.
Clause 12.26 The computer-readable medium of clause 8.26, wherein the artificial intelligence engine comprises one or more computer-implemented models that are trained using data related to the one or more probabilities of the user progressing towards completing the treatment plan.
Clause 13.26 The computer-readable medium of clause 8.26, wherein the electromechanical machine includes at least one pedal.
Clause 14.26 The computer-readable medium of clause 8.26, further comprising, responsive to determining that the individualized pain threshold is not satisfied, continuing to use similar operating parameters of the electromechanical machine.
Clause 15.26 A system comprising:
   one or more memory devices storing instructions; and
   one or more processing devices communicatively coupled to the one or more memory devices, wherein the one or more processing devices executes the instructions to:
   determine one or more individualized pain thresholds associated with one or more respective probabilities of a user progressing towards completing a treatment plan;
   automatically cause, using an artificial intelligence engine, one or more modifications to a component parameter of an electromechanical machine to reduce a user's pain within an individualized pain threshold level throughout a treatment session included in a treatment plan; receive one or more pain measurement inputs; and
   determine, based on the one or more pain measurement inputs, whether the individualized pain threshold level is satisfied.
Clause 16.26 The system of clause 15.26, wherein the user inputs, into a computing device, the one or more pain measurements inputs.
Clause 17.26 The system of clause 15.26, wherein, based on the individualized pain threshold being satisfied, the one or more processing devices modifies a range of motion of at least one pedal by 0.1 inch to 0.5 inches.
Clause 18.26 The system of clause 15.26, wherein, based on the individualized pain threshold being satisfied, the one or more processing devices gradually modifies one or more operating parameters of the electromechanical machine.
Clause 19.26 The system of clause 15.26, wherein the artificial intelligence engine comprises one or more computer-implemented models that are trained using data related to the one or more probabilities of the user progressing towards completing the treatment plan.
Clause 20.26 The system of clause 15.26, wherein the electromechanical machine includes at least one pedal.
   Systems and Methods for Performing AI-Driven Interventions which Combine Patient-Reported Outcome Measures and Objective Measurements

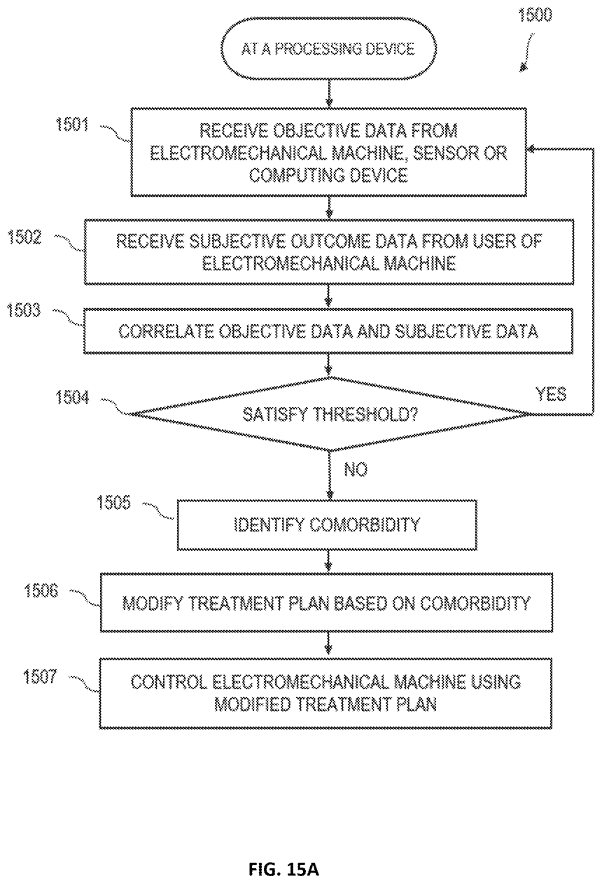

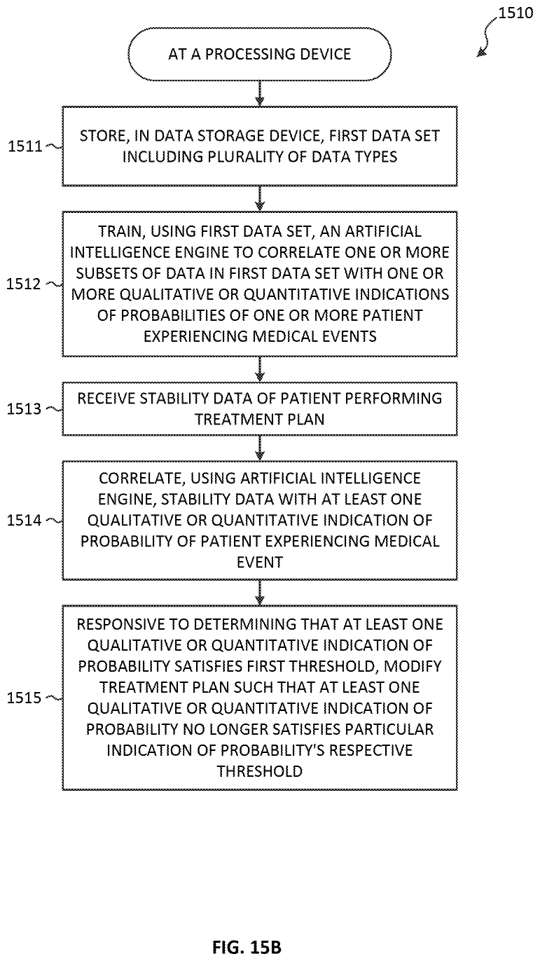

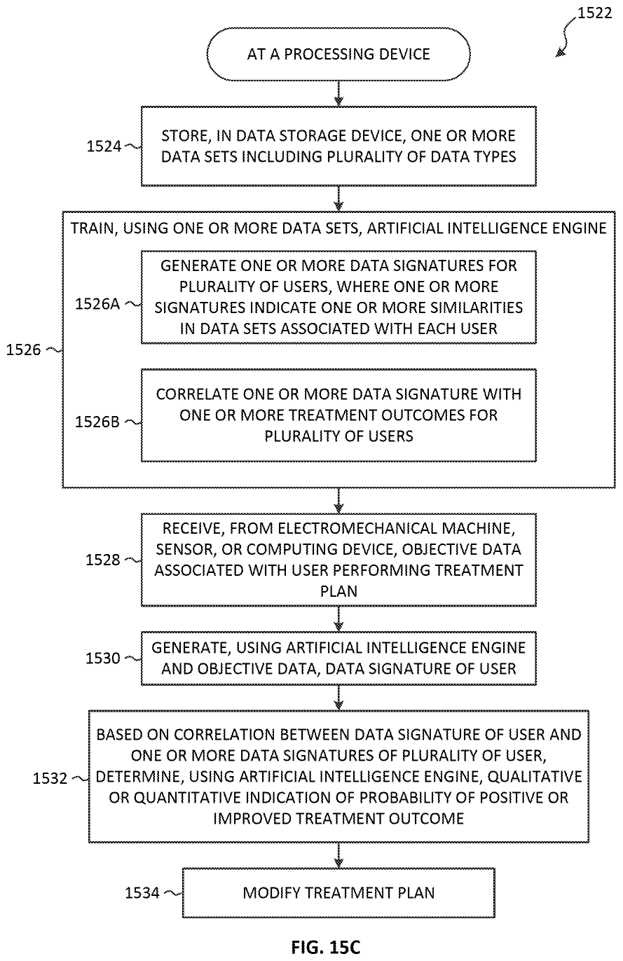

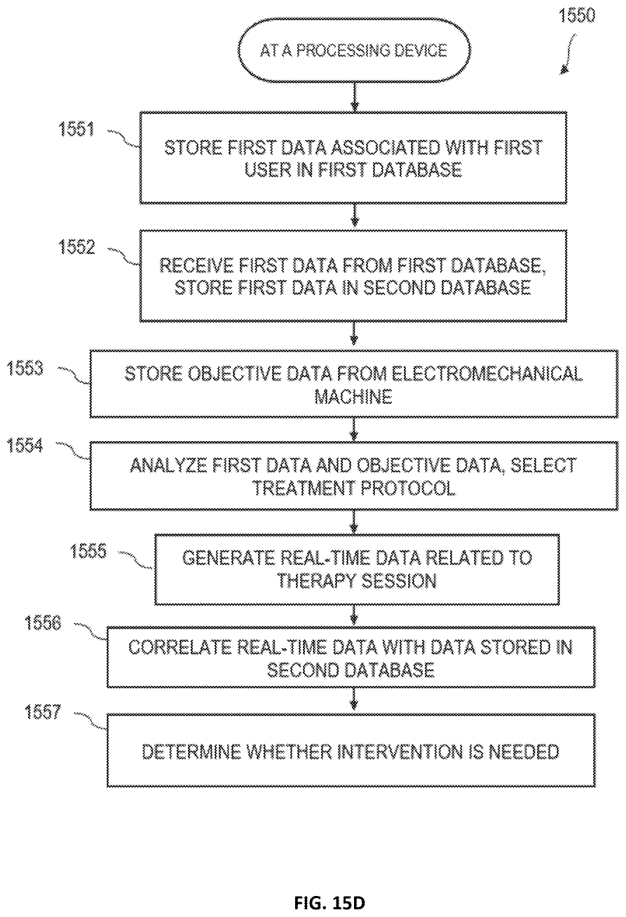

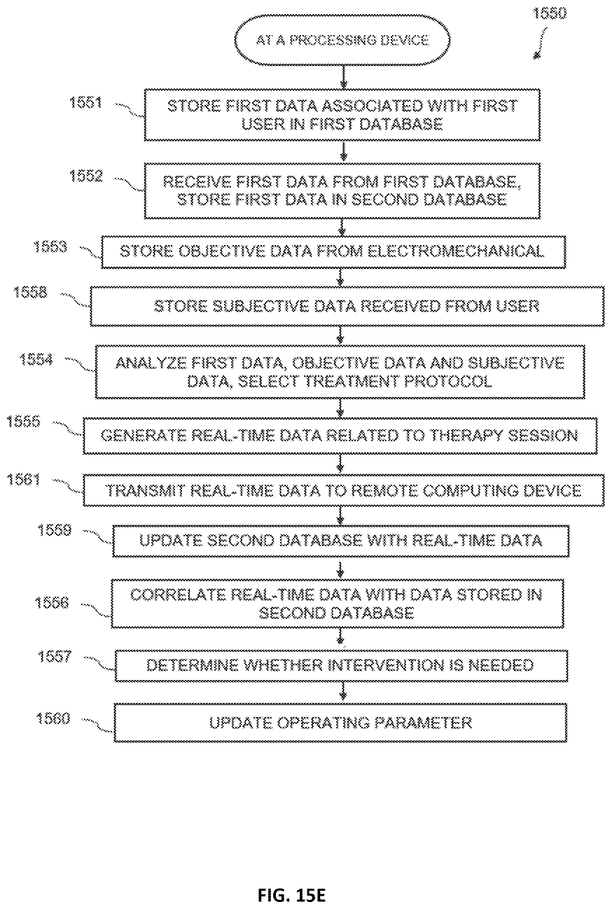

A further embodiment of the present application will now be described in connection with FIG. 15A. As described in further detail below, FIG. 15A describes a method 1500 of modifying a treatment plan based on at least one analysis and correlation of both objective data and subjective data related to a user using an electromechanical machine, such as the exercise bike described above, and controlling the electromechanical machine based on the modified treatment plan. The method 1500 is preferably implemented by a processing device such as one or more computer processors executing non-transitory computer-readable instructions stored in a memory device. More preferably, the method is performed in connection with an artificial intelligence engine to analyze data and determine correlations, the foregoing to identify comorbidities and modify treatment plans to improve treatment outcomes as will be discussed in further detail below. In some embodiments, the artificial intelligence engine may train one or more computer-implemented models that use generative artificial intelligence to create and/or modify treatment plans based on data the computer-implemented models are trained on. In some embodiments, the generative AI may use one or more foundation models that are large, pre-trained AI models that can be adapted for various tasks, such as correlating subjective and/or objective outcome data, generating treatment plans, modifying treatment plans, and the like. The generative AI may use various techniques, including deep learning, generative adversarial networks (GANs), and variational autoencoders.

An exemplary method will now be described in connection with the system previously described in connection with FIGS. 1-6. At step 1501, the processor receives objective data from one or more of an electromechanical machine, a sensor, or a computing device. The electromechanical machine may, for example, be exercise bike 70, 100. Exercise bike 70, 100, as previously described, preferably communicates with a remote server, and a user uses the exercise bike according to a treatment plan. The treatment plan may include a prescribed schedule for use of the electromechanical machine. The treatment plan may also include operating parameters of the electromechanical machine. In the case that the electromechanical machine is an exercise bike, the operating parameters may include pedal radius, resistance to rotation of the pedals, and so on. As examples of a sensor, any device that measures a parameter and provides objective data representative of that parameter are contemplated, and specific examples include an ambulation sensor, a goniometer to measure joint angle such as the knee angle sensor shown in FIG. 4, a pressure sensor measuring pressure applied to a pedal of the exercise bike, a position sensor such as a rotational position sensor that detects a rotational position of the pedals of the exercise bike, a velocity sensor such as a rotational velocity sensor that detects a rotational position of the pedals of the exercise bike, a biometric sensor, a blood oxygen sensor that measures blood oxygen level of the electromechanical machine user, a respiration rate sensor, a heart rate sensor, or any combination thereof. Blood oxygen, heart rate, and other user biometric data may be obtained, for example, from a wearable device such as a smart watch worn by the user. Examples of objective data received from a wearable device worn by the user include a knee angle from a knee angle measurement device worn by the user, a step count of the user, a pulse of the user, or any combination thereof. Furthermore, in addition to receiving contemporaneous objective data from a device during use of the electromechanical machine used according to a treatment plan, the objective data may also include historical objective data from the same or separate data sources. That is, for example, in addition to receiving data such as the current pedal radius setting, the current user heart rate, real time pressure-data from the exercise boke pedals, and so on, the objective data may also include historical data from the same or other sources recorded from past exercise sessions.

At step 1502 of the method, subjective outcome data and/or qualitative data is received from the user of the electromechanical machine. The subjective outcome data may be obtained in any manner, but in the exemplary method, a tablet computer device having a patient interface is used by the user, as illustrated in FIG. 4, to input the subjective outcome data. The subjective outcome data input by the user is transmitted to a remote server to be added to accumulated data and analyzed in conjunction with other data. The subjective outcome data may be a self-evaluated pain level experienced while using the electromechanical machine according to the treatment plan. The pain level may be a pain level experienced at the onset of a treatment session, an average beginning pain for one or more sessions of the treatment plan, a pain level after a final session, an average pain after one or more sessions, or any combination thereof. The subjective outcome data may be the self-evaluated mood of the user. Other examples, while not intended to be limiting, of subjective outcome data include an exertion level, biometric characteristic of the user, a vital sign of the user, or any combination thereof. Furthermore, in addition to receiving contemporaneous subjective outcome data from a user during use of the electromechanical machine used according to a treatment plan, the subjective outcome data may also include historical subjective outcome data, including data pertaining to comorbidities, from the same or separate data sources. That is, for example, in addition to receiving data such as the subjective pain level and mood of the user for the current exercise session, the subjective outcome data may also include historical data on pain and mood and/or other types of subjective outcome data recorded from past exercise sessions.

Once objective data and subjective outcome data as described above are received, the method proceeds to step 1503. At step 1503, the received objective data and subjective data is correlated to determine if a correlation threshold is satisfied. The correlation may be a single data point meeting or exceeding a threshold, but more preferably, the objective and subjective outcome data is analyzed by an artificial intelligence engine as described previously, together with historical objective and subjective data. Preferably, the artificial intelligence engine correlates the received objective and subjective outcome data with historical objective and subjective outcome data, wherein the AI engine performs robust pattern recognition and/or uses generative AI algorithms or other AI methods, the foregoing based on a large data set and machine learning techniques, as discussed previously. If the one or both of the objective data and the subjective outcome data does not satisfy a threshold correlation level, then the method returns to step 1501 such that further objective and subjective outcome data is obtained. If, however, both the objective and subjective outcome data satisfy a threshold correlation level (e.g. correlation coefficient), then the method continues to step 1505. At step 1505, the presence of at least one comorbidity is determined to exist. Next, at step 1506, a modified treatment plan is generated based on the at least one identified comorbidity. Finally, at step 1507, the electromechanical machine is controlled using the modified treatment plan, preferably while the user uses the electromechanical machine.

In step 1506, the method modifies the treatment plan. This can include modifying the schedule, frequency and/or intensity of exercise sessions. This can also include parameters of the electromechanical machine, including pedal radius or rotational velocity of the pedals. Preferably, modifying the treatment plan includes modifying a parameter or activity associated with at least two of the pain measurement, the pedal radius, the rotational velocity of the electromechanical machine, and an indicator of medicine utilization. Furthermore, modifying the treatment plan may include, without limitation, modifying an operating parameter of the electromechanical machine, stopping the electromechanical machine, contacting emergency services, or any combination thereof. It should be appreciated that the foregoing is provided as an example of an embodiments of the present application, and should not be considered limiting. Modifications, as will be appreciated by those of ordinary skill in the art, may be made without departing from the scope and sprit of the above described embodiment.

Consistent with the above disclosure, the examples of systems and methods enumerated in the following clauses are specifically contemplated and are intended as a non-limiting set of examples.
Clause 1.8 A computer-implemented method comprising:
   receiving, from one or more of an electromechanical machine, a sensor, and a computing device, objective data associated with a user that uses the electromechanical machine to perform a treatment plan;
   receiving, from a user of the electromechanical machine, subjective outcome data;
   based on the objective and subjective data, determining, using an artificial intelligence engine, a presence of at least one comorbidity associated with the user, wherein the determining comprises:
correlating at least one objective datum and at least one subjective datum, wherein each of the correlations satisfies a threshold correlation level;
   based on each of the correlations satisfying the threshold correlation level, identifying at least one comorbidity associated with the user;
   based on the at least one identified comorbidity associated with the user, generating, using the artificial intelligence engine, a modified treatment plan for the user; and
   controlling, while the user uses the electromechanical machine and using the modified treatment plan, the electromechanical machine.
Clause 2.8 The computer-implemented method of clause 1.8, wherein the objective data is received from a sensor.
Clause 3.8 The computer-implemented method of clause 2.8, wherein the sensor is an ambulation sensor, a goniometer, a pressure sensor, a position sensor, a velocity sensor, a biometric sensor, a blood oxygen sensor, a respiration rate sensor, a heart rate sensor, or some combination thereof.
Clause 4.8 The computer-implemented method of clause 1.8, wherein the objective data is an operating parameter setting.
Clause 5.8 The computer-implemented method of clause 4.8, wherein the operating parameter is a pedal radius.
Clause 6.8 The computer-implemented method of clause 1.8, wherein the subjective data is received from a patient interface.
Clause 7.8 The computer-implemented method of clause 6.8, wherein the subjective data comprises a pain level input by the user using an input peripheral associated with the computing device.
Clause 8.8 The computer-implemented method of clause 6.8, wherein the subjective data comprises a mood of the user.
Clause 9.8 The computer-implemented method of clause 6.8, wherein the subjective data comprises an exertion level, a biometric characteristic of the user, a vital sign of the user, or some combination thereof.
Clause 10.8 The computer-implemented method of clause 1.8, wherein the objective data is received from a wearable device worn by the user.
Clause 11.8 The computer-implemented method of clause 10.8, wherein the objective data received from the wearable device comprises a knee angle received from a knee angle measurement device worn by the user, a step count of the user, a pulse of the user, or some combination thereof.
Clause 12.8 The computer-implemented method of clause 1.8, further comprising receiving additional historical objective data and the correlating comprises correlations associated with the additional historical objective data.
Clause 13.8 The computer-implemented method of clause 1.8, further comprising receiving additional historical subjective data and the correlating comprises correlations associated with the additional historical subjective data.
Clause 14.8 The computer-implemented method of clause 1.8, further comprising generating the modified treatment plan by modifying a parameter or activity associated with the at least two of the pain measurement, the pedal radius, the measurement of revolutions per minute, and the indicator of medicine utilization.
Clause 15.8 The computer-implemented method of clause 1.8, wherein the computing device transmits, using an input peripheral associated with the computing device, the pain measurement input by the user.
Clause 16.8 The computer-implemented method of clause 1.8, wherein the pain level comprises a pain level at onset of a treatment session, an average beginning pain for one or more sessions of the treatment plan, a pain level after a final session, an average pain after the one or more sessions, or some combination thereof.
Clause 17.8 The computer-implemented method of clause 1.8, wherein the modified treatment plan comprises modifying an operating parameter of the electromechanical machine, stopping the electromechanical machine, contacting emergency services, or some combination thereof.
Clause 18.8 A tangible, non-transitory computer-readable medium storing instructions that, when executed, cause one or more processing devices to:
   receive, from one or more of an electromechanical machine, a sensor, and a computing device, objective data associated with a user that uses the electromechanical machine to perform a treatment plan;
      receive, from a user of the electromechanical machine, subjective outcome data;
      based on the objective and subjective data, determine, using an artificial intelligence engine, a presence of at least one comorbidity associated with the user, wherein the determining comprises:
   correlate at least one objective datum and at least one subjective datum, wherein each of the correlations satisfies a threshold correlation level;
      based on each of the correlations satisfying the threshold correlation level, identify at least one comorbidity associated with the user;
      based on the at least one identified comorbidity associated with the user, generate, using the artificial intelligence engine, a modified treatment plan for the user; and
      control, while the user uses the electromechanical machine and using the modified treatment plan, the electromechanical machine.
Clause 19.8 The computer-readable medium of clause 18.8, wherein the objective data is received from a sensor.
Clause 20.8 A system comprises one or more memory devices storing instructions and one or more processing devices communicatively coupled to the one or more memory devices wherein the one or more processing devices execute the instructions to:
   receive, from one or more of an electromechanical machine, a sensor, and a computing device, objective data associated with a user that uses the electromechanical machine to perform a treatment plan;
      receive, from a user of the electromechanical machine, subjective outcome data;
      based on the objective and subjective data, determine, using an artificial intelligence engine, a presence of at least one comorbidity associated with the user, wherein the determining comprises:
   correlate at least one objective datum and at least one subjective datum, wherein each of the correlations satisfies a threshold correlation level;
   based on each of the correlations satisfying the threshold correlation level, identify at least one comorbidity associated with the user;
      based on the at least one identified comorbidity associated with the user, generate, using the artificial intelligence engine, a modified treatment plan for the user; and
      control, while the user uses the electromechanical machine and using the modified treatment plan, the electromechanical machine.
         Methods and Systems Based on Data Lakes for using AI/ML to Reduce a Probability for An Event Where a Patient Falls

FIG. 15B is a flow diagram generally illustrating a method 1510 for, based on data lakes, using artificial intelligence and machine learning to reduce a probability for an event where a patient falls according to principles of the present disclosure. The method 1510 is performed by processing logic that may include hardware (circuitry, dedicated logic, etc.), software (such as is run on a general-purpose computer system or a dedicated machine), or a combination of both. The method 1510 and/or each of its individual functions, routines, subroutines, methods (as the term is used in object-oriented programming), or operations may be performed by one or more processing devices of a computing device (e.g., any component of FIG. 1, such as the assistant interface 94, patient interface 50, and/or server 30 executing the artificial intelligence engine 11, training engine 9, machine learning models 13, etc.) implementing the method 1502. The method 1502 may be implemented as computer instructions stored on a memory device and able to be executed by the one or more processing devices. In certain implementations, the method 1510 may be performed by a single processing thread. Alternatively, the method 1510 may be performed by two or more processing threads, each thread implementing one or more individual functions, routines, subroutines, methods or operations of the methods. In some embodiments, one or more accelerators may be used to increase the performance of a processing device by offloading various functions, routines, subroutines, methods or operations from the processing device.

For simplicity of explanation, the method 1510 is depicted in FIG. 15B and described as a series of operations. However, operations in accordance with this disclosure can occur in various orders and/or concurrently, and/or with other operations not presented and described herein. For example, the operations depicted in the method 1510 in FIG. 15B may occur in combination with any other operation of any other method disclosed herein. Furthermore, not all illustrated operations may be required to implement the method 1510 in accordance with the disclosed subject matter. In addition, those skilled in the art will understand and appreciate that the method 1510 could alternatively be represented as a series of interrelated states via a state diagram or event diagram.

In some embodiments, one or more computer-implemented models (e.g., machine learning models, neural networks, expert systems, etc.) may be generated and trained by the artificial intelligence engine 11 and/or the training engine 9 to perform one or more of the operations of the methods described herein. For example, to perform the one or more operations, the processing device may execute the one or more computer-implemented models. In some embodiments, the one or more computer-implemented models may be iteratively retrained to select different features capable of enabling optimization of output. The features that may be modified may include a number of nodes included in each layer of the models, an objective function executed at each node, a number of layers, various weights associated with outputs of each node, and the like.

At step 1511, the processing device may store, in a data storage device, a first data set including a diverse set of data types. The data types may be included in a centralized repository in structured, semi-structured, and/or unstructured data that is capable of storing large volumes of diverse data types. This may be beneficial for big data and machine learning applications described herein. The diverse set of data types may be associated with a multitude of patients and may include medical professionals' notes, lab results, test results, medical histories, comorbidities, demographic information, psychographic information, biometric information, physical assessment information, questionnaire information, and the like.

At step 1512, the processing device may train, using the first data set, an artificial intelligence engine to correlate one or more subsets of data in the first data set with one or more qualitative or quantitative indications of probabilities of one or more patients experiencing medical events. The artificial intelligence engine may execute one or more computer-implemented models (e.g., machine learning models) that are trained using training data to correlate the one or more subsets of data in the first data set with one or more qualitative or quantitative indications of probabilities of one or more patients experiencing medical events (e.g., a fall event, a physiological event, a biological event, a mental-related event, etc.). In some embodiments, the qualitative indications may be associated with probabilities of likelihoods of an event to occur. In some embodiments, the qualitative indication may refer to some event that is more probable or less probable than another event without specifying exact numerical probabilities.

At 1513, the processing device may receive stability data of a patient performing the treatment plan. In some embodiments, the stability data may include a gait of the patient, a balance of the patient, or both. In some embodiments, the gait of the patient may include conditions such as ataxia. In some embodiments, the stability data may be received from a sensor on an electromechanical machine used by the patient. In some embodiments, the stability data may be received from a wearable device worn by the patient. in some embodiments, the stability data may include gait deviations from an expected gait. In some embodiments, the expected gait may be determined based on data lakes of gait information for people having characteristics similar to those of the current patient. In some embodiments, the stability data may include balance deviations from an expected balance. In some embodiments, the expected balance may be determined based on data lakes of balance information for people having characteristics similar to those of the current patient. In some embodiments, the stability data may include gait and balance deviations from an expected gait and balance. In some embodiments, the stability data may include data indicating a Fall Event for a User (FEU) representing that the patient has fallen or has begun to fall down. An FEU may refer to any detected, determined, reported, sensed, visualized, etc. event representing that the patient has fallen (e.g., sensor measurements in a wearable exceed a threshold movement measure in a certain direction, sensor measurement in a floor tile detects a sudden increase in pressure, video data indicates the patient has begun to fall or has fallen).

At 1514, the processing device may correlate, using the artificial intelligence engine, the stability data with at least one qualitative or quantitative indication of a probability of the patient experiencing a medical event. In some embodiments, the artificial intelligence engine may use one or more computer-implemented models (e.g., machine learning models) to correlate stability data with at least one qualitative or quantitative indication of a probability of the patient experiencing the medical event.

At 1515, responsive to determining that the at least one qualitative or quantitative indication of the probability satisfies a first threshold, the processing device may modify the treatment plan such that the at least one qualitative or quantitative indication of the probability no longer satisfies that particular indication of the probability's respective threshold. In some embodiments, modifying the treatment plan may include, by modifying an operating parameter of an electromechanical machine used by the patient to perform the treatment plan, causing operation of the electromechanical machine to be controlled. In some embodiments, the modified treatment plan may include modifying at least one of the gait of the patient and the balance of the patient.

Consistent with the above disclosure, the examples of systems and methods enumerated in the following clauses are specifically contemplated and are intended as a non-limiting set of examples.
Clause 1.12 A computer-implemented method comprising:
   storing, in a data storage device, a first data set comprising a plurality of data types;
   training, using the first data set, an artificial intelligence engine to correlate one or more subsets of data in the first data set with one or more qualitative or quantitative indications of probabilities of one or more patients experiencing medical events;
   receiving stability data of a patient performing the treatment plan, wherein the stability data comprises a gait of the patient, a balance of the patient, or both;
   correlating, using the artificial intelligence engine, the stability data with at least one qualitative or quantitative indication of a probability of the patient experiencing a medical event;
   responsive to determining that the at least one qualitative or quantitative indication of the probability satisfies a first threshold, modifying the treatment plan such that the at least one qualitative or quantitative indication of the probability no longer satisfies that particular indication of the probability's respective threshold.
Clause 2.12 The computer-implemented method of clause 1.12, wherein modifying the treatment plan comprises, by modifying an operating parameter of an electromechanical machine used by the patient to perform the treatment plan, causing operation of the electromechanical machine to be controlled.
Clause 3.12 The computer-implemented method of clause 1.12, wherein the stability data is received from a sensor on an electromechanical machine used by the patient.
Clause 4.12 The computer-implemented method of clause 1.12, wherein the stability data is received from a wearable device worn by the patient.
Clause 5.12 The computer-implemented method of clause 1.12, wherein the stability data comprises gait deviance from an expected gait.
Clause 6.12 The computer-implemented method of clause 1.12, wherein the stability data comprises balance deviance from an expected balance.
Clause 7.12 The computer-implemented method of clause 1.12, wherein the stability data comprises gait and balance deviance from an expected gait and balance.
Clause 8.12 The computer-implemented method of clause 1.12, wherein the stability data comprises data indicating a Fall Event for a User (FEU) representing that the patient has fallen or has begun to fall down.
Clause 9.12 The computer-implemented method of clause 1.12, wherein the modified treatment plan modifies at least one of the gait of the patient and the balance of the patient.
Clause 10.12 A tangible, non-transitory computer-readable medium storing instructions that, when executed cause a processing device to:
   store, in a data storage device, a first data set comprising a plurality of data types;
   train, using the first data set, an artificial intelligence engine to correlate one or more subsets of data in the first data set with one or more qualitative or quantitative indications of probabilities of one or more patients experiencing medical events;
   receive stability data of a patient performing the treatment plan, wherein the stability data comprises a gait of the patient, a balance of the patient, or both;
   correlate, using the artificial intelligence engine, the stability data with at least one qualitative or quantitative indication of a probability of the patient experiencing a medical event; and
   responsive to determining that the at least one qualitative or quantitative indication of the probability satisfies a first threshold, modify the treatment plan such that the at least one qualitative or quantitative indication of the probability no longer satisfies that particular indication of the probability's respective threshold.
Clause 11.12 The computer-readable medium of clause 10.12, wherein modifying the treatment plan comprises, by modifying an operating parameter of an electromechanical machine used by the patient to perform the treatment plan, causing operation of the electromechanical machine to be controlled.
Clause 12.12 The computer-readable medium of clause 10.12, wherein the stability data is received from a sensor on an electromechanical machine used by the patient.
Clause 13.12 The computer-readable medium of clause 10.12, wherein the stability data is received from a wearable device worn by the patient.
Clause 14.12 The computer-readable medium of clause 10.12, wherein the stability data comprises gait deviance from an expected gait.
Clause 15.12 The computer-readable medium of clause 10.12, wherein the stability data comprises balance deviance from an expected balance.
Clause 16.12 The computer-readable medium of clause 10.12, wherein the stability data comprises gait and balance deviance from an expected gait and balance.
Clause 17.12 The computer-readable medium of clause 10.12, wherein the stability data comprises data indicating a Fall Event for a User (FEU) representing that the patient has fallen or has begun to fall down.
Clause 18.12 The computer-readable medium of clause 10.12, wherein the modified treatment plan modifies at least one of the gait of the patient and the balance of the patient.
Clause 19.12 A system comprising:
   one or more memory devices storing instructions; and
   one or more processing devices communicatively coupled to the one or more memory devices, wherein the one or more processing devices execute the instructions to: store, in a data storage device, a first data set comprising a plurality of data types;
   train, using the first data set, an artificial intelligence engine to correlate one or more subsets of data in the first data set with one or more qualitative or quantitative indications of probabilities of one or more patients experiencing medical events;
   receive stability data of a patient performing the treatment plan, wherein the stability data comprises a gait of the patient, a balance of the patient, or both;
   correlate, using the artificial intelligence engine, the stability data with at least one qualitative or quantitative indication of a probability of the patient experiencing a medical event; and
   responsive to determining that the at least one qualitative or quantitative indication of the probability satisfies a first threshold, modify the treatment plan such that the at least one qualitative or quantitative indication of the probability no longer satisfies that particular indication of the probability's respective threshold.
Clause 20.12 The system of clause 19.12, wherein modifying the treatment plan comprises, by modifying an operating parameter of an electromechanical machine used by the patient to perform the treatment plan, causing operation of the electromechanical machine to be controlled.
   Methods for Obtaining and Using Objective Information Pertaining to a Performance of a Patient Performing a Treatment Plan in a Physical Therapy or Rehabilitative Session on a Rehabilitation Device AI/ML to Reduce a Probability for An Event Where a Patient Falls

FIG. 15C is a flow diagram generally illustrating a method 1522 for obtaining and using objective information pertaining to a performance of a patient performing a treatment plan on a rehabilitative device. The method 1522 is performed by processing logic that may include hardware (circuitry, dedicated logic, etc.), software (such as is run on a general-purpose computer system or a dedicated machine), or a combination of both. The method 1522 and/or each of its individual functions, routines, subroutines, methods (as the term is used in object-oriented programming/design) or operations may be performed by one or more processing devices of a computing device (e.g., any component of FIG. 1, such as the assistant interface 94, patient interface 50, and/or server 30 executing the artificial intelligence engine 11, training engine 9, machine learning models 13, etc.) implementing the method 1522. The method 1522 may be implemented as computer instructions stored on a memory device and able to be executed by the one or more processing devices. In certain implementations, the method 1522 may be performed by a single processing thread. Alternatively, the method 1522 may be performed by two or more processing threads, each thread implementing one or more individual functions, routines, subroutines, methods or operations of the methods. In some embodiments, one or more accelerators may be used to increase the performance of a processing device by offloading various functions, routines, subroutines, methods or operations from the processing device.

For simplicity of explanation, the method 1522 is depicted in FIG. 15C and described as a series of operations. However, operations in accordance with this disclosure can occur in various orders and/or concurrently, and/or with other operations not presented and described herein. For example, the operations depicted in the method 1522 in FIG. 15C may occur in combination with any other operation of any other method disclosed herein. Furthermore, not all illustrated operations may be required to implement the method 1522 in accordance with the disclosed subject matter. In addition, those skilled in the art will understand and appreciate that the method 1522 could alternatively be represented as a series of interrelated states via a state diagram or event diagram.

In some embodiments, one or more computer-implemented models (e.g., machine learning models, neural networks, expert systems, etc.) may be generated and trained by the artificial intelligence engine 11 and/or the training engine 9 to perform one or more of the operations of the methods described herein. For example, to perform the one or more operations, the processing device may execute the one or more computer-implemented models. In some embodiments, the one or more computer-implemented models may be iteratively retrained to select different features capable of enabling optimization of output. The features that may be modified may include a number of nodes included in each layer of the models, an objective function executed at each node, a number of layers, various weights associated with outputs of each node, and the like.

The method begins at step 1524, where one or more data sets, each including a plurality of data types, are stored in a data storage device. The data types may be included in a centralized repository in structured, semi-structured, and/or unstructured data that is capable of storing large volumes of diverse data types. This may be beneficial for big data and machine learning applications described herein. At step 1526, an artificial intelligence engine, such as the one discussed above, is trained on the one or more data sets stored in the data storage device. First, at step 1526A, one or more data signatures are generated for a plurality of users. The one or more data signatures indicate one or more similarities in data sets associated with each user. Next, at step 1526B, the one or more data signatures are correlated with one or more treatment outcomes for the plurality of users.

At step 1528, objective data is received from an electromechanical machine, (e.g., exercise bike), a sensor, a computing device, or some combination thereof. The objective data may be associated with a user performing a treatment plan. At step 1530, using the objective data and the trained artificial intelligence engine, a data signature for the user is generated. Using training data including inputs related to a corpus of objective data and outputs related to data signatures, the artificial intelligence engine may train one or more computer-implemented models (e.g., machine learning models) to generate the data signature.

At step 1532, based on the correlation between the data signature of the user, and one or more data signatures stored previously for the plurality of users, the artificial intelligence engine determines a qualitative (e.g., unlikely, possible, likely, high likely) or quantitative indication of a probability of a positive or improved treatment outcome. Then at step 1534, responsive to the qualitative or quantitative indication of a probability of a positive treatment outcome satisfying a threshold correlation level, the treatment plan may be modified to increase the probability of achieving the positive or improved treatment outcome.

In some embodiments, modifying the treatment plan may include executing the artificial intelligence engine to modify one or more operating parameters of the electromechanical machine. The objective data referred to above preferably comprises real-time sensor data received from the electromechanical machine while the user is using the electromechanical machine in accordance with the current treatment plan.

In addition to, or in place of, objective data, subjective data may be received from a user interface (e.g., patient interface 50) of a computing device operated by the user, such as a tablet shown in FIG. 4. The subjective data may include a pain level input by the user, a mood of the user, an exertion level of the user, a biometric characteristic of the user, a vital sign of the user, or any combination thereof.

The electromechanical machine is preferably a machine having at least one pedal, and the treatment plan preferably comprises operating parameters of the electromechanical machine. The operating parameters preferably include at least one of a pedal radius, a rotation rate of the at least one pedal, and a pressure exerted by the user on the at least one pedal.

In some embodiments, the objective data described above is received during a treatment session from a wearable device, such as the smart watch shown in FIG. 4, worn by the user. The objective data is preferably real-time data. The real-time data preferably includes at least one of a heart rate of the user, a blood pressure of the user, a blood oxygen level of the user, a respiration rate of the user, any other vital sign of the user, or any combination thereof.

Consistent with the above disclosure, the examples of systems and methods enumerated in the following clauses are specifically contemplated and are intended as a non-limiting set of examples.
Clause 1.13 A computer-implemented method comprising:
   storing, in a data storage device, one or more data sets corresponding to a plurality of users, wherein the one or more data sets comprise a plurality of data types;
   training, using the one or more data sets, an artificial intelligence engine to:
      (i) generate one or more data signatures for the plurality of users, wherein the one or more data signatures indicate one or more similarities in the data sets associated with each user, and
      (ii) correlate the one or more data signatures with one or more treatment outcomes for the plurality of users;
   receiving, from an electromechanical machine, a sensor, or a computing device, objective data associated with a user performing a treatment plan;
   generating, using the artificial intelligence engine and the objective data, a data signature of the user;
   based on a correlation between the data signature of the user and the one or more data signatures of the plurality of users, determining, using the artificial intelligence engine, a qualitative or quantitative indication of a probability of a positive or improved treatment outcome; and
   responsive to the qualitative or quantitative indication of a probability of the positive or improved treatment outcome satisfying a threshold level, modifying the treatment plan to increase the probability of achieving the positive or improved treatment outcome.
Clause 2.13 The computer-implemented method of clause 1.13, wherein modifying the treatment plan to improve the qualitative or quantitative indication of a probability of the treatment outcome comprises modifying, using the artificial intelligence engine, one or more operating parameters of the electromechanical machine.
Clause 3.13 The computer-implemented method of clause 1.13, wherein the objective data comprises real-time sensor data received from the electromechanical machine while the user is using the electromechanical machine in accordance with the treatment plan.
Clause 4.13 The computer-implemented method of clause 1.13, further comprising receiving subjective data from a user interface of a computing device operated by the user, wherein the subjective data comprises a pain level input by the user, a mood of the user, an exertion level of the user, a biometric characteristic of the user, a vital sign of the user, or some combination thereof.
Clause 5.13 The computer-implemented method of clause 1.13, wherein the treatment plan comprises operating parameters for an electromechanical machine operated by the user, wherein the electromechanical machine comprises a machine with at least one pedal, and the operating parameters comprise at least one of a pedal radius of the at least one pedal, a rotation rate of the at least one pedal, and a pressure exerted by the user on the at least one pedal.
Clause 6.13 The computer-implemented method of clause 1.13, wherein the objective data is received, during a treatment session, from a wearable device worn by the user, and the objective data comprises real-time data.
Clause 7.13 The computer-implemented method of clause 1.13, wherein the real-time data received from the wearable device comprises at least one of a heart rate of the user, a blood pressure of the user, a blood oxygen of the user, a respiration rate of the user, and another vital sign of the user.
Clause 8.13 A tangible, non-transitory computer-readable medium storing computer instructions that, when executed, cause one or more processing devices to:
   store, in a data storage device, one or more data sets corresponding to a plurality of users, wherein the one or more data sets comprise a plurality of data types;
   train, using the one or more data sets, an artificial intelligence engine to:
      (i) generate one or more data signatures for the plurality of users, wherein the one or more data signatures indicate one or more similarities in the data sets associated with each user, and
      (ii) correlate the one or more data signatures with one or more treatment outcomes for the plurality of users;
   receive, from an electromechanical machine, a sensor, or a computing device, objective data associated with a user performing a treatment plan;
   generate, using the artificial intelligence engine and the objective data, a data signature of the user;
   based on a correlation between the data signature of the user and the one or more data signatures of the plurality of users, determining, use the artificial intelligence engine, a qualitative or quantitative indication of a probability of a positive or improved treatment outcome; and
   responsive to the qualitative or quantitative indication of a probability of the positive or improved treatment outcome satisfying a threshold level, modify the treatment plan to increase the probability of achieving the positive or improved treatment outcome.
Clause 9.13 The computer-readable medium of clause 8.13, wherein modifying the treatment plan to improve the qualitative or quantitative indication of a probability of the treatment outcome comprises modifying, using the artificial intelligence engine, one or more operating parameters of the electromechanical machine.
Clause 10.13 The computer-readable medium of clause 8.13, wherein the objective data comprises real-time sensor data received from the electromechanical machine while the user is using the electromechanical machine in accordance with the treatment plan.
Clause 11.13 The computer-readable medium of clause 8.13, further comprising receiving subjective data from a user interface of a computing device operated by the user, wherein the subjective data comprises a pain level input by the user, a mood of the user, an exertion level of the user, a biometric characteristic of the user, a vital sign of the user, or some combination thereof.
Clause 12.13 The computer-readable medium of clause 8.13, wherein the treatment plan comprises operating parameters for an electromechanical machine operated by the user, wherein the electromechanical machine comprises a machine with at least one pedal, and the operating parameters comprise at least one of a pedal radius of the at least one pedal, a rotation rate of the at least one pedal, and a pressure exerted by the user on the at least one pedal.
Clause 13.13 The computer-readable medium of clause 8.13, wherein the objective data is received, during a treatment session, from a wearable device worn by the user, and the objective data comprises real-time data.
Clause 14.13 The computer-readable medium of clause 8.13, wherein the real-time data received from the wearable device comprises at least one of a heart rate of the user, a blood pressure of the user, a blood oxygen of the user, a respiration rate of the user, and another vital sign of the user.
Clause 15.13 A system comprising one or more memory devices storing instructions and one or more processing devices communicatively coupled to the one or more memory devices, wherein the one or more processing devices execute the instructions to:
   store, in a data storage device, one or more data sets corresponding to a plurality of users, wherein the one or more data sets comprise a plurality of data types;
   train, using the one or more data sets, an artificial intelligence engine to:
      (i) generate one or more data signatures for the plurality of users, wherein the one or more data signatures indicate one or more similarities in the data sets associated with each user, and
      (ii) correlate the one or more data signatures with one or more treatment outcomes for the plurality of users;
   receive, from an electromechanical machine, a sensor, or a computing device, objective data associated with a user performing a treatment plan;
   generate, using the artificial intelligence engine and the objective data, a data signature of the user;
   based on a correlation between the data signature of the user and the one or more data signatures of the plurality of users, determining, use the artificial intelligence engine, a qualitative or quantitative indication of a probability of a positive or improved treatment outcome; and
   responsive to the qualitative or quantitative indication of a probability of the positive or improved treatment outcome satisfying a threshold level, modify the treatment plan to increase the probability of achieving the positive or improved treatment outcome.
Clause 16.13 The system of clause 15.13, wherein modifying the treatment plan to improve the qualitative or quantitative indication of a probability of the treatment outcome comprises modifying, using the artificial intelligence engine, one or more operating parameters of the electromechanical machine.
Clause 17.13 The system of clause 15.13, wherein the objective data comprises real-time sensor data received from the electromechanical machine while the user is using the electromechanical machine in accordance with the treatment plan.
Clause 18.13 The system of clause 15.13, further comprising receiving subjective data from a user interface of a computing device operated by the user, wherein the subjective data comprises a pain level input by the user, a mood of the user, an exertion level of the user, a biometric characteristic of the user, a vital sign of the user, or some combination thereof.
Clause 19.13 The system of clause 15.13, wherein the treatment plan comprises operating parameters for an electromechanical machine operated by the user, wherein the electromechanical machine comprises a machine with at least one pedal, and the operating parameters comprise at least one of a pedal radius of the at least one pedal, a rotation rate of the at least one pedal, and a pressure exerted by the user on the at least one pedal.
Clause 20.13 The system of clause 15.13, wherein the objective data is received, during a treatment session, from a wearable device worn by the user, and the objective data comprises real-time data.

### Systems and Methods to Guide Rehabilitation by Inputting Data and Using Generative-AI to Generate Optimized Data Structures

A further embodiment of the present application will now be described in connection with FIG. 15D. As described in further detail below, FIG. 15D describes a method 1550 of determining whether intervention is recommended or needed in connection with a treatment plan based on at least one analysis and correlation of both first data associated with a user of an electromechanical machine and objective data received from the electromechanical machine, such as the exercise bike described above. The method 1550 is preferably implemented by a processing device such as one or more computer processors executing non-transitory computer-readable instructions stored in a memory device. More preferably, the method is performed in connection with an artificial intelligence engine to analyze data and determine correlations, the foregoing to determine whether modification of a treatment plan is recommended or needed as will be discussed in further detail below. In some embodiments, the artificial intelligence engine may train one or more computer-implemented models that use generative artificial intelligence to create new treatment plans based on data the computer-implemented models are trained on.

An exemplary method will now be described in connection with the system previously described in connection with FIGS. 1-6. At step 1551, the processor receives and stores first data associated with a first user in a first database, the first database having a first level of access control. At step 1552, the first data is received from the first database and stored in a second database having a second level of access control. Such an exemplary arrangement of storing a subset of data stored in a first database having a first level of access control in a second database having a second level of access control was previously described in connection with FIG. 13F, but the disclosure is not limited to that example. At step 1553, objective data received from the electromechanical machine is stored in the second database. The objective data can include sensor values or operating parameters of the electromechanical machine. At step 1554, the first data associated with the first user and objective data received from the electromechanical machine are analyzed, and a treatment plan for the first user is selected. Preferably the analysis is performed using an artificial intelligence engine trained on a large dataset as outlined above. At step 1555, a real-time therapy session of the first user, performed according to the selected treatment protocol, is monitored and real-time data related to the therapy session is generated. At step 1556, the generated real-time data is correlated with the data stored in the second database. The resulting one or more correlations are preferably determined by using the artificial intelligence engine. Based on the one or more correlations, the method determines whether intervention is recommended or needed at step 1557. It should be appreciated that correlations may be determined by using any known scientific, mathematical, statistical, psychological or other model or method. For example, a correlation coefficient may be determined that is a value R=−1, 0, 1, or any value in between. Examples include determining correlations between the real-time generated data and the data stored in the second database. The electromechanical machine may, for example, be exercise bike 70 or 100. Exercise bike 70 or 100, as previously described, preferably communicates with a remote server, and a user uses the exercise bike according to a treatment plan. The treatment plan may include a prescribed schedule for use of the electromechanical machine. The treatment plan may also include values or instructions pertaining to the establishment, use or restriction of operating parameters of the electromechanical machine. In the case that the electromechanical machine is an exercise bike, the operating parameters may include a pedal radius, range of motion, resistance to rotation of the pedals, centripetal force, velocity, acceleration, and so on. Objective data is preferably generated by a sensor incorporated into the electromechanical machine, or by an associated device such as a wearable device worn by the user during a treatment session (e.g., a goniometer), or by any biometric sensor, including any device that measures a parameter and provides an objective data representative of that parameter. Specific examples include an ambulation sensor, a goniometer to measure joint angle such as the knee angle sensor shown in FIG. 4, a pressure sensor measuring pressure applied to a pedal of the exercise bike, a position sensor such as a rotational position sensor that detects a rotational position of the pedals of the exercise bike, a velocity sensor such as a rotational velocity sensor that detects a rotational position of the pedals of the exercise bike, a biometric sensor, a blood oxygen sensor that measures blood oxygen level of the electromechanical machine user, a respiration rate sensor, a heart rate sensor, or any combination thereof. Blood oxygen, heart rate, and other user biometric data may be obtained, for example, from a wearable device such as a smart watch worn by the user. Examples of objective data received from a wearable device worn by the user may include a knee angle from a knee angle measurement device worn by the user, a step count of the user, a pulse of the user, or any combination thereof. Furthermore, in addition to receiving contemporaneous objective data from a device during use of the electromechanical machine used according to a treatment plan, the objective data may also include historical objective data including, without limitation, data pertaining to comorbidities, from the same or separate data sources. That is, for example, in addition to receiving data such as the current pedal radius setting, the current user heart rate, real time pressure-data from the exercise bike pedals, and so on, the objective data may also include historical data from the same or other sources recorded from past exercise sessions.

FIG. 15E illustrates additional steps which may be incorporated into the method described above in connection with FIG. 15D. In addition to storing objective data received from the electromechanical machine at step 1553, at step 1558, under this optional addition, subjective data may also be received from a user of the electromechanical machine is stored, preferably in the second database. At step 1559, the real-time data related to the therapy session is stored in the second database, thus updating the second database. At step 1560, based on determining that an intervention is recommended or needed, at least one operating parameter of the electromechanical machine is updated. Embodiments of the present method can advantageously permit remote monitoring of the generated real-time data. As shown at step 1561, the real-time data generated at the electromechanical machine is transmitted to a remote computing device to be monitored. At the remote computing device, the display is updated to reflect the received real-time data. The remote computing device used for real-time monitoring may be as described above in connection with FIG. 13G, for example. However, the real-time monitoring remote computing device should not be considered limited to this example, which is provided for illustrative purposes.

The subjective data reported by or received from the user may be obtained in any manner, but in the exemplary method, a tablet computer device having a patient interface is used by the user, as illustrated in FIG. 4, to input the subjective outcome data. The subjective data input by the user is transmitted to a remote server to be added to accumulated data and analyzed in conjunction with objective data and first data at step 1554. The subjective data may be a self-evaluated pain level experienced while using the electromechanical machine according to the treatment plan. The pain level may be a pain level experienced at the onset of a treatment session, an average beginning pain for one or more sessions of the treatment plan, a pain level after a final session, an average pain after one or more sessions, or any combination thereof. The subjective outcome data may be the self-evaluated mood of the user. Other examples, while not intended to be limiting, of subjective data include an exertion level, biometric characteristics of the user, a vital sign of the user, or any combination thereof. Furthermore, in addition to receiving contemporaneous subjective outcome data from a user during use of the electromechanical machine used according to a treatment plan, the subjective data may also include historical subjective data, including data pertaining to comorbidities, from the same or separate data sources. That is, for example, in addition to receiving data such as the subjective pain level and mood of the user for the current exercise session, the subjective data may also include historical data on pain and mood and/or other types of subjective data recorded from past exercise sessions.

As discussed above, at step 1556, real-time data is correlated with data stored in the second database to determine whether an intervention is recommended or needed. The correlation may be a single data point meeting or exceeding a threshold, but more preferably, the real-time data, the objective and, when suitable, the subjective data, are analyzed by an artificial intelligence engine as described previously, together with historical objective and subjective data. Preferably, the artificial intelligence engine correlates the received real-time data, and the objective and subjective outcomes with historical objective and subjective outcome data, wherein the AI engine performs robust pattern recognition and/or uses generative AI algorithms or other AI methods, the foregoing based on a large data set and machine learning techniques, as discussed previously. If the correlation satisfies a threshold correlation level, the method continues to step 1560.

In some embodiments, the generative AI may use one or more foundation models that are large, pre-trained AI models that can be adapted for various tasks, such as correlating subjective and/or objective outcome data, generating treatment plans, modifying treatment plans, and the like. The generative AI may use various techniques, including deep learning, generative adversarial networks (GANs), and variational autoencoders.

Operating parameters updating in step 1560 can include parameters of the electromechanical machine, including pedal radius or rotational velocity of the pedals. Preferably, modifying the treatment plan includes modifying a parameter or activity associated with at least two of the pain measurement, the pedal radius, the range of motion, the rotational velocity of the electromechanical machine, and an indicator of medicine utilization (e.g., prescription and over-the-counter types of pharmaceutical or drug used to treat pain, high blood pressure, diabetes, infections, etc.). Furthermore, modifying an operating parameter of the electromechanical machine, may include, without limitation, stopping the electromechanical machine, contacting emergency services, or any combination thereof. It should be appreciated that the foregoing is provided as an example of only one embodiment of the present application and should not be considered limiting. Modifications, as will be appreciated by those of ordinary skill in the art, may be made without departing from the scope and sprit of the above described embodiment.

Consistent with the above disclosure, the examples of systems and methods enumerated in the following clauses are specifically contemplated and are intended as a non-limiting set of examples.
Clause 1.11 A computer-implemented method comprising
   storing, in a first database, first data associated with a first user, the first database having a first level of access control;
   receiving from the first database, the first data, and storing the first data in a second database having a second level of access control;
   storing, in the second database, objective data received from an electromechanical machine;
   selecting, using an artificial intelligence engine, a treatment protocol for the first user by analyzing at least the first data and the objective data;
   generating real-time data related to the therapy session by monitoring a therapy session of the first user, wherein the therapy session is performed according to the selected treatment protocol;
   correlating, using the artificial intelligence engine, the generated real-time data with data stored in the second database; and
   based on the correlation, determining whether intervention is needed.
Clause 2.11 The computer-implemented method of clause 1.11, further comprising storing, in the second database, subjective data reported by or received from a user using the electromechanical machine, and long-term outcome data.
Clause 3.11 The computer-implemented method of clause 1.11, further comprising updating, by storing the generated real-time data associated with the therapy session, the second database.
Clause 4.11 The computer-implemented method of clause 1.11, wherein the monitoring comprises transmitting, to a remote computing device, the generated real-time data to cause a display of the remote computing device to be updated with the generated real-time data.
Clause 5.11 The computer-implemented method of clause 1.11, further comprising, responsive to a determination that the intervention is needed, updating at least one operating parameter of the electromechanical machine.
Clause 6.11 The computer-implemented method of clause 1.11, wherein the objective data is received from a sensor comprising an ambulation sensor, a goniometer, a pressure sensor, a position sensor, a velocity sensor, a biometric sensor, a blood oxygen sensor, a respiration rate sensor, a heart rate sensor, or some combination thereof.
Clause 7.11 The computer-implemented method of clause 1.11, wherein the objective data comprises an operating parameter.
Clause 8.11 The computer-implemented method of clause 7.11, wherein the operating parameter comprises a pedal radius.
Clause 9.11 The computer-implemented method of clause 2.11, wherein the subjective data is received from a patient interface.
Clause 10.11 The computer-implemented method of clause 9.11, wherein the subjective data is received via an input peripheral, and the subjective data comprises a pain level of the user, a mood of the user, an exertion level of the user, a biometric characteristic of the user, a vital sign of the user, or some combination thereof.
Clause 11.11 The computer-implemented method of clause 1.11, wherein the objective data is received from a wearable device worn by the user, and the objective data comprises a knee angle received from a knee angle measurement device worn by the user, a step count of the user, a pulse of the user, another vital sign of the user, or some combination thereof.
Clause 12.11 A tangible, non-transitory computer-readable medium storing instructions that, when executed, cause one or more processing devices to:
   store, in a first database, first data associated with a first user, the first database having a first level of access control;
      receive from the first database, the first data, and storing the first data in a second database having a second level of access control;
      store, in the second database, objective data received from an electromechanical machine;
      select, using an artificial intelligence engine, a treatment protocol for the first user by analyzing at least the first data and the objective data;
      generate real-time data related to the therapy session by monitoring a therapy session of the first user, wherein the therapy session is performed according to the selected treatment protocol;
      correlate, using the artificial intelligence engine, the generated real-time data with data stored in the second database; and
      based on the correlation, determine whether intervention is needed.
Clause 13.11 The computer-readable medium of clause 12.11, further comprising storing, in the second database, subjective data reported by or received from a user using the electromechanical machine, and long-term outcome data.
Clause 14.11 The computer-readable medium of clause 12.11, further comprising updating, by storing the generated real-time data associated with the therapy session, the second database.
Clause 15.11 The computer-readable medium of clause 12.11, wherein the monitoring comprises transmitting, to a remote computing device, the generated real-time data to cause a display of the remote computing device to be updated with the generated real-time data.
Clause 16.11 The computer-readable medium of clause 12.11, further comprising, responsive to a determination that the intervention is needed, updating at least one operating parameter of the electromechanical machine.
Clause 17.11 The computer-readable medium of clause 12.11, wherein the objective data is received from a sensor comprising an ambulation sensor, a goniometer, a pressure sensor, a position sensor, a velocity sensor, a biometric sensor, a blood oxygen sensor, a respiration rate sensor, a heart rate sensor, or some combination thereof.
Clause 18.11. The computer-readable medium of clause 12.11, wherein the objective data comprises an operating parameter.
Clause 19.11 The computer-readable medium of clause 18.11, wherein the operating parameter comprises a pedal radius.
Clause 20. A system comprising one or more memory devices storing instructions and one or more processing devices communicatively coupled to the one or more memory devices, wherein the one or more processing devices execute the instructions to:
   store, in a first database, first data associated with a first user, the first database having a first level of access control;
   receive from the first database, the first data, and storing the first data in a second database having a second level of access control;
   store, in the second database, objective data received from an electromechanical machine;
   select, using an artificial intelligence engine, a treatment protocol for the first user by analyzing at least the first data and the objective data;
   generate real-time data related to the therapy session by monitoring a therapy session of the first user, wherein the therapy session is performed according to the selected treatment protocol;
   correlate, using the artificial intelligence engine, the generated real-time data with data stored in the second database; and
   based on the correlation, determine whether intervention is needed.

### Systems and Methods for Analyzing Objective Information Pertaining to the Performance of a Patient Performing a Treatment Plan and for Modifying the Treatment Plan Using AI/ML

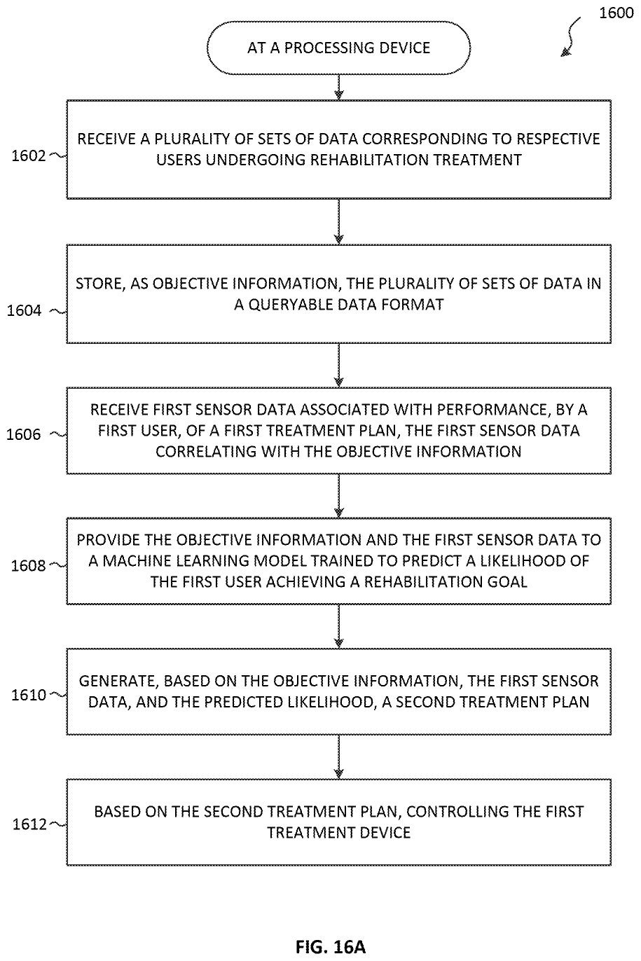

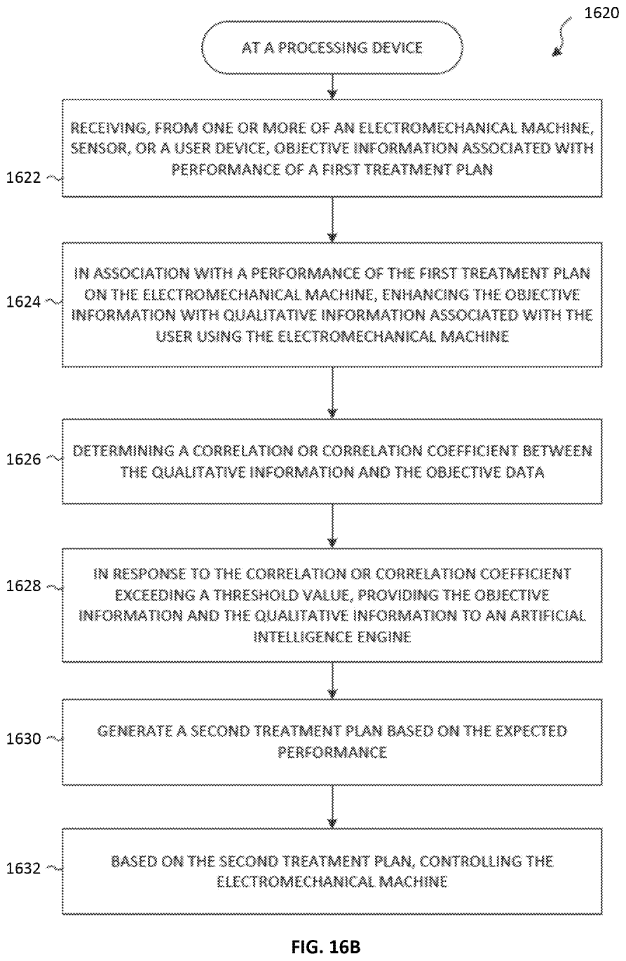

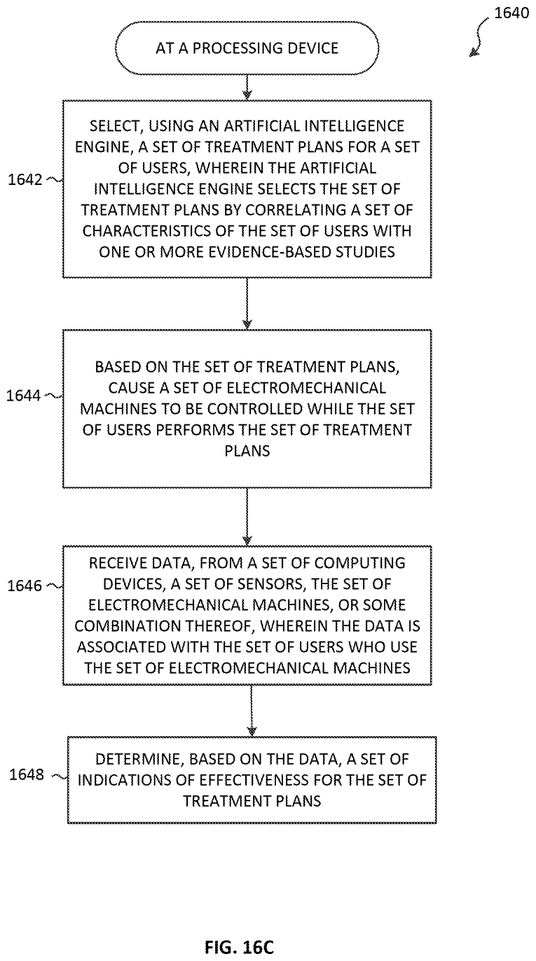

FIG. 16A shows an example embodiment of a method 1600 for analyzing objective information pertaining to the performance of a patient performing a treatment plan and for modifying the treatment plan using AI/ML. “Objective information” may include, without limitation, factual, observable, and measurable information that may not be influenced by personal feelings or opinions, such as sensor data obtained from one or more sensors associated with a patient or user, user device, treatment device, etc. The objective information may include information determined through various interactions with an electromechanical machine (e.g., the treatment device), sensor, or user device, including information associated with adjusting an operating parameter of the treatment device (e.g., adjusting a pedal radius setting, a resistance setting, a target RPM, etc.), feedback received regarding the first treatment plan, objective information input by the user and/or medical professional (e.g. treatment dates, durations, schedules, missed or performed treatment sessions, numbers of sessions, etc.), and/or other observable and measurable data.

As used herein, “objective information” is contrasted with subjective or qualitative information, which may include subjective narrations or other information provided by various users, medical professionals, etc. For example, subjective narrations may be provided by a therapist or other medical professional based on observations and personal assessments of a patient's performance, progress, pain levels, and so on. Such narrations may be provided in a non-standardized format or nomenclature that is not queryable (i.e., by a computing device, AI/ML model, etc.). In some examples, qualitative information may include patient reported outcomes.

Conversely, systems and methods according to the present disclosure are configured to obtain and store objective information, in real time, corresponding to sensor data generated while users actually perform various treatment plans using respective treatment devices. The sensor data that is stored as the objective information may correspond specifically to data that is relevant to diagnosis or other requirements for a particular rehabilitation treatment or protocol. Further, the data is stored in a standardized, queryable format (e.g., in a database structured for real time artificial intelligence/machine learning (AI/ML) access, retrieval, and analysis). In some examples, qualitative information (including patient reported outcomes or other subjective narratives) may be converted to objective information (“second objective information”) that can be stored in the standardized, queryable format.

The method 1600 can be performed by processing logic that may include hardware (circuitry, dedicated logic, etc.), software (such as is run on a general-purpose computer system or a dedicated machine), or a combination of both. The method 1600 and/or each of its individual functions, routines, subroutines, methods (as the term is used in object-oriented programming), or operations may be performed by one or more processing devices of a computing device (e.g., any component of FIG. 1, such as the assistant interface 94, patient interface 50, and/or server 30 executing the artificial intelligence engine 11, training engine 9, machine learning models 13, etc.) implementing the method 1600. The method 1600 may be implemented as computer instructions stored on a memory device and able to be executed by the one or more processing devices. In certain implementations, the method 1600 may be performed by a single processing thread. Alternatively, the method 1600 may be performed by two or more processing threads, each thread implementing one or more individual functions, routines, subroutines, methods or operations of the methods. In some embodiments, one or more accelerators may be used to increase the performance of a processing device by offloading various functions, routines, subroutines, methods or operations from the processing device.

For simplicity of explanation, the method 1600 is depicted in FIG. 16A and described as a series of operations. However, operations in accordance with this disclosure can occur in various orders and/or concurrently, and/or with other operations not presented and described herein. For example, the operations depicted in the method 1600 in FIG. 16A may occur in combination with any other operation of any other method disclosed herein. Furthermore, not all illustrated operations may be required to implement the method 1600 in accordance with the disclosed subject matter. In addition, those skilled in the art will understand and appreciate that the method 1600 could alternatively be represented as a series of interrelated states via a state diagram or event diagram.

In some embodiments, one or more computer-implemented models illustrated in FIG. 16A (e.g., machine learning models, neural networks, expert systems, etc.) may be generated and trained by the artificial intelligence engine 11 and/or the training engine 9 to perform one or more of the operations of the methods described herein. For example, to perform the one or more operations, the processing device may execute the one or more computer-implemented models. In some embodiments, the one or more computer-implemented models may be iteratively retrained to select different features capable of enabling optimization of output. The features that may be modified may include a number of nodes included in each layer of the models, an objective function executed at each node, a number of layers, various weights associated with outputs of each node, and the like.

At step 1602, the method 1600 includes receiving a plurality of sets of data corresponding to respective users of a plurality of users undergoing rehabilitation treatment using respective treatment devices. In an example, each of the sets of data includes sensor data obtained while the respective users perform respective treatment plans. The plurality of sets of data is received in real-time or near real-time as the respective users perform the respective treatment plans. The sets of data, which may be referred to objective data or objection information, may be received from one or more of an electromechanical machine (e.g., a treatment device), a sensor, a user device, etc. The data may correspond to objective information associated with performance of the treatment plans. The treatment plans may include one or more exercise routines for the patient or user to perform on the treatment device. In some examples, the one or more exercise routines for the patient or user may be completed during one or more treatment sessions. In some examples, the sensor data may be received in a time series data format. The time series data format may associate a time stamp with a time period or duration of time that the objective information was received.

At step 1604, the method 1600 includes storing the sets of data as objective information in a standardized, queryable data format (e.g., in a database, one or more cloud computing devices, etc.). Example standardized data formats may include, but are not limited to, comma-separated values (CSV), JavaScript Object Notation, Extensible Markup Language, Columnar Storage Formats, structured query language (SQL) databases, and so on.

The stored objective information may correspond to diagnostic or other requirements associated with the rehabilitation treatment being performed. For example, the obtained sensor or other data may be selective received, filtered, etc. based on the diagnostic requirements. In an example, sensor data may be assigned to various categories, labelled, tagged, etc. based on the rehabilitation treatment such that sensor data that is not relevant to the diagnostic requirements is not received, is received but not stored as the objective data, filtered, and so on.

At 1606, the method 1600 includes receiving sensor data (“first sensor data”) associated with performance of a first treatment plan by a first user using a first treatment device. For example, the first user may correspond to a user being actively monitored during rehabilitation treatment in accordance with the method 1600. The first treatment plan may be a standardized treatment plan assigned to the user based on a classification of the user. The first sensor data correlates with the objective information stored in the standardized, queryable data format. In other words, the first sensor data includes observable or measurable data in various categories analogous to or consistent with the stored objective information for a particular rehabilitation treatment. For example, for rehabilitation treatment for a knee or other joint or portion of a lower limb, the first sensor data may include selected or sensed operating characteristics of the treatment device associated with lower limb movement (e.g., pedal radius, resistance, force, speed, etc.), general operating characteristics of the treatment device and/or other observable characteristics of the treatment plan (e.g., duration, start and end times, pauses or breaks taken, changes in intensity, speed, or force, etc.), and/or user characteristics (e.g., heart rate, breathing rate, body temperature, etc.). The first sensor data may also be stored as the objective information in the database.

At 1608, the method 1600 includes predicting, based on the objective information (i.e., the stored objective information) and the first sensor data (i.e., sensor data or corresponding objective information corresponding specifically to the first user), a likelihood of the first user achieving a rehabilitation goal associated with the rehabilitation treatment. For example, the objective information and the first sensor data may be provided to an artificial intelligence engine (e.g., the artificial intelligence engine 11). Output of the artificial intelligence engine may predict the likelihood of achieving the rehabilitation goal based on a state of the user's progression in one or more categories, where the state describes or represents a result of the user's rehabilitation and the prediction is based on the result of the user's rehabilitation relative to the rehabilitation goal.

The artificial intelligence engine may be implemented in various forms to calculate the state of the user's progression and predict the likelihood of achieving the rehabilitation goal, including a model trained to perform a time-to-event prediction or longitudinal modeling task. The model may include, without limitation, a Random Forest or Random Survival Forest (e.g., where the Kaplan-Meier estimator in each leaf node is determined using the objective information to estimate the survival curve for observations in that node), a Recurrent Neural Network (RNN) (e.g., using a rectified linear unit (ReLU) activation function with the objective information), Bayesian Machine Learning or Bayesian Network (e.g., where the model learns from new objective information while also incorporating prior knowledge and quantifying uncertainty in the predictions), Gaussian Process (GP), Hidden Markov Model (HMM), Linear Regression (e.g., where the dependent variable can be a particular qualitative information (e.g., pain level) and the independent variables can be the objective information received in a time series that are fit to a linear equation), Random Forest Regression, Gradient Boosting Model (e.g., XGBoost, light gradient boosting machine (LightGBM)), or other model.

The artificial intelligence engine may be trained using a training process. For example, when an RNN is implemented as the artificial intelligence engine, the training process may comprise, without limitation, converting the objective information into sequences or vectors and implementing a forward pass to update the hidden step of the RNN (e.g., using a ReLU activation function). The loss may be calculated between predicted outputs generated by the forward pass and true targets. The training process may also implement a backpropagation and compute gradients through the time series data. Once gradients are computed, the parameters may be updated using an optimizer (e.g., stochastic gradient descent (SGD), Adam). The training process may be repeated over one or more epochs until the RNN converges or the loss stops improving more than a threshold value.

Once the artificial intelligence engine is trained, the artificial intelligence engine may be used to generate outputs (e.g., a state of the user's progression, where the state describes or represents a result of the user's rehabilitation, and the prediction of the likelihood of achieving the rehabilitation goal).

At step 1610, the method 1600 includes generating a second treatment plan (e.g., a modification of the first treatment plan) based on the prediction. For example, the second treatment plan may be generated by the artificial intelligence engine to optimize patient or user outcomes. In some examples, the second treatment plan may include one or more different medical procedures, tests, rehabilitation exercises on the treatment device, or some combination thereof.

At step 1612, the method 1600 includes controlling the treatment device based on the second treatment plan. The controlling can be based on parameters of the second treatment plan. For example, the artificial intelligence engine may be configured to modify the first treatment plan such that the treatment device changes a radius of rotation of one or more of the pedals, a level of assistance applied by the electric motor to assist the patient with cycling, an amount of resistance the electric motor applies to the one or more pedals, any other desired control, or combination thereof.

In some examples, the method 1600 may include generating a prompt in response to a determination that a component of the first sensor data exceeds a threshold, determining, based on the objective information and the first sensor data, a cause of the component exceeding the threshold, and, based on the determined cause, at least one of controlling the first treatment device to mitigate the cause and providing an instruction to the first user. In some examples, the prompt is provided to a user interface associated with a medical professional (e.g., via a display configured to display rehabilitation treatment information associated with one or more users currently performing respective treatment plans). The user interface may be configured to receive, from the medical professional, inputs for modifying control parameters of the first treatment device, the instructions for the first user, etc.

In some examples, the method 1600 may include receiving, in addition to the sensor data, qualitative information (including patient reported outcomes or other subjective narratives) and converting the qualitative information to objective information (“second objective information”) that can be stored in the standardized, queryable format or enhancing the objective information with the qualitative information. FIG. 16B shows an example embodiment of another method 1620 for analyzing objective information pertaining to the performance of a patient performing a treatment plan and for modifying the treatment plan (e.g., using AI/ML). In this example, the objective information may be enhanced with qualitative information. “Qualitative information” may include, without limitation data, that describes qualities or characteristics of the user (e.g., as perceived by the user). For example, the qualitative information may include a rating or feedback from a user in response to a request for information (e.g., a pain level experienced or reported by the user, perceived exertion, satisfaction with the treatment plan or treatment device, etc.). The method 1620 is performed by processing logic, one or more processing devices of a computing device, by executing computer instructions stored on a memory device and able to be executed by the one or more processing devices, etc. as described above with respect to FIG. 16A.

At step 1622, the method 1620 includes receiving, from one or more of an electromechanical machine, sensor, or a user device, objective information associated with performance of a first treatment plan. The first treatment plan may include one or more exercise routines for the patient or user to perform on the electromechanical machine. In some examples, the one or more exercise routines for the patient or user may be completed during one or more treatment sessions.

In some examples, the objective information may be received in a time series data format. The time series data format may associate a time stamp with a time period or duration of time that the objective information was received.

At step 1624, the method 1620 includes enhancing the objective information with qualitative information associated with the user using the electromechanical machine. For example, the objective information may be matched to the qualitative information based on the patient or user performing the first treatment plan.

In some examples, the qualitative information may be matched to the objective information based on the time stamp in the time series data format. In this way, the qualitative information can enhance the objective information at a particular time that the objective information was received. In some examples, adding the qualitative information may enhance the objective information.

At step 1626, the method 1620 includes determining a correlation between the qualitative information and the objective information, which in some examples, can be represented by a correlation coefficient. For example, qualitative information associated with a particular time stamp may be compared with objective information associated with the same particular time stamp.

In some examples, a correlation coefficient may be determined in accordance with Pearson's correlation coefficient r as follows:

$$r = \frac{\sum ( {x}_{i} - \overline{x} ) ( {y}_{i} - \overline{y} )}{\sqrt{\sum {( {x}_{i} - \overline{x} )}^{2} \cdot {( {y}_{i} - \overline{y} )}^{2}}}$$

Where xᵢ, yᵢ are individual data points (e.g., objective information and qualitative information) and x̅, y̅ are the means of X and Y. The correlation coefficient (r) may be between positive one and negative one. The closer the correlation coefficient is to a negative one value may identify stronger negative correlation compared to correlations between other negative values. The closer the correlation coefficient is to a positive one value may identify stronger positive correlation compared to correlations between other positive values.

At step 1628, the method 1620 includes providing the objective information and the qualitative information to an artificial intelligence engine (e.g., the artificial intelligence engine 11). Output of the artificial intelligence engine may predict a state of the user's final progression, where the state describes or represents a result of the user's rehabilitation.

The artificial intelligence engine may be implemented in various forms to predict the state of the user's final progression, including a model trained to perform a time-to-event prediction or longitudinal modeling task. The model may include, without limitation, a Random Forest or Random Survival Forest (e.g., where the Kaplan-Meier estimator in each leaf node is determined using the objective information and the qualitative information to estimate the survival curve for observations in that node), a Recurrent Neural Network (RNN) (e.g., using a ReLU activation function with the objective information and the qualitative information), Bayesian Machine Learning or Bayesian Network (e.g., where the model learns from new objective information and qualitative information while also incorporating prior knowledge and quantifying uncertainty in the predictions), Gaussian Process (GP), Hidden Markov Model (HMM), Linear Regression (e.g., where the dependent variable can be a particular qualitative information (e.g., pain level) and the independent variables can be the objective information and the qualitative information received in a time series that are fit to a linear equation), Random Forest Regression, Gradient Boosting Model (e.g., extreme gradient boosting (XGBoost), LightGBM), or other model.

The artificial intelligence engine may be trained using a training process. For example, when an RNN is implemented as the artificial intelligence engine, the training process may comprise, without limitation, converting the objective information enhanced with the qualitative information into sequences or vectors and implementing a forward pass to update the hidden step of the RNN (e.g., using a ReLU activation function). The loss may be calculated between predicted outputs generated by the forward pass and true targets. The training process may also implement a backpropagation and compute gradients through the time series data. Once gradients are computed, the parameters may be updated using an optimizer (e.g., SGD, Adam). The training process may be repeated over one or more epochs until the RNN converges or the loss stops improving more than a threshold value.

Once the artificial intelligence engine is trained, the artificial intelligence engine may be used to generate the output (e.g., the prediction of the state of the user's final progression, where the state describes or represents a result of the user's rehabilitation). In some examples, the objective information and the qualitative information may be provided to the trained artificial intelligence engine in response to the correlation or correlation coefficient exceeding a threshold value (e.g., as a positive number).

At step 1630, the method 1620 includes generating a second treatment plan based on the state of the user's final progression. For example, the second treatment plan may be generated by the artificial intelligence engine to optimize patient or user outcomes. In some examples, the second treatment plan may include one or more different medical procedures, tests, rehabilitation exercises on the electromechanical machine, or some combination thereof. The outcome may be partially based on the qualitative information reported during performance of the first treatment plan.

At step 1632, the method 1620 includes controlling the electromechanical machine based on the second treatment plan. The controlling can be based on parameters of the second treatment plan. For example, the artificial intelligence engine may be configured to modify the first treatment plan such that the electromechanical machine changes a radius of rotation of one or more of the pedals, a level of assistance applied by the electric motor to assist the patient with cycling, an amount of resistance the electric motor applies to the one or more pedals, any other desired control, or combination thereof.

Consistent with the above disclosure, the examples of systems and methods enumerated in the following clauses are specifically contemplated and are intended as a non-limiting set of examples.
Clause 1.14 A computer-implemented method comprising, using one or more processing devices: receiving a plurality of sets of data corresponding to respective users of a plurality of users undergoing rehabilitation treatment using respective treatment devices, wherein each of the sets of data includes sensor data obtained while the respective users perform respective treatment plans and the plurality of sets of data is received in real-time or near real-time as the respective users perform the respective treatment plans; storing, as objective information, the plurality of sets of data in a queryable data format; receiving first sensor data associated with the performance, by a first user using a first treatment device, of a first treatment plan, wherein the first treatment plan corresponds to a standardized treatment plan assigned to the user based on a classification of the user, and wherein the first sensor data correlate with the objective information stored in the queryable data format; providing the objective information and the first sensor data to a machine learning model trained to predict, based on the objective information and the first sensor data, a likelihood of the first user achieving a rehabilitation goal associated with the rehabilitation treatment; generating, based on the objective information, the first sensor data, and the likelihood of the first user achieving the rehabilitation goal, a second treatment plan; and controlling, based on the second treatment plan, the first treatment device.
Clause 2.14 The computer-implemented method of clause 1, wherein the objective information is stored based on diagnostic requirements associated with the rehabilitation treatment.
Clause 3.14 The computer-implemented method of clause 1, wherein the objective information is stored in a standardized format.

Clause 4.14 The computer-implemented method of clause 1, further comprising:

generating a prompt in response to a determination that a component of the sensor data exceeds a threshold; determining, based on the objective information and the sensor data, a cause of the component exceeding the threshold; and based on the determined cause, at least one of (i) controlling the first treatment device to mitigate the cause and (ii) providing an instruction to the first user.
Clause 5.14 The computer-implemented method of clause 4, further comprising providing the prompt to a user interface associated with a medical professional and receiving, via the user interface, at least one of (i) control parameters for the first treatment device and (ii) the instruction to the first user.
Clause 6.14 The computer-implemented method of clause 1, further comprising: receiving qualitative information corresponding to the respective users; converting the qualitative information to second objective information; and storing the second objective information in the queryable data format.
Clause 7.14 The computer-implemented method of clause 6, further comprising determining a correlation between the qualitative information and the second objective information and converting, based on the correlation, the qualitative information to the second objective information.
Clause 8.14 The computer-implemented method of clause 7, wherein the qualitative information corresponds to pain levels reported by the respective users.
Clause 9.14 A system, comprising: memory that stores a plurality of instructions; and
one or more processing devices configured to execute the plurality of instructions, wherein executing the plurality of instructions causes the one or more processing devices to receive a plurality of sets of data corresponding to respective users of a plurality of users undergoing rehabilitation treatment using respective treatment devices, wherein each of the sets of data includes sensor data obtained while the respective users perform respective treatment plans and the plurality of sets of data is received in real-time or near real-time as the respective users perform the respective treatment plans, store, as objective information, the plurality of sets of data in a queryable data format, receive first sensor data associated with the performance, by a first user using a first treatment device, of a first treatment plan, wherein the first treatment plan corresponds to a standardized treatment plan assigned to the user based on a classification of the user, and wherein the first sensor data correlate with the objective information stored in the queryable data format, provide the objective information and the first sensor data to a machine learning model trained to predict, based on the objective information and the first sensor data, a likelihood of the first user achieving a rehabilitation goal associated with the rehabilitation treatment, generate, based on the objective information, the first sensor data, and the likelihood of the first user achieving the rehabilitation goal, a second treatment plan, and control, based on the second treatment plan, the first treatment device.
Clause 10.14 The system of clause 9, wherein the objective information is stored based on diagnostic requirements associated with the rehabilitation treatment.
Clause 11.14 The system clause 9, wherein the objective information is stored in a standardized format.
Clause 12.14 The system of clause 9, wherein executing the plurality of instructions further causes the one or more processing devices to: generate a prompt in response to a determination that a component of the sensor data exceeds a threshold; determine, based on the objective information and the sensor data, a cause of the component exceeding the threshold; and based on the determined cause, at least one of (i) control the first treatment device to mitigate the cause and (ii) provide an instruction to the first user.
Clause 13.14 The system of clause 12, wherein executing the plurality of instructions further causes the one or more processing devices to provide the prompt to a user interface associated with a medical professional and receive, via the user interface, at least one of (i) control parameters for the first treatment device and (ii) the instruction to the first user.
Clause 14.14 The system of clause 9, wherein executing the plurality of instructions further causes the one or more processing devices to: receive qualitative information corresponding to the respective users; convert the qualitative information to second objective information; and store the second objective information in the queryable data format.
Clause 15.14 The system of clause 14, wherein executing the plurality of instructions further causes the one or more processing devices to determine a correlation between the qualitative information and the second objective information and convert, based on the correlation, the qualitative information to the second objective information.
Clause 16.14 The system of clause 15, wherein the qualitative information corresponds to pain levels reported by the respective users.

### Systems and Methods for Enabling Evidence-Based Studies to Determine the Efficacy of Different Rehabilitation Approaches

FIG. 16C is a flow diagram generally illustrating a method 1640 for enabling evidence-based studies to determine the efficacy of different rehabilitation approaches according to principles of the present disclosure. The method 1640 is performed by processing logic that may include hardware (circuitry, dedicated logic, etc.), software (such as is run on a general-purpose computer system or a dedicated machine), or a combination of both. The method 1640 and/or each of its individual functions, routines, subroutines, methods (as the term is used in object-oriented programming), or operations may be performed by one or more processing devices of a computing device (e.g., any component of FIG. 1, such as the assistant interface 94, patient interface 50, and/or server 30 executing the artificial intelligence engine 11, training engine 9, machine learning models 13, etc.) implementing the method 1640. The method 1640 may be implemented as computer instructions stored on a memory device and able to be executed by the one or more processing devices. In certain implementations, the method 1640 may be performed by a single processing thread. Alternatively, the method 1640 may be performed by two or more processing threads, each thread implementing one or more individual functions, routines, subroutines, methods or operations of the methods. In some embodiments, one or more accelerators may be used to increase the performance of a processing device by offloading various functions, routines, subroutines, methods or operations from the processing device.

For simplicity of explanation, the method 1640 is depicted in FIG. 16C and described as a series of operations. However, operations in accordance with this disclosure can occur in various orders and/or concurrently, and/or with other operations not presented and described herein. For example, the operations depicted in the method 1640 in FIG. 16C may occur in combination with any other operation of any other method disclosed herein. Furthermore, not all illustrated operations may be required to implement the method 1640 in accordance with the disclosed subject matter. In addition, those skilled in the art will understand and appreciate that the method 1640 could alternatively be represented as a series of interrelated states via a state diagram or event diagram.

In some embodiments, one or more computer-implemented models (e.g., machine learning models, neural networks, expert systems, etc.) may be generated and trained by the artificial intelligence engine 11 and/or the training engine 9 to perform one or more of the operations of the methods described herein. For example, to perform the one or more operations, the processing device may execute the one or more computer-implemented models. In some embodiments, the one or more computer-implemented models may be iteratively retrained to select different features capable of enabling optimization of output. The features that may be modified may include a number of nodes included in each layer of the models, an objective function executed at each node, a number of layers, various weights associated with outputs of each node, and the like.

At step 1642, the processing device may select, using an artificial intelligence engine, a set of treatment plans for a set of users. The artificial intelligence engine selects the set of treatment plans by correlating a set of characteristics of the set of users with one or more evidence-based studies. In some embodiments, the set of characteristics may include at least one of a neurological, oncological, cardiac, cardiovascular, orthopedic, pulmonary, neurological, or other physiological or anatomical system or condition of the set of users. In some embodiments, the evidence-based studies may enable medical professionals to incorporate the best available scientific evidence into individual patient care decisions.

Evidence-based studies may be generated using systematic reviews based on clinical trials, and/or using validated outcome measurements. The evidence-based studies may undergo an approval process where a certain number or percentage of certified medical professionals has to agree that the results and/or guidance included in the evidence-based studies is accurate and should be used to treat patients. The efficacy of the evidence-based studies may depend on the quality of evidence provided by clinical research. Some embodiments of the present disclosure may, in order to provide optimized care and/or desired results for the users, leverage the evidence-based studies to select treatment plans for users having certain characteristics.

At step 1644, the processing device may, based on the set of treatment plans, cause a set of electromechanical machines to be controlled while the set of users performs the set of treatment plans. In some embodiments, the set of electromechanical machines may be communicatively coupled to a server that enables one or more medical professionals to monitor one or more operating parameters of the set of electromechanical machines, one or more measurements associated with the set of users, or both in real-time or near real-time. In some embodiments, the one or more medical professionals may view one or more operating parameters of the electromechanical machines and/or the one or more measurements associated with the set of users via user interfaces of computing devices associated with the medical professionals. The medical professionals may use the computing devices to initiate a telemedicine session with any one or more of the set of users being monitored while they perform the treatment plans.

At step 1646, the processing device may receive data, from a set of computing devices, a set of sensors, the set of electromechanical machines, or some combination thereof. The data may be associated with the set of users who use the set of electromechanical machines. In some embodiments, the data may include objective data, qualitative data, or both. In some embodiments, the objective data may include a set of revolutions per minute measurements, speed measurements, range of motion measurements, measurements of pressure exerted on a plurality of pedals of the plurality of electromechanical machines, or some combination thereof. In some embodiments, the qualitative data may include at least one of a set of pain levels experienced by the set of users, a set of moods associated with the set of users, a set of biometric measurements associated with the set of users, a set of vital signs associated with the set of users, or some combination thereof.

At step 1648, the processing device may determine, based on the data, one or more sets of indications of effectiveness for the set of treatment plans. In some embodiments, the processing device may determine the at least one set of indications of effectiveness by determining a set of results associated with the set of treatment plans performed by the set of users, and a set of recovery percentages associated with the at least one set of treatment plans performed by the set of users, or both. The at least one set of indications of effectiveness may refer to a numerical representation, an alphabetical representation, an alphanumerical representation, a grade, a range of values, a description, or the like, wherein any of the foregoing quantify or qualify a level of effectiveness. For example, an indication may consist of numbers from 0 to 100 where number 0 represents completely ineffective and number 100 represents completely effective. In another example, the indications may be descriptions, such as “the treatment plan is moderately effective by resulting in 75% range of motion recovery.” In some embodiments, one or more large language models may be trained to output the set of indications of effectiveness.

In some embodiments, the processing device may, based on the set of indications of effectiveness, rank the set of treatment plans. For example, in some embodiments, the processing device may rank the set of indications by providing a highest rank to a treatment plan with a highest indication of effectiveness (e.g., complete recovery or highest percentage of recovery) and a lowest rank to a treatment plan with a lowest indication of effectiveness (e.g., no change in recovery since beginning the treatment plan or negative recovery since beginning the treatment plan). In some embodiments, based on the ranking of the set of treatment plans, the processing device may retrain the artificial intelligence engine to enable a subsequent selection of other treatment plans.

Consistent with the above disclosure, the examples of systems and methods enumerated in the following clauses are specifically contemplated and are intended as a non-limiting set of examples.
Clause 1.15 A computer-implemented method comprising:
   selecting, using an artificial intelligence engine, a plurality of treatment plans for a plurality of users, wherein the artificial intelligence engine selects the plurality of treatment plans by correlating a plurality of characteristics of the plurality of users with one or more evidence-based studies;
   based on the plurality of treatment plans, causing a plurality of electromechanical machines to be controlled while the plurality of users performs the plurality of treatment plans;
   receiving data, from a plurality of computing devices, a plurality of sensors, the plurality of electromechanical machines, or some combination thereof, wherein the data is associated with the plurality of users who use the plurality of electromechanical machines;
   determining, based on the data, a plurality of indications of effectiveness for the plurality of treatment plans.
Clause 2.15 The computer-implemented method of clause 1.15, wherein the data comprises objective data.
Clause 3.15 The computer-implemented method of clause 1.15, wherein the data comprises qualitative data.
Clause 4.15 The computer-implemented method of clause 2.15, wherein:

the objective data comprises a plurality of revolutions per minute measurements, speed measurements, range of motion measurements, measurements of pressure exerted on a plurality of pedals of the plurality of electromechanical machines, or some combination thereof.
Clause 5.15 The computer-implemented method of clause 3.15, wherein:

the qualitative data comprises at least one of a plurality of pain levels experienced by the plurality of users.
Clause 6.15 The computer-implemented method of clause 1.15, wherein the determining, based on the data, of the plurality of indications of effectiveness for the plurality of treatment plans further comprises determining a plurality of results associated with the plurality of treatment plans performed by the plurality of users, and a plurality of recovery percentages associated with the plurality of treatment plans performed by the plurality of users, or both.
Clause 7.15 The computer-implemented method of clause 1.15, further comprising, based on the plurality of indications of effectiveness, ranking the plurality of treatment plans.
Clause 8.15 The computer-implemented method of clause 5.15, further comprising, based on the ranking of the plurality of treatment plans, retraining the artificial intelligence engine to enable a subsequent selection of other treatment plans.
Clause 9.15 The computer-implemented method of clause 1.15, wherein the plurality of characteristics comprises at least one of a neurological, oncological, cardiac, orthopedic, pulmonary, or other physiological or anatomical system or condition of the plurality of users.
Clause 10.15 A tangible, non-transitory computer-readable medium storing instructions that, when executed, cause one or more processing devices to:
   select, using an artificial intelligence engine, a plurality of treatment plans for a plurality of users, wherein the artificial intelligence engine selects the plurality of treatment plans by correlating a plurality of characteristics of the plurality of users with one or more evidence-based studies;
   based on the plurality of treatment plans, cause a plurality of electromechanical machines to be controlled while the plurality of users performs the plurality of treatment plans;
   receive data, from a plurality of computing devices, a plurality of sensors, the plurality of electromechanical machines, or some combination thereof, wherein the data is associated with the plurality of users who use the plurality of electromechanical machines;
   determine, based on the data, a plurality of indications of effectiveness for the plurality of treatment plans.
Clause 11.15 The computer-readable medium of clause 10.15, wherein the data comprises objective data.
Clause 12.15 The computer-readable medium of clause 10.15, wherein the data comprises qualitative data.
Clause 13.15 The computer-readable medium of clause 11.15, wherein:

the objective data comprises a plurality of revolutions per minute measurements, speed measurements, range of motion measurements, measurements of pressure exerted on a plurality of pedals of the plurality of electromechanical machines, or some combination thereof.
Clause 14.15 The computer-readable medium of clause 12.15, wherein:
   the qualitative data comprises at least one of a plurality of pain levels experienced by the plurality of users.
Clause 15.15 The computer-readable medium of clause 10.15, wherein the determining, based on the data, of the plurality of indications of effectiveness for the plurality of treatment plans further comprises determining a plurality of results associated with the plurality of treatment plans performed by the plurality of users, and a plurality of recovery percentages associated with the plurality of treatment plans performed by the plurality of users, or both.
Clause 16.15 The computer-readable medium of clause 10.15, further comprising, based on the plurality of indications of effectiveness, ranking the plurality of treatment plans.
Clause 17.15 The computer-readable medium of clause 16.15, further comprising, based on the ranking of the plurality of treatment plans, retraining the artificial intelligence engine to enable a subsequent selection of other treatment plans.
Clause 18.15 The computer-readable medium of clause 10.15, wherein the plurality of characteristics comprises at least one of a neurological, oncological, cardiac, orthopedic, pulmonary, or other physiological or anatomical system or condition of the plurality of users.
Clause 19.15 A system comprising:
   one or more memory devices storing instructions; and
   one or more processing devices communicatively coupled to the one or more memory devices, wherein the one or more processing devices executes the instructions to:
   select, using an artificial intelligence engine, a plurality of treatment plans for a plurality of users, wherein the artificial intelligence engine selects the plurality of treatment plans by correlating a plurality of characteristics of the plurality of users with one or more evidence-based studies;
   based on the plurality of treatment plans, cause a plurality of electromechanical machines to be controlled while the plurality of users performs the plurality of treatment plans;
   receive data, from a plurality of computing devices, a plurality of sensors, the plurality of electromechanical machines, or some combination thereof, wherein the data is associated with the plurality of users who use the plurality of electromechanical machines;
   determine, based on the data, a plurality of indications of effectiveness for the plurality of treatment plans.
Clause 20.15 The system of clause 19.15, wherein the data comprises objective data and qualitative data.

### Systems and Methods for Objectively Measuring Phantom Limb Pain and Associated Body Kinesthetic Impingements in Order to Modify a Treatment Plan to Improve Body-Related Ranges of Motion and Other Performance and Flexibility Metrics

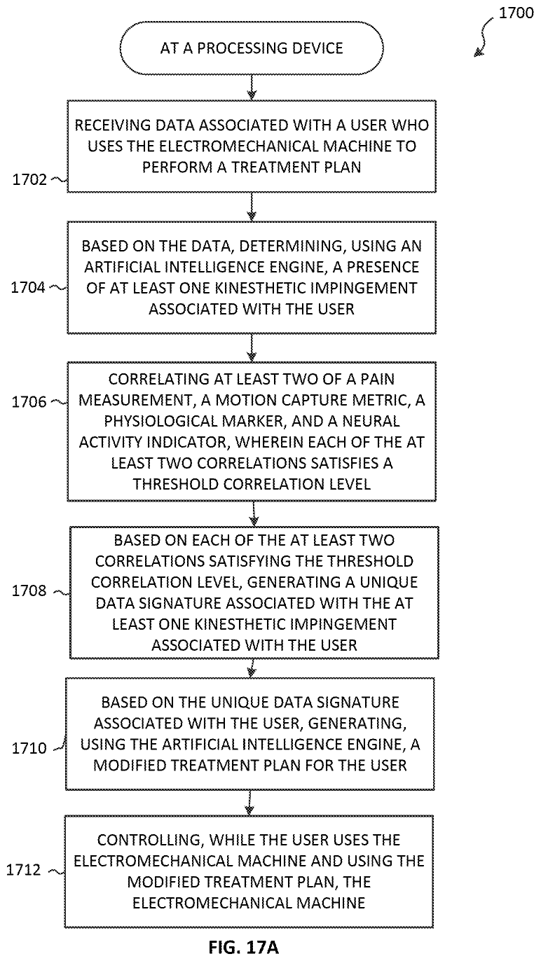

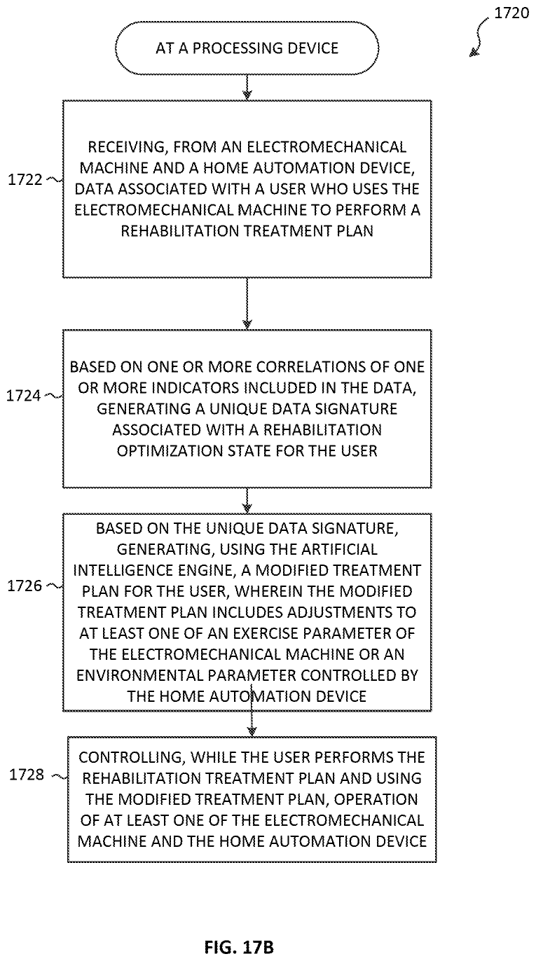

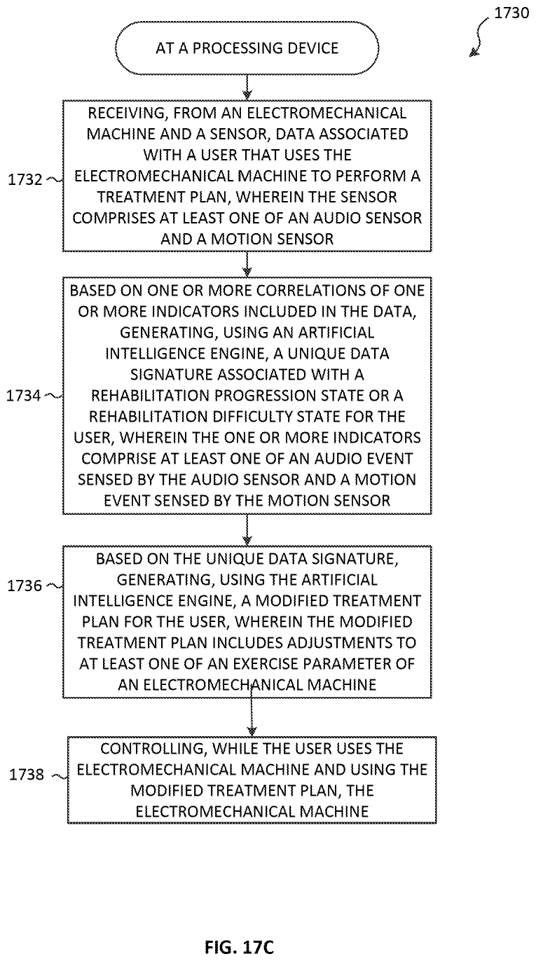

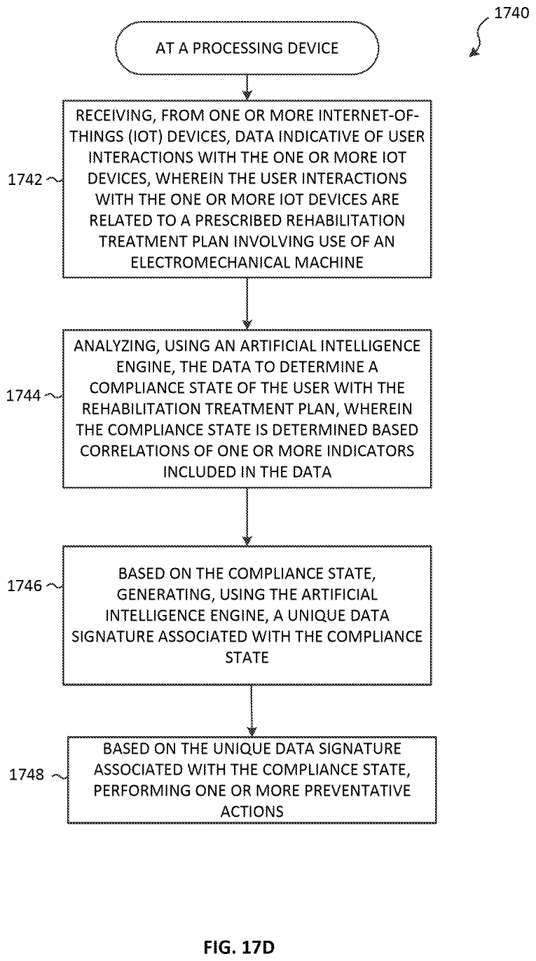

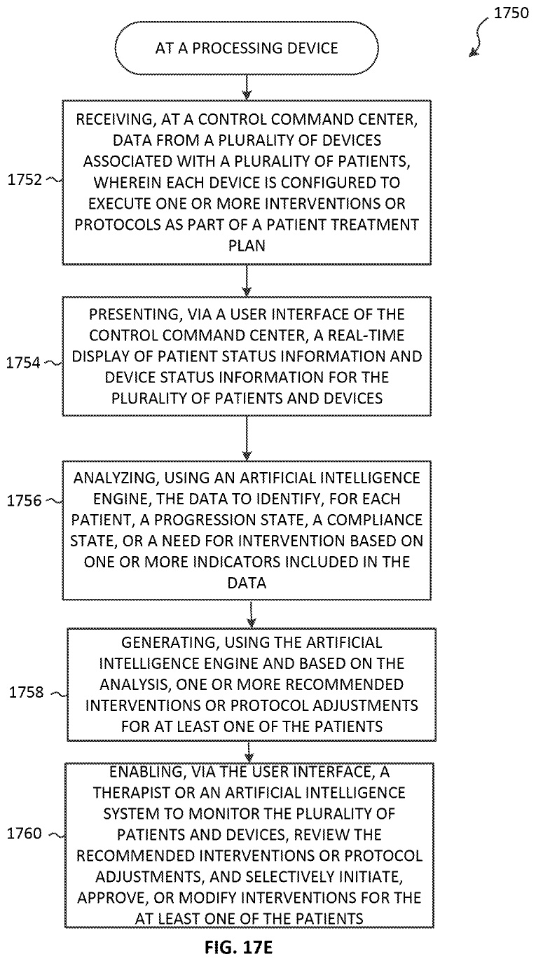

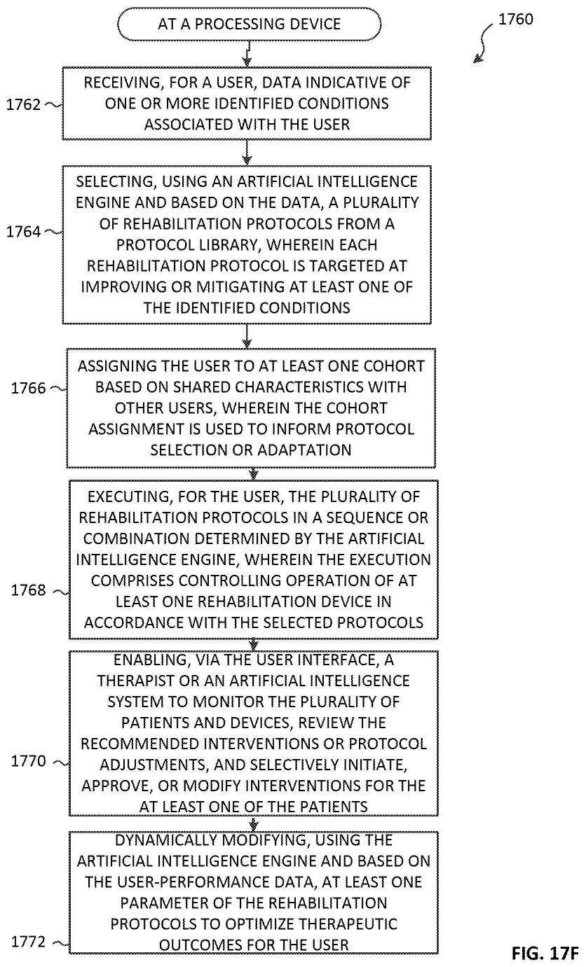

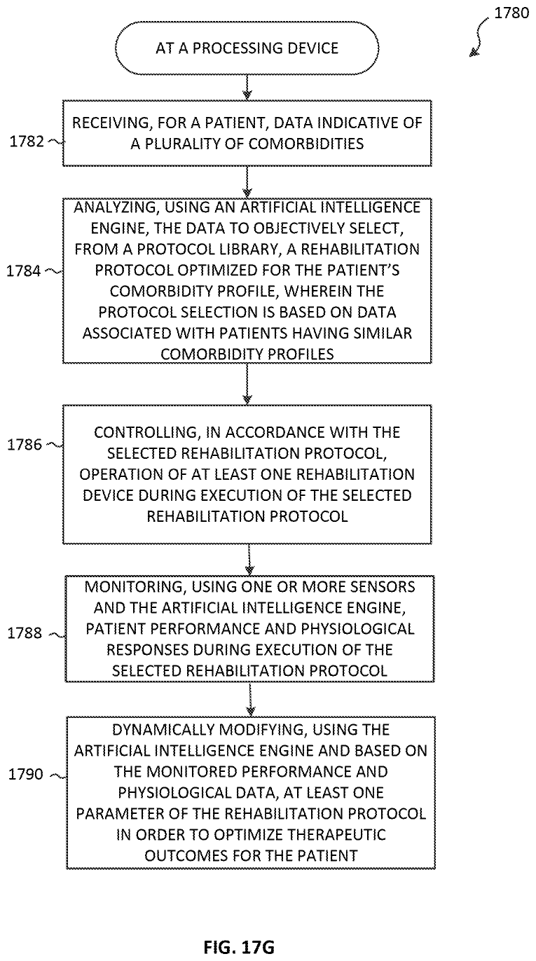

FIG. 17A illustrates an embodiment of a computer-implemented method for objectively measuring phantom limb pain and associated body kinesthetic impingements to enable dynamic modification of a rehabilitation treatment plan. In step 1702, the system receives data associated with a user who uses an electromechanical machine to perform a treatment plan. The data may be collected from a variety of sources, including wearable sensors, haptic gloves, motion capture systems, physiological monitors (such as heart rate or skin conductance sensors), and neural activity indicators (such as electroencephalogram (EEG) or Functional Near-Infrared Spectroscopy (fNIRS) devices). The data may include real-time pain measurements, motion metrics, muscle activity, and other physiological or neurological signals relevant to the user's condition.

In step 1704, the system determines, using an artificial intelligence engine, the presence of at least one kinesthetic impingement associated with the user. This determination may involve analyzing the received data to identify patterns or anomalies that indicate restricted movement, compensatory behaviors, or abnormal physiological responses. For example, the AI engine may detect a correlation between increased muscle tension and reported pain during specific movements, or identify neural activity patterns consistent with phantom limb pain episodes. In some embodiments, the system may compare real-time motion capture data to normative movement profiles or the user's historical baseline to identify reductions in range of motion, joint stiffness, or abnormal movement trajectories. For instance, if the user's knee flexion during cycling is consistently less than expected, or if the user exhibits abrupt halts or hesitations during repetitive tasks, the system may flag these as potential impingements.

The determination of kinesthetic impingement may also, by analyzing multi-sensor data streams, include detecting compensatory movement strategies, such as increased reliance on the contralateral limb, trunk leaning, or altered gait patterns. For example, if wearable sensors indicate that the user is shifting weight away from the affected limb or using excessive upper body motion to complete a lower limb task, the AI engine may interpret this as evidence of a kinesthetic impingement. Additionally, the system may integrate physiological signals such as heart rate variability, skin conductance, or electromyography (EMG) with motion data to identify episodes where increased physiological stress or muscle co-contraction coincides with specific activities or positions. For example, a spike in EMG activity in the residual limb during a particular phase of movement, coupled with a decrease in movement smoothness, may indicate the onset of an impingement.

Furthermore, the system may utilize user-reported outcomes, such as pain scores or perceived exertion, in conjunction with objective sensor data to identify activities or movement patterns that consistently provoke discomfort or difficulty. If the user reports increased pain or fatigue following certain exercises, and this aligns with observed changes in movement quality or physiological markers, the system may determine that a kinesthetic impingement is present. These approaches enable the system to provide a comprehensive and objective assessment of kinesthetic impingements, supporting more precise and individualized modifications to the user's rehabilitation treatment plan.

Step 1706 involves correlating at least two of a pain measurement, a motion capture metric, a physiological marker, and a neural activity indicator, with each correlation evaluated against a threshold correlation level. For instance, the system may correlate a spike in pain score with a decrease in range of motion or an increase in abnormal electromyography (EMG) activity, and only generate a positive finding if both exceed predetermined thresholds. This multi-modal correlation increases the objectivity and reliability of the assessment.

In step 1708, based on each of the at least two correlations satisfying the threshold correlation level, the system generates a unique data signature associated with the kinesthetic impingement. This data signature may be a composite profile that uniquely characterizes the user's pain and movement limitations, and the data signature can be used to track progression or regression over time.

Step 1710 involves generating, based on the unique data signature and using the artificial intelligence engine, a modified treatment plan for the user. The modified plan may include changes to exercise routines, adjustments to machine parameters (such as resistance or range of motion), or the introduction of new therapeutic modalities (such as VR (virtual reality)-based mirror therapy or targeted neurostimulation).

Finally, in step 1712, the system controls the electromechanical machine in real time as the user performs the treatment plan, ensuring that the machine's operation is continuously adapted to the user's current needs and limitations. This may include dynamically adjusting resistance, modifying the range of motion, and/or altering the speed and duration of exercise cycles in response to detected kinesthetic impingements or changes in pain levels. In some embodiments, the system may provide targeted haptic feedback through wearable devices, such as haptic gloves, to guide the user's movements or encourage proper form. The system may also prompt the user to rest, switch to a different activity, or perform specific therapeutic exercises if sensor data indicates excessive fatigue, abnormal physiological responses, or a risk of injury. By continuously monitoring and responding to real-time data, the system supports a highly personalized and responsive rehabilitation experience that promotes optimal recovery and minimizes discomfort.

Consistent with the above disclosure, the examples of systems and methods enumerated in the following clauses are specifically contemplated and are intended as a non-limiting set of examples.
Clause 1.18 A computer-implemented method comprising:
   receiving, from one or more of an electromechanical machine, a sensor, and a computing device, data associated with a user who uses the electromechanical machine to perform a treatment plan;
   based on the data, determining, using an artificial intelligence engine, a presence of at least one kinesthetic impingement associated with the user, wherein the determining comprises:
   correlating at least two of a pain measurement, a motion capture metric, a physiological marker, and a neural activity indicator, wherein each of the at least two correlations satisfies a threshold correlation level;
   based on each of the at least two correlations satisfying the threshold correlation level, generating a unique data signature associated with the at least one kinesthetic impingement associated with the user;
   based on the unique data signature associated with the user, generating, using the artificial intelligence engine, a modified treatment plan for the user; and
   controlling, while the user uses the electromechanical machine and using the modified treatment plan, the electromechanical machine.
Clause 2.18 The computer-implemented method of Clause 1.18, wherein the at least one kinesthetic impingement is associated with phantom limb pain experienced by the user following limb amputation.
Clause 3.18 The computer-implemented method of Clause 2.18, further comprising, based on the unique data signature associated with the at least one kinesthetic impingement, performing, using the artificial intelligence engine, one or more AI-driven interventions while the user performs the treatment plan to mitigate or ameliorate phantom limb pain and to optimize a rehabilitation progression state of the user.
Clause 4.18 The computer-implemented method of Clause 1.18, wherein the modified treatment plan comprises generating, transmitting, presenting, or controlling a virtual or augmented reality environment that simulates mirror imaging of a lost limb, such that the user perceives movement or presence of the phantom limb to reduce ghost pain associated with phantom limb syndrome.
Clause 5.18 The computer-implemented method of Clause 1.18, wherein the data comprises sensor data from a haptic glove configured to provide tactile feedback and to detect hand or residual limb movements, and wherein the modified treatment plan includes controlling the haptic glove to deliver adaptive tactile feedback during therapeutic exercises.
Clause 6.18 The computer-implemented method of Clause 1.18, wherein the data comprises sensor data from a haptic glove.
Clause 7.18 The computer-implemented method of Clause 1.18, wherein the electromechanical machine includes a stationary bike
Clause 8.18 A system comprising:
   one or more memory devices storing instructions; and
   one or more processing devices communicatively coupled to the one or more memory devices, wherein the one or more processing devices execute the instructions to:
   receive, from one or more of an electromechanical machine, a sensor, and a computing device, data associated with a user who uses the electromechanical machine to perform a treatment plan;
   based on the data, determine, using an artificial intelligence engine, a presence of at least one kinesthetic impingement associated with the user, wherein the determining comprises:
   correlating at least two of a pain measurement, a motion capture metric, a physiological marker, and a neural activity indicator, wherein each of the at least two correlations satisfies a threshold correlation level;
   based on each of the at least two correlations satisfying the threshold correlation level, generate a unique data signature associated with the at least one kinesthetic impingement associated with the user;
   based on the unique data signature associated with the user, generate, using the artificial intelligence engine, a modified treatment plan for the user; and
   control, while the user uses the electromechanical machine and using the modified treatment plan, the electromechanical machine.
Clause 9.18 The system of Clause 8.18, wherein the at least one kinesthetic impingement is associated with phantom limb pain experienced by the user following limb amputation.
Clause 10.18 The system of Clause 9.18, wherein the one or more processing devices are further configured to, based on the unique data signature associated with the at least one kinesthetic impingement, perform, using the artificial intelligence engine, one or more AI-driven interventions while the user performs the treatment plan to mitigate or ameliorate phantom limb pain and to optimize a rehabilitation progression state of the user.
Clause 11.18 The system of Clause 8.18, wherein the modified treatment plan comprises generating, transmitting, presenting, or controlling a virtual or augmented reality environment that simulates mirror imaging of a lost limb, such that the user perceives movement or presence of the phantom limb to reduce ghost pain associated with phantom limb syndrome.
Clause 12.18 The system of Clause 8.18, wherein the data comprises sensor data from a haptic glove configured to provide tactile feedback and to detect hand or residual limb movements, and wherein the modified treatment plan includes controlling the haptic glove to deliver adaptive tactile feedback during therapeutic exercises.
Clause 13.18 The system of Clause 8.18, wherein the data comprises sensor data from a haptic glove.
Clause 14.18 The system of Clause 8.18, wherein the electromechanical machine includes a stationary bike.
Clause 15.18 A non-transitory computer-readable medium storing instructions that, when executed by one or more processing devices, cause the one or more processing devices to:
   receive, from one or more of an electromechanical machine, a sensor, and a computing device, data associated with a user who uses the electromechanical machine to perform a treatment plan;
   based on the data, determine, using an artificial intelligence engine, a presence of at least one kinesthetic impingement associated with the user, wherein the determining comprises:
   correlating at least two of a pain measurement, a motion capture metric, a physiological marker, and a neural activity indicator, wherein each of the at least two correlations satisfies a threshold correlation level;
   based on each of the at least two correlations satisfying the threshold correlation level, generate a unique data signature associated with the at least one kinesthetic impingement associated with the user;
   based on the unique data signature associated with the user, generate, using the artificial intelligence engine, a modified treatment plan for the user; and
   control, while the user uses the electromechanical machine and using the modified treatment plan, the electromechanical machine.
Clause 16.18 The non-transitory computer-readable medium of Clause 15.18, wherein the at least one kinesthetic impingement is associated with phantom limb pain experienced by the user following limb amputation.
Clause 17.18 The non-transitory computer-readable medium of Clause 16.18, further comprising instructions that, when executed, cause the one or more processing devices to, based on the unique data signature associated with the at least one kinesthetic impingement, perform, using the artificial intelligence engine, one or more AI-driven interventions while the user performs the treatment plan to mitigate or ameliorate phantom limb pain and to optimize a rehabilitation progression state of the user.
Clause 18.18 The non-transitory computer-readable medium of Clause 15.18, wherein the modified treatment plan comprises instructions that, when executed, cause the one or more processing devices to generate, transmit, present, or control a virtual or augmented reality environment that simulates mirror imaging of a lost limb, such that the user perceives movement or presence of the phantom limb to reduce ghost pain associated with phantom limb syndrome.
Clause 19.18 The non-transitory computer-readable medium of Clause 15.18, wherein the data comprises sensor data from a haptic glove configured to provide tactile feedback and to detect hand or residual limb movements, and wherein the modified treatment plan includes instructions that, when executed, cause the one or more processing devices to control the haptic glove to deliver adaptive tactile feedback during therapeutic exercises.
Clause 20.18 The non-transitory computer-readable medium of Clause 15.18, wherein the data comprises sensor data from a haptic glove.
   Systems and Methods to Use Home Automation Devices to Control or Improve a Rehabilitation Treatment Plan which Uses an Electromechanical Machine

FIG. 17B depicts a method for integrating home automation devices with rehabilitation (which, herein, shall be deemed to also include prehabilitation) treatment plans to enhance user outcomes and experience. In step 1722, the system receives data from both an electromechanical machine and a home automation device while the user performs a rehabilitation treatment plan. The home automation device may include smart speakers, lighting systems, thermostats, or other IoT (Internet of Things) devices capable of monitoring or influencing the user's environment. For example, a smart speaker may be used to deliver audio prompts, reminders, or therapeutic instructions to the user, or to receive spoken feedback and pain reports from the user during rehabilitation sessions. Smart lighting systems can be automatically adjusted to create an optimal environment for exercise, such as dimming lights to promote relaxation or increasing brightness to enhance alertness and motivation. Thermostats may be controlled to maintain a comfortable room temperature that supports physical activity and recovery. Additional IoT devices, such as smart blinds, air purifiers, or environmental sensors, may also be integrated to further tailor the rehabilitation environment to the user's needs. These devices can work in concert to reduce distractions, improve comfort, and encourage adherence to the prescribed treatment plan, thereby enhancing the overall effectiveness of the rehabilitation process. The data may include environmental parameters (such as room temperature or lighting), user activity metrics (such as movement detected by smart sensors), and self-reported or passively collected pain or comfort levels.

In step 1724, the system generates a unique data signature associated with a rehabilitation optimization state for the user, based on one or more correlations of indicators included in the data. For example, the system may correlate increased pain reports with high (or low) room temperature or low (or high) lighting, or identify that the user is more compliant with the treatment plan when audio prompts are delivered at certain times of the day.

Step 1726 involves generating a modified treatment plan for the user using the artificial intelligence engine, where the plan includes adjustments to at least one of an exercise parameter of the electromechanical machine or an environmental parameter controlled by the home automation device. Embodiments may include automatically lowering the resistance of a stationary bike if the user's sleep quality (as detected by a smart bed) was poor, or dimming the lights and playing calming music during exercise sessions to reduce anxiety and pain. In other embodiments, the system may increase the frequency of audio reminders or motivational prompts delivered through a smart speaker if the user's activity levels, as detected by motion sensors, fall below a target threshold. The modified treatment plan may also include adjusting the room temperature via a smart thermostat to optimize comfort and reduce muscle stiffness during rehabilitation, or automatically closing smart blinds to minimize glare and distractions during exercise. In some cases, the system may schedule rehabilitation sessions at times when the user is most active or alert, as determined by analyzing patterns from occupancy sensors or wearable devices. Additionally, the system may temporarily disable notifications (e.g., push notifications) on the user's mobile devices or entertainment systems to minimize interruptions and promote focus during critical rehabilitation activities. The notifications may pertain to any software applications installed on the mobile devices or entertainment systems (e.g., updates, alerts, text messages, emails, etc.) and temporarily disabling the notifications may cause them to not be displayed on a user interface, to not cause audio to be emitted, and/or haptic feedback to be produced. By integrating and coordinating these various environmental and exercise-related adjustments, the modified treatment plan can be highly personalized and responsive to the user's real-time needs and preferences, thereby supporting improved adherence, comfort, and rehabilitation outcomes.

In step 1728, the system controls, in real time, the operation of at least one of the electromechanical machine and the home automation device while the user performs the rehabilitation treatment plan. This may include sending commands to the machine to adjust exercise parameters, or to the home automation system to optimize environmental conditions for rehabilitation. In additional embodiments, the system may automatically pause, resume or restart the electromechanical machine based on detected user fatigue or pain levels, ensuring that the user does not overexert themself. The system may also initiate a pre-programmed warm-up or cool-down sequence on the electromechanical machine in response to environmental cues, such as changes in room temperature or lighting, to further support safe, successful and effective rehabilitation sessions.

Consistent with the above disclosure, the examples of systems and methods enumerated in the following clauses are specifically contemplated and are intended as a non-limiting set of examples.
Clause 1.19 A computer-implemented method comprising:
   receiving, from an electromechanical machine and a home automation device, data associated with a user who uses the electromechanical machine to perform a rehabilitation treatment plan;
   based on one or more correlations of one or more indicators included in the data, generating a unique data signature associated with a rehabilitation optimization state for the user;
   based on the unique data signature, generating, using the artificial intelligence engine, a modified treatment plan for the user, wherein the modified treatment plan includes adjustments to at least one of an exercise parameter of the electromechanical machine or an environmental parameter controlled by the home automation device; and
   controlling, while the user performs the rehabilitation treatment plan and using the modified treatment plan, operation of at least one of the electromechanical machine and the home automation device.
Clause 2.19 The computer-implemented method of Clause 1.19, wherein the one or more indicators comprise at least one of a pain measurement, a user activity metric obtained from the home automation device, a compliance indicator, and an environmental parameter.
Clause 3.19 The computer-implemented method of Clause 1.19, wherein the controlling includes controlling operation of the home automation device.
Clause 4.19 The computer-implemented method of Clause 3.19 wherein the home automation device comprises a smart speaker, and the controlling includes causing the smart speaker to deliver audio prompts, reminders, or therapeutic instructions to the user in accordance with the modified treatment plan.
Clause 5.19 The computer-implemented method of Clause 3.19, wherein the home automation device comprises an audio input configured to receive spoken instructions or commands, and the controlling includes causing a natural language processor to analyze the instructions or commands from the user when the user is performing the modified treatment plan.
Clause 6.19 The computer-implemented method of Clause 3.19, wherein the home automation device comprises a home lighting system, and the controlling includes automatically adjusting a lighting parameter of the home lighting system during a rehabilitation session based on the modified treatment plan.
Clause 7.19 The computer-implemented method of Clause 1.19, further comprising:
   receiving, from the user and via the home automation device, an indicator associated with user pain; and
   generating, using the artificial intelligence engine, the modified treatment plan based on the indicator associated with user pain.
Clause 8.19 The computer-implemented method of Clause 7.19, wherein the indicator of user pain is at least one of a qualitative indicator provided by the user and a proxy indicator based on a measurement made by the electromechanical device.
Clause 9.19 The computer-implemented method of Clause 1.19, wherein the home automation device comprises a smart thermostat, and the controlling includes adjusting a temperature setting to optimize user comfort or recovery conditions during the rehabilitation session based on the modified treatment plan.
Clause 10.19 The computer-implemented method of Clause 1.19, wherein the controlling includes turning off or deactivating one or more notifications of a mobile device of the user while the user performs the rehabilitation treatment plan.
Clause 11.19 The computer-implemented method of Clause 1.19, wherein the modified treatment plan includes scheduling a rehabilitation session at a time based on user activity patterns detected by the home automation device.
Clause 12.19 The computer-implemented method of Clause 1.19, further comprising:

initializing a rehabilitation session based on a user instruction received via the home automation device.
Clause 13.19 A system comprising:
   one or more memory devices storing instructions; and
   one or more processing devices communicatively coupled to the one or more memory devices, wherein the one or more processing devices execute the instructions to:
   receive, from an electromechanical machine and a home automation device, data associated with a user who uses the electromechanical machine to perform a rehabilitation treatment plan;
   based on one or more correlations of one or more indicators included in the data, generate a unique data signature associated with a rehabilitation optimization state for the user;
   based on the unique data signature, generate, using an artificial intelligence engine, a modified treatment plan for the user, wherein the modified treatment plan includes adjustments to at least one of an exercise parameter of the electromechanical machine or an environmental parameter controlled by the home automation device; and
   control, while the user performs the rehabilitation treatment plan and using the modified treatment plan, operation of at least one of the electromechanical machine and the home automation device.
Clause 14.19 The system of Clause 13.19, wherein the one or more indicators comprise at least one of a pain measurement, a user activity metric obtained from the home automation device, a compliance indicator, and an environmental parameter.
Clause 15.19 The system of Clause 13.19, wherein the one or more processing devices are further configured to control operation of the home automation device.
Clause 16.19 The system of Clause 15.19, wherein the home automation device comprises a smart speaker, and the one or more processing devices are configured to cause the smart speaker to deliver audio prompts, reminders, or therapeutic instructions to the user in accordance with the modified treatment plan.
Clause 17.19 The system of Clause 15.19, wherein the home automation device comprises an audio input configured to receive spoken instructions or commands, and the one or more processing devices are configured to cause a natural language processor to analyze the instructions or commands from the user when the user is performing the modified treatment plan.
Clause 18.19 The system of Clause 15.19, wherein the home automation device comprises a home lighting system, and the one or more processing devices are configured to automatically adjust a lighting parameter of the home lighting system during a rehabilitation session based on the modified treatment plan.
Clause 19.19 The system of Clause 13.19, wherein the one or more processing devices are further configured to:
   receive, from the user and via the home automation device, an indicator associated with user pain; and
   generate, using the artificial intelligence engine, the modified treatment plan based on the indicator associated with user pain.
Clause 20.19 The system of Clause 19.19, wherein the indicator of user pain is at least one of a qualitative indicator provided by the user and a proxy indicator based on a measurement made by the electromechanical device.
Clause 21.19 The system of Clause 13.19, wherein the home automation device comprises a smart thermostat, and the one or more processing devices are configured to adjust a temperature setting to optimize user comfort or recovery conditions during the rehabilitation session based on the modified treatment plan.
Clause 22.19 The system of Clause 13.19, wherein the one or more processing devices are configured to turn off or deactivate one or more notifications of a mobile device of the user while the user performs the rehabilitation treatment plan.
Clause 23.19 The system of Clause 13.19, wherein the modified treatment plan includes scheduling a rehabilitation session at a time based on user activity patterns detected by the home automation device.
Clause 24.19 The system of Clause 13.19, wherein the one or more processing devices are further configured to initialize a rehabilitation session based on a user instruction received via the home automation device.
Clause 25.19 A non-transitory computer-readable medium storing instructions that, when executed by one or more processing devices, cause the one or more processing devices to:
   receive, from an electromechanical machine and a home automation device, data associated with a user who uses the electromechanical machine to perform a rehabilitation treatment plan;
   based on one or more correlations of one or more indicators included in the data, generate a unique data signature associated with a rehabilitation optimization state for the user;
   based on the unique data signature, generate, using an artificial intelligence engine, a modified treatment plan for the user, wherein the modified treatment plan includes adjustments to at least one of an exercise parameter of the electromechanical machine or an environmental parameter controlled by the home automation device; and
   control, while the user performs the rehabilitation treatment plan and using the modified treatment plan, operation of at least one of the electromechanical machine and the home automation device.
Clause 26.19 The non-transitory computer-readable medium of Clause 25.19, wherein the one or more indicators comprise at least one of a pain measurement, a user activity metric obtained from the home automation device, a compliance indicator, and an environmental parameter.
Clause 27.19 The non-transitory computer-readable medium of Clause 25.19, wherein the instructions, when executed, further cause the one or more processing devices to control operation of the home automation device.
Clause 28.19 The non-transitory computer-readable medium of Clause 27.19, wherein the home automation device comprises a smart speaker, and the instructions, when executed, cause the one or more processing devices to cause the smart speaker to deliver audio prompts, reminders, or therapeutic instructions to the user in accordance with the modified treatment plan.
Clause 29.19 The non-transitory computer-readable medium of Clause 27.19, wherein the home automation device comprises an audio input configured to receive spoken instructions or commands, and the instructions, when executed, cause the one or more processing devices to cause a natural language processor to analyze the instructions or commands from the user when the user is performing the modified treatment plan.
Clause 30.19 The non-transitory computer-readable medium of Clause 27.19, wherein the home automation device comprises a home lighting system, and the instructions, when executed, cause the one or more processing devices to automatically adjust a lighting parameter of the home lighting system during a rehabilitation session based on the modified treatment plan.
Clause 31.19 The non-transitory computer-readable medium of Clause 25.19, wherein the instructions, when executed, further cause the one or more processing devices to:
   receive, from the user and via the home automation device, an indicator associated with user pain; and
   generate, using the artificial intelligence engine, the modified treatment plan based on the indicator associated with user pain.
Clause 32.19 The non-transitory computer-readable medium of Clause 31.19, wherein the indicator of user pain is at least one of a qualitative indicator provided by the user and a proxy indicator based on a measurement made by the electromechanical device.
Clause 33.19 The non-transitory computer-readable medium of Clause 25.19, wherein the home automation device comprises a smart thermostat, and the instructions, when executed, cause the one or more processing devices to adjust a temperature setting to optimize user comfort or recovery conditions during the rehabilitation session based on the modified treatment plan.
Clause 34.19 The non-transitory computer-readable medium of Clause 25.19, wherein the instructions, when executed, cause the one or more processing devices to turn off or deactivate one or more notifications of a mobile device of the user while the user performs the rehabilitation treatment plan.
Clause 35.19 The non-transitory computer-readable medium of Clause 25.19, wherein the modified treatment plan includes scheduling a rehabilitation session at a time based on user activity patterns detected by the home automation device.
Clause 36.19 The non-transitory computer-readable medium of Clause 25.19, wherein the instructions, when executed, cause the one or more processing devices to initialize a rehabilitation session based on a user instruction received via the home automation device.
   Systems and Methods to Identify User Motion and Background Audio Associated with the User in Order to Objectively Measure the Progress of a User's Rehabilitation and Identify Factors Mitigating the Progress, for the Purpose of Modifying the User's Treatment Plan

FIG. 17C illustrates a method for using motion and audio sensing to objectively measure rehabilitation progress and identify factors that may hinder recovery. In step 1732, the system receives data from an electromechanical machine and a sensor, where the sensor comprises at least one of an audio sensor and a motion sensor. The audio sensor may be a smart speaker or dedicated microphone, while the motion sensor may be a wearable accelerometer, gyroscope, or environmental motion detector. The data may include audio events (such as groans, sighs, or verbal pain reports) and motion events (such as walking, standing, or transitions between postures).

In step 1734, the system generates, using an artificial intelligence engine, a unique data signature associated with a rehabilitation progression state or a rehabilitation difficulty state for the user, based on one or more correlations of indicators in the data. For example, the system may detect that the user hesitates or vocalizes pain when climbing stairs, or that their gait speed has decreased over time, indicating a regression in mobility. In further embodiments, the system may identify patterns such as increased frequencies of pauses or changes in cadence during walking, which may signal growing fatigue or discomfort. The data signature may also reflect the presence of abnormal movement patterns, such as limping, shuffling, or compensatory use of the upper body, as detected by motion sensors or wearable devices. Additionally, the system may correlate background audio events-such as sighs, groans, or requests for assistance—with specific activities or times of day, providing a more nuanced understanding of when, how and why rehabilitation difficulties arise. In some cases, the data signature may incorporate physiological signals, such as elevated heart rate or skin conductance, that coincide with challenging activities, further refining the assessment of the user's rehabilitation state.

Step 1736 involves generating a modified treatment plan for the user, where the plan includes adjustments to at least one exercise parameter of the electromechanical machine. The system may recommend more frequent rest breaks, lower resistance, or alternative exercises if increased pain or difficulty is detected during certain activities. In additional embodiments, the modified treatment plan may include gradually increasing the duration or intensity of exercises as the user demonstrates improved performance, or introducing new types of movements to address specific deficits identified through motion or audio analysis. The system may also suggest changes in exercise scheduling, such as shifting sessions to times of day when the user is most active or alert, or incorporating adaptive feedback mechanisms-such as real-time audio cues or visual prompts—to encourage proper technique and reduce compensatory behaviors. In some cases, the system may coordinate with caregivers or clinicians to recommend supervised sessions or telehealth check-ins if persistent difficulties or safety concerns are detected.

In step 1738, the system controls the electromechanical machine in accordance with the modified treatment plan, ensuring that the user's rehabilitation is continuously adapted to their real-world performance and comfort.

Consistent with the above disclosure, the examples of systems and methods enumerated in the following clauses are specifically contemplated and are intended as a non-limiting set of examples.
Clause 1.20 A computer-implemented method comprising:
   receiving, from an electromechanical machine and a sensor, data associated with a user that uses the electromechanical machine to perform a treatment plan, wherein the sensor comprises at least one of an audio sensor and a motion sensor;
   based on one or more correlations of one or more indicators included in the data, generating, using an artificial intelligence engine, a unique data signature associated with a rehabilitation progression state or a rehabilitation difficulty state for the user, wherein the one or more indicators comprise at least one of an audio event sensed by the audio sensor and a motion event sensed by the motion sensor;
   based on the unique data signature, generating, using the artificial intelligence engine, a modified treatment plan for the user, wherein the modified treatment plan includes adjustments to at least one of an exercise parameter of an electromechanical machine; and
   controlling, while the user uses the electromechanical machine and using the modified treatment plan, the electromechanical machine.
Clause 2.20 The computer-implemented method of Clause 1.20, wherein the sensor comprises the audio sensor, and the one or more indicators comprise at least one of a pain-related vocalization and a verbal report of pain or discomfort associated with the user.
Clause 3.20 The computer-implemented method of Clause 1.20, wherein the one or more indicators comprise at least one of a qualitative indicator provided by the user and a proxy indicator based on a measurement made by the electromechanical device.
Clause 4.20 The computer-implemented method of Clause 1.20, wherein the sensor comprises the audio sensor, and the one or more indicators comprise at least one of a change in speech cadence, an increase in vocal pitch or volume, a pattern of interrupted speech, a request for assistance.
Clause 5.20 The computer-implemented method of Clause 1.20, wherein the sensor comprises the audio sensor, and the one or more indicators comprise background sounds indicative of exertion or distress
Clause 6.20 The computer-implemented method of Clause 1.20, wherein the sensor comprises the motion sensor, and the one or more indicators comprise at least one of a movement pattern, a transition between postures, and a measure of activity difficulty.
Clause 7.20 The computer-implemented method of Clause 1.20, wherein the sensor comprises the motion sensor, and wherein the one or more indicators comprise at least one of a duration required to complete a movement, a number of pauses or hesitations during the activity, a statistically-significant deviation from a normative movement pattern, and a comparison of the user's current activity performance to a historical baseline for the user.
Clause 8.20 The computer-implemented method of Clause 1.20, wherein the modified treatment plan further comprises scheduling a rest period or recommending a change in activity based on detected increases in user exertion or pain-related audio events.
Clause 9.20 The computer-implemented method of Clause 1.20, wherein the sensor comprises both the motion sensor and the audio sensor, and wherein the artificial intelligence engine generates the unique data signature by using data from both the motion sensor and the audio sensor.
Clause 10.20 A system comprising:
   one or more memory devices storing instructions; and
   one or more processing devices communicatively coupled to the one or more memory devices, wherein the one or more processing devices execute the instructions to:
   receive, from an electromechanical machine and a sensor, data associated with a user that uses the electromechanical machine to perform a treatment plan, wherein the sensor comprises at least one of an audio sensor and a motion sensor;
   based on one or more correlations of one or more indicators included in the data, generate, using an artificial intelligence engine, a unique data signature associated with a rehabilitation progression state or a rehabilitation difficulty state for the user, wherein the one or more indicators comprise at least one of an audio event sensed by the audio sensor and a motion event sensed by the motion sensor;
   based on the unique data signature, generate, using the artificial intelligence engine, a modified treatment plan for the user, wherein the modified treatment plan includes adjustments to at least one of an exercise parameter of an electromechanical machine; and
   control, while the user uses the electromechanical machine and using the modified treatment plan, the electromechanical machine.
Clause 11.20 The system of Clause 10.20, wherein the sensor comprises the audio sensor, and the one or more indicators comprise at least one of a pain-related vocalization and a verbal report of pain or discomfort associated with the user.
Clause 12.20 The system of Clause 10.20, wherein the one or more indicators comprise at least one of a qualitative indicator provided by the user and a proxy indicator based on a measurement made by the electromechanical device.
Clause 13.20 The system of Clause 10.20, wherein the sensor comprises the audio sensor, and the one or more indicators comprise at least one of a change in speech cadence, an increase in vocal pitch or volume, a pattern of interrupted speech, or a request for assistance.
Clause 14.20 The system of Clause 10.20, wherein the sensor comprises the audio sensor, and the one or more indicators comprise background sounds indicative of exertion or distress.
Clause 15.20 The system of Clause 10.20, wherein the sensor comprises the motion sensor, and the one or more indicators comprise at least one of a movement pattern, a transition between postures, and a measure of activity difficulty.
Clause 16.20 The system of Clause 10.20, wherein the sensor comprises the motion sensor, and wherein the one or more indicators comprise at least one of a duration required to complete a movement, a number of pauses or hesitations during the activity, a statistically-significant deviation from a normative movement pattern, and a comparison of the user's current activity performance to a historical baseline for the user.
Clause 17.20 The system of Clause 10.20, wherein the modified treatment plan further comprises scheduling a rest period or recommending a change in activity based on detected increases in user exertion or pain-related audio events.
Clause 18.20 The system of Clause 10.20, wherein the sensor comprises both the motion sensor and the audio sensor, and wherein the artificial intelligence engine generates the unique data signature by using data from both the motion sensor and the audio sensor.
Clause 19.20 A non-transitory computer-readable medium storing instructions that, when executed by one or more processing devices, cause the one or more processing devices to:
   receive, from an electromechanical machine and a sensor, data associated with a user that uses the electromechanical machine to perform a treatment plan, wherein the sensor comprises at least one of an audio sensor and a motion sensor;
   based on one or more correlations of one or more indicators included in the data, generate, using an artificial intelligence engine, a unique data signature associated with a rehabilitation progression state or a rehabilitation difficulty state for the user, wherein the one or more indicators comprise at least one of an audio event sensed by the audio sensor and a motion event sensed by the motion sensor;
   based on the unique data signature, generate, using the artificial intelligence engine, a modified treatment plan for the user, wherein the modified treatment plan includes adjustments to at least one of an exercise parameter of an electromechanical machine; and
   control, while the user uses the electromechanical machine and using the modified treatment plan, the electromechanical machine.
Clause 20.20 The non-transitory computer-readable medium of Clause 19.20, wherein the sensor comprises the audio sensor, and the one or more indicators comprise at least one of a pain-related vocalization and a verbal report of pain or discomfort associated with the user.
Clause 21.20 The non-transitory computer-readable medium of Clause 19.20, wherein the one or more indicators comprise at least one of a qualitative indicator provided by the user and a proxy indicator based on a measurement made by the electromechanical device.
Clause 22.20 The non-transitory computer-readable medium of Clause 19.20, wherein the sensor comprises the audio sensor, and the one or more indicators comprise at least one of a change in speech cadence, an increase in vocal pitch or volume, a pattern of interrupted speech, or a request for assistance.
Clause 23.20 The non-transitory computer-readable medium of Clause 19.20, wherein the sensor comprises the audio sensor, and the one or more indicators comprise background sounds indicative of exertion or distress.
Clause 24.20 The non-transitory computer-readable medium of Clause 19.20, wherein the sensor comprises the motion sensor, and the one or more indicators comprise at least one of a movement pattern, a transition between postures, and a measure of activity difficulty.
Clause 25.20 The non-transitory computer-readable medium of Clause 19.20, wherein the sensor comprises the motion sensor, and wherein the one or more indicators comprise at least one of a duration required to complete a movement, a number of pauses or hesitations during the activity, a statistically-significant deviation from a normative movement pattern, and a comparison of the user's current activity performance to a historical baseline for the user.
Clause 26.20 The non-transitory computer-readable medium of Clause 19.20, wherein the modified treatment plan further comprises scheduling a rest period or recommending a change in activity based on detected increases in user exertion or pain-related audio events.
Clause 27.20 The non-transitory computer-readable medium of Clause 19.20, wherein the sensor comprises both the motion sensor and the audio sensor, and wherein the artificial intelligence engine generates the unique data signature by using data from both the motion sensor and the audio sensor.
   Systems and Methods to Monitor Use of One or More Smart Devices to Objectively Measure Treatment Plan Compliance for the Purpose of Modifying a Treatment Plan that Uses an Electromechanical Machine

FIG. 17D presents a method for leveraging IoT (Internet of Things) devices to monitor compliance with a rehabilitation treatment plan and to trigger preventative actions when non-compliance is detected. In step 1742, the system receives data from one or more IoT devices, such as smart kitchen appliances, medication dispensers, thermostats, televisions, motion sensors, or wearable fitness trackers. The data is indicative of user interactions relevant to the prescribed rehabilitation plan, such as food preparation, medication intake, activity level, or screen time.

In step 1744, the system analyzes the data using an artificial intelligence engine to determine a compliance state of the user with the rehabilitation treatment plan. This may involve correlating multiple indicators, such as whether the user opened the refrigerator to retrieve prescribed foods, took medications on schedule, or exceeded recommended screen time limits. In further embodiments, the system may assess compliance by monitoring activity levels through wearable fitness trackers or motion sensors to verify that the user is meeting prescribed physical activity goals. The system may also evaluate environmental conditions, such as room temperature or lighting, to ensure that the user is performing exercises in an optimal environment as recommended by the treatment plan. Additionally, the system can analyze data from smart medication dispensers to confirm that medications have been, are or will be taken at the correct times and dosages, or use smart kitchen appliances to track whether the user is preparing and consuming meals that align with dietary restrictions or nutritional guidelines. In some cases, the system may integrate data from multiple IoT devices to detect patterns of non-compliance, such as frequent late-night television usage combined with reduced morning activity, which may indicate poor sleep hygiene that affects rehabilitation progress.

Step 1746 involves generating a unique data signature associated with the compliance state, which may be used to track adherence trends or to identify specific areas of non-compliance. The data signature may encapsulate patterns such as missed medication doses, irregular exercise times, or deviations (statistical, ad hoc, or incidental or anecdotal) from recommended dietary or activity guidelines. In some embodiments, the data signature may also include temporal information, allowing the system to detect recurring issues at certain times of day or days of the week. Additionally, the data signature can be compared against historical compliance data for the same user or benchmarked against population-level data to identify emerging risks or persistent barriers to adherence. This comprehensive signature enables the system to provide targeted feedback, generate personalized compliance reports, and support proactive intervention strategies to improve overall rehabilitation outcomes.

In step 1748, the system performs one or more preventative actions based on the unique data signature. Preventative actions may include sending alerts to the user or caregiver, modifying the rehabilitation plan, adjusting machine parameters, or scheduling follow-up interventions to address compliance issues. Additional preventative actions may include increasing the frequency or specificity of reminders delivered through smart speakers or mobile devices, temporarily disabling or limiting access to entertainment devices such as smart TVs during prescribed exercise periods, providing educational content or motivational messages tailored to the user's specific compliance challenges, notifying a designated emergency contact if critical non-compliance is detected (such as missed medication doses or prolonged inactivity), logging compliance events and preventative actions for future review and quality assurance, and recommending environmental adjustments-such as changing room temperature, lighting, or noise levels—to support better adherence to the rehabilitation protocol. In some embodiments, the system may also prompt the user to complete a self-assessment or pain survey, or automatically initiate a telehealth session with a clinician if persistent or severe non-compliance is identified.

Consistent with the above disclosure, the examples of systems and methods enumerated in the following clauses are specifically contemplated and are intended as a non-limiting set of examples.
Clause 1.21 A computer-implemented method comprising:
   receiving, from one or more internet-of-things (IOT) devices, data indicative of user interactions with the one or more IoT devices, wherein the user interactions with the one or more IoT devices are related to a prescribed rehabilitation treatment plan involving use of an electromechanical machine;
   analyzing, using an artificial intelligence engine, the data to determine a compliance state of the user with the rehabilitation treatment plan, wherein the compliance state is determined based correlations of one or more indicators included in the data;
   based on the compliance state, generating, using the artificial intelligence engine, a unique data signature associated with the compliance state; and
   based on the unique data signature associated with the compliance state, performing one or more preventative actions.
Clause 2.21 The computer-implemented method of Clause 1.21, wherein the indicators comprise at least one of dietary compliance, medication adherence, activity level, screen time, and environmental condition.
Clause 3.21 The computer-implemented method of Clause 1.21, wherein the one or more preventative actions comprise at least one of modifying a parameter of the electromechanical machine and modifying the rehabilitation treatment plan.
Clause 4.21 The computer-implemented method of Clause 1.21, wherein the one or more IoT devices comprise at least one of a smart kitchen appliance, a smart medication dispenser, a smart thermostat, a smart television, a motion sensor, and a wearable fitness tracker.
Clause 5.21 The computer-implemented method of Clause 4.21, wherein the IOT device is a smart kitchen appliance.
Clause 6.21 The computer-implemented method of clause 5.21, wherein the smart kitchen appliance is a smart refrigerator.
Clause 7.21 The computer-implemented method of Clause 6.21, wherein the data is generated via sensors indicating one or more of opening of a door of the smart refrigerator and an amount of food or one or more characteristics pertaining to food removed from the smart refrigerator.
Clause 8.21 The computer-implemented method of Clause 1.21, wherein the one or more IoT devices comprise a sensor configured to monitor handle pressure applied by the user to the one or more IoT devices, and wherein the data indicative of user interactions includes measurements of handle pressure.
Clause 9.21 The computer-implemented method of Clause 1.21, wherein the one or more IoT devices comprise a sensor configured to monitor user gait, and wherein the data indicative of user interactions includes gait analysis measurements to assess the compliance state.
Clause 10.21 The computer-implemented method of Clause 1.21, wherein the one or more IoT devices comprise one or more of an oven and a stove, wherein the data indicative of user interactions includes data indicative of use of the oven or stove.
Clause 11.21 The computer-implemented method of Clause 1.21, wherein the one or more IoT devices comprise a display device including one or more of a television, cell phone, tablet, and wearable device, wherein the data indicative of user interactions includes information related to screen time usage to assess the compliance state.
Clause 12.21. The computer-implemented method of Clause 1.21, further comprising:
   based on the compliance state, generating, using the artificial intelligence engine, a modified rehabilitation treatment plan for the user.
Clause 13.21 The computer-implemented method of Clause 11.21, further comprising:
   controlling, while the user uses the electromechanical machine and using the modified treatment plan, the electromechanical machine.
Clause 14.21 A system comprising:
   one or more memory devices storing instructions; and
   one or more processing devices communicatively coupled to the one or more memory devices, wherein the one or more processing devices execute the instructions to:
   receive, from one or more internet-of-things (IOT) devices, data indicative of user interactions with the one or more IoT devices, wherein the user interactions with the one or more IoT devices are related to a prescribed rehabilitation treatment plan involving use of an electromechanical machine;
   analyze, using an artificial intelligence engine, the data to determine a compliance state of the user with the rehabilitation treatment plan, wherein the compliance state is determined based on correlations of one or more indicators included in the data;
   based on the compliance state, generate, using the artificial intelligence engine, a unique data signature associated with the compliance state; and
   based on the unique data signature associated with the compliance state, perform one or more preventative actions.
Clause 15.21 The system of Clause 14.21, wherein the indicators comprise at least one of dietary compliance, medication adherence, activity level, screen time, and environmental condition.
Clause 16.21 The system of Clause 14.21, wherein the one or more processing devices are further configured to perform one or more preventative actions comprising at least one of modifying a parameter of the electromechanical machine and modifying the rehabilitation treatment plan.
Clause 17.21 The system of Clause 14.21, wherein the one or more IOT devices comprise at least one of a smart kitchen appliance, a smart medication dispenser, a smart thermostat, a smart television, a motion sensor, and a wearable fitness tracker.
Clause 18.21 The system of Clause 17.21, wherein the IoT device is a smart kitchen appliance.
Clause 19.21 The system of Clause 18.21, wherein the smart kitchen appliance is a smart refrigerator.
Clause 20.21 The system of Clause 18.21, wherein the data is generated via sensors indicating one or more of opening of a door of the smart refrigerator and an amount of food or one or more characteristics pertaining to food removed from the smart refrigerator.
Clause 21.21 The system of Clause 14.21, wherein the one or more IoT devices comprise a sensor configured to monitor handle pressure applied by the user to the one or more IoT devices, and wherein the data indicative of user interactions includes measurements of handle pressure.
Clause 22.21 The system of Clause 14.21, wherein the one or more IoT devices comprise a sensor configured to monitor user gait, and wherein the data indicative of user interactions includes gait analysis measurements to assess the compliance state.
Clause 23.21 The system of Clause 14.21, wherein the one or more IoT devices comprise one or more of an oven and a stove, wherein the data indicative of user interactions includes data indicative of use of the oven or stove.
Clause 24.21 The system of Clause 14.21, wherein the one or more IoT devices comprise a display device including one or more of a television, cell phone, tablet, and wearable device, wherein the data indicative of user interactions includes information related to screen time usage to assess the compliance state.
Clause 25.21 The system of Clause 14.21, wherein the one or more processing devices are further configured to, based on the compliance state, generate, using the artificial intelligence engine, a modified rehabilitation treatment plan for the user.
Clause 26.21 The system of Clause 25.21, wherein the one or more processing devices are further configured to control, while the user uses the electromechanical machine and using the modified treatment plan, the electromechanical machine.
Clause 27.21 A non-transitory computer-readable medium storing instructions that, when executed by one or more processing devices, cause the one or more processing devices to:
   receive, from one or more internet-of-things (IOT) devices, data indicative of user interactions with the one or more IOT devices, wherein the user interactions with the one or more IoT devices are related to a prescribed rehabilitation treatment plan involving use of an electromechanical machine;
   analyze, using an artificial intelligence engine, the data to determine a compliance state of the user with the rehabilitation treatment plan, wherein the compliance state is determined based on correlations of one or more indicators included in the data;
   based on the compliance state, generate, using the artificial intelligence engine, a unique data signature associated with the compliance state; and
   based on the unique data signature associated with the compliance state, perform one or more preventative actions.
Clause 28.21 The non-transitory computer-readable medium of Clause 27.21, wherein the indicators comprise at least one of dietary compliance, medication adherence, activity level, screen time, and environmental condition.
Clause 29.21 The non-transitory computer-readable medium of Clause 27.21, wherein the instructions, when executed, further cause the one or more processing devices to perform one or more preventative actions comprising at least one of modifying a parameter of the electromechanical machine and modifying the rehabilitation treatment plan.
Clause 30.21 The non-transitory computer-readable medium of Clause 27.21, wherein the one or more IoT devices comprise at least one of a smart kitchen appliance, a smart medication dispenser, a smart thermostat, a smart television, a motion sensor, and a wearable fitness tracker.
Clause 31.21 The non-transitory computer-readable medium of Clause 30.21, wherein the IoT device is a smart kitchen appliance.
Clause 32.21 The non-transitory computer-readable medium of Clause 31.21, wherein the smart kitchen appliance is a smart refrigerator.
Clause 33.21 The non-transitory computer-readable medium of Clause 32.21, wherein the data is generated via sensors indicating one or more of opening of a door of the smart refrigerator and an amount of food or one or more characteristics pertaining to food removed from the smart refrigerator.
Clause 34.21 The non-transitory computer-readable medium of Clause 27.21, wherein the one or more IoT devices comprise a sensor configured to monitor handle pressure applied by the user to the one or more IoT devices, and wherein the data indicative of user interactions includes measurements of handle pressure.
Clause 35.21 The non-transitory computer-readable medium of Clause 27.21, wherein the one or more IoT devices comprise a sensor configured to monitor user gait, and wherein the data indicative of user interactions includes gait analysis measurements to assess the compliance state.
Clause 36.21 The non-transitory computer-readable medium of Clause 27.21, wherein the one or more IoT devices comprise one or more of an oven and a stove, wherein the data indicative of user interactions includes data indicative of use of the oven or stove.
Clause 37.21 The non-transitory computer-readable medium of Clause 27.21, wherein the one or more IoT devices comprise a display device including one or more of a television, cell phone, tablet, and wearable device, wherein the data indicative of user interactions includes information related to screen time usage to assess the compliance state.
Clause 38.21 The non-transitory computer-readable medium of Clause 27.21, wherein the instructions, when executed, further cause the one or more processing devices to, based on the compliance state, generate, using the artificial intelligence engine, a modified rehabilitation treatment plan for the user.
Clause 39.21 The non-transitory computer-readable medium of Clause 38.21, wherein the instructions, when executed, further cause the one or more processing devices to control, while the user uses the electromechanical machine and using the modified treatment plan, the electromechanical machine.
   Systems and Methods for an Enhanced User Interface Presenting a Control Command Center Enabling Monitoring of a Plurality of Patients and Devices that are Executing Interventions And Evidence-Based Guided Protocols

FIG. 17E depicts a method for providing an enhanced user interface as a control command center for monitoring and managing a plurality of patients and devices executing interventions and evidence-based protocols. In step 1752, the system receives data at a control command center from a plurality of devices associated with a plurality of patients, where each device is configured to execute one or more interventions or protocols as part of a patient treatment plan. The data may include device status, patient compliance, physiological measurements, and intervention outcomes.

In step 1754, the system presents, via a user interface of the control command center, a real-time display of patient status information and device status information for the plurality of patients and devices. The interface may include dashboards, patient tiles, device health indicators, and cohort-level analytics.

Step 1756 involves analyzing the data using an artificial intelligence engine to identify, for each patient, a progression state, a compliance state, or a need for intervention based on one or more indicators included in the data. The AI engine may flag patients at risk, recommend protocol adjustments, or detect device malfunctions.

In step 1758, the system generates, using the artificial intelligence engine and based on the analysis, one or more recommended interventions or protocol adjustments for at least one of the patients. These recommendations may be based on evidence-based protocols, historical outcomes, or real-time risk assessments.

Finally, in step 1760, the system enables, via the user interface, a therapist or an artificial intelligence system to monitor the plurality of patients and devices, review the recommended interventions or protocol adjustments, and selectively initiate, approve, or modify interventions for the at least one of the patients. This enables scalable, data-driven, and collaborative management of patient care across large populations and multiple care settings.

Consistent with the above disclosure, the examples of systems and methods enumerated in the following clauses are specifically contemplated and are intended as a non-limiting set of examples.
Clause 1.24 A computer-implemented method comprising:
   receiving, at a control command center, data from a plurality of devices associated with a plurality of patients, wherein each device is configured to execute one or more interventions or protocols as part of a patient treatment plan;
   presenting, via a user interface of the control command center, a real-time display of patient status information and device status information for the plurality of patients and devices;
   analyzing, using an artificial intelligence engine, the data to identify, for each patient, a progression state, a compliance state, or a need for intervention based on one or more indicators included in the data;
   generating, using the artificial intelligence engine and based on the analysis, one or more recommended interventions or protocol adjustments for at least one of the patients; and
   enabling, via the user interface, a therapist or an artificial intelligence system to monitor the plurality of patients and devices, review the recommended interventions or protocol adjustments, and selectively initiate, approve, or modify interventions for the at least one of the patients.
Clause 2.24 The computer-implemented method of Clause 1.24, wherein the user interface enables a therapist to monitor the plurality of patients and devices in real time, review patient-specific data, and manually initiate or approve interventions.
Clause 3.24 The computer-implemented method of Clause 2.24, wherein the therapist is at least one of a human, a computer application, and clinician, an administrator, an artificial intelligence agent based on a large language model, and a pre-programmed protocol or script.
Clause 4.24 The computer-implemented method of Clause 1.24, wherein the user interface enables the artificial intelligence engine to autonomously monitor the plurality of patients and devices and to automatically initiate or modify interventions based on predefined criteria or learned patterns.
Clause 5.24 The computer-implemented method of Clause 1.24, wherein the artificial intelligence engine is trained on historical patient data and evidence-based protocols to provide the recommended interventions or protocol adjustments.
Clause 6.24 The computer-implemented method of Clause 1.24, wherein the enabling includes enabling the artificial intelligence system to monitor patients and devices at scale, including across multiple geographic regions, healthcare facilities, healthcare systems, or national and global deployments.
Clause 7.24 The computer-implemented method of Clause 1.24, wherein, based on risk level, intervention status, diagnosis, or protocol adherence, the user interface provides filtering and sorting capabilities to allow users to view patient cohorts.
Clause 8.24 The computer-implemented method of Clause 1.24, further comprising:
displaying, via the user display and using the artificial-intelligence engine, real-time alerts for protocol non-compliance, device malfunctions, abnormal vital signs, atypical vital signs, or risk states.
Clause 9.24 The computer-implemented method of Clause 1.24, further comprising: displaying, via the user display and using the artificial-intelligence engine, visualizations including trend graphs, heat maps, and compliance charts for rapid assessment of patient population health and protocol adherence.
Clause 10.24 The computer-implemented method of Clause 1.24, wherein the control command center supports role-based access control, enabling different levels of access and functionality for therapists, clinicians, administrators, and AI systems.
Clause 11.24 The computer-implemented method of Clause 1.24, wherein all actions and interventions performed via the control command center are logged to generate an audit trail for regulatory compliance and quality assurance.
Clause 12.24 A system comprising:
   one or more memory devices storing instructions; and
   one or more processing devices communicatively coupled to the one or more memory devices, wherein the one or more processing devices execute the instructions to:
   receive, at a control command center, data from a plurality of devices associated with a plurality of patients, wherein each device is configured to execute one or more interventions or protocols as part of a patient treatment plan;
   present, via a user interface of the control command center, a real-time display of patient status information and device status information for the plurality of patients and devices;
   analyze, using an artificial intelligence engine, the data to identify, for each patient, a progression state, a compliance state, or a need for intervention based on one or more indicators included in the data;
   generate, using the artificial intelligence engine and based on the analysis, one or more recommended interventions or protocol adjustments for at least one of the patients; and
   enable, via the user interface, a therapist or an artificial intelligence system to monitor the plurality of patients and devices, review the recommended interventions or protocol adjustments, and selectively initiate, approve, or modify interventions for the at least one of the patients.
Clause 13.24 The system of Clause 12.24, wherein the user interface enables a therapist to monitor the plurality of patients and devices in real time, review patient-specific data, and manually initiate or approve interventions.
Clause 14.24 The system of Clause 12.24, wherein the therapist is at least one of a human, a computer application, a clinician, an administrator, an artificial intelligence agent based on a large language model, or a pre-programmed protocol or script.
Clause 15.24 The system of Clause 12.24, wherein the user interface enables the artificial intelligence engine to autonomously monitor the plurality of patients and devices and to automatically initiate or modify interventions based on predefined criteria or learned patterns.
Clause 16.24 The system of Clause 12.24, wherein the artificial intelligence engine is trained on historical patient data and evidence-based protocols to provide the recommended interventions or protocol adjustments.
Clause 17.24 The system of Clause 12.24, wherein the enabling includes enabling the artificial intelligence system to monitor patients and devices at scale, including across multiple geographic regions, healthcare facilities, healthcare systems, or national and global deployments.
Clause 18.24 The system of Clause 12.24, wherein, based on risk level, intervention status, diagnosis, or protocol adherence, the user interface provides filtering and sorting capabilities to allow users to view patient cohorts.
Clause 19.24 The system of Clause 12.24, wherein the one or more processing devices are further configured to display, via the user display and using the artificial intelligence engine, real-time alerts for protocol non-compliance, device malfunctions, abnormal vital signs, atypical vital signs, or risk states.
Clause 20.24 The system of Clause 12.24, wherein the one or more processing devices are further configured to display, via the user display and using the artificial intelligence engine, visualizations including trend graphs, heat maps, and compliance charts for rapid assessment of patient population health and protocol adherence.
Clause 21.24 The system of Clause 12.24, wherein the control command center supports role-based access control, enabling different levels of access and functionality for therapists, clinicians, administrators, and AI systems.
Clause 22.24 The system of Clause 12.24, wherein all actions and interventions performed via the control command center are logged to generate an audit trail for regulatory compliance and quality assurance.
Clause 23.24 A non-transitory computer-readable medium storing instructions that, when executed by one or more processing devices, cause the one or more processing devices to:
   receive, at a control command center, data from a plurality of devices associated with a plurality of patients, wherein each device is configured to execute one or more interventions or protocols as part of a patient treatment plan;
   present, via a user interface of the control command center, a real-time display of patient status information and device status information for the plurality of patients and devices;
   analyze, using an artificial intelligence engine, the data to identify, for each patient, a progression state, a compliance state, or a need for intervention based on one or more indicators included in the data;
   generate, using the artificial intelligence engine and based on the analysis, one or more recommended interventions or protocol adjustments for at least one of the patients; and
   enable, via the user interface, a therapist or an artificial intelligence system to monitor the plurality of patients and devices, review the recommended interventions or protocol adjustments, and selectively initiate, approve, or modify interventions for the at least one of the patients.
Clause 24.24 The non-transitory computer-readable medium of Clause 23.24, wherein the user interface enables a therapist to monitor the plurality of patients and devices in real time, review patient-specific data, and manually initiate or approve interventions.
Clause 25.24 The non-transitory computer-readable medium of Clause 24.24, wherein the therapist is at least one of a human, a computer application, a clinician, an administrator, an artificial intelligence agent based on a large language model, or a pre-programmed protocol or script.
Clause 26.24 The non-transitory computer-readable medium of Clause 23.24, wherein the user interface enables the artificial intelligence engine to autonomously monitor the plurality of patients and devices and to automatically initiate or modify interventions based on predefined criteria or learned patterns.
Clause 27.24 The non-transitory computer-readable medium of Clause 23.24, wherein the artificial intelligence engine is trained on historical patient data and evidence-based protocols to provide the recommended interventions or protocol adjustments.
Clause 28.24 The non-transitory computer-readable medium of Clause 23.24, wherein the enabling includes enabling the artificial intelligence system to monitor patients and devices at scale, including across multiple geographic regions, healthcare facilities, healthcare systems, or national and global deployments.
Clause 29.24 The non-transitory computer-readable medium of Clause 23.24, wherein, based on risk level, intervention status, diagnosis, or protocol adherence, the user interface provides filtering and sorting capabilities to allow users to view patient cohorts.
Clause 30.24 The non-transitory computer-readable medium of Clause 23.24, wherein the instructions, when executed, further cause the one or more processing devices to display, via the user display and using the artificial intelligence engine, real-time alerts for protocol non-compliance, device malfunctions, abnormal vital signs, atypical vital signs, or risk states.
Clause 31.24 The non-transitory computer-readable medium of Clause 23.24, wherein the instructions, when executed, further cause the one or more processing devices to display, via the user display and using the artificial intelligence engine, visualizations including trend graphs, heat maps, and compliance charts for rapid assessment of patient population health and protocol adherence.
Clause 32.24 The non-transitory computer-readable medium of Clause 23.24, wherein the control command center supports role-based access control, enabling different levels of access and functionality for therapists, clinicians, administrators, and AI systems.
Clause 33.24 The non-transitory computer-readable medium of Clause 23.24, wherein all actions and interventions performed via the control command center are logged to generate an audit trail for regulatory compliance and quality assurance.
   Systems and Methods for Optimizing Rehabilitation via a Plurality of Rehabilitation Protocols Targeted at Improving and/or Mitigating One or More Identified Conditions

FIG. 17F illustrates an embodiment of a computer-implemented method for optimizing rehabilitation by dynamically selecting and executing a plurality of rehabilitation protocols, each targeted at improving or mitigating one or more identified conditions in a user. In step 1762, the system receives, for a user, data indicative of one or more identified conditions associated with the user. This data may include clinical diagnoses, sensor measurements, user self-reports, and historical health records, including but not limited to comorbidities. For example, the system may receive information indicating that a user is recovering from knee surgery, other surgeries, including but not limited to orthopedic, cardiovascular, pulmonary, neurological, oncological, throracic, abdominal, and renal surgeries, has a history of diabetes, or is experiencing chronic pain. The data may further include results from recent or earlier imaging studies, medication usage history, records of previous rehabilitation outcomes, wearable sensor data capturing daily activity levels, or clinician-entered notes regarding observed gait abnormalities or balance deficits.

In step 1764, the system selects, using an artificial intelligence engine and based on the received data, a plurality of rehabilitation protocols from a protocol library. Each rehabilitation protocol in the library is designed to address specific conditions or therapeutic goals. The AI engine may analyze the user's data to determine which protocols are most likely to improve or mitigate the identified conditions. For instance, the system may select a pain management protocol, a range-of-motion protocol, and a strength-building protocol for a user with post-surgical pain and limited mobility. If the data indicates the user has a neurological impairment, such as post-stroke hemiparesis, the system may select protocols focused on motor retraining, balance exercises, and cognitive rehabilitation. For a user with a history of diabetes and peripheral neuropathy, the system may select protocols that emphasize low-impact aerobic activity, foot care education, and blood glucose monitoring. In cases where the user's data reveals both cardiovascular procedures and/or limitations and musculoskeletal injuries, the AI engine may recommend a combination of low-intensity cardiovascular training, joint-friendly resistance exercises, and flexibility routines. The protocol selection process is thus highly individualized, ensuring that each user receives a tailored combination of interventions that address their unique clinical profile and rehabilitation goals.

Step 1766 involves assigning the user to at least one cohort based on shared characteristics with other users. The cohort assignment is used to inform protocol selection or adaptation. The system may analyze a variety of user attributes-such as age, gender, primary diagnosis, comorbidities, baseline functional status, previous rehabilitation outcomes, and even psychosocial and psychological/mental health factors—to identify a group of users with similar profiles. For example, the system may group the user with other individuals of similar age, diagnosis, or comorbidity profile, and use outcome data from that cohort to further refine the recommended protocols. In some embodiments, the cohort may be dynamically defined using clustering algorithms or machine learning models that identify patterns in large datasets of historical patient information. The system may also consider factors such as adherence rates, recovery trajectories and the user's response to prior interventions when determining the most appropriate cohort. By leveraging aggregated data and best practices from users within the same cohort, the system can recommend protocols that have demonstrated higher efficacy for individuals with comparable characteristics, thereby personalizing and optimizing the rehabilitation process for each user.

In step 1768, the system executes, for the user, the plurality of rehabilitation protocols in a sequence or combination determined by the artificial intelligence engine. Execution comprises controlling operation of at least one rehabilitation device in accordance with the selected protocols. For example, the system may schedule alternating sessions of low-impact cycling and resistance training, or combine balance exercises with cognitive tasks for users with neurological impairments. In some embodiments, the system may coordinate the use of multiple devices-such as a treadmill for gait training, a smart resistance machine for strength building, and a virtual reality platform for upper limb rehabilitation-ensuring that each device is activated at the appropriate time and with parameters tailored to the user's current needs. The AI engine may also adapt the sequence or combination of protocols in real time, such as introducing flexibility or stretching routines after periods of intense activity, or integrating rest intervals based on detected fatigue or physiological stress. Additionally, the system may provide the user with real-time feedback, prompts, or motivational cues through a user interface or connected device, helping to guide the user through each stage of the rehabilitation process and maximize engagement and adherence. This dynamic and coordinated execution of multiple protocols allows for a comprehensive, holistic approach to rehabilitation that addresses the user's evolving clinical profile and therapeutic goals.

Step 1770 includes monitoring, using one or more sensors and the artificial intelligence engine, user-performance data generated during execution of the rehabilitation protocols. The user-performance data may include objective measurements such as changes in pedal radius, heart rate, or activity duration, as well as qualitative data such as user-reported pain levels or fatigue. Other types of user-performance data disclosed herein can be generated and used for monitoring. The system may continuously or periodically collect and analyze this data to assess the user's progress and response to the protocols.

Finally, in step 1770, the system dynamically modifies, using the artificial intelligence engine and based on the user-performance data, at least one parameter of the rehabilitation protocols to optimize therapeutic outcomes for the user. This may include adjusting exercise intensity, duration, frequency, or modality in real time or near real time, switching between different protocols, or combining multiple protocols as the user's condition evolves or changes, whether for the better or worse, or if it remains stable. For example, if the user demonstrates improved strength but increased pain, the system may reduce resistance while increasing range-of-motion activities, or recommend a rest period before resuming more intensive exercises.

Consistent with the above disclosure, the examples of systems and methods enumerated in the following clauses are specifically contemplated and are intended as a non-limiting set of examples.
Clause 1.16 A computer-implemented method comprising:
   receiving, for a user, data indicative of one or more identified conditions associated with the user;
   selecting, using an artificial intelligence engine and based on the data, a plurality of rehabilitation protocols from a protocol library, wherein each rehabilitation protocol is targeted at improving or mitigating at least one of the identified conditions;
   assigning the user to at least one cohort based on shared characteristics with other users, wherein the cohort assignment is used to inform protocol selection or adaptation;
   executing, for the user, the plurality of rehabilitation protocols in a sequence or combination determined by the artificial intelligence engine, wherein the execution comprises controlling operation of at least one rehabilitation device in accordance with the selected protocols;
   monitoring, using one or more sensors and the artificial intelligence engine, user-performance data generated during execution of the rehabilitation protocols; and
   dynamically modifying, using the artificial intelligence engine and based on the user-performance data, at least one parameter of the rehabilitation protocols to optimize therapeutic outcomes for the user.
Clause 2.16 The computer-implemented method of Clause 1.16, wherein the user-performance data comprises objective data, including one or more proxy indicators of pain such as changes in pedal radius, sudden increases in heart rate, or other physiological or performance-based measurements obtained from the one or more sensors.
Clause 3.16. The computer-implemented method of Clause 1.16, wherein the user-performance data comprises qualitative data, including user-reported pain levels or subjective assessments of discomfort provided by the user before, during or after execution of the rehabilitation protocols.
Clause 4.16 The computer-implemented method of Clause 1.16, wherein dynamically modifying at least one parameter of the rehabilitation protocols comprises adjusting exercise intensity, duration, frequency, or modality in real time or near real time based on trends or anomalies detected in the user-performance data.
Clause 5.16 The computer-implemented method of Clause 1.16, wherein the artificial intelligence engine is further configured to recommend switching between different rehabilitation protocols or combining multiple protocols in response to changes in the user's condition as reflected in the user-performance data.
Clause 6.16 The computer-implemented method of Clause 5.16, wherein the combined multiple protocols comprise one of at least portions of two or more of the multiple protocols or at least portions of all of the multiple protocols
Clause 7.16 The computer-implemented method of Clause 1.16, wherein the protocol library comprises evidence-based protocols that are periodically updated based on aggregated user outcomes and one or more cohort analyses.
Clause 8.16 The computer-implemented method of Clause 1.16, further comprising generating and presenting, via a user interface, progress reports or alerts to a clinician or the user based on analysis of the user-performance data and protocol modifications.
Clause 9.16 The computer-implemented method of Clause 1.16, wherein assigning the user to at least one cohort comprises analyzing the user's demographic, clinical, or performance characteristics and grouping the user with other users exhibiting similar characteristics, such that protocol selection is informed by outcomes observed within the assigned cohort relative to the other users in the same cohort.
Clause 10.16 A system comprising:
   one or more memory devices storing instructions; and
   one or more processing devices communicatively coupled to the one or more memory devices, wherein the one or more processing devices execute the instructions to:
   receive, for a user, data indicative of one or more identified conditions associated with the user;
   select, using an artificial intelligence engine and based on the data, a plurality of rehabilitation protocols from a protocol library, wherein each rehabilitation protocol is targeted at improving or mitigating at least one of the identified conditions;
   assign the user to at least one cohort based on shared characteristics with other users, wherein the cohort assignment is used to inform protocol selection or adaptation;
   execute, for the user, the plurality of rehabilitation protocols in a sequence or combination determined by the artificial intelligence engine, wherein the execution comprises controlling operation of at least one rehabilitation device in accordance with the selected protocols;
   monitor, using one or more sensors and the artificial intelligence engine, user-performance data generated during execution of the rehabilitation protocols; and
   dynamically modify, using the artificial intelligence engine and based on the user-performance data, at least one parameter of the rehabilitation protocols to optimize therapeutic outcomes for the user.
Clause 11.16 The system of Clause 10.16, wherein the user-performance data comprises objective data, including one or more proxy indicators of pain such as changes in pedal radius, sudden increases in heart rate, or other physiological or performance-based measurements obtained from the one or more sensors.
Clause 12.16 The system of Clause 10.16, wherein the user-performance data comprises qualitative data, including user-reported pain levels or subjective assessments of discomfort provided by the user before, during or after execution of the rehabilitation protocols.
Clause 13.16 The system of Clause 10.16, wherein dynamically modifying at least one parameter of the rehabilitation protocols comprises adjusting exercise intensity, duration, frequency, or modality in real time or near real time based on trends or anomalies detected in the user-performance data.
Clause 14.16 The system of Clause 10.16, wherein the artificial intelligence engine is further configured to recommend switching between different rehabilitation protocols or combining multiple protocols in response to changes in the user's condition as reflected in the user-performance data.
Clause 15.16 The system of Clause 10.16, wherein the combined multiple protocols comprise one of at least portions of two or more of the multiple protocols or at least portions of all of the multiple protocols.
Clause 16.16 The system of Clause 10.16, wherein the protocol library comprises evidence-based protocols that are periodically updated based on aggregated user outcomes and one or more cohort analyses.
Clause 17.16 The system of Clause 10.16, wherein the one or more processing devices are further configured to generate and present, via a user interface, progress reports or alerts to a clinician or the user based on analysis of the user-performance data and protocol modifications.
Clause 18.16 The system of Clause 10.16, wherein assigning the user to at least one cohort comprises analyzing the user's demographic, clinical, or performance characteristics and grouping the user with other users exhibiting similar characteristics, such that protocol selection is informed by outcomes observed within the assigned cohort relative to the other users in the same cohort.
Clause 19.16 A non-transitory computer-readable medium storing instructions that, when executed by one or more processing devices, cause the one or more processing devices to:
   receive, for a user, data indicative of one or more identified conditions associated with the user;
   select, using an artificial intelligence engine and based on the data, a plurality of rehabilitation protocols from a protocol library, wherein each rehabilitation protocol is targeted at improving or mitigating at least one of the identified conditions;
   assign the user to at least one cohort based on shared characteristics with other users, wherein the cohort assignment is used to inform protocol selection or adaptation;
   execute, for the user, the plurality of rehabilitation protocols in a sequence or combination determined by the artificial intelligence engine, wherein the execution comprises controlling operation of at least one rehabilitation device in accordance with the selected protocols;
   monitor, using one or more sensors and the artificial intelligence engine, user-performance data generated during execution of the rehabilitation protocols; and
   dynamically modify, using the artificial intelligence engine and based on the user-performance data, at least one parameter of the rehabilitation protocols to optimize therapeutic outcomes for the user.
Clause 20.16 The non-transitory computer-readable medium of Clause 19.16, wherein the user-performance data comprises objective data, including one or more proxy indicators of pain such as changes in pedal radius, sudden increases in heart rate, or other physiological or performance-based measurements obtained from the one or more sensors.
Clause 21.16 The non-transitory computer-readable medium of Clause 19.16, wherein the user-performance data comprises qualitative data, including user-reported pain levels or subjective assessments of discomfort provided by the user before, during or after execution of the rehabilitation protocols.
Clause 22.16 The non-transitory computer-readable medium of Clause 19.16, wherein dynamically modifying at least one parameter of the rehabilitation protocols comprises adjusting exercise intensity, duration, frequency, or modality in real time or near real time based on trends or anomalies detected in the user-performance data.
Clause 23.16 The non-transitory computer-readable medium of Clause 19.16, wherein the artificial intelligence engine is further configured to recommend switching between different rehabilitation protocols or combining multiple protocols in response to changes in the user's condition as reflected in the user-performance data.
Clause 24.16 The non-transitory computer-readable medium of Clause 23.16, wherein the combined multiple protocols comprise one of at least portions of two or more of the multiple protocols or at least portions of all of the multiple protocols.
Clause 25.16 The non-transitory computer-readable medium of Clause 19.16, wherein the protocol library comprises evidence-based protocols that are periodically updated based on aggregated user outcomes and one or more cohort analyses.
Clause 26.16 The non-transitory computer-readable medium of Clause 19.16, wherein the instructions, when executed, further cause the one or more processing devices to generate and present, via a user interface, progress reports or alerts to a clinician or the user based on analysis of the user-performance data and protocol modifications.
Clause 27.16 The non-transitory computer-readable medium of Clause 19.16, wherein assigning the user to at least one cohort comprises analyzing the user's demographic, clinical, or performance characteristics and grouping the user with other users exhibiting similar characteristics, such that protocol selection is informed by outcomes observed within the assigned cohort relative to the other users in the same cohort.
   Systems and Methods for Optimizing Rehabilitation by Performing AI-Driven Interventions for Patients with Multiple Comorbidities

FIG. 17G illustrates an embodiment of a computer-implemented method for optimizing rehabilitation by performing AI-driven interventions for patients with multiple comorbidities. In step 1782, the system receives, for a patient, data indicative of a plurality of comorbidities and associated physiological and performance information defining a comorbidity profile. This data may include laboratory test results, vital sign measurements, sensor-derived physiological parameters, device-recorded performance metrics, and clinical diagnoses. For example, the system may receive data indicating that a patient has both heart failure and osteoarthritis, along with recent blood pressure readings and activity levels.

In step 1784, the system analyzes, using an artificial intelligence engine, the data to objectively select, from a protocol library, a rehabilitation protocol optimized for the patient's comorbidity profile. The protocol selection is based on data associated with patients having similar comorbidity profiles, leveraging statistical models and outcome data to identify the most effective evidence-based protocols. For instance, the AI engine may select a protocol that integrates cardiovascular exercise with joint-friendly strength training for a patient with both cardiac and orthopedic conditions.

Step 1786 involves controlling, in accordance with the selected rehabilitation protocol, operation of at least one rehabilitation device during execution of the selected protocol. The system may automatically adjust device parameters such as range of motion, resistance, speed, or exercise type to match the requirements of the protocol and the patient's current health status.

In step 1788, the system monitors, using one or more sensors and the artificial intelligence engine, patient performance and physiological responses during execution of the selected rehabilitation protocol. This monitoring may include tracking heart rate, range of motion, pain levels, and adherence to prescribed activities. The system may also collect data from wearable devices, smart home sensors, or direct user input. The data can be collected in any of the modalities, by any of the methods, and by any other lawful means of obtaining the data, without limitation, as further disclosed herein.

Finally, in step 1790, the system dynamically modifies, using the artificial intelligence engine and based on the monitored performance and physiological data, at least one parameter of the rehabilitation protocol to optimize therapeutic outcomes for the patient. This may include adjusting exercise intensity, duration, frequency, or modality in real time, switching to alternative protocols if the patient's condition changes, or integrating additional interventions such as virtual reality (VR) or augmented reality (AR) activities. For example, if the system detects that a patient's heart rate is elevated beyond a safe threshold during exercise, it may automatically reduce the intensity or prompt the patient to rest, while also notifying a clinician if necessary. The system may also recommend new protocol combinations and/or schedule telehealth check-ins based on ongoing analysis of the patient's progress and risk factors.

Consistent with the above disclosure, the examples of systems and methods enumerated in the following clauses are specifically contemplated and are intended as a non-limiting set of examples.
Clause 1.17 A computer-implemented method comprising:
   receiving, for a patient, data indicative of a plurality of comorbidities and associated physiological and performance information defining a comorbidity profile;
   analyzing, using an artificial intelligence engine, the data to objectively select, from a protocol library, a rehabilitation protocol optimized for the patient's comorbidity profile, wherein the protocol selection is based on data associated with patients having similar comorbidity profiles;
   controlling, in accordance with the selected rehabilitation protocol, operation of at least one rehabilitation device during execution of the selected rehabilitation protocol;
   monitoring, using one or more sensors and the artificial intelligence engine, patient performance and physiological responses during execution of the selected rehabilitation protocol; and
   dynamically modifying, using the artificial intelligence engine and based on the monitored performance and physiological data, at least one parameter of the rehabilitation protocol in order to optimize therapeutic outcomes for the patient.
Clause 2.17 The computer-implemented method of Clause 1.17, wherein the data indicative of a plurality of comorbidities comprises objective data, including at least one of laboratory test results, vital sign measurements, biometric data or measurements, sensor-derived physiological parameters, or device-recorded performance metrics.
Clause 3.17 The computer-implemented method of Clause 1.17, wherein the protocol library comprises a plurality of evidence-based rehabilitation protocols, each protocol being associated with outcome data for different comorbidity profiles.
Clause 4.17 The computer-implemented method of Clause 1.17, wherein the artificial intelligence engine is further configured to recommend switching between different rehabilitation protocols or combining at least portions of multiple protocols into a new protocol in response to changes in the patient's comorbidity profile or monitored performance.
Clause 5.17 The computer-implemented method of Clause 1.17, wherein dynamically modifying at least one parameter of the rehabilitation protocol comprises adjusting exercise intensity, duration, frequency, or modality in real time based on trends or anomalies detected in the monitored performance and physiological data.
Clause 6.17 The computer-implemented method of Clause 1.17, further comprising generating and presenting, via a user interface, progress reports or alerts to a clinician or the patient based on analysis of the monitored performance and protocol modifications.
Clause 7.17 The computer-implemented method of Clause 1.17, wherein the selected rehabilitation protocol includes at least one therapeutic activity delivered via a virtual reality (VR) or augmented reality (AR) environment, and wherein the artificial intelligence engine adjusts VR/AR content or difficulty based on the patient's comorbidity profile and monitored responses.
Clause 8.17 The computer-implemented method of Clause 1.17, wherein the protocol selection is further based on cohort analysis, such that the artificial intelligence engine references outcomes from groups of patients with similar comorbidity profiles to optimize protocol assignment for the patient.
Clause 9.17 A system comprising: one or more memory devices storing instructions; and one or more processing devices communicatively coupled to the one or more memory devices, wherein the one or more processing devices execute the instructions to:
   receive, for a patient, data indicative of a plurality of comorbidities and associated physiological and performance information defining a comorbidity profile;
   analyze, using an artificial intelligence engine, the data to objectively select, from a protocol library, a rehabilitation protocol optimized for the patient's comorbidity profile, wherein the protocol selection is based on data associated with patients having similar comorbidity profiles;
   control, in accordance with the selected rehabilitation protocol, operation of at least one rehabilitation device during execution of the selected rehabilitation protocol;
   monitor, using one or more sensors and the artificial intelligence engine, patient performance and physiological responses during execution of the selected rehabilitation protocol; and
   dynamically modify, using the artificial intelligence engine and based on the monitored performance and physiological data, at least one parameter of the rehabilitation protocol to optimize therapeutic outcomes for the patient.
Clause 10.17 The system of Clause 9.17, wherein the data indicative of a plurality of comorbidities comprises objective data, including at least one of laboratory test results, vital sign measurements, sensor-derived physiological parameters, or device-recorded performance metrics.
Clause 11.17 The system of Clause 9.17, wherein the protocol library comprises a plurality of evidence-based rehabilitation protocols, each protocol being associated with outcome data for different comorbidity profiles.
Clause 12.17 The system of Clause 9.17, wherein the artificial intelligence engine is further configured to recommend switching between different rehabilitation protocols or combining multiple protocols in response to changes in the patient's comorbidity profile or monitored performance.
Clause 13.17 The system of Clause 9.17, wherein dynamically modifying at least one parameter of the rehabilitation protocol comprises adjusting exercise intensity, duration, frequency, or modality in real time based on trends or anomalies detected in the monitored performance and physiological data.
Clause 14.17 The system of Clause 9.17, wherein the one or more processing devices are further configured to generate and present, via a user interface, progress reports or alerts to a clinician or the patient based on analysis of the monitored performance and protocol modifications.
Clause 15.17 The system of Clause 9.17, wherein the selected rehabilitation protocol includes at least one therapeutic activity delivered via a virtual reality (VR) or augmented reality (AR) environment, and wherein the artificial intelligence engine adjusts VR/AR content or difficulty based on the patient's comorbidity profile and monitored responses.
Clause 16.17 The system of Clause 9.17, wherein the protocol selection is further based on cohort analysis, such that the artificial intelligence engine references outcomes from groups of patients with similar comorbidity profiles to optimize protocol assignment for the patient.
Clause 17.17 A non-transitory computer-readable medium storing instructions that, when executed by one or more processing devices, cause the one or more processing devices to:
   receive, for a patient, data indicative of a plurality of comorbidities and associated physiological and performance information defining a comorbidity profile;
   analyze, using an artificial intelligence engine, the data to objectively select, from a protocol library, a rehabilitation protocol optimized for the patient's comorbidity profile, wherein the protocol selection is based on data associated with patients having similar comorbidity profiles;
   control, in accordance with the selected rehabilitation protocol, operation of at least one rehabilitation device during execution of the selected rehabilitation protocol;
   monitor, using one or more sensors and the artificial intelligence engine, patient performance and physiological responses during execution of the selected rehabilitation protocol; and
   dynamically modify, using the artificial intelligence engine and based on the monitored performance and physiological data, at least one parameter of the rehabilitation protocol to optimize therapeutic outcomes for the patient.
Clause 18.17 The non-transitory computer-readable medium of Clause 17.17, wherein the data indicative of a plurality of comorbidities comprises objective data, including at least one of laboratory test results, vital sign measurements, sensor-derived physiological parameters, or device-recorded performance metrics.
Clause 19.17 The non-transitory computer-readable medium of Clause 17.17, wherein the protocol library comprises a plurality of evidence-based rehabilitation protocols, each protocol being associated with outcome data for different comorbidity profiles.
Clause 20.17 The non-transitory computer-readable medium of Clause 17.17, wherein the artificial intelligence engine is further configured to recommend switching between different rehabilitation protocols or combining multiple protocols in response to changes in the patient's comorbidity profile or monitored performance.
Clause 21.17 The non-transitory computer-readable medium of Clause 17.17, wherein dynamically modifying at least one parameter of the rehabilitation protocol comprises adjusting exercise intensity, duration, frequency, or modality in real time based on trends or anomalies detected in the monitored performance and physiological data.
Clause 22.17 The non-transitory computer-readable medium of Clause 17.17, wherein the instructions, when executed, further cause the one or more processing devices to generate and present, via a user interface, progress reports or alerts to a clinician or the patient based on analysis of the monitored performance and protocol modifications.
Clause 23.17 The non-transitory computer-readable medium of Clause 17.17, wherein the selected rehabilitation protocol includes at least one therapeutic activity delivered via a virtual reality (VR) or augmented reality (AR) environment, and wherein the artificial intelligence engine adjusts VR/AR content or difficulty based on the patient's comorbidity profile and monitored responses.
Clause 24.17 The non-transitory computer-readable medium of Clause 17.17, wherein the protocol selection is further based on cohort analysis, such that the artificial intelligence engine references outcomes from groups of patients with similar comorbidity profiles to optimize protocol assignment for the patient.
   Systems and Methods for Using a Generative LLM with NLP-Assisted Interactions to Interact with a User During a Treatment Session in Order to Guide the User in the User's Use Of the Electromechanical Machine

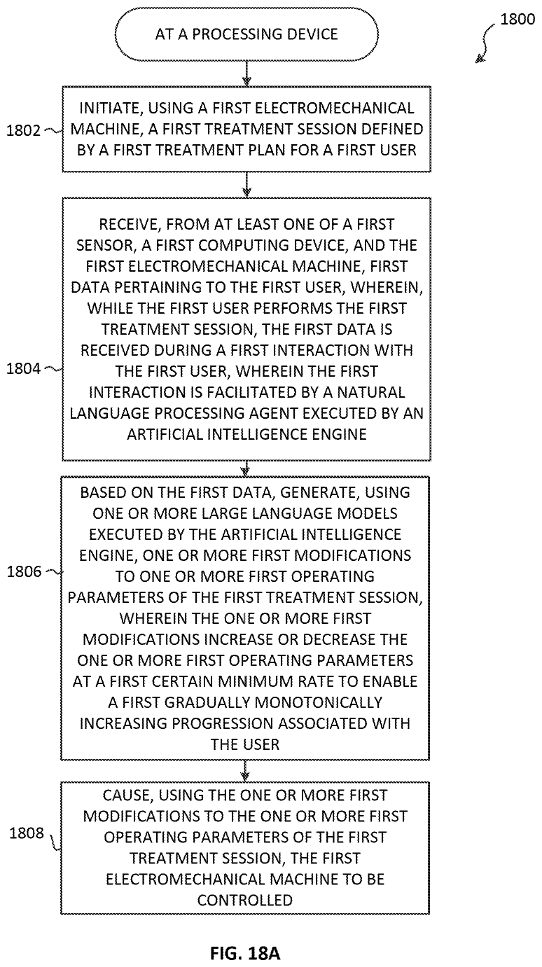

FIG. 18A is a flow diagram generally illustrating a method 1800 for using a generative LLM with natural language processing (NLP)-assisted interactions to interact with a user during a treatment session in order to guide the user in the user's use of the electromechanical machine according to principles of the present disclosure. The method 1800 is performed by processing logic that may include hardware (circuitry, dedicated logic, etc.), software (such as is run on a general-purpose computer system or a dedicated machine), or a combination of both. The method 1800 and/or each of its individual functions, routines, subroutines, methods (as the term is used in object-oriented programming), or operations may be performed by one or more processing devices of a computing device (e.g., any component of FIG. 1, such as the assistant interface 94, patient interface 50, and/or server 30 executing the artificial intelligence engine 11, training engine 9, machine learning models 13, etc.) implementing the method 1800. The method 1800 may be implemented as computer instructions stored on a memory device and able to be executed by the one or more processing devices. In certain implementations, the method 1800 may be performed by a single processing thread. Alternatively, the method 1800 may be performed by two or more processing threads, each thread implementing one or more individual functions, routines, subroutines, methods or operations of the methods. In some embodiments, one or more accelerators may be used to increase the performance of a processing device by offloading various functions, routines, subroutines, methods or operations from the processing device.

For simplicity of explanation, the method 1800 is depicted in FIG. 18A and described as a series of operations. However, operations in accordance with this disclosure can occur in various orders and/or concurrently, and/or with other operations not presented and described herein. For example, the operations depicted in the method 1800 in FIG. 18A may occur in combination with any other operation of any other method disclosed herein. Furthermore, not all illustrated operations may be required to implement the method 1800 in accordance with the disclosed subject matter. In addition, those skilled in the art will understand and appreciate that the method 1800 could alternatively be represented as a series of interrelated states via a state diagram or event diagram.

In some embodiments, one or more computer-implemented models (e.g., machine learning models, neural networks, expert systems, etc.) may be generated and trained by the artificial intelligence engine 11 and/or the training engine 9 to perform one or more of the operations of the methods described herein. For example, to perform the one or more operations, the processing device may execute the one or more computer-implemented models. In some embodiments, the one or more computer-implemented models may be iteratively retrained to select different features capable of enabling optimization of output. The features that may be modified may include a number of nodes included in each layer of the models, an objective function executed at each node, a number of layers, various weights associated with outputs of each node, and the like.

At step 1802, the processing device may initiate, using a first electromechanical machine, a first treatment session defined by a first treatment plan for a first user.

At step 1804, the processing device may receive, from at least one of a first sensor, a first computing device, and the first electromechanical machine, first data pertaining to the first user. While the first user performs the first treatment session, the first data may be received during a first interaction with the first user. In some embodiments, the first interaction may be facilitated by a natural language processing agent executed by an artificial intelligence engine.

The natural language processing agent may be implemented as computer instructions stored on one or more memory device and executed by one or more processing devices. The natural language processing agent may include one or more computer-implemented models (e.g., large language models) that receive (via one or more input peripherals (e.g., keyboard, mouse, touchscreen, microphone, etc.)), understand, interpret, and generate human language. The natural language processing agent may interact with the user via text or speech, enabling the agent to perform tasks, such as answering questions, translating languages, summarizing text, generating human-like responses, and receiving feedback to a query (e.g., “What is your pain level?”).

In some embodiments, the one or more computer-implemented models may perform natural language processing by executing one or more functions related to tokenization (breaking down text into individual words or units), parsing (analyzing grammatical structure of sentences), lemmatization/stemming (reducing words to their base or root form), named entity recognition (identifying and classifying named entities like people, organizations, and locations), sentiment analysis (determining the emotional tone of text), machine translation (converting text from one language to another), and the like.

The first data may include quantitative data, qualitative data, or both. The quantitative data may include a revolutions per minute, a pressure, a speed, a range of motion, or some combination thereof. The qualitative data may include at least a pain level of the user, a mood of the user, a biometric datum of the user, a vital sign of the user, or some combination thereof.

At step 1806, the processing device may, based on the first data, generate, using one or more large language models executed by the artificial intelligence engine, one or more first modifications to one or more first operating parameters of the first treatment session. The one or more first modifications may increase or decrease the one or more first operating parameters at a first certain minimum rate to enable a first gradually monotonically increasing progression associated with the user. In some embodiments, the one or more first operating parameters may include a resistance applied by the electromechanical machine, a speed of the electromechanical machine, a range of motion provided by the electromechanical machine, or some combination thereof.

At step 1808, the processing device may cause, using the one or more first modifications to the one or more first operating parameters of the first treatment session, the first electromechanical machine to be controlled. In some embodiments, the processing device may cause, while the user performs the first treatment session, presentation on a display or emission of audio. The presentation and the audio may be related to the one or more first modifications of the one or more first operating parameters.

In some embodiments, the processing device may, based on the first data, determine, using the natural language processing agent and the one or more large language models, actual physical pain experienced by the user and non-physical pain experienced by the user. In some embodiments, the processing device may cause the natural language processing agent to receive the first data at a first time by interacting with the user prior to the first treatment session, at a second time during the first treatment session, and at a third time after the first treatment session. In some embodiments, the non-physical pain experienced by the user may include feared, anticipated, and/or emotional pain.

In some embodiments, the processing device may determine, based on the first data received at the first time, the second time, and/or the third time, the actual physical pain experienced by the user. In some embodiments, based on the determined actual physical pain experienced by the user, the processing device may modify the first treatment session to enable optimizing for a more desired result, a more desired recovery rate, or both.

In some embodiments, one or more computer-implemented models (e.g., machine learning models) may be trained with labeled input (e.g., user-reported pain levels at the three times) mapped to output (e.g., actual physical pain experienced by the user associated with the quantitative data and the qualitative data). In some embodiments, the actual physical pain may be determined based on stored correlations between actual pain levels of users and one or more combinations of a progression state of the user, recovery state of the user, a recovery level of the user, a medical procedure performed on the user, a user characteristic (e.g., psychographic and/or demographic information of the user), a medical history of the user, and the like.

In some embodiments, the processing device may determine, based on the first data received at the first time, the second time, and/or the third time, the non-physical pain experienced by the user. In some embodiments, based on the determined non-physical pain of the user, the processing device may modify the first treatment session to enable optimizing for a more desired result, a more desired recovery rate, or both.

In some embodiments, one or more computer-implemented models (e.g., machine learning models) may be trained with labeled input (e.g., user reported pain levels at the three times) mapped to output (e.g., non-physical pain experienced by the user associated with the quantitative data and the qualitative data). In some embodiments, the actual physical pain may be determined based on stored correlations between actual pain levels of users and one or more combinations of a progression state of the user, recovery state of the user, a recovery level of the user, a medical procedure performed on the user, a user characteristic (e.g., psychographic and/or demographic information of the user), a medical history of the user, and the like.

In some embodiments, the processing device may initiate, using a second electromechanical machine, a second treatment session defined by a second treatment plan for a second user. The processing device may receive, from a second sensor, a second computing device, the second electromechanical machine, second data pertaining to the second user. While the second user performs the second treatment session, the second data may be received during a second interaction with the second user. The second interaction may be facilitated by the natural language processing agent. In some embodiments, based on the second data, the processing device may generate, using the one or more large language models executed by the artificial intelligence engine, one or more second modifications to one or more second operating parameters of the second treatment session. The one or more modifications may increase or decrease the one or more second operating parameters at a second certain minimum rate to enable a second gradually monotonically increasing progression associated with the user. In some embodiments, the processing device may cause, using the one or more second modifications to the one or more second operating parameters of the second treatment session, the second electromechanical machine to be controlled. In some embodiments, the processing device may determine, by comparing a first result associated with the first user and a second result associated with the second user, which of the one or more first modifications and the one or more second modifications produced a more desired result, a more desired recovery rate, or both.

Consistent with the above disclosure, the examples of systems and methods enumerated in the following clauses are specifically contemplated and are intended as a non-limiting set of examples.
Clause 1.27 A computer-implemented method comprising:
   initiating, using a first electromechanical machine, a first treatment session defined by a first treatment plan for a first user;
   receiving, from at least one of a first sensor, a first computing device, and the first electromechanical machine, first data pertaining to the first user, wherein, while the first user performs the first treatment session, the first data is received during a first interaction with the first user, wherein the first interaction is facilitated by a natural language processing agent executed by an artificial intelligence engine;
   based on the first data, generating, using one or more large language models executed by the artificial intelligence engine, one or more first modifications to one or more first operating parameters of the first treatment session, wherein the one or more first modifications increase or decrease the one or more first operating parameters at a first certain minimum rate to enable a first gradually monotonically increasing progression associated with the user; and
   causing, using the one or more first modifications to the one or more first operating parameters of the first treatment session, the first electromechanical machine to be controlled.
Clause 2.27 The computer-implemented method of clause 1.27, further comprising:
   initiating, using a second electromechanical machine, a second treatment session defined by a second treatment plan for a second user;
   receiving, from a second sensor, a second computing device, the second electromechanical machine, second data pertaining to the second user, wherein, while the second user performs the second treatment session, the second data is received during a second interaction with the second user, wherein the second interaction is facilitated by the natural language processing agent;
   based on the second data, generating, using the one or more large language models executed by the artificial intelligence engine, one or more second modifications to one or more second operating parameters of the second treatment session, wherein the one or more second modifications increase or decrease the one or more second operating parameters at a second certain minimum rate to enable a second gradually monotonically increasing progression associated with the user; and
   causing, using the one or more second modifications to the one or more second operating parameters of the second treatment session, the second electromechanical machine to be controlled.
Clause 3.27 The computer-implemented method of clause 2.27, further comprising determining, by comparing a first result associated with the first user and a second result associated with the second user, which of the one or more first modifications and the one or more second modifications produced a more desired result, a more desired recovery rate, or both.
Clause 4.27 The computer-implemented method of clause 1.27, wherein the first data comprises quantitative data, qualitative data, or both, and wherein the quantitative data comprises a revolutions per minute, a pressure, a speed, a range of motion, or some combination thereof, and the qualitative data comprises at least a pain level of the user.
Clause 5.27 The computer-implemented method of clause 1.27, further comprising, based on the first data, determining, using the natural language processing agent and the one or more large language models, actual physical pain experienced by the user and non-physical pain experienced by the user.
Clause 6.27 The computer-implemented method of clause 5.27, further comprising causing the natural language processing agent to receive the first data at a first time by interacting with the user prior to the first treatment session, at a second time during the first treatment session, and at a third time after the first treatment session.
Clause 7.27 The computer-implemented method of clause 6.27, further comprising determining, based on the first data received at the first time, the second time, and the third time, the actual physical pain experienced by the user.
Clause 8.27 The computer-implemented method of clause 6.27, further comprising determining, based on the first data received at the first time, the second time, and the third time, the non-physical pain experienced by the user.
Clause 9.27 The computer-implemented method of clause 7.27, further comprising, based on the determined actual physical pain experienced by the user, modifying the first treatment session to enable optimizing for a more desired result, a more desired recovery rate, or both.
Clause 10.27 The computer-implemented method of clause 8.27, further comprising, based on the non-physical pain experienced by the user, modifying the first treatment session to enable optimizing for a more desired result, a more desired recovery rate, or both.
Clause 11.27 The computer-implemented method of clause 1.27, further comprising causing, while the user performs the first treatment session, presentation on a display or emission of audio, wherein the presentation and the audio are related to the one or more first modifications of the one or more first operating parameters.
Clause 12.27 The computer-implemented method of clause 1.27, wherein the one or more first operating parameters comprise a resistance applied by the electromechanical machine, a speed of the electromechanical machine, a range of motion provided by the electromechanical machine, or some combination of both.
Clause 13.27 A tangible, non-transitory computer-readable medium storing instructions that, when executed, cause one or more processing devices to:
   initiate, using a first electromechanical machine, a first treatment session defined by a first treatment plan for a first user;
   receive, from at least one of a first sensor, a first computing device, and the first electromechanical machine, first data pertaining to the first user, wherein, while the first user performs the first treatment session, the first data is received during a first interaction with the first user, wherein the first interaction is facilitated by a natural language processing agent executed by an artificial intelligence engine;
   based on the first data, generate, using one or more large language models executed by the artificial intelligence engine, one or more first modifications to one or more first operating parameters of the first treatment session, wherein the one or more first modifications increase or decrease the one or more first operating parameters at a first certain minimum rate to enable a first gradually monotonically increasing progression associated with the user; and
   cause, using the one or more first modifications to the one or more first operating parameters of the first treatment session, the first electromechanical machine to be controlled.
Clause 14.27 The computer-readable medium of clause 13.27, wherein the one or more processing devices are configured to:
   initiate, using a second electromechanical machine, a second treatment session defined by a second treatment plan for a second user;
   receive, from a second sensor, a second computing device, the second electromechanical machine, second data pertaining to the second user, wherein, while the second user performs the second treatment session, the second data is received during a second interaction with the second user, wherein the second interaction is facilitated by the natural language processing agent;
   based on the second data, generate, using the one or more large language models executed by the artificial intelligence engine, one or more second modifications to one or more second operating parameters of the second treatment session, wherein the one or more second modifications increase or decrease the one or more second operating parameters at a second certain minimum rate to enable a second gradually monotonically increasing progression associated with the user; and
   cause, using the one or more second modifications to the one or more second operating parameters of the second treatment session, the second electromechanical machine to be controlled.
Clause 15.27 The computer-readable medium of clause 14.27, wherein the one or more processing devices are configured to determine, by comparing a first result associated with the first user and a second result associated with the second user, which of the one or more first modifications and the one or more second modifications produced a more desired result, a more desired recovery rate, or both.
Clause 16.27 The computer-readable medium of clause 13.27, wherein the first data comprises quantitative data, qualitative data, or both, and wherein the quantitative data comprises a revolutions per minute, a pressure, a speed, a range of motion, or some combination thereof, and the qualitative data comprises at least a pain level of the user.
Clause 17.27 The computer-readable medium of clause 13.27, wherein the one or more processing devices are configured to, based on the first data, determine, using the natural language processing agent and the one or more large language models, actual physical pain experienced by the user and non-physical pain experienced by the user.
Clause 18.27 The computer-readable medium of clause 17.27, wherein the one or more processing devices are configured to cause the natural language processing agent to receive the first data at a first time by interacting with the user prior to the first treatment session, at a second time during the first treatment session, and at a third time after the first treatment session.
Clause 19.27 The computer-readable medium of clause 18.27, wherein the one or more processing devices are configured to determine, based on the first data received at the first time, the second time, and the third time, the actual physical pain experienced by the user.
Clause 20.27 A system comprising:
   one or more memory devices storing instructions; and
   one or more processing devices communicatively coupled to the one or more memory devices, wherein the one or more processing devices execute the instructions to:
   initiate, using a first electromechanical machine, a first treatment session defined by a first treatment plan for a first user;
   receive, from at least one of a first sensor, a first computing device, and the first electromechanical machine, first data pertaining to the first user, wherein, while the first user performs the first treatment session, the first data is received during a first interaction with the first user, wherein the first interaction is facilitated by a natural language processing agent executed by an artificial intelligence engine;
   based on the first data, generate, using one or more large language models executed by the artificial intelligence engine, one or more first modifications to one or more first operating parameters of the first treatment session, wherein the one or more first modifications increase or decrease the one or more first operating parameters at a first certain minimum rate to enable a first gradually monotonically increasing progression associated with the user; and
   cause, using the one or more first modifications to the one or more first operating parameters of the first treatment session, the first electromechanical machine to be controlled.
      Systems and Methods for Determining and Performing Interventions Using Objective-Based Measurements and Specifying the Interventions Using a Medical Description Language that is Capable of Serving as Input to a Large Language Model

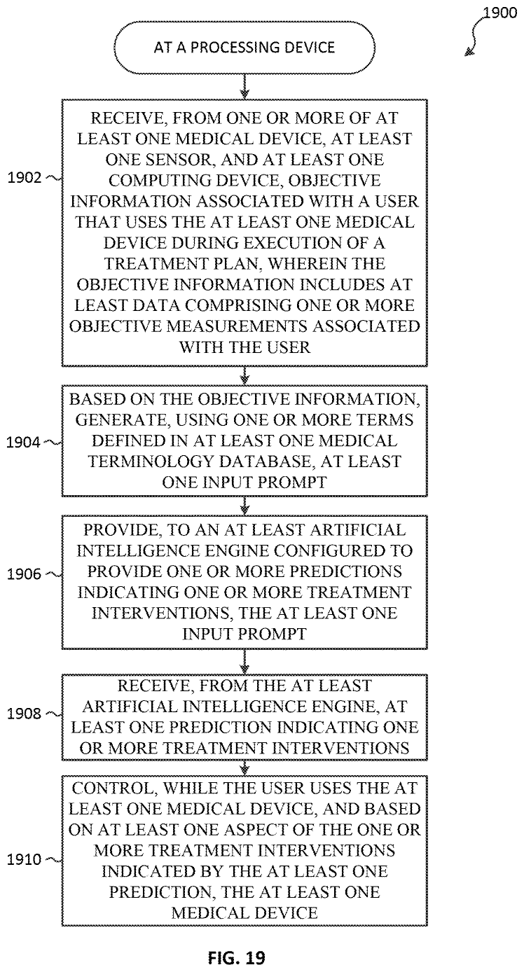

FIG. 19 is a flow diagram generally illustrating a method 1900 for determining and performing interventions using objective-based measurements (e.g., objective information) and specifying the interventions using a medical description language that is capable of serving as input to a large language model, according to the principles of the present disclosure. “Objective information” may include, without limitation, factual, observable, and measurable information that may not be influenced by personal feelings or opinions, such as sensor data obtained from one or more sensors associated with a patient or user, user device, treatment device, etc. The objective information may include information determined through various interactions with an electromechanical machine (e.g., the treatment device), sensor, or user device, including information associated with adjusting an operating parameter of the treatment device (e.g., adjusting a pedal radius setting, a resistance setting, a target RPM, a target ROM (range of motion), etc.), feedback received regarding the first treatment plan, objective information input by the user and/or medical professional (e.g. treatment dates, durations, schedules, missed or performed treatment sessions, numbers of sessions, etc.), and/or other observable and measurable data.

As used herein, “objective information” is contrasted with subjective or qualitative information, which may include subjective narrations or other information provided by various users, medical professionals, etc. For example, subjective narrations may be provided by a therapist or other medical professional based on observations and personal assessments of a patient's performance, progress, pain levels, and so on. Such narrations may be provided in a non-standardized format or nomenclature that is not queryable (i.e., by a computing device, AI/ML model, etc.). In some examples, qualitative information may include patient reported outcomes, reported pain level of the user, and/or any other suitable qualitative information.

Conversely, systems and methods according to the present disclosure are configured to obtain and store objective information, in real time, corresponding to sensor data generated while users actually perform various treatment plans using respective treatment devices. The sensor data that is stored as the objective information may correspond specifically to data that is relevant to diagnosis, rehabilitation, prehabilitation or other requirements for a particular rehabilitation treatment or protocol. Further, the data is stored in a standardized, queryable format (e.g., in a database structured for real time AI/ML access, retrieval, and analysis). In some examples, qualitative information (including patient-reported outcomes or other subjective narratives) may be converted to objective information (“second objective information”) that can be stored in the standardized, queryable format.

The method 1900 can be performed by processing logic that may include hardware (circuitry, dedicated logic, etc.), software (such as is run on a general-purpose computer system or a dedicated machine), or a combination of both. The method 1900 and/or each of its individual functions, routines, subroutines, methods (as the term is used in object-oriented design/programming) or operations may be performed by one or more processing devices of a computing device (e.g., any component of FIG. 1, such as the assistant interface 94, patient interface 50, and/or server 30 executing the artificial intelligence engine 11, training engine 9, machine learning models 13, etc.) implementing the method 1900. The method 1900 may be implemented as computer instructions stored on a memory device and able to be executed by the one or more processing devices. In certain implementations, the method 1900 may be performed by a single processing thread. Alternatively, the method 1900 may be performed by two or more processing threads, each thread implementing one or more individual functions, routines, subroutines, methods or operations of the methods. In some embodiments, one or more accelerators may be used to increase the performance of a processing device by offloading various functions, routines, subroutines, methods or operations from the processing device.

For simplicity of explanation, the method 1900 is depicted in FIG. 19 and described as a series of operations. However, operations in accordance with this disclosure can occur in various orders and/or concurrently, and/or with other operations not presented and described herein. For example, the operations depicted in the method 1900 in FIG. 19 may occur in combination with any other operation of any other method disclosed herein. Furthermore, not all illustrated operations may be required to implement the method 1900 in accordance with the disclosed subject matter. In addition, those skilled in the art will understand and appreciate that the method 1900 could alternatively be represented as a series of interrelated states via a state diagram or event diagram.

In some embodiments, one or more computer-implemented models illustrated in FIG. 19 (e.g., machine learning models, neural networks, expert systems, etc.) may be generated and trained by the artificial intelligence engine 11 and/or the training engine 9 to perform one or more of the operations of the methods described herein. For example, to perform the one or more operations, the processing device may execute the one or more computer-implemented models. In some embodiments, the one or more computer-implemented models may be iteratively retrained to select different features capable of enabling optimization of output. The features that may be modified may include a number of nodes included in each layer of the models, an objective function executed at each node, a number of layers, various weights associated with outputs of each node, and the like.

At step 1902, the method 1900 receives, from one or more of at least one medical device, at least one sensor, and at least one computing device, objective information associated with a user that uses the at least one medical device during execution of a treatment plan or at least a portion of the treatment plan. The objective information may include at least data comprising one or more objective measurements associated with the user. The one or more objective measurements may include, without limitation, any measurement described herein, or any other suitable measurement.

The at least one medical device may include at least one treatment device, at least one life support device, at least one therapeutic support device, at least one other suitable device, and/or the combination thereof.

In some embodiments, the at least one life support device may include at least one respiratory support device. The at least one respiratory support device may include one or more mechanical ventilators, one or more extracorporeal membrane oxygenation (ECMO) systems, one or more non-invasive positive pressure ventilation devices (e.g., including, but not limited to, a continuous positive airwave pressure (CPAP) machine, a BiPAP machine, and/or the like), one or more other suitable devices, and/or a combination thereof.

In some embodiments, the at least one life support device may include at least one cardiovascular support device. The at least one cardiovascular support device may include one or more intra-aortic balloon pumps (IABPs), one or more ventricular assist devices (VADs), one or more total artificial hearts (TAHs), one or more pacemakers, one or more implantable cardioverter-defibrillators (ICDs), one or more other suitable devices, and/or a combination thereof.

In some embodiments, the at least one life support device may include at least one neurological support device. The at least one neurological support device may include one or more intracranial pressure monitors, one or more therapeutic hypothermia control systems, one or more other suitable devices, and/or a combination thereof.

In some embodiments, the at least one life support device may include at least one renal support system. The at least one renal support system may include one or more hemodialysis machines, one or more continuous renal replacement therapy (CRRT) devices, one or more other suitable devices, and/or a combination thereof.

In some embodiments, the at least one life support device may include at least one nutritional and metabolic support device. The at least one nutritional and metabolic support device may include one or more total parenteral nutrition (TPN) systems, one or more enteral feeding tubes, one or more other suitable devices, and/or a combination thereof.

In some embodiments, the at least one life support device may include at least one adjunct critical care system. The at least one adjunct critical care system may include one or more infusion pumps for life-sustaining medications, one or more external defibrillators, one or more automated mechanical chest compression devices, one or more other suitable devices, and/or a combination thereof.

In some embodiments, the at least one life support device may include at least one internal or invasive life support device. The at least one internal or invasive life support device may include one or more an insulin pumps, one or more ventricular assist pumps, one or more other suitable devices, and/or a combination thereof.

In some embodiments, the at least one therapeutic support device may include a standard hospital bed, a rotational therapy bed (e.g., such as a kinetic bed), an air fluidized therapy bed, a tilt table, an intensive care unit (ICU) stretcher, a procedural stretcher, a reclining chair, a specialized neurological positioning bed, a supportive wheelchair for minimally conscious patients, one or more other suitable devices, and/or a combination thereof.

At 1904, the method 1900, based on the objective information, generates, using one or more terms (e.g., which may include keywords, prompts, text strings, tokens, syntactic segments, phrases, clauses, semantic segments or terms, and/or the like) defined in at least one medical terminology database, at least one input prompt. In some embodiments, at least one of the one or more medical terminology databases may be configured to correlate one or more medical terms with one or more medical definitions. Additionally, or alternatively, the method 1900 may include using one or more inputs received from the at least one medical terminology database to generate the at least one prompt.

At 1906, the method 1900 provides, to an at least artificial intelligence engine configured to provide one or more predictions indicating one or more treatment interventions, the at least one input prompt. The artificial intelligence engine may use one or more trained computer-implemented models to generate the at least one prediction indicating the one or more treatment interventions.

At 1908, the method 1900 receives, from the at least artificial intelligence engine, at least one prediction indicating one or more treatment interventions. The one or more treatment interventions indicated by the at least one prediction may include adjusting at least one aspect of at least one of the at least one medical device, and/or any other suitable intervention.

At 1910, the method 1900 controls, while the user uses the at least one medical device, and based on at least one aspect of the one or more treatment interventions indicated by the at least one prediction, the at least one medical device.
Clause 1.23 A computer-implemented method comprising: receiving, from one or more of at least one medical device, at least one sensor, and at least one computing device, objective information associated with a user that uses the at least one medical device during execution of a treatment plan, wherein the objective information includes at least data comprising one or more objective measurements associated with the user; based on the objective information, generating, using one or more terms defined in at least one medical terminology database, at least one input prompt; providing, to an at least artificial intelligence engine configured to provide one or more predictions indicating one or more treatment interventions, the at least one input prompt; receiving, from the at least artificial intelligence engine, at least one prediction indicating one or more treatment interventions; and controlling, while the user uses the at least one medical device, and based on at least one aspect of the one or more treatment interventions indicated by the at least one prediction, the at least one medical device.
Clause 2.23 The computer-implemented method of any of the clauses herein, wherein, based on diagnostic requirements associated with the treatment plan, the objective information is stored.
Clause 3.23 The computer-implemented method of any of the clauses herein, wherein the objective information is stored in a standardized format.
Clause 4.23 The computer-implemented method of any of the clauses herein, wherein the artificial intelligence engine uses one or more trained computer-implemented models to generate the at least one prediction indicating the one or more treatment interventions.
Clause 5.23 The computer-implemented method of any of the clauses herein, wherein the at least one medical device includes at least one of: at least one treatment device, at least one life support device, and at least one therapeutic support device.
Clause 6.23 The computer-implemented method of any of the clauses herein, wherein the at least one treatment device includes at least one pedal.
Clause 7.23 The computer-implemented method of any of the clauses herein, wherein the at least one life support device includes at least one respiratory support device.
Clause 8.23 The computer-implemented method of any of the clauses herein, wherein the at least one respiratory support device includes at least one of: one or more mechanical ventilators; one or more extracorporeal membrane oxygenation (ECMO) systems; and one or more non-invasive positive pressure ventilation devices.
Clause 9.23 The computer-implemented method of any of the clauses herein, wherein the at least one life support device includes at least one cardiovascular support device.
Clause 10.23 The computer-implemented method of any of the clauses herein, wherein the at least one cardiovascular support device includes at least one of: one or more intra-aortic balloon pumps (IABPs); one or more ventricular assist devices (VADs); one or more total artificial hearts (TAHs); one or more pacemakers; and one or more implantable cardioverter-defibrillators (ICDs).
Clause 11.23 The computer-implemented method of any of the clauses herein, wherein the at least one life support device includes at least one neurological support device.
Clause 12.23 The computer-implemented method of any of the clauses herein, wherein the at least one neurological support device includes at least one of: one or more intracranial pressure monitors; and one or more therapeutic hypothermia control systems.
Clause 13.23 The computer-implemented method of any of the clauses herein, wherein the at least one life support device includes at least one renal support system.
Clause 14.23 The computer-implemented method of any of the clauses herein, wherein the at least one renal support system includes at least one of: one or more hemodialysis machines; and one or more continuous renal replacement therapy (CRRT) devices.
Clause 15.23 The computer-implemented method of any of the clauses herein, wherein the at least one life support device includes at least one nutritional and metabolic support device.
Clause 16.23 The computer-implemented method of any of the clauses herein, wherein the at least one nutritional and metabolic support device includes at least one of: one or more total parenteral nutrition (TPN) systems; and one or more enteral feeding tubes.
Clause 17.23 The computer-implemented method of any of the clauses herein, wherein the at least one life support device includes at least one adjunct critical care system.
Clause 18.23 The computer-implemented method of any of the clauses herein, wherein the at least one adjunct critical care system includes at least one of: one or more infusion pumps for life-sustaining medications; one or more external defibrillators; and one or more automated mechanical chest compression devices.
Clause 19.23 The computer-implemented method of any of the clauses herein, wherein the at least one life support device includes at least one internal or invasive life support device.
Clause 20.23 The computer-implemented method of any of the clauses herein, wherein the at least one internal or invasive life support device includes at least one of: one or more an insulin pumps; and one or more ventricular assist pumps.
Clause 21.23 The computer-implemented method of any of the clauses herein, wherein the at least one therapeutic support device includes: a standard hospital bed; a rotational therapy bed, such as a kinetic bed; an air fluidized therapy bed; a tilt table; an intensive care unit (ICU) stretcher; a procedural stretcher; a reclining chair; a specialized neurological positioning bed; and a supportive wheelchair for minimally conscious patients.
Clause 22.23 The computer-implemented method of any of the clauses herein, wherein one of the one or more medical terminology databases correlate one or more medical terms with one or more medical definitions.
Clause 23.23 The computer-implemented method of any of the clauses herein, wherein the one or more treatment interventions indicated by the at least one prediction includes adjusting at least one aspect of at least one of the at least one medical device.
Clause 24.23 The computer-implemented method of any of the clauses herein, further comprising: receiving qualitative information corresponding to at least the user; converting the qualitative information to second objective information; and storing the second objective information in a queryable data format.
Clause 25.23 The computer-implemented method of any of the clauses herein, further comprising generating, using the one or more terms defined in the medical terminology database, the at least one input prompt, further based on the second objective information.
Clause 26.23 The computer-implemented method of any of the clauses herein, wherein the qualitative information comprises a pain level of the user.
Clause 27.23 A system, comprising: memory that stores a plurality of instructions; and one or more processing devices configured to execute the plurality of instructions, wherein executing the plurality of instructions causes the one or more processing devices to: receive, from one or more of at least one medical device, at least one sensor, and at least one computing device, objective information associated with a user that uses the at least one medical device during execution of a treatment plan, wherein the objective information includes at least data comprising one or more objective measurements associated with the user; based on the objective information, generate, using one or more terms defined in at least one medical terminology database, at least one input prompt; provide, to an at least artificial intelligence engine configured to provide one or more predictions indicating one or more treatment interventions, the at least one input prompt; receive, from the at least artificial intelligence engine, at least one prediction indicating one or more treatment interventions; and control, while the user uses the at least one medical device, and based on at least one aspect of the one or more treatment interventions indicated by the at least one prediction, the at least one medical device.
Clause 28.23 The system of any of the clauses herein, wherein, based on diagnostic requirements associated with the treatment plan, the objective information is stored.
Clause 29.23 The system of any of the clauses herein, wherein the objective information is stored in a standardized format.
Clause 30.23 The system of any of the clauses herein, wherein the artificial intelligence engine uses one or more trained computer-implemented models to generate the at least one prediction indicating the one or more treatment interventions.
Clause 31.23 The system of any of the clauses herein, wherein the at least one medical device includes at least one of: at least one treatment device, at least one life support device, and at least one therapeutic support device.
Clause 32.23 The system of any of the clauses herein, wherein the at least one treatment device includes at least one pedal.
Clause 33.23 The system of any of the clauses herein, wherein the at least one life support device includes at least one respiratory support device.
Clause 34.23 The system of any of the clauses herein, wherein the at least one respiratory support device includes at least one of: one or more mechanical ventilators; one or more extracorporeal membrane oxygenation (ECMO) systems; and one or more non-invasive positive pressure ventilation devices.
Clause 35.23 The system of any of the clauses herein, wherein the at least one life support device includes at least one cardiovascular support device.
Clause 36.23 The system of any of the clauses herein, wherein the at least one cardiovascular support device includes at least one of: one or more intra-aortic balloon pumps (IABPs); one or more ventricular assist devices (VADs); one or more total artificial hearts (TAHs); one or more pacemakers; and one or more implantable cardioverter-defibrillators (ICDs).
Clause 37.23 The system of any of the clauses herein, wherein the at least one life support device includes at least one neurological support device.
Clause 38.23 The system of any of the clauses herein, wherein the at least one neurological support device includes at least one of: one or more intracranial pressure monitors; and one or more therapeutic hypothermia control systems.
Clause 39.23 The system of any of the clauses herein, wherein the at least one life support device includes at least one renal support system.
Clause 40.23 The system of any of the clauses herein, wherein the at least one renal support system includes at least one of: one or more hemodialysis machines; and one or more continuous renal replacement therapy (CRRT) devices.
Clause 41.23 The system of any of the clauses herein, wherein the at least one life support device includes at least one nutritional and metabolic support device.
Clause 42.23 The system of any of the clauses herein, wherein the at least one nutritional and metabolic support device includes at least one of: one or more total parenteral nutrition (TPN) systems; and one or more enteral feeding tubes.
Clause 43.23 The system of any of the clauses herein, wherein the at least one life support device includes at least one adjunct critical care system.
Clause 44.23 The system of any of the clauses herein, wherein the at least one adjunct critical care system includes at least one of: one or more infusion pumps for life-sustaining medications; one or more external defibrillators; and one or more automated mechanical chest compression devices.
Clause 45.23 The system of any of the clauses herein, wherein the at least one life support device includes at least one internal or invasive life support device.
Clause 46.23 The system of any of the clauses herein, wherein the at least one internal or invasive life support device includes at least one of: one or more an insulin pumps; and one or more ventricular assist pumps.
Clause 47.23 The system of any of the clauses herein, wherein the at least one therapeutic support device includes: a standard hospital bed; a rotational therapy bed, such as a kinetic bed; an air fluidized therapy bed; a tilt table; an intensive care unit (ICU) stretcher; a procedural stretcher; a reclining chair; a specialized neurological positioning bed; and a supportive wheelchair for minimally conscious patients.
Clause 48.23 The system of any of the clauses herein, wherein one of the one or more medical terminology databases correlate one or more medical terms with one or more medical definitions.
Clause 49.23 The system of any of the clauses herein, wherein the one or more treatment interventions indicated by the at least one prediction includes adjusting at least one aspect of at least one of the at least one medical device.
Clause 50.23 The system of any of the clauses herein, wherein the plurality of instructions further cause the one or more processing devices to: receiving qualitative information corresponding to at least the user; converting the qualitative information to second objective information; and storing the second objective information in a queryable data format.
Clause 51.23 The system of any of the clauses herein, wherein the plurality of instructions further cause the one or more processing devices to generating, using the one or more terms defined in the medical terminology database, the at least one input prompt, further based on the second objective information.
Clause 52.23 The system of any of the clauses herein, wherein the qualitative information comprises a pain level of the user.
   Method and System for Using AI-Driven Interventions Via Optimization of Post-Surgical Rehabilitation for Users with External Life Support Devices where the Rehabilitation Uses Selective Data Signature-Based Data Injection

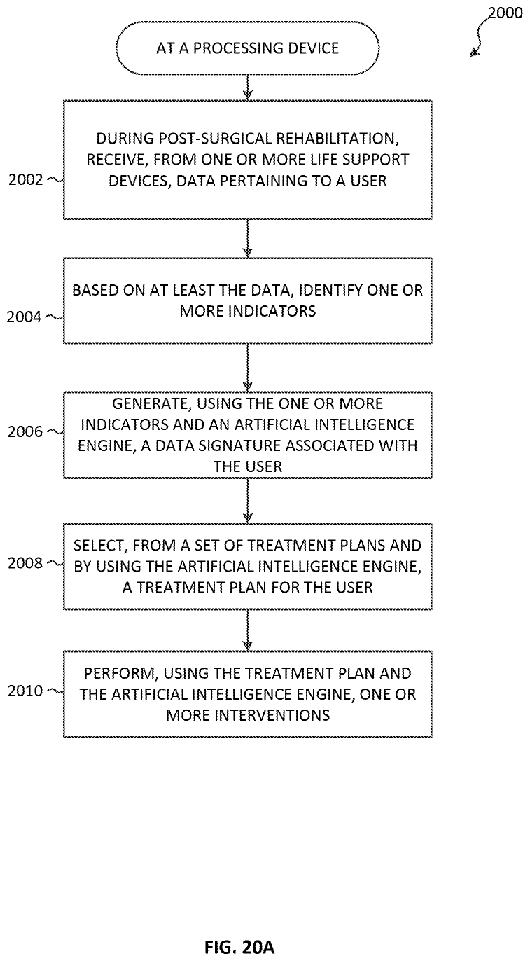

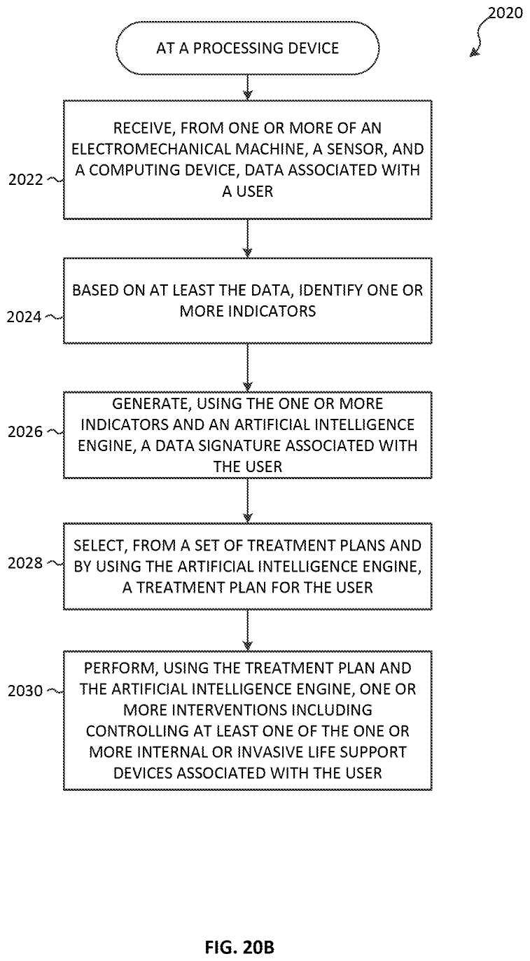

FIG. 20A is a flow diagram generally illustrating a method 2000 for using AI-driven devices where the rehabilitation uses selective data signature-based data injection according to principles of the present disclosure. The method 2000 is performed by processing logic that may include hardware (circuitry, dedicated logic, etc.), software (such as is run on a general-purpose computer system or a dedicated machine), or a combination of both. The method 2000 and/or each of its individual functions, routines, subroutines, methods (as the term is used in object-oriented programming), or operations may be performed by one or more processing devices of a computing device (e.g., any component of FIG. 1, such as the assistant interface 94, patient interface 50, and/or server 30 executing the artificial intelligence engine 11, training engine 9, machine learning models 13, etc.) implementing the method 2000. The method 2000 may be implemented as computer instructions stored on a memory device and able to be executed by the one or more processing devices. In certain implementations, the method 2000 may be performed by a single processing thread. Alternatively, the method 2000 may be performed by two or more processing threads, each thread implementing one or more individual functions, routines, subroutines, methods or operations of the methods. In some embodiments, one or more accelerators may be used to increase the performance of a processing device by offloading various functions, routines, subroutines, methods or operations from the processing device.

For simplicity of explanation, the method 2000 is depicted in FIG. 20A and described as a series of operations. However, operations in accordance with this disclosure can occur in various orders and/or concurrently, and/or with other operations not presented and described herein. For example, the operations depicted in the method 2000 in FIG. 20A may occur in combination with any other operation of any other method disclosed herein. Furthermore, not all illustrated operations may be required to implement the method 2000 in accordance with the disclosed subject matter. In addition, those skilled in the art will understand and appreciate that the method 2000 could alternatively be represented as a series of interrelated states via a state diagram or event diagram.

In some embodiments, one or more computer-implemented models (e.g., machine learning models, neural networks, expert systems, etc.) may be generated and trained by the artificial intelligence engine 11 and/or the training engine 9 to perform one or more of the operations of the methods described herein. For example, to perform the one or more operations, the processing device may execute the one or more computer-implemented models. In some embodiments, the one or more computer-implemented models may be iteratively retrained to select different features capable of enabling optimization of output. The features that may be modified may include a number of nodes included in each layer of the models, an objective function executed at each node, a number of layers, various weights associated with outputs of each node, and the like.

At step 2002, the processing device may, during post-surgical rehabilitation, receive, from one or more life support devices, data pertaining to a user. In some embodiments, the one or more life support devices may include an extracorporeal membrane oxygenation (ECMO), a ventilator, or some combination thereof. In some embodiments, the data may be further received from one or more of an electromechanical machine, a computing device, a sensor, or some combination thereof. In some embodiments, the computing device may include a wearable device, a smartphone, a tablet, an electrode, a device including a user-attachable cable, or some combination thereof.

In some embodiments, the life support devices may include any apparatus or system designed to sustain or replace vital physiological functions in critically ill or incapacitated patients, including but not limited to: respiratory support devices such as mechanical ventilators, extracorporeal membrane oxygenation (ECMO) systems, and non-invasive positive pressure ventilation devices (e.g., CPAP and BiPAP); cardiovascular support devices such as intra-aortic balloon pumps (IABPs), ventricular assist devices (VADs), total artificial hearts (TAHs), pacemakers, and implantable cardioverter-defibrillators (ICDs); neurological support devices including intracranial pressure monitors and therapeutic hypothermia control systems; renal support systems such as hemodialysis machines and continuous renal replacement therapy (CRRT) equipment; nutritional and metabolic support devices including total parenteral nutrition (TPN) systems and enteral feeding tubes; and adjunct critical care systems such as infusion pumps for life-sustaining medications, external defibrillators, and automated mechanical chest compression devices.

At step 2004, the processing device, based on at least the data, may identify one or more indicators. In some embodiments, the one or more indicators may include a pain measurement, a performance measurement (e.g., measurement of revolutions per minute, a session pedaling time, a pedaling speed, etc.), a user characteristic (e.g., demographic, psychographic, medical history, etc.), and the like.

At step 2006, the processing device may generate, using the one or more indicators and an artificial intelligence engine, a data signature associated with the user. The artificial intelligence engine may be trained with training data including inputs (e.g., indicators) and labeled outputs (e.g., data signatures). In some embodiments, the artificial intelligence engine may train one or more computer-implemented models (e.g., machine learning models) to generate the unique data signature.

At step 2008, the processing device may select, from a set of treatment plans and by using the artificial intelligence engine, a treatment plan for the user. The artificial intelligence engine may select the treatment plan by correlating the data signature with at least one of one or more data signatures obtained from one or more evidence-based selection protocols. In some embodiments, the treatment plan may include modifying one or more operating parameters of an electromechanical machine associated with one or more exercises for the user to perform by using the electromechanical machine. In some embodiments, the artificial intelligence engine may train one or more computer-implemented models (e.g., machine learning models) to select the treatment plan.

In some embodiments, correlation may refer to a process for establishing the relationship between two variables, data signatures, properties, values, measurements, identifiers, or the like. In some embodiments, correlating may refer to any statistical relationship, whether causal or not, between two variables, statistical metrics (parametric or non-parametric), data signatures, properties, values, measurements, identifiers, or the like. In some embodiments, correlating may include correlation coefficients but is not limited to them.

At step 2010, the processing device may perform, using the treatment plan and the artificial intelligence engine, one or more interventions. In some embodiments, the artificial intelligence engine may train one or more computer-implemented models (e.g., machine learning models) to perform the one or more interventions. In some embodiments, performing the one or more interventions may include controlling, using the treatment plan, operation of an electromechanical machine. In some embodiments, the data may be received while the user is in a coma, and the electromechanical machine may be a bed. In some embodiments, the bed may include standard hospital beds, rotational therapy beds (kinetic beds), air fluidized therapy beds, tilt tables, intensive care unit (ICU) stretchers, procedural stretchers, reclining chairs, specialized neurological positioning beds, supportive wheelchairs for minimally conscious patients, and the like.

In some embodiments, the coma may be medically-induced, or due to trauma, brain injury, and the like. In some embodiments, the coma may be associated with a vegetative state. The term “coma” may in some embodiments, be short for “comatose” and in such cases, the term may refer to a deep state of unconsciousness in which a person cannot be awakened, shows no purposeful response to stimuli, and lacks sleep-wake cycles. Non-comatose may refer to a term indicating a person is not in a coma. This may include states ranging from full wakefulness to reduced but present awareness (e.g., asleep, vegetative, or minimally conscious).

A vegetative state may refer to a condition of wakefulness without awareness, where the person has sleep-wake cycles and may open their eyes or make reflexive movements but lacks purposeful interaction with the environment. Asleep may refer to a reversible, physiologic state of reduced consciousness with preserved brain activity and the ability to awaken in response to stimuli and the state of being asleep is therefore part of the normal sleep-wake cycle. Awake may refer to a state of full consciousness with awareness of self and environment, where the person is capable of purposeful response to stimuli. A minimally conscious state may refer to a condition of severely altered consciousness in which the patient demonstrates intermittent, minimal but definite behavioral evidence of awareness of self or environment.

In some embodiments, the data may be received while the user is awake, and the electromechanical machine may include at least one pedal.

In some embodiments, the one or more interventions may include executing a virtual agent on a computing device that, while the user uses an electromechanical machine, uses natural language processing or generative artificial intelligence to communicate with the user.

In some embodiments, a set of tiles may be presented on a graphical user interface of a computing device associated with a second user. In some embodiments, at least one of the set of tiles may include a multimedia feed associated with the user's performance of the treatment plan while the user is using an electromechanical machine.

Consistent with the above disclosure, the examples of systems and methods enumerated in the following clauses are specifically contemplated and are intended as a non-limiting set of examples.
Clause 1.28 A computer-implemented method comprising:
   during post-surgical rehabilitation, receiving, from one or more life support devices, data pertaining to a user;
   based on at least the data, identifying one or more indicators;
   generating, using the one or more indicators and an artificial intelligence engine, a data signature associated with the user;
   selecting, from a plurality of treatment plans and by using the artificial intelligence engine, a treatment plan for the user, wherein the artificial intelligence engine selects the treatment plan by correlating the data signature with at least one of one or more data signatures obtained from one or more evidence-based selection protocols; and
   performing, using the treatment plan and the artificial intelligence engine, one or more interventions.
Clause 2.28 The computer-implemented method of clause 1.28, wherein performing the one or more interventions comprises controlling, using the treatment plan, operation of an electromechanical machine.
Clause 3.28 The computer-implemented method of clause 2.28, wherein the data is received while the user is in a coma, and the electromechanical machine comprises a bed.
Clause 4.28 The computer-implemented method of clause 2.28, wherein the data is received while the user is awake, and the electromechanical machine comprises at least one pedal.
Clause 5.28 The computer-implemented method of clause 1.28, wherein the one or more life support devices comprise an extracorporeal membrane oxygenation (ECMO), a ventilator, or some combination thereof.
Clause 6.28 The computer-implemented method of clause 1.28, wherein the data is further received from one or more of an electromechanical machine, a computing device, a sensor, or some combination thereof.
Clause 7.28 The computer-implemented method of clause 6.28, wherein the computing device comprises a wearable device, a smartphone, a tablet, an electrode, a device comprising a user-attachable cable, or some combination thereof.
Clause 8.28 The computer-implemented method of clause 1.28, wherein the one or more interventions comprise executing a virtual agent on a computing device that, while the user uses an electromechanical machine, uses natural language processing or generative artificial intelligence to communicate with the user.
Clause 9.28 The computer-implemented method of clause 1.28, wherein the treatment plan comprises modifying one or more operating parameters of an electromechanical machine associated with one or more exercises for the user to perform by using the electromechanical machine.
Clause 10.28 The computer-implemented method of clause 1.28, wherein a plurality of tiles is presented on a graphical user interface of a computing device associated with a second user, wherein at least one of the plurality of tiles comprises a multimedia feed associated with the user's performance of the treatment plan while the user is using an electromechanical machine.
Clause 11.28 A tangible, non-transitory computer-readable medium storing instructions that, when executed, cause one or more processing devices to:
   during post-surgical rehabilitation, receive, from one or more life support devices, data pertaining to a user;
   based on at least the data, identify one or more indicators;
generate, using the one or more indicators and an artificial intelligence engine, a data signature associated with the user;
select, from a plurality of treatment plans and by using the artificial intelligence engine, a treatment plan for the user, wherein the artificial intelligence engine selects the treatment plan by correlating the data signature with at least one of one or more data signatures obtained from one or more evidence-based selection protocols; and
   perform, using the treatment plan and the artificial intelligence engine, one or more interventions.
Clause 12.28 The computer-readable medium of clause 11.28, wherein performing the one or more interventions comprises controlling, using the treatment plan, operation of an electromechanical machine.
Clause 13.28 The computer-readable medium of clause 12.28, wherein the data is received while the user is in a coma, and the electromechanical machine comprises a bed.
Clause 14.28 The computer-readable medium of clause 12.28, wherein the data is received while the user is awake, and the electromechanical machine comprises at least one pedal.
Clause 15.28 The computer-readable medium of clause 11.28, wherein the one or more life support devices comprise an extracorporeal membrane oxygenation (ECMO), a ventilator, or some combination thereof.
Clause 16.28 The computer-readable medium of clause 11.28, wherein the data is further received from one or more of an electromechanical machine, a computing device, a sensor, or some combination thereof.
Clause 17.28 The computer-readable medium of clause 16.28, wherein the computing device comprises a wearable device, a smartphone, a tablet, an electrode, a device comprising a user-attachable cable, or some combination thereof.
Clause 18.28 The computer-readable medium of clause 11.28, wherein the one or more interventions comprise executing a virtual agent on a computing device that, while the user uses an electromechanical machine, uses natural language processing or generative artificial intelligence to communicate with the user.
Clause 19.28 The computer-readable medium of clause 11.28, wherein the treatment plan comprises modifying one or more operating parameters of an electromechanical machine associated with one or more exercises for the user to perform by using the electromechanical machine.
Clause 20.28 A system comprising:
   one or more memory devices storing instructions; and
   one or more processing devices communicatively coupled to the one or more memory devices, wherein the one or more processing devices execute the instructions to:
during post-surgical rehabilitation, receive, from one or more life support devices, data pertaining to a user;
   based on at least the data, identify one or more indicators;
generate, using the one or more indicators and an artificial intelligence engine, a data signature associated with the user;
select, from a plurality of treatment plans and by using the artificial intelligence engine, a treatment plan for the user, wherein the artificial intelligence engine selects the treatment plan by correlating the data signature with at least one of one or more data signatures obtained from one or more evidence-based selection protocols; and
   perform, using the treatment plan and the artificial intelligence engine, one or more interventions.
      Method and System for Using AI-Driven Interventions Via Optimization of Rehabilitation That Uses Selective Data Signature-Based Data Injection for Users with Internal or Invasive Life Support Devices

FIG. 20B is a flow diagram generally illustrating a method 2020 for using AI-driven interventions via optimization of rehabilitation that uses selective data signature-based data injection for users with internal or invasive life support devices according to principles of the present disclosure. The method 2020 is performed by processing logic that may include hardware (circuitry, dedicated logic, etc.), software (such as is run on a general-purpose computer system or a dedicated machine), or a combination of both. The method 2020 and/or each of its individual functions, routines, subroutines, methods (as the term is used in object-oriented programming), or operations may be performed by one or more processing devices of a computing device (e.g., any component of FIG. 1, such as the assistant interface 94, patient interface 50, and/or server 30 executing the artificial intelligence engine 11, training engine 9, machine learning models 13, etc.) implementing the method 2020. The method 2020 may be implemented as computer instructions stored on a memory device and able to be executed by the one or more processing devices. In certain implementations, the method 2020 may be performed by a single processing thread. Alternatively, the method 2020 may be performed by two or more processing threads, each thread implementing one or more individual functions, routines, subroutines, methods or operations of the methods. In some embodiments, one or more accelerators may be used to increase the performance of a processing device by offloading various functions, routines, subroutines, methods or operations from the processing device.

For simplicity of explanation, the method 2020 is depicted in FIG. 20B and described as a series of operations. However, operations in accordance with this disclosure can occur in various orders and/or concurrently, and/or with other operations not presented and described herein. For example, the operations depicted in the method 2020 in FIG. 20B may occur in combination with any other operation of any other method disclosed herein. Furthermore, not all illustrated operations may be required to implement the method 2020 in accordance with the disclosed subject matter. In addition, those skilled in the art will understand and appreciate that the method 2020 could alternatively be represented as a series of interrelated states via a state diagram or event diagram.

In some embodiments, one or more computer-implemented models (e.g., machine learning models, neural networks, expert systems, etc.) may be generated and trained by the artificial intelligence engine 11 and/or the training engine 9 to perform one or more of the operations of the methods described herein. For example, to perform the one or more operations, the processing device may execute the one or more computer-implemented models. In some embodiments, the one or more computer-implemented models may be iteratively retrained to select different features capable of enabling optimization of output. The features that may be modified may include a number of nodes included in each layer of the models, an objective function executed at each node, a number of layers, various weights associated with outputs of each node, and the like.

At step 2022, the processing device may receive, from one or more of an electromechanical machine, a sensor, and a computing device, data associated with a user. In some embodiments, the computing device may include a wearable device, a smartphone, a tablet, an electrode, a computer, a device comprising a user-attached cable, an Internet of Things device or sensor, or some combination thereof. In some embodiments, the data may include information associated with the one or more internal or invasive life support devices associated with the user.

At step 2024, the processing device, based on at least the data, may identify one or more indicators.

At step 2026, the processing device may generate, using the one or more indicators and an artificial intelligence engine, a data signature associated with the user. In some embodiments, the artificial intelligence engine may train one or more computer-implemented models (e.g., machine learning models) to generate the unique data signature.

At step 2028, the processing device may select, from a set of treatment plans and by using the artificial intelligence engine, a treatment plan for the user. The artificial intelligence engine may select the treatment plan by correlating the data signature with at least one of one or more data signatures obtained from one or more evidence-based selection protocols. In some embodiments, the treatment plan may include treatment information pertaining to one or more internal or invasive life support devices associated with the user. In some embodiments, the one or more internal or invasive life support devices may include an insulin pump, a ventricular assist pump, or some combination thereof. In some embodiments, the artificial intelligence engine may train one or more computer-implemented models (e.g., machine learning models) to select the treatment plan.

In some embodiments, the processing device may generate a first dataset by receiving user data related to one or more medical histories, diagnoses, measurements, real-time monitoring prompts, reports, outcomes, progressions, one or more types of compliance-related information, documents, one or more types of billing information, or some combination thereof. In some embodiments, the processing device may generate a second dataset by importing (e.g., selectively injecting) a subset of data from the first dataset and, further, by receiving data related to one or more risk-adjusted outputs associated with quality improvement, user populations, experiment environments, experiment outcomes, libraries of prompts for large language models, or some combination thereof. Based on the first dataset and the second dataset, the processing device may select, from the set of treatment plans and by using the artificial intelligence engine, the treatment plan for the user.

At step 2030, the processing device may perform, using the treatment plan and the artificial intelligence engine, one or more interventions including controlling at least one of the one or more internal or invasive life support devices associated with the user. In some embodiments, the artificial intelligence engine may train one or more computer-implemented models (e.g., machine learning models) to perform the one or more interventions. In some embodiments, performing the one or more interventions may include controlling, using the treatment plan and the artificial intelligence engine, the electromechanical machine.

In some embodiments, a set of tiles may be presented on a graphical user interface of a computing device associated with a second user. At least one of the set of tiles may include a multimedia feed associated with the user's performance of the treatment plan while the user is using an electromechanical machine.

In some embodiments, the processing device may receive, from at least one or more life support devices, second data pertaining to the user. The one or more life support devices may include an extracorporeal membrane oxygenation (ECMO), a ventilator, or some combination thereof. Based on at least the data and the second data, the processing device may identify the one or more indicators.

Consistent with the above disclosure, the examples of systems and methods enumerated in the following clauses are specifically contemplated and are intended as a non-limiting set of examples.
Clause 1.29 A computer-implemented method comprising:
   receiving, from one or more of an electromechanical machine, a sensor, and a computing device, data associated with a user;
   based on at least the data, identifying one or more indicators;
generating, using the one or more indicators and an artificial intelligence engine, a data signature associated with the user;
selecting, from a plurality of treatment plans and by using the artificial intelligence engine, a treatment plan for the user, wherein:

the artificial intelligence engine selects the treatment plan by correlating the data signature with at least one of one or more data signatures obtained from one or more evidence-based selection protocols, and
the treatment plan comprises treatment information pertaining to one or more internal or invasive life support devices associated with the user; and
   performing, using the treatment plan and the artificial intelligence engine, one or more interventions comprising controlling at least one of the one or more internal or invasive life support devices associated with the user.
Clause 2.29 The computer-implemented method of clause 1.29, wherein performing the one or more interventions comprises controlling, using the treatment plan and the artificial intelligence engine, the electromechanical machine.
Clause 3.29 The computer-implemented method of clause 1.29, wherein the one or more internal or invasive life support devices comprise an insulin pump, a ventricular assist pump, or some combination thereof.
Clause 4.29 The computer-implemented method of clause 1.29, wherein a plurality of tiles is presented on a graphical user interface of a computing device associated with a second user, wherein at least one of the plurality of tiles comprises a multimedia feed associated with the user's performance of the treatment plan while the user is using an electromechanical machine.
Clause 5.29 The computer-implemented method of clause 1.29, further comprising:
   receiving, from at least one or more life support devices, second data pertaining to the user, wherein the one or more life support devices comprise an extracorporeal membrane oxygenation (ECMO), a ventilator, or some combination thereof; and
   based on at least the data and the second data, identifying the one or more indicators.
Clause 6.29 The computer-implemented method of clause 1.29, wherein the computing device comprises a wearable device, a smartphone, a tablet, an electrode, a device comprising a user-attachable cable, an Internet of Things device or sensor, or some combination thereof.
Clause 7.29 The computer-implemented method of clause 1.29, further comprising:
   generating a first dataset by receiving user data related to one or more medical histories, diagnoses, measurements, real-time monitoring prompts, reports, outcomes, progressions, one or more types of compliance-related information, documents, one or more types of billing information, or some combination thereof;
   generating a second dataset by importing a subset of data from the first dataset and, further, by receiving data related to one or more risk-adjusted outputs associated with quality improvement, user populations, experiment environments, experiment outcomes, libraries of prompts for large language models, or some combination thereof; and
based on the first dataset and the second dataset, selecting, from the plurality of treatment plans and by using the artificial intelligence engine, the treatment plan for the user.
Clause 8.29 The computer-implemented method of clause 1.29, wherein the data further comprises information associated with the one or more internal or invasive life support devices associated with the user.
Clause 9.29 A tangible, non-transitory computer-readable medium storing instructions that, when executed, cause one or more processing devices to:
   receive, from one or more of an electromechanical machine, a sensor, and a computing device, data associated with a user;
   based on at least the data, identify one or more indicators;
generate, using the one or more indicators and an artificial intelligence engine, a data signature associated with the user;
select, from a plurality of treatment plans and by using the artificial intelligence engine, a treatment plan for the user, wherein:

the artificial intelligence engine selects the treatment plan by correlating the data signature with at least one of one or more data signatures obtained from one or more evidence-based selection protocols, and
the treatment plan comprises treatment information pertaining to one or more internal or invasive life support devices associated with the user; and
   perform, using the treatment plan and the artificial intelligence engine, one or more interventions comprising controlling at least one of the one or more internal or invasive life support devices associated with the user.
Clause 10.29 The computer-readable medium of clause 9.29, wherein performing the one or more interventions comprises controlling, using the treatment plan and the artificial intelligence engine, the electromechanical machine.
Clause 11.29 The computer-readable medium of clause 9.29, wherein the one or more internal or invasive life support devices comprise an insulin pump, a ventricular assist pump, or some combination thereof.
Clause 12.29 The computer-readable medium of clause 9.29, wherein a plurality of tiles is presented on a graphical user interface of a computing device associated with a second user, wherein at least one of the plurality of tiles comprises a multimedia feed associated with the user's performance of the treatment plan while the user is using an electromechanical machine.
Clause 13.29 The computer-readable medium of clause 9.29, wherein the one or more processing devices:
receive, from at least one or more life support devices, second data pertaining to the user, wherein the one or more life support devices comprise an extracorporeal membrane oxygenation (ECMO), a ventilator, or some combination thereof; and based on at least the data and the second data, identify the one or more indicators.
Clause 14.29 The computer-readable medium of clause 9.29, wherein the computing device comprises a wearable device, a smartphone, a tablet, an electrode, a device comprising a user-attachable cable, an Internet of Things device or sensor, or some combination thereof.
Clause 15.29 The computer-readable medium of clause 9.29, wherein the one or more processing devices:
   generate a first dataset by receiving user data related to one or more medical histories, diagnoses, measurements, real-time monitoring prompts, reports, outcomes, progressions, one or more types of compliance-related information, documents, one or more types of billing information, or some combination thereof;
      generate a second dataset by importing a subset of data from the first dataset and, further, by receiving data related to one or more risk-adjusted outputs associated with quality improvement, user populations, experiment environments, experiment outcomes, libraries of prompts for large language models, or some combination thereof; and
   based on the first dataset and the second dataset, select, from the plurality of treatment plans and by using the artificial intelligence engine, the treatment plan for the user.
Clause 16.29 The computer-readable medium of clause 9.29, wherein the data further comprises information associated with the one or more internal or invasive life support devices associated with the user.
Clause 17.29 A system comprising:
   one or more memory devices storing instructions; and
   one or more processing devices communicatively coupled to the one or more memory devices, wherein the one or more processing devices execute the instructions to:
receive, from one or more of an electromechanical machine, a sensor, and a computing device, data associated with a user;
   based on at least the data, identify one or more indicators;
generate, using the one or more indicators and an artificial intelligence engine, a data signature associated with the user;
select, from a plurality of treatment plans and by using the artificial intelligence engine, a treatment plan for the user, wherein:
the artificial intelligence engine selects the treatment plan by correlating the data signature with at least one of one or more data signatures obtained from one or more evidence-based selection protocols, and
the treatment plan comprises treatment information pertaining to one or more internal or invasive life support devices associated with the user; and
   perform, using the treatment plan and the artificial intelligence engine, one or more interventions comprising controlling at least one of the one or more internal or invasive life support devices associated with the user.
Clause 18.29 The system of clause 17.29, wherein performing the one or more interventions comprises controlling, using the treatment plan and the artificial intelligence engine, the electromechanical machine.
Clause 19.29 The system of clause 17.29, wherein the one or more internal or invasive life support devices comprise an insulin pump, a ventricular assist pump, or some combination thereof.
Clause 20.29 The system of clause 17.29, wherein a plurality of tiles is presented on a graphical user interface of a computing device associated with a second user, wherein at least one of the plurality of tiles comprises a multimedia feed associated with the user's performance of the treatment plan while the user is using an electromechanical machine.

No part of the description in this application should be read as implying that any particular element, step, or function is an essential element that must be included in the claim scope. The scope of patented subject matter is defined only by the claims. Moreover, none of the claims is intended to invoke 25 U.S.C. § 104 (f) unless the exact words “means for” are followed by a participle.

The foregoing description, for purposes of explanation, use specific nomenclature to provide a thorough understanding of the described embodiments. However, it should be apparent to one skilled in the art that the specific details are not required to practice the described embodiments. Thus, the foregoing descriptions of specific embodiments are presented for purposes of illustration and description. They are not intended to be exhaustive or to limit the described embodiments to the precise forms disclosed. It should be apparent to one of ordinary skill in the art that many modifications and variations are possible in view of the above teachings.

The above discussion is meant to be illustrative of the principles and various embodiments of the present techniques. Once the above disclosure is fully appreciated, numerous variations and modifications will become apparent to those skilled in the art. It is intended that the following claims be interpreted to embrace all such variations and modifications.

1. A computer-implemented method comprising:
receiving, from one or more of an electromechanical machine, a sensor, and a computing device, data associated with a user who uses the electromechanical machine to perform a treatment plan;
based on the data, generating, using an artificial intelligence engine executing one or more large language models, one or more action interventions to perform, wherein at least one of the one or more action interventions comprise modifying one or more operating parameters of the electromechanical machine, and the one or more action interventions are generated based on one or more measures of efficacy; and
performing the one or more action interventions.

2. The computer-implemented method of claim 1, further comprising ranking, by the artificial intelligence engine, the one or more action interventions.

3. The computer-implemented method of claim 2, wherein the artificial intelligence engine uses one or more secondary conditions identified and explained by the large language model.

4. The computer-implemented method of claim 3, wherein the data is occluded, transformed, or otherwise de-identified due to the use of privacy enhancing techniques.

5. The computer-implemented method of claim 1, wherein the artificial intelligence engine executes at least one or more computer-implemented models comprising a machine learning model.

6. The computer-implemented method of claim 1, wherein the data is processed to remove any personally identifiable information.

7. The computer-implemented method of claim 1, wherein the one or more action interventions comprises causing operation of the electromechanical machine to be controlled.

8. A tangible, non-transitory computer-readable medium storing instructions that, when executed, cause one or more processing devices to:
receive, from one or more of an electromechanical machine, a sensor, and a computing device, data associated with a user who uses the electromechanical machine to perform a treatment plan;
based on the data, generate, using an artificial intelligence engine executing one or more large language models, one or more action interventions to perform, wherein at least one of the one or more action interventions comprise modifying one or more operating parameters of the electromechanical machine, and the one or more action interventions are generated based on one or more measures of efficacy; and
perform the one or more action interventions.

9. The computer-readable medium of claim 8, wherein the instructions, when executed, cause the one or more processing devices to rank, by the artificial intelligence engine, the one or more action interventions.

10. The computer-readable medium of claim 9, wherein the artificial intelligence engine uses one or more secondary conditions identified and explained by the large language model.

11. The computer-readable medium of claim 10, wherein the data is occluded, transformed, or otherwise de-identified due to the use of privacy enhancing techniques.

12. The computer-readable medium of claim 8, wherein the artificial intelligence engine executes at least one or more computer-implemented models comprising a machine learning model.

13. The computer-readable medium of claim 8, wherein the data is processed to remove any personally identifiable information.

14. The computer-readable medium of claim 8, wherein the one or more action interventions comprises causing operation of the electromechanical machine to be controlled.

15. A system comprising:
one or more memory devices storing instructions; and
one or more processing devices communicatively coupled to the one or more memory devices, wherein the one or more processing devices executes the instructions to:
receive, from one or more of an electromechanical machine, a sensor, and a computing device, data associated with a user who uses the electromechanical machine to perform a treatment plan;
based on the data, generate, using an artificial intelligence engine executing one or more large language models, one or more action interventions to perform, wherein at least one of the one or more action interventions comprise modifying one or more operating parameters of the electromechanical machine, and the one or more action interventions are generated based on one or more measures of efficacy; and
perform the one or more action interventions.

16. The system of claim 15, wherein the one or more processing devices executes the instructions to rank, by the artificial intelligence engine, the one or more action interventions.

17. The system of claim 16, wherein the artificial intelligence engine uses one or more secondary conditions identified and explained by the large language model.

18. The system of claim 17, wherein the data is occluded, transformed, or otherwise de-identified due to the use of privacy enhancing techniques.

19. The system of claim 15, wherein the artificial intelligence engine executes at least one or more computer-implemented models comprising a machine learning model.

20. The system of claim 15, wherein the data is processed to remove any personally identifiable information.
